(19) United States
(12) Patent Application Publication
(10) Pub. No.: US 2015/0246938 A1
(43) Pub. Date: Sep. 3, 2015

(54) NOVEL OLEFIN DERIVATIVE

(71) Applicants: Akira Matsumura, Toyonaka-shi (JP); Naotake Kobayashi, Toyonaka-shi (JP); Yuji Nishiura, Toyonaka-shi (JP); Sachie Tagashira, Toyonaka-shi (JP); Shiro Kida, Toyonaka-shi (JP); Kana Kurahashi, Toyonaka-shi (JP); Mitsuhiro Yonehara, Toyonaka-shi (JP)

(72) Inventors: Akira Matsumura, Toyonaka-shi (JP); Naotake Kobayashi, Toyonaka-shi (JP); Yuji Nishiura, Toyonaka-shi (JP); Sachie Tagashira, Toyonaka-shi (JP); Shiro Kida, Toyonaka-shi (JP); Kana Kurahashi, Toyonaka-shi (JP); Mitsuhiro Yonehara, Toyonaka-shi (JP)

(73) Assignee: SHIONOGI & CO., LTD., Osaka-shi, Osaka (JP)

(21) Appl. No.: 14/343,992

(22) PCT Filed: Sep. 7, 2012
(86) PCT No.: PCT/JP2012/072859

§ 371 Date: Mar. 10, 2014

(30) Foreign Application Priority Data
Sep. 9, 2011 (JP) 2011-196847
Jul. 11, 2012 (JP) 2012-155263

(51) Int. Cl. C07F 7/18; C07D 213/643; C07D 401/12; C07D 417/04; C07D 213/40; C07D 401/06; C07D 239/34; C07D 237/14; C07D 277/34; C07D 213/69

(52) U.S. Cl. CPC C07F 7/1852; C07D 277/34; C07D 213/643; C07D 401/12; C07D 213/69; C07D 213/40; C07D 401/06; C07D 239/34; C07D 237/14; C07D 417/04

(57) ABSTRACT

The object of the present invention is to provide novel compounds having ACC2 inhibiting activity. In addition, the object of the present invention is to provide a pharmaceutical composition comprising the compound.

A compound of formula (I′):

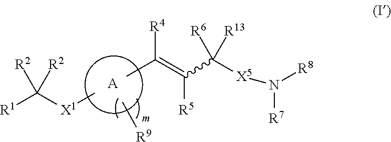

wherein R¹ is substituted or unsubstituted aryl etc.,
   R² is each independently hydrogen, substituted or unsubstituted alkyl etc.,
   R³ is each independently hydrogen, substituted or unsubstituted alkyl etc.,
   n is an integer from 0 to 3,
   R¹² is hydrogen, substituted or unsubstituted alkyl etc.,
   Ring A is aromatic carbocycle or aromatic heterocycle,
   R⁹ is substituted or unsubstituted alkyl, substituted or unsubstituted alkenyl etc.,
   m is an integer from 0 to 4,
   R⁴ and R⁵ is each independently hydrogen, substituted or unsubstituted alkyl etc.,
   R⁶ is substituted or unsubstituted alkyl, substituted or unsubstituted alkenyl etc.,
   R¹³ is hydrogen, substituted or unsubstituted alkyl etc.,
   X⁵ is bond etc.,
   R⁷ is hydrogen or substituted or unsubstituted alkyl,
   R⁸ is substituted or unsubstituted alkylcarbonyl, substituted or unsubstituted alkenylcarbonyl etc.

### FIELD OF THE INVENTION

The present invention relates to a compound having an acetyl CoA carboxylase 2 (hereinafter referred to as ACC2) antagonistic activity.

### BACKGROUND

Acetyl-CoA carboxylase (hereinafter referred to as ACC) is an enzyme that converts malonyl-CoA by carboxylation of acetyl-CoA. It is involved in the metabolism of fatty acids. The ACC has two isoforms of acetyl-CoA carboxylase 1 (hereinafter referred to as ACC1) and ACC2.

ACC2 is mainly expressed in heart and skeletal muscle, and malonyl-CoA produced by ACC2 inhibits the oxidation of fatty acids by inhibiting carnitine palmitoyl transferase I (CPT-I).

ACC2 deficient mice reduce the amount of malonyl-CoA in heart and skeletal muscle. As a result, fatty acids in the mice continuously are oxidized, and the mice lose their weight regardless of the increase in food intake. In addition, it is reported that ACC2 deficient mice develop tolerance to diabetes and obesity induced by the administration of high fatty/high carbohydrate food.

In view of the above information, ACC relates to disorders such as diabetes, obesity and the like, It is suggested that the inhibitor is expected as an anti-diabetes and anti-obesity drug.

On the other hand, since ACC1 deficient mice are fetal in fetal life, the drug inhibiting ACC2 selectively without inhibiting ACC1 is anticipated.

ACC2 inhibitors are disclosed in Patent Documents 1 to 7. For example, two compounds having olefinic structure are disclosed in Patent Document 1.

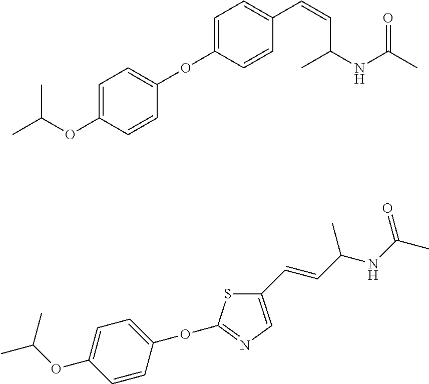

A compound having olefinic structure is disclosed in Patent Document 3.

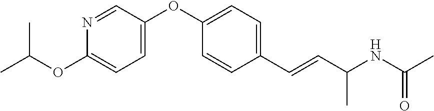 [SMILES: CC(=O)NC(C)/C=C/c1ccc(Oc2ccc(OC(C)C)nc2)cc1]

Thiazole phenyl ether derivatives specifically-inhibiting ACC2 are disclosed in non-Patent Documents 1 to 5. Biphenyl or 3-phenyl-pyridine derivatives exhibiting an ACC1 and ACC2 receptor antagonistic activity are disclosed in Non-Patent Document 6. The compound depicted below exhibiting an ACC2 receptor antagonistic activity and having preferable pharmacokinetic parameters is disclosed in Non-Patent Document 7.

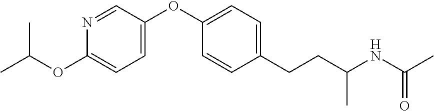 [SMILES: CC(=O)NC(C)CCc1ccc(Oc2ccc(OC(C)C)nc2)cc1]

The compounds having olefinic structure are disclosed in Patent Documents 8 to 19 and Non-Patent Documents 8 to 14.

A compound shown below is disclosed in Patent Document 8.

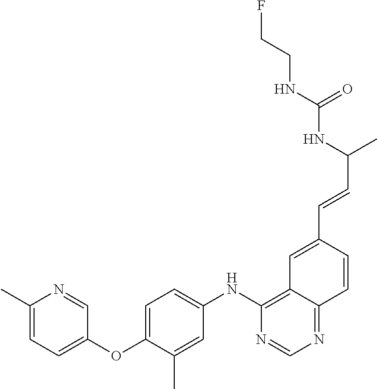 [SMILES: Cc1ccc(Oc2ccc(Nc3ncnc4ccc(/C=C/C(C)NC(=O)NCCF)cc34)cc2C)cn1]

A compound shown below is disclosed in Patent Document 9.

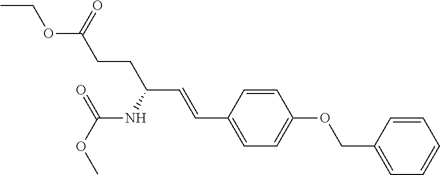 [SMILES: CCOC(=O)CC[C@H](/C=C/c1ccc(OCc2ccccc2)cc1)NC(=O)OC]

A compound shown below is disclosed in Patent Document 10.

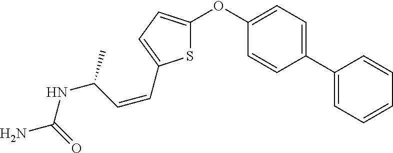 [SMILES: C[C@H](/C=C\c1ccc(Oc2ccc(-c3ccccc3)cc2)s1)NC(N)=O]

Two compounds shown below are disclosed in Patent Document 11.

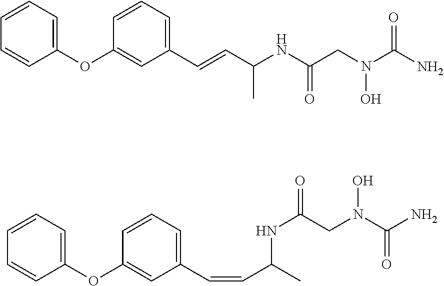 [SMILES: CC(/C=C/c1cccc(Oc2ccccc2)c1)NC(=O)CN(O)C(N)=O ; CC(/C=C\c1cccc(Oc2ccccc2)c1)NC(=O)CN(O)C(N)=O]

A compound shown below is disclosed in Patent Document 12.

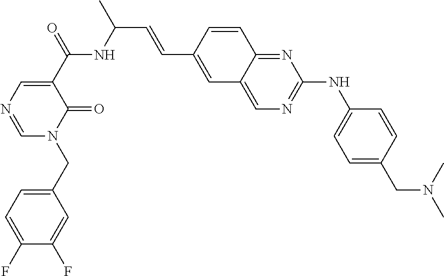 [SMILES: CC(/C=C/c1ccc2nc(Nc3ccc(CN(C)C)cc3)ncc2c1)NC(=O)c1cncn(Cc2ccc(F)c(F)c2)c1=O]

Two compounds shown below are disclosed in Non-Patent Document 8.

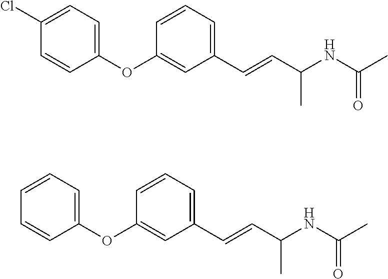 [SMILES: CC(=O)NC(C)/C=C/c1cccc(Oc2ccc(Cl)cc2)c1 ; CC(=O)NC(C)/C=C/c1cccc(Oc2ccccc2)c1]

A compound shown below is disclosed in Non-Patent Document 9.

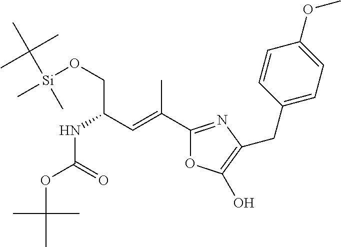 [SMILES: COc1ccc(Cc2nc(/C(C)=C/[C@@H](CO[Si](C)(C)C(C)(C)C)NC(=O)OC(C)(C)C)oc2O)cc1]

A compound shown below is disclosed in Non-Patent Document 10.

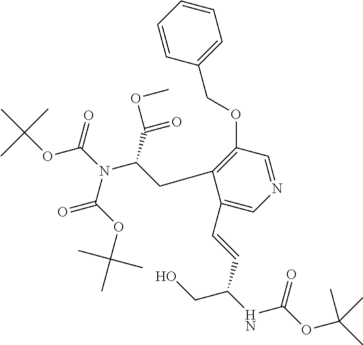 [SMILES: COC(=O)[C@H](Cc1c(/C=C/[C@@H](CO)NC(=O)OC(C)(C)C)cncc1OCc1ccccc1)N(C(=O)OC(C)(C)C)C(=O)OC(C)(C)C]

A compound shown below is disclosed in Non-Patent Document 11.

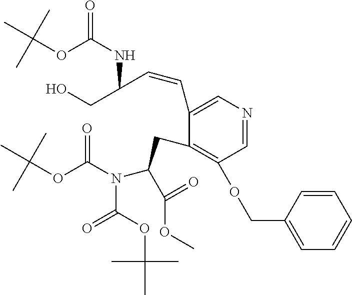 [SMILES: COC(=O)[C@H](Cc1c(/C=C\[C@H](CO)NC(=O)OC(C)(C)C)cncc1OCc1ccccc1)N(C(=O)OC(C)(C)C)C(=O)OC(C)(C)C]

A compound shown below is disclosed in non-Patent Document 12.

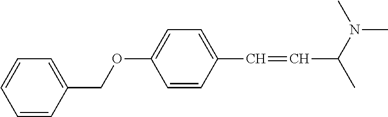 [SMILES: CC(C=Cc1ccc(OCc2ccccc2)cc1)N(C)C]

A compound shown below is disclosed in Patent Document 13.

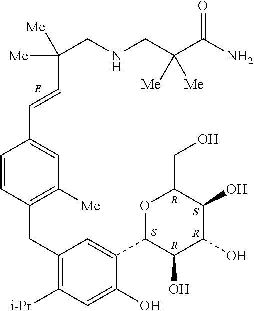

Six compounds shown below are disclosed in Patent Document 14.

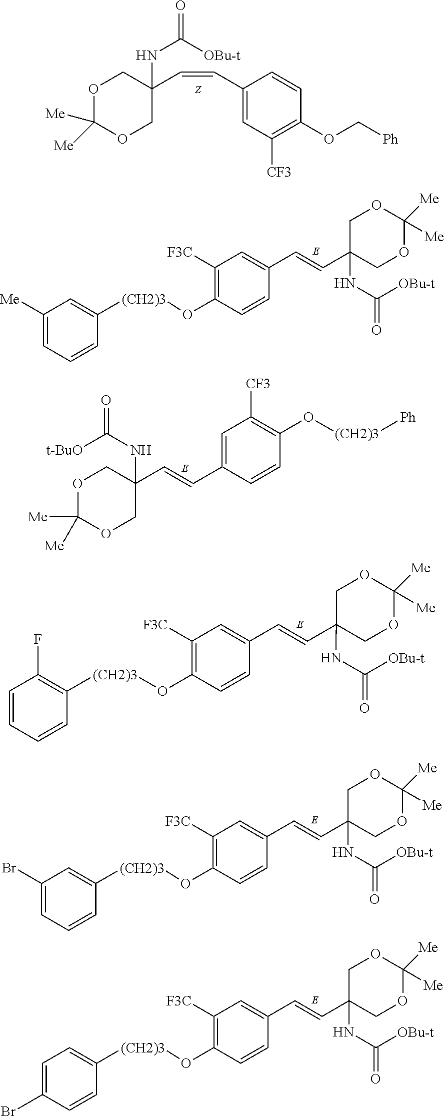

Three compounds shown below are disclosed in Patent Document 15.

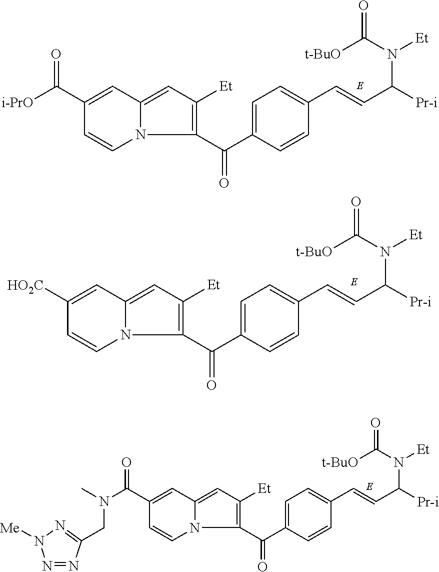

Two compounds shown below are disclosed in Patent Document 16.

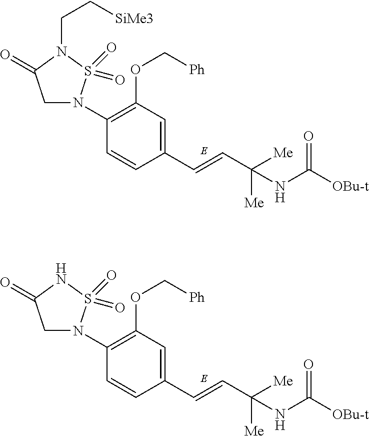

Three compounds shown below are disclosed in Patent Documents 17 and 18.

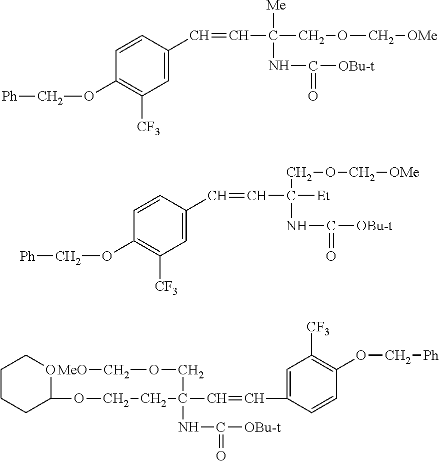

Two compounds shown below are disclosed in Patent Document 19 and non-Patent Document 14.

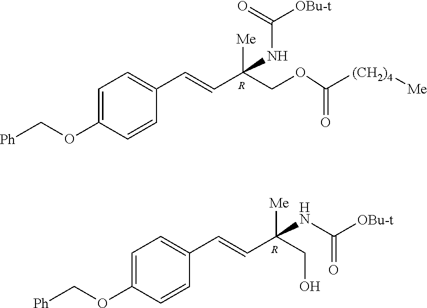

A compound shown below is disclosed in non-Patent Document 13.

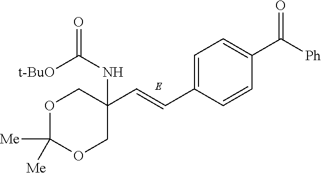 [SMILES: CC(C)(C)OC(=O)NC1(/C=C/c2ccc(C(=O)c3ccccc3)cc2)COC(C)(C)OC1]

However, the present invention is not disclosed nor suggested in the above prior arts.

### PRIOR ART DOCUMENTS

### Patent Documents

[Patent Document 1] WO2008/079610
[Patent Document 2] WO2010/050445
[Patent Document 3] WO2010/003624
[Patent Document 4] WO2007/095601
[Patent Document 5] WO2007/095602
[Patent Document 6] WO2007/095603
[Patent Document 7] US2006/178400
[Patent Document 8] WO2005/044302
[Patent Document 9] WO2002/008189
[Patent Document 10] WO1993/02037
[Patent Document 11] WO1995/30671
[Patent Document 12] WO2010/007114
[Patent Document 13] WO2012/023582
[Patent Document 14] WO2008/153159
[Patent Document 15] WO2012/066234
[Patent Document 16] WO2007/115058
[Patent Document 17] WO2007/069712
[Patent Document 18] WO2008/153159
[Patent Document 19] JP2003/267936

### Non-patent Documents

[Non-patent Document 1] Bioorganic & Medicinal Chemistry Letters, (2006), Vol. 16, 6078-6081
[Non-patent Document 2] Journal of Medicinal Chemistry, (2006), Vol. 49, 3770-3773
[Non-patent Document 3] Bioorganic & Medicinal Chemistry Letters, (2007), Vol. 17, 1803-1807
[Non-patent Document 4] Bioorganic & Medicinal Chemistry Letters, (2007), Vol. 17, 1961-1965
[Non-patent Document 5] Journal of Medicinal Chemistry, (2007), Vol. 50, 1078-1082
[Non-patent Document 6] Bioorganic & Medicinal Chemistry Letters, Vol. 19, 5872-5876
[Non-patent Document 7] Journal of Medicinal Chemistry, (2010), Vol. 53, 8679-8687
[Non-patent Document 8] Journal of Chromatography B: Biomedical Sciences and Applications, (1991), Vol. 562, 249-256
[Non-patent Document 9] Chemistry-An Asian Journal, (2009), Vol. 4, 111-125
[Non-patent Document 10] Tetrahedron, (2000), Vol. 56, 2379-2390
[Non-patent Document 11] Tetrahedron: Asymmetry, (1999), Vol. 10, 3107-3110
[Non-patent Document 12] Yakugaku Zasshi, (1965), Vol. 85, 9-13
[Non-patent Document 13] Synlett, (2007), Vol. 18, 2841-2846
[Non-patent Document 14] Tetrahedron: Asymmetry, (2006), Vol. 17, 2781-2792

### DISCLOSURE OF INVENTION

### Problems to be Solved by the Invention

The object of the present invention is to provide novel compounds having ACC2 inhibiting activity. In addition, the object of the present invention is to provide a pharmaceutical composition comprising the compound.

### Means for Solving the Problem

This invention includes the followings.

(1) A compound of formula (I′):

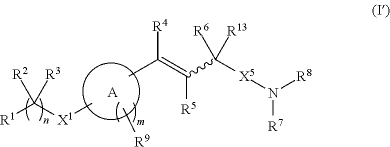

wherein R¹ is substituted or unsubstituted aryl or substituted or unsubstituted heteroaryl,

X¹ is —O—, —S—, —N(—R¹²)—, —C(═O)—, —C(—R²)(—R³)—, —O—C(—R²)(—R³)—, —S—C(—R²)(—R³)— or —N(—R¹²)—C(—R²)(—R³)—,

R² is each independently hydrogen, substituted or unsubstituted alkyl or halogen,

R³ is each independently hydrogen, substituted or unsubstituted alkyl or halogen,

R² and R³ on the same carbon atom may be taken together with the carbon atom to which they are attached to form substituted or unsubstituted ring,

R² and R³ may be taken together with the substituent on the aryl or heteroaryl ring of R¹ and the atom to which each R² and R³ are attached to form substituted or unsubstituted ring,

n is an integer from 0 to 3,

R¹² is hydrogen, substituted or unsubstituted alkyl, substituted or unsubstituted alkenyl or substituted or unsubstituted alkynyl,

R¹² may be taken together with the substituent on the aryl or heteroaryl ring of R¹ and the atom to which each is attached to form substituted or unsubstituted ring,

Ring A is aromatic carbocycle or aromatic heterocycle,

R⁹ is each independently substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted alkylsulfanyl, substituted or unsubstituted alkenylsulfanyl, substituted or unsubstituted alkynylsulfanyl, halogen, hydroxy, cyano, substituted or unsubstituted amino, substituted or unsubstituted carbamoyl, substituted or unsubstituted sulfamoyl, carboxy, substituted or unsubstituted alkylcarbonyl or substituted or unsubstituted alkyloxycarbonyl,

m is an integer from 0 to 4,

R⁴ and R⁵ is each independently hydrogen, substituted or unsubstituted alkyl, substituted or unsubstituted alkenyl, substituted or unsubstituted alkynyl, halogen, substituted or unsubstituted alkyloxy or substituted or unsubstituted alkyloxycarbonyl,

R⁶ is substituted or unsubstituted alkyl, substituted or unsubstituted alkenyl or substituted or unsubstituted alkynyl,

R¹³ is hydrogen, substituted or unsubstituted alkyl, substituted or unsubstituted alkenyl or substituted or unsubstituted alkynyl, or

R⁶ and R¹³ may be taken together with the adjacent carbon atom to form substituted or unsubstituted ring,

X⁵ is bond or —C(—R¹⁶)(—R¹⁷)—,

R¹⁶ and R¹⁷ is each independently hydrogen, substituted or unsubstituted alkyl or halogen,

R⁷ is hydrogen or substituted or unsubstituted alkyl,

R⁸ is substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted cycloalkenylcarbonyl, alkyloxycarbonyl, substituted or unsubstituted alkenyloxycarbonyl, substituted or unsubstituted alkynyloxycarbonyl, substituted or unsubstituted carbamoyl, substituted or unsubstituted sulfamoyl, substituted or unsubstituted amidino, substituted or unsubstituted arylcarbonyl, substituted or unsubstituted heteroarylcarbonyl, substituted or unsubstituted non-aromatic heterocyclyl carbonyl, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted amino, substituted or unsubstituted aryl, or substituted or unsubstituted heteroaryl, substituted or unsubstituted non-aromatic heterocyclyl, substituted or unsubstituted aryloxycarbonyl or substituted or unsubstituted sulfino,

the wavy line means that the group of formura:

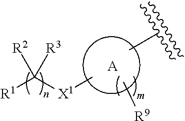

and the group of formura:

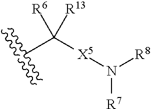

are located at E configuration, Z configuration or the mixture of these configulations in regard to the double bond between the carnon atom bonding R⁴ and the carbon atom bonding R⁵,

provided that the group of formula:

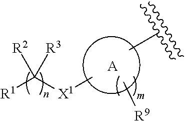

is not a group of formula:

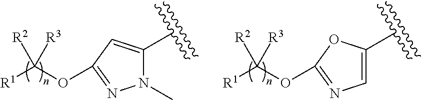

and the following compounds are excluded,

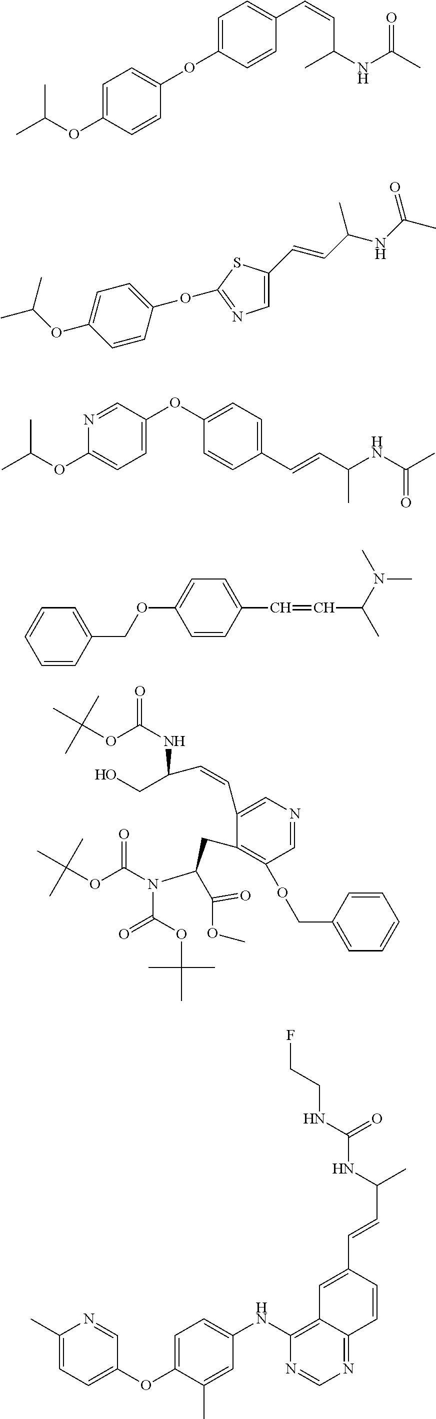

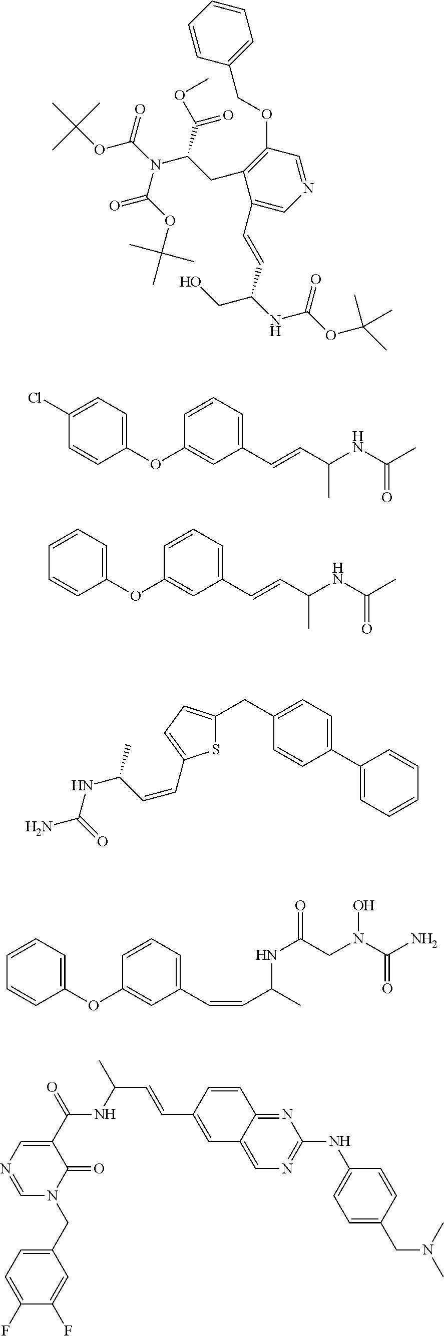 [SMILES: COC(=O)[C@H](Cc1c(/C=C/[C@@H](CO)NC(=O)OC(C)(C)C)cncc1OCc1ccccc1)N(C(=O)OC(C)(C)C)C(=O)OC(C)(C)C ; CC(=O)NC(C)/C=C/c1cccc(Oc2ccc(Cl)cc2)c1 ; CC(=O)NC(C)/C=C/c1cccc(Oc2ccccc2)c1 ; C[C@H](/C=C\c1ccc(Cc2ccc(-c3ccccc3)cc2)s1)NC(N)=O ; CC(/C=C\c1cccc(Oc2ccccc2)c1)NC(=O)CN(O)C(N)=O ; CC(/C=C/c1ccc2nc(Nc3ccc(CN(C)C)cc3)ncc2c1)NC(=O)c1cncn(Cc2ccc(F)c(F)c2)c1=O]

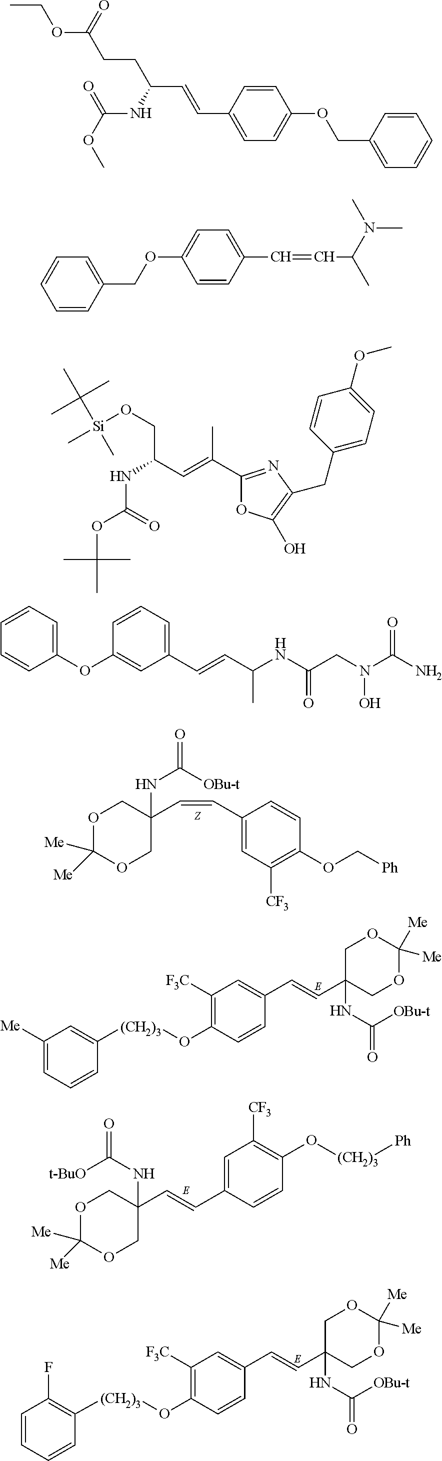

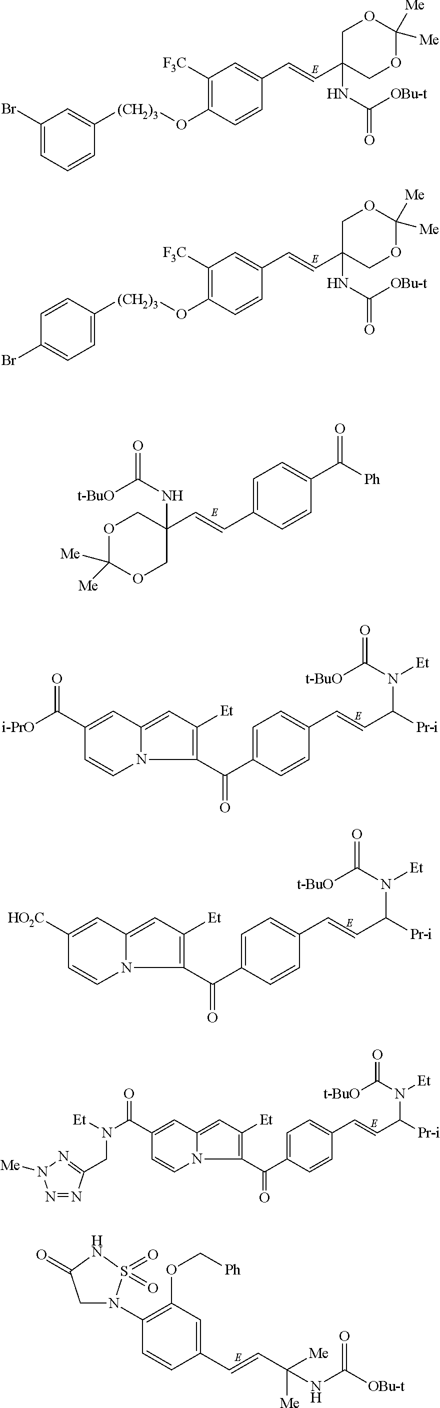

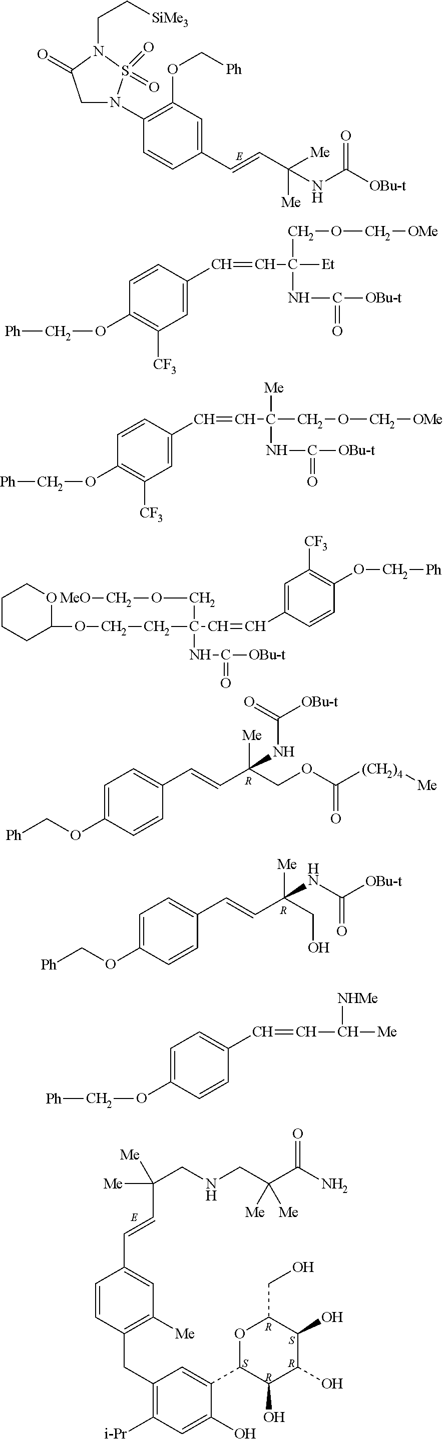

(2) The compound or its pharmaceutically acceptable salt of the above (1), wherein R¹ is substituted or unsubstituted fused aryl or substituted or unsubstituted fused heteroaryl.
(3) The compound or its pharmaceutically acceptable salt of the above (1), wherein R¹ is a group of formula:

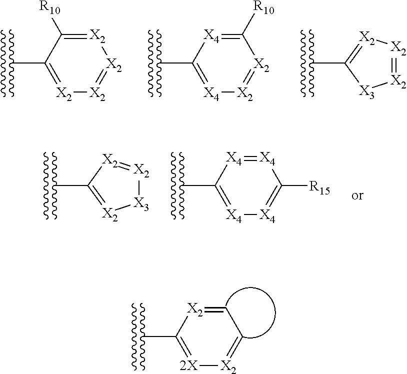

wherein X² is each independently —N═, —C(H)═ or —C(—R¹⁰)═,

X³ is—S—, —O—, —N(H)— or —N(—R¹¹)—,

X⁴ is each independently —N═ or —C(H)═,

R¹⁰ is each independently halogen, substituted or unsubstituted alkyl, substituted or unsubstituted alkenyl, substituted or unsubstituted alkynyl, substituted or unsubstituted amino, hydroxy, substituted or unsubstituted alkyloxy, substituted or unsubstituted alkylcarbonyloxy, mercapto, substituted or unsubstituted alkylsulfonyl, substituted or unsubstituted alkylamino, substituted or unsubstituted alkylcarbonylsulfanyl, cyano, substituted or unsubstituted non-aromatic heterocyclyl, trialkylsilyloxy, substituted or unsubstituted aryloxy, substituted or unsubstituted aryl, substituted or unsubstituted heteroaryl, substituted or unsubstituted cycloalkyl, substituted or unsubstituted cycloalkeyl, substituted or unsubstituted alkylsulfonyl or substituted or unsubstituted alkylsulfonyloxy,

R¹¹ is each independently substituted or unsubstituted alkyl, substituted or unsubstituted alkeyl or substituted or unsubstituted alkynyl,

R¹⁵ is substituted or unsubstituted C2 or more alkyl, substituted or unsubstituted aryl, substituted or unsubstituted aryloxy or substituted or unsubstituted non-aromatic heterocyclyl,

Ring P is substituted or unsubstituted 5-membered aromatic heterocycle, substituted or unsubstituted 5-membered non-aromatic carbocycle, substituted or unsubstituted 5-membered non-aromatic heterocycle, substituted or unsubstituted 6-membered non-aromatic carbocycle or substituted or unsubstituted 6-membered non-aromatic heterocycle.

(4) The compound or its pharmaceutically acceptable salt of the above (3), wherein R¹ is a group of formula:

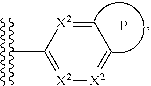

and the above formula:

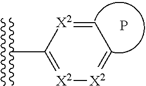

is a group of formula:

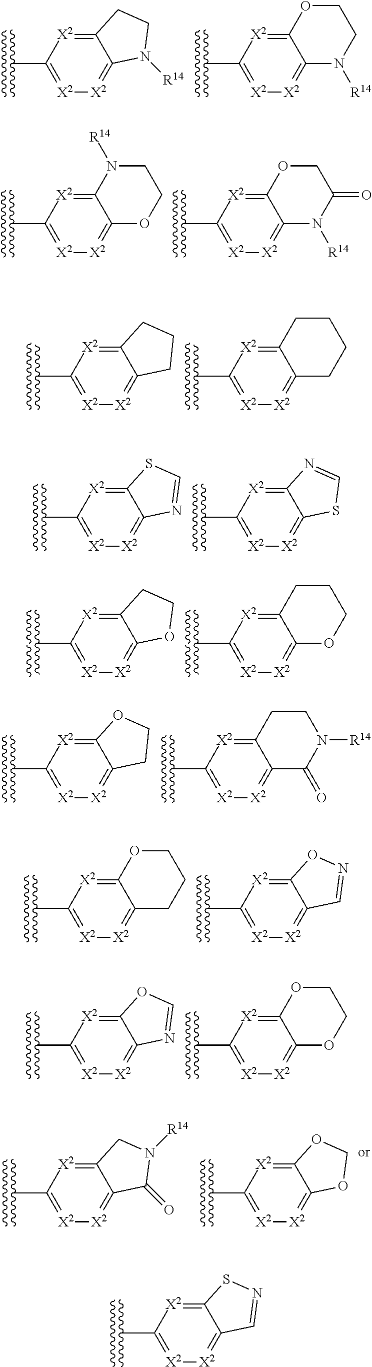

wherein X³ has the same meaning as the above (3),
R¹⁴ is hydrogen, substituted or unsubstituted alkyl, substituted or unsubstituted alkenyl or substituted or unsubstituted alkynyl,
the carbon atom on Ring P may be further substituted.
(5) The compound or its pharmaceutically acceptable salt of the above (4), wherein X² is —C(H)═ or —C(—R¹⁰)═.
(6) The compound or its pharmaceutically acceptable salt of the above (3), wherein R¹ is a group of formula:

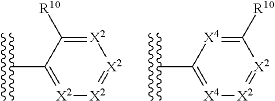

wherein R¹⁰, X² and X⁴ have the same meaning as the above (3).
(7) The compound or its pharmaceutically acceptable salt of the above (6), wherein R¹ is a group of formula:

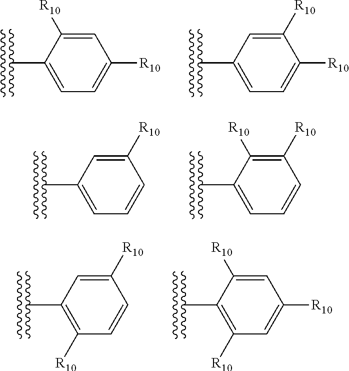

wherein R¹⁰ has the same meaning as the above (6).
(8) The compound or its pharmaceutically acceptable salt of any one of the above (3) to (7), wherein R¹⁰ is each independently halogen, substituted or unsubstituted alkyl, substituted or unsubstituted amino, substituted or unsubstituted alkyloxy, cyano, trialkylsilyloxy or substituted or unsubstituted aryloxy.
(9) The compound or its pharmaceutically acceptable salt of any one of the above (1) to (8), wherein R¹³ is hydrogen.
(10) The compound or its pharmaceutically acceptable salt of any one of the above (1) to (9), wherein R⁶ is substituted or unsubstituted alkyl.
(11) The compound or its pharmaceutically acceptable salt of the above (10), wherein R⁶ is unsubstituted alkyl.
(12) The compound or its pharmaceutically acceptable salt of the above (11), wherein R⁶ is methyl.
(13) The compound or its pharmaceutically acceptable salt of any one of the above (1) to (12), wherein R⁸ is substituted or unsubstituted alkylcarbonyl, substituted or unsubstituted alkyloxycarbonyl, substituted or unsubstituted carbamoyl, substituted or unsubstituted arylcarbonyl, substituted or unsubstituted heteroarylcarbonyl, substituted or unsubstituted non-aromatic heterocyclylcarbonyl, substituted or unsubstituted heteroaryl or substituted or unsubstituted aryloxycarbonyl.
(14) The compound or its pharmaceutically acceptable salt of the above (13), wherein R⁸ is acetyl.
(15) The compound or its pharmaceutically acceptable salt of any one of the above (1) to (14), wherein X¹ is —O—.
(16) The compound or its pharmaceutically acceptable salt of any one of the above (1) to (15), wherein n is an integer from 1 to 3.
(17) The compound or its pharmaceutically acceptable salt of any one of the above (1) to (15), wherein n is 0.
(18) The compound or its pharmaceutically acceptable salt of any one of the above (1) to (17), wherein ring A is aromatic heterocycle.
(19) The compound or its pharmaceutically acceptable salt of the above (18), wherein ring A is 6-membered aromatic heterocycle.
(20) The compound or its pharmaceutically acceptable salt of any one of the above (1) to (17), wherein ring A is pyrazole, thiazole, pyridine, pyrimidine, pyridazine, pyrazine or benzene.
(21) The compound or its pharmaceutically acceptable salt of any one of the above (1) to (20), wherein R⁴ and R⁵ is hydrogen.
(22) The compound or its pharmaceutically acceptable salt of any one of the above (1) to (21), wherein R⁷ is hydrogen.
(23) The compound or its pharmaceutically acceptable salt of any one of the above (1) to (22), wherein m is 0.
(24) The compound or its pharmaceutically acceptable salt of any one of the above (1) to (23), wherein X⁵ is bond.
(25) The compound or its pharmaceutically acceptable salt of any one of the above (1) to (24), wherein the configuration of the group of formula:

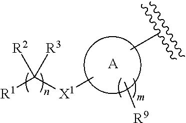

and the group of formula:

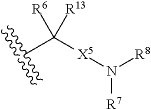

in the compound of formula (I′) is E configuration.
(26) The compound or its pharmaceutically acceptable salt of any one of the above (1) to (25), wherein the compound of formula (I′) is a group of formula (II′):

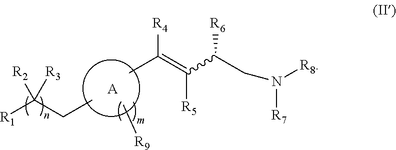

(27) The compound or its pharmaceutically acceptable salt of the above (1), wherein the compound of formula (I′) is a compound of formula (III):

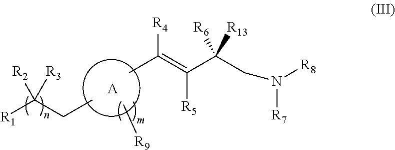

R¹ is a group of formula:

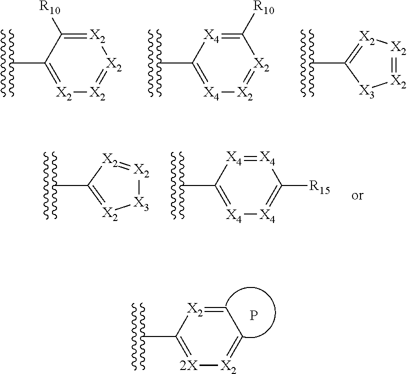

wherein X², X³, X⁴ and R¹⁰ have the same meaning as the above (3),
wherein X¹ is —O—,
n is 0,
R⁴ and R⁵ are hydrogen,
R¹³ is hydrogen,
X⁵ is bond,
R⁷ is hydrogen.
(28) The compound or its pharmaceutically acceptable salt of the above (27), wherein R⁶ is alkyl.
(29) The compound or its pharmaceutically acceptable salt of the above (27) or (28), wherein R⁸ is substituted or unsubstituted alkylcarbonyl.
(30) A pharmaceutical composition comprising the compound or its pharmaceutically acceptable salt of any one of the above (1) to (29).
(31) The pharmaceutical composition of the above (30) for treatment or prevention of a disease associated with ACC2.
(32) A method for treatment or prevention of a disease associated with ACC2 characterized by administering the compound or its pharmaceutically acceptable salt of any one of the above (1) to (29).
(33) Use of the compound or its pharmaceutically acceptable salt of any one of the above (1) to (29) for treatment or prevention of a disease associated with ACC2.
(34) The compound or its pharmaceutically acceptable salt of any one of the above (1) to (29) for treatment or prevention of a disease associated with ACC2.

Substituents on the nitrogen atom in “substituted or unsubstituted amino”, “substituted or unsubstituted carbamoyl”, “substituted or unsubstituted sulfamoyl”, and “substituted or unsubstituted amidino” include the following substituents. Hydrogen on the nitrogen atom can be replaced with one or two substituents selected from the following substitutents.

### Substitutents:

alkyl, alkenyl, alkynyl, haloalkyl, haloalkenyl, halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azide, hydrazino, ureido, amidino, guanidino, trialkylsilyl, alkyloxy, alkyloxyalkyloxy, alkenyloxy, alkynyloxy, haloalkyloxy, trialkylsilyloxy, cyanoalkyl, cyanoalkyloxy, alkylcarbonyl, alkenylcarbonyl, alkynylcarbonyl, monoalkylamino, dialkylamino, alkylsulfonyl, alkenylsulfonyl, alkynylsulfonyl, monoalkylcarbonylamino, dialkylcarbonylamino, monoalkylsulfonylamino, dialkylsulfonylamino, monoalkyloxycarbonylamino, dialkyloxycarbonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkylcarbonylsulfanyl, alkenylsulfynyl, alkynylsulfinyl, monoalkylcarbamoyl, mono(hydroxyalkyl)carbamoyl, dialkylcarbamoyl, hydroxycarbamoyl, cyanocarbamoyl, carboxyalkylcarbamoyl, mono(dialkylaminoalkyl)carbamoyl, cycloalkylcarbamoyl, non-aromatic heterocyclylalkylcarbamoyl, non-aromatic heterocyclylcarbamoyl, alkyloxycarbamoyl, alkyloxycarbonylalkylcarbamoyl, monoalkylsulfamoyl, dialkylsulfamoyl, aryl, cycloalkyl, cycloalkenyl, heteroaryl, heteroaryl substituted with alkyloxycarbonyl, non-aromatic heterocyclyl, non-aromatic heterocyclyl substituted with alkyl, non-aromatic heterocyclyl substituted with alkyloxycarbonyl, aryloxy, cycloalkyloxy, cycloalkenyloxy, heteroaryloxy, non-aromatic heterocyclyloxy, arylcarbonyl, cycloalkylcarbonyl, cycloalkenylcarbonyl, heteroarylcarbonyl, heteroarylcarbonyl substituted with alkylcarbonyl, non-aromatic heterocyclylcarbonyl, non-aromatic heterocyclylcarbonyl substituted with alkyloxycarbonyl, aryloxycarbonyl, cycloalkyloxycarbonyl, cycloalkenyloxycarbonyl, heteroaryloxycarbonyl, non-aromatic heterocyclyloxycarbonyl, arylalkyl, cycloalkylalkyl, cycloalkenylalkyl, heteroarylalkyl, non-aromatic heterocyclylalkyl, arylalkyloxy, cycloalkylalkyloxy, cycloalkenylalkyloxy, heteroarylalkyloxy, non-aromatic heterocyclylalkyloxy, arylalkyloxycarbonyl, cycloalkylalkyloxycarbonyl, cycloalkenylalkyloxycarbonyl, heteroarylalkyloxycarbonyl, non-aromatic heterocyclylalkyloxycarbonyl, arylalkylamino, cycloalkylalkylamino, cycloalkenylalkylamino, heteroarylalkylamino, non-aromatic heterocyclylalkylamino, arylsulfanyl, cycloalkylsulfanyl, cycloalkenylsulfanyl, heteroarylsulfanyl, non-aromatic heterocyclylsulfanyl, arylsulfonyl, cycloalkylsulfonyl, cycloalkenylsulfonyl, heteroarylsulfonyl, non-aromatic heterocyclylsulfonyl, alkyloxycarbonylalkyl, carboxyalkyl, hydroxyalkyl, dialkylaminoalkyl, hydroxyalkyl, alkyloxyalkyl, arylalkyloxyalkyl, cycloalkylalkyloxyalkyl, cycloalkenylalkyloxyalkyl, heteroarylalkyloxyalkyl and non-aromatic heterocyclylalkyloxyalkyl.

Substituents of “substituted or unsubstituted alkyl”, “substituted or unsubstituted alkenyl”, “substituted or unsubstituted alkynyl”, “substituted or unsubstituted alkyloxy”, “substituted or unsubstituted alkenyloxy”, “substituted or unsubstituted alkynyloxy”, “substituted or unsubstituted alkylsulfanyl”, “substituted or unsubstituted alkenylsulfanyl”, “substituted or unsubstituted alkynylsulfanyl”, “substituted or unsubstituted alkylcarbonyl”, “substituted or unsubstituted alkenylcarbonyl”, “substituted or unsubstituted alkynylcarbonyl”, “substituted or unsubstituted alkyloxycarbonyl”, “substituted or unsubstituted alkenyloxycarbonyl”, “substituted or unsubstituted alkynyloxycarbonyl”, “substituted or unsubstituted alkylcarbonyloxy”, “substituted or unsubstituted alkylcarbonylsulfanyl” include the following substituents. Hydrogen on the nitrogen atom can be replaced with one or more substituents selected from the following substitutents.

### Substitutents:

halogen, hydroxy, carboxy, amino, imino, hydroxy amino, hydroxy imino, formyl, formyloxy, carbamoyl, sulfamoyl, sufanyl, sulfino, sulfo, thioformyl, thiocarboxy, dithiocarboxy, thiocarbamoyl, cyano, nitro, nitroso, azide, hydrazino, ureido, amidino, guanidine, trialkylsilyl, alkyloxy, alkyloxyalkyloxy, alkenyloxy, alkynyloxy, haloalkyloxy, trialkylsilyloxy, cyanoalkyloxy, alkylcarbonyl, alkenylcarbonyl, alkynylcarbonyl, monoalkylamino, dialkylamino, alkylsulfonyl, alkenylsulfonyl, alkynylsulfonyl, monoalkylcarbonylamino, dialkylcarbonylamino, monoalkylsulfonylamino, dialkylsulfonylamino, monoalkyloxycarbonylamino, dialkyloxycarbonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, dialkylaminocarbonyl, alkylsulfanyl, alkenylsulfanyl, alkynylsulfanyl, alkylcarbonylsulfanyl, alkylsulfinyl, alkenylsulfinyl, alkynylsulfinyl, monoalkylcarbamoyl, mono(hydroxyalkyl)carbamoyl, dialkylcarbamoyl, hydroxycarbamoyl, cyanocarbamoyl, carboxyalkycarbamoyl, carboxyalkylcarbamoyl, mono(dialkylaminoalkyl)carbamoyl, cycloalkylcarbamoyl, non-aromatic heterocyclylalkylcarbamoyl, non-aromatic heterocyclylcarbamoyl, alkyloxycarbamoyl, alkyloxycarbonylalkylcarbamoyl, monoalkylsulfamoyl, dialkylsulfamoyl, aryl, cycloalkyl, cycloalkenyl, heteroaryl, heteroaryl substituted with alkyloxycarbonyl, non-aromatic heterocyclyl, non-aromatic heterocyclyl substituted with alkyl, non-aromatic heterocyclyl substituted with alkyloxycarbonyl, aryloxy, cycloalkyloxy, cycloalkenyloxy, heteroaryloxy, non-aromatic heterocyclyloxy, arylcarbonyl, cycloalkylcarbonyl, cycloalkenylcarbonyl, heteroarylcarbonyl, heteroarylcarbonyl substituted with alkylcarbonyl, non-aromatic heterocyclylcarbonyl, non-aromatic heterocyclylcarbonyl substituted with alkyloxycarbonyl, aryloxycarbonyl, cycloalkyloxycarbonyl, cycloalkenyloxycarbonyl, heteroaryloxycarbonyl, non-aromatic heterocyclyloxycarbonyl, arylalkyloxy, cycloalkylalkyloxy, cycloalkenylalkyloxy, heteroarylalkyloxy, non-aromatic heterocyclylalkyloxy, arylalkyloxycarbonyl, cycloalkylalkyloxycarbonyl, cycloalkenylalkyloxycarbonyl, heteroarylalkyloxycarbonyl, non-aromatic heterocyclylalkyloxycarbonyl, arylalkylamino, cycloalkylalkylamino, cycloalkenylalkylamino, heteroarylalkylamino, non-aromatic heterocyclylalkylamino, arylsulfanyl, cycloalkylsulfanyl, cycloalkenylsulfanyl, heteroarylsulfanyl, non-aromatic heterocyclylsulfanyl, cycloalkylsulfonyl, cycloalkenylsulfonyl, arylsulfonyl, heteroarylsulfonyl and non-aromatic heterocyclylsulfonyl.

Substituents in the ring of “Substituted or unsubstituted cycloalkyl”, “substituted or unsubstituted cycloalkenyl”, “substituted or unsubstituted aryl”, “substituted or unsubstituted heteroaryl”, “substituted or unsubstituted non-aromatic heterocyclyl”, “substituted or unsubstituted cycloalkylcarbonyl”, “substituted or unsubstituted cycloalkenylcarbonyl”, “substituted or unsubstituted arylcarbonyl”, “substituted or unsubstituted heteroarylcarbonyl”, “substituted or unsubstituted non-aromatic heterocyclylcarbonyl”,

“substituted or unsubstituted ring that R² or R³ on the same carbon atom may be taken together with the carbon atom to which they are attached to form”, “substituted or unsubstituted ring that R² and R³ may be taken together with the substituent on the aryl or heteroaryl ring on R¹ and the atom and to which each is attached to form”,
“substituted or unsubstituted ring that R¹² may be taken together with the substituent on the aryl or heteroaryl ring on R¹ and the atom and to which each is attached to form”, “substituted or unsubstituted ring that R⁶ and R¹³ may be taken together with the adjacent carbon atom to form”, “substituted or unsubstituted aryloxycarbonyl” or “substituted or unsubstituted aryloxy” include the following substituents. Hydrogen atom on the ring at arbitrary position(s) can be substituted with one or more group(s) selected from the following substituents.

### Substitutent:

substituted or unsubstituted alkyl (for example, haloalkyl, cycloalkylalkyl, cycloalkenylalkyl, heteroarylalkyl, non-aromatic heterocyclylalkyl, arylalkyloxyalkyl, cycloalkylalkyloxyalkyl, cycloalkenylalkyloxyalkyl, heteroarylalkyloxyalkyl, non-aromatic heterocyclylalkyloxyalkyl, alkyloxyalkyl, arylalkyl, hydroxyalkyl, alkyl substituted with alkyloxyimino), substituted or unsubstituted alkenyl (for example, alkyloxycarbonylalkenyl, carboxyalkenyl), substituted or unsubstituted alkynyl, halogen, hydroxy, carboxy, substituted or unsubstituted amino (for example, hydroxyamino, monoalkylamino, dialkylamino, monoalkylcarbonylamino, dialkylcarbonylamino, monoalkylsulfonylamino, dialkylsulfonylamino, arylalkylamino, cycloalkylalkylamino, cycloalkenylalkylamino, heteroarylalkylamino, non-aromatic heterocyclylalkylamino, monoalkyloxycarbonylamino, dialkyloxycarbonylamino, monohydroxyalkylamino, monocarboxyalkylamino, mono(alkyloxycarbonylalkyl)amino, mono(cycloalkylalkylcarbonyl)amino, cycloalkylcarbamoylamino, cycloalkylamino), imino, hydroxyimino, formyl, formyloxy, substituted or unsubstituted carbamoyl (for example, hydroxycarbamoyl, cyanocarbamoyl, alkyloxycarbonylalkylcarbamoyl, carboxyalkylcarbamoyl, mono(hydroxyalkyl)carbamoyl, mono(dialkylaminoalkyl)carbamoyl, cycloalkylcarbamoyl, cycloalkylcarbonyl substituted with alkyloxycarbonyl, non-aromatic heterocyclylalkylcarbamoyl, non-aromatic heterocyclylcarbamoyl, non-aromatic heterocyclylcarbamoyl substituted with alkyloxycarbonyl, monoalkylcarbamoyl, dialkylcarbamoyl, alkyloxycarbamoyl, monoalkylcarbamoylalkyloxy, mono(hydroxyalkyl)carbamoyl, monoalkyloxycarbonylalkylcarbamoyl, cycloalkylalkylcarbamoyl), sulfamoyl, sulfanyl, sulfino, sulfo, thioformyl, thiocarboxy, dithiocarboxy, thiocarbamoyl, cyano, nitro, nitroso, azide, hydrazino, ureido, amidino, guanidine, trialkylsilyl, substituted or unsubstituted alkyloxy (for example, arylalkyloxy, cycloalkylalkyloxy, cycloalkylalkyloxy substituted with hydroxy, cycloalkenylalkyloxy, heteroarylalkyloxy, non-aromatic heterocyclylalkyloxy, non-aromatic heterocyclyloxyalkyloxy, alkyloxyalkyloxy, cyanoalkyloxy, haloalkyloxy, alkyloxycarbonylalkyloxy, carboxyalkyloxy, dialkylaminoalkyloxy, hydroxyalkyloxy), alkenyloxy, alkynyloxy, haloalkyloxy, haloalkylsulfonyloxy, alkylcarbonyl, alkenylcarbonyl, alkynylcarbonyl, haloalkylcarbonyl, alkylsulfonyl, alkenylsulfonyl, alkynylsulfonyl, alkylimino, alkenylimino, alkynylimino, alkylcarbonylimino, alkenylcarbonylimino, alkynylcarbonylimino, alkyloxyimino, alkynyloxyimino, alkynyloxyimino, substituted or unsubstituted alkylcarbonyloxy, alkenylcarbonyloxy, alkynylcarbonyloxy, alkyloxycarbonyl, alkenyloxycarbonyl, alkynyloxycarbonyl, substituted or unsubstituted alkylsulfanyl, alkenylsulfanyl, alkynylsulfanyl, substituted or unsubstituted alkylcarbonylsulfanyl, alkylsulfinyl, alkenylsulfinyl, alkynylsulfinyl, monoalkylsulfamoyl, dialkylsulfamoyl, substituted or unsubstituted aryl (for example, aryl substituted with alkyl, aryl substituted with cyano), substituted or unsubstituted cycloalkyl (for example, cycloalkyl substituted with one or more substituents selected from carboxy, alkyl and halogen), cycloalkenyl, substituted or unsubstituted heteroaryl (for example, heteroaryl substituted with alkyloxycarbonyl, heteroaryl substituted with alkyl, heteroaryl substituted with haloalkyl, heteroaryl substituted with alkyloxyalkyl, and heteroaryl substituted with halogen), substituted or unsubstituted non-aromatic heterocyclyl (for example, non-aromatic heterocyclyl substituted with alkyl, non-aromatic heterocyclyl substituted with alkyloxycarbonyl, non-aromatic heterocyclyl substituted with halogen), substituted or unsubstituted aryloxy (for example, aryloxy substituted with nitro, aryloxy substituted with cyano), substituted or unsubstituted heteroaryloxy, cycloalkyloxy, cycloalkenyloxy, heteroaryloxy, non-aromatic heterocyclyloxy, arylcarbonyl, cycloalkylcarbonyl, cycloalkenylcarbonyl, substituted or unsubstituted heteroarylcarbonyl (for example, heteroarylcarbonyl substituted with alkylcarbonyl), substituted or unsubstituted non-aromatic heterocyclylcarbonyl (for example, non-aromatic heterocyclylcarbonyl substituted with alkyloxycarbonyl), aryloxycarbonyl, cycloalkyloxycarbonyl, cycloalkenyloxycarbonyl, heteroaryloxycarbonyl, non-aromatic heterocyclyloxycarbonyl, arylalkyloxycarbonyl, cycloalkylalkyloxycarbonyl, cycloalkenylalkyloxycarbonyl, heteroarylalkyloxycarbonyl, non-aromatic heterocyclylalkyloxycarbonyl, alkylsulfanyl, arylsulfanyl, cycloalkylsulfanyl, cycloalkenylsulfanyl, heteroarylsulfanyl, non-aromatic heterocyclylsulfanyl, alkylsulfonyl, arylsulfonyl, cycloalkylsulfonyl, cycloalkenylsulfonyl, heteroarylsulfonyl and non-aromatic heterocyclylsulfonyl.

The above “substituted or unsubstituted cycloalkyl”, “substituted or unsubstituted cycloalkenyl” and “substituted or unsubstituted non-aromatic heterocyclyl” can be substituted with oxo. In this case, two hydrogens on carbon atom are replaced with ═O group.

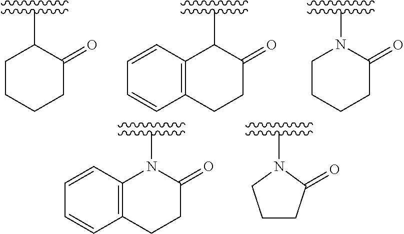

The cycloalkyl, cycloalkenyl and non-aromatic heterocyclyl part in the above “substituted or unsubstituted cycloalkyloxy”, “substituted or unsubstituted cycloalkenyloxy”, “substituted or unsubstituted non-aromatic heterocyclyloxy”, “substituted or unsubstituted cycloalkylcarbonyl”, “substituted or unsubstituted cycloalkenylcarbonyl”, “substituted or unsubstituted non-aromatic heterocyclylcarbonyl”, “substituted or unsubstituted cycloalkylsulfanyl”, “substituted or unsubstituted non-aromatic heterocyclylsulfanyl”, “substituted or unsubstituted cycloalkylsulfonyl” and “substituted or unsubstituted non-aromatic heterocyclylsulfonyl” can be substituted with “oxo”.

### Effect of the Invention

The compound of this invention has ACC2 antagonistic activity. A pharmaceutical composition comprising the compound of this invention is very useful as a medicine for preventing or treating a disease associated with ACC2, e.g. metabolic syndrome, obesity, diabetes, insulin resistance, abnormal glucose tolerance, diabetic peripheral neuropathy, diabetic nephropathy, diabetic retinal disease, diabetic macroangiopathy, hyperlipidemia, hypertension, cardiovascular illness, arterial sclerosis, atherosclerotic cardiovascular disease, cardiac arrest, cardiac infarction, infectious disease, neoplasm and the like (Journal of Cellular Biochemistry, (2006), vol. 99, 1476-1488, EXPERT OPINION ON THERAPEUTIC Targets, (2005), Vol. 9, 267-281, WO2005/108370, JP2009-196966, JP2010-081894, JP2009-502785), especially for preventing or treating diabetes and/or obesity.

### MODE FOR CARRYING OUT THE INVENTION

Terms used in the present description are explained below. Each term has the same meaning alone or together with other terms in this description.

“Halogen” includes fluorine atom, chlorine atom, bromine atom and iodine atom. Especially preferred is fluorine atom or chlorine atom.

“Alkyl” includes C1 to C15, preferably C1 to C10, more preferably C1 to C6, even more preferably C1 to C4 straight or branched alkyl group. Examples include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, n-decyl and the like.

A preferable embodiment of “alkyl” includes methyl, ethyl, n-propyl, isopropyl, n-butyl, isobutyl, sec-butyl, tert-butyl, n-pentyl and the like. A more preferable embodiment of “alkyl” includes methyl, ethyl, n-propyl, isopropyl, tert-butyl and the like.

A preferable embodiment of alkyl at the ring of “substituted or unsubstituted aryl” or “substituted or unsubstituted heteroaryl” for R¹ includes methyl, ethyl, n-propyl, isopropyl tert-butyl.

A preferable embodiment of “alkyl” of R² or R³ includes especially methyl and ethyl of the above alkyl. Furthermore, methyl is preferable.

A preferable embodiment of “alkyl” of R⁶ or R¹³ includes especially methyl and ethyl of the above alkyl. Furthermore, methyl is preferable.

A preferable embodiment of “alkyl” of R⁷ includes especially methyl of the above alkyl.

“Alkenyl” includes straight or branched alkenyl containing one or more double bond at any position having C2 to C15, preferably C2 to C10, more preferably C2 to C6, even more preferably C2 to C4. Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl, pentadecenyl and the like.

A preferable embodiment of “alkenyl” includes vinyl, allyl, propenyl, isopropenyl, butneyl and the like.

“Alkynyl” includes straight or branched alkenyl containing one or more triple bond at any position having C2 to C10, preferably C2 to C8, more preferably C2 to C6, even more preferably C2 to C4

Examples include ethynyl, propynyl, butynyl, pentynyl, hexynyl, heptynyl, octynyl, nonynyl, decynyl and the like. Alkynyl can have double bond(s) at any arbitrary position(s).

A preferable embodiment of “alkynyl” includes ethynyl, propynyl, butynyl and pentynyl.

“Aromatic carbocycle” includes monocyclic or two or more cyclic aromatic carbocycle. Examples are benzene, naphthalene, anthracene, phenanthrene and the like. A preferable embodiment of “aromatic carbocycle” includes benzene.

“Aromatic heterocycle” means monocyclic or polycyclic aromatic heterocycle containing one or more heteroatom(s) arbitrarily selected from O, S and N on the ring. Examples are monocyclic aromatic heterocycle such as pyrrole, imidazole, pyrazole, pyridine, pyridazine, pyrimidine, pyrazine, triazole, triazine, tetrazole, isoxazole, oxazole, oxadiazole, isothiazole, thiazole, thiadiazole, furan, thiophene and the like; bicyclic aromatic heterocycle such as indole, isoindole, indazole, indolizine, quinoline, isoquinoline, cinnoline, phthalazine, quinazoline, naphthyridine, quinoxaline, purine, pteridine, benzimidazole, benzisoxazole, benzoxazole, benzoxadiazole, benzisothiazole, benzothiazole, benzothiadiazole, benzofuran, isobenzofuran, benzothiophene, benzotriazole, imidazopyridine, triazolopyridine, imidazothiazole, pyradinopyridazine, oxazoropyridine, thiazoropyridine and the like; ticyclic aromatic heterocycle such as carbazole, acridine, xanthene, phenothiazine, penoxazine, dibenzofuran and the like. Especially preferable example is 5 or 6-membered aromatic heterocycle. Furthermore, pyridine, pyrimidine, pyridazine, thiazole, pyrazole, pyrazine and the like are preferable.

“Cycloalkyl” means C3 to C8 cyclic saturated carbocyclyl and the cyclic saturated carbocyclyl fused with one or two C3 to C8 cyclic group(s). Examples of C3 to C8 cyclic saturated carbocyclyl include cyclopropyl, cyclobutyl, cyclopentyl, cyclohexyl, cycloheptyl, cyclooctyl and the like. Especially preferable examples include C3 to C6 cycloalkyl, or C5 or C6 cycloalkyl. Furthermore, C3 cycloalkyl is preferable.

The ring fused with C3 to C8 cyclic saturated carbocyclyl includes cycloalkane ring (example: cyclohexane ring, cyclopentane ring and the like), cycloalkene ring (example: cyclohexene ring, cyclopentene ring and the like), non-aromatic heterocycle (example: piperidine ring, piperazine ring, morpholine ring and the like). At the above ring, the bond(s) can be attached to C3 to C8 cyclic saturated carbocyclyl.

For example, the following groups are also exemplified as a cycloalkyl, and included in cycloalkyl. These groups can be substituted at any arbitrary position(s). When cycloalkyl is substituted, the substituent(s) on the cycloalkyl can be substituted on either C3 to C8 cyclic saturated cyclocyclyl or C3 to C8 ring fused C3 to C8 cyclic saturated cyclocyclyl.

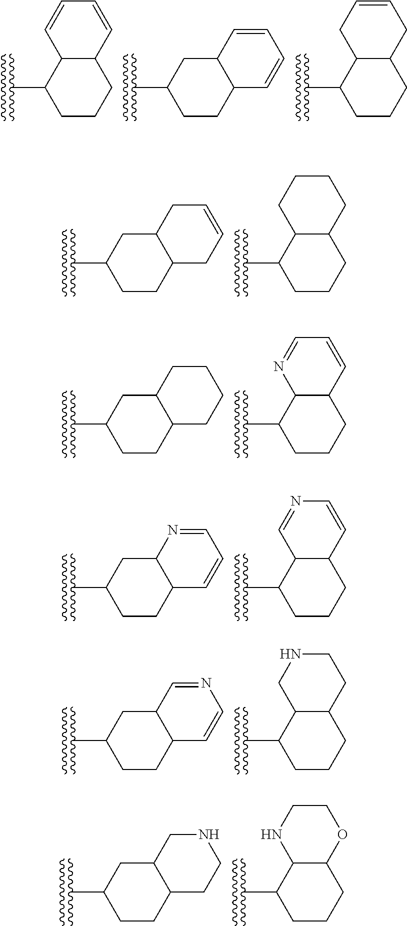

Furthermore, “cycloalkyl” includes a bridged group or a group formed Spiro ring as follows.

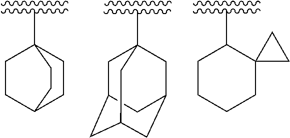

“Cycloalkyl substituted with carboxy” means the above “cycloalkyl” substituted with one or more carboxy.

“Cycloalkenyl” means C3 to C8 cyclic unsaturated aliphatic hydrocarbon group and the cyclic unsaturated aliphatic hydrocarbon group fused with one or two C3 to C8 cyclic group(s). “C3 to C8 cyclic unsaturated aliphatic hydrocarbon group” preferably means that C3 to C8 cyclic unsaturated aliphatic hydrocarbon group has 1 to 3 double bond(s) between carbon atom and carbon atom in the ring. Specifically, Preferred is cyclopropenyl, cyclobutenyl, cyclopentenyl, cyclohexenyl, cycloheptenyl, cyclohexadienyl and the like. Especially preferred is C3 to C6 cycloalkenyl, C5 to C6 cycloalkenyl.

The ring fused with C3 to C8 cyclic unsaturated aliphatic hydrocarbon group includes carbocycle (aromatic hydrocarbon ring (example: benzene ring, naphalene ring etc.), cycloalkane ring (example: cyclohexane ring, cyclopentane ring etc.), cycloalkane ring (example: cyclohexene ring, cyclopentene ring etc.)), heterocycle (aromatic heterocycle (pyridine ring, pyrimidine ring, pyrrole ring, imidazole ring etc.), non-aromatic heterocycle (example: piperidine ring,) piperazine ring, morpholine ring etc.).

At the above ring, the bond(s) can be attached to C3 to C8 cyclic unsaturated aliphatic hydrocarbon group.

For example, the following groups are also exemplified as a cycloalkenyl and included in cycloalkenyl. These groups can be substituted at any arbitrary position(s). When cycloalkenyl is substituted, the substituent(s) on the cycloalkenyl can be substituted on either C3 to C8 cyclic unsaturated hydrocarbon group or C3 to C8 ring fused C3 to C8 cyclic unsaturated hydrocarbon group.

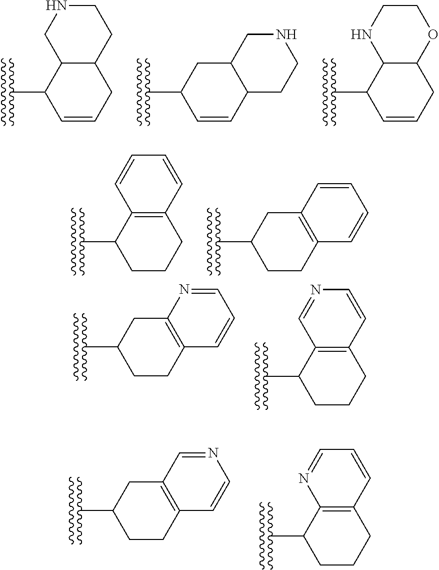

In addition, the “cycloalkenyl” also includes a group to form a spiro ring as follows:

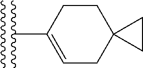

“Aryl” includes monocyclic or polycyclic aromatic carbocyclyl and monocyclic or polycyclic aromatic carbocyclyl fused with one or two 3- to 8-membered cyclic group(s). Examples of monocyclic or polycyclic aromatic carbocyclyl include phenyl, naphthyl, anthryl, phenanthryl and the like. Especially preferable example is phenyl.

The ring fused with monocyclic or polycyclic aromatic carbocyclyl includes non-aromatic carbocycle (For example, cycloalkane ring (example: cyclohexane ring, cyclopentane ring and the like), cycloalkene ring (example: cyclohexene ring, cyclopentene ring and the like) and the like), non-aromatic heterocycle (For example, piperidine ring, piperazine ring, morpholine ring and the like). At the above ring, the bond(s) can be attached to monocyclic or polycyclic aromatic carbocyclyl.

For example, the following groups are also exemplified as an aryl and included in aryl. These groups can be substituted at any arbitrary position(s). When aryl is substituted, the substituent(s) on the aryl can be substituted on either monocycliy or polycyclyl aromatic carbocyclyl group or C3 to C8 ring fused monocycliy or polycyclyl aromatic carbocyclyl group.

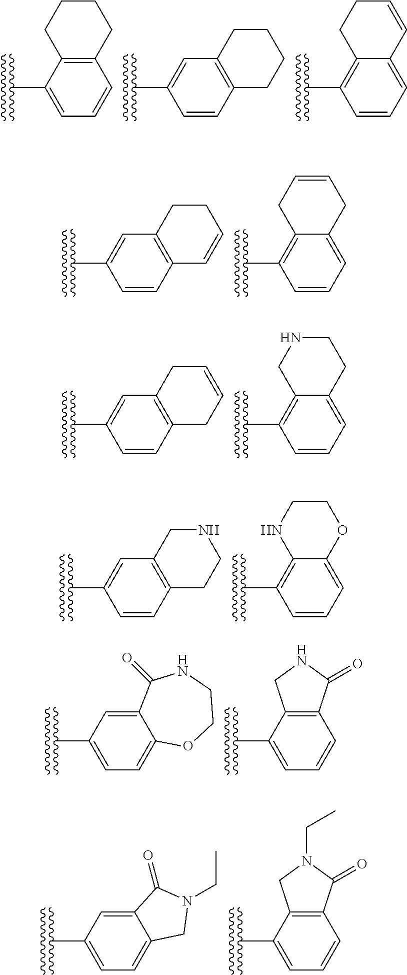

“Substituted aryl” includes an aryl substituted with oxo. “Aryl substituted with oxo” means that two hydrogen atoms on 3-8 membered ring fused monocyclic or polycyclic aromatic carbocycle constituting aryl are substituted with ═O group. As a “aryl substituted with oxo”, the following formula:

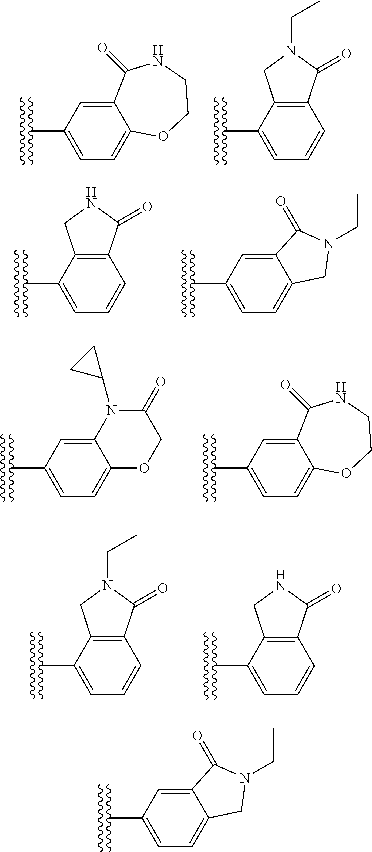

are exemplified.

“Heteroaryl” means monocyclic or polycyclic aromatic heterocyclyl containing one or more heteroatom(s) arbitrarily selected from O, S and N on the ring or the monocyclic or polycyclic aromatic heterocyclyl with one or two 3- to 8-membered cyclic group(s).

Especially preferable examples of “monocyclic aromatic heterocyclyl” are 5- or 6-membered heteroaryl. Examples are pyrrolyl, imidazolyl, pyrazolyl, pyridyl, pyridazinyl, pyrimidinyl, pyrazinyl, triazolyl, triazinyl, tetrazolyl, isoxazolyl, oxazolyl, oxadiazolyl, isothiazolyl, thiazolyl, thiadiazolyl, furyl, thienyl and the like.

Especially preferable examples of “polycyclic aromatic heterocyclyl” are heteroaryl fused with 5- to 6-membered cyclic group(s).

For example, bicyclic aromatic heterocyclyls such as indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzoisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl, thiazolopyridyl and the like, or tricyclic aromatic heterocyclyl such as carbazolyl, acridinyl, xanthenyl, phenothiazinyl, phenoxathinyl, phenoxazinyl, dibenzofuryl and the like are exemplified. When “heteroaryl” means “polycyclic aromatic heterocyclyl”, the bond(s) can be attached to any of the rings.

The ring fused with monocyclic or polycyclic aromatic heterocyclyl includes cycloalkane ring (example: cyclohexane ring, cyclopentane ring and the like), cycloalkene ring (example: cyclohexene ring, cyclopentene ring and the like), non-aromatic heterocycle (example: piperidine ring, piperazine ring, morpholine ring and the like). At the above ring, the bond(s) can be attached to monocyclic or polycyclic aromatic heterocyclyl group.

For example, the following groups are also exemplified as a heteroaryl and included in heteroaryl. These groups can be substituted at any arbitrary position(s). When heteroaryl is substituted, the substituent(s) on the heteroaryl can be substituted on either monocyclic, polycyclyl aromatic heterocyclyl, C3 to C8 ring fused monocyclic or polycyclyl aromatic heterocyclyl.

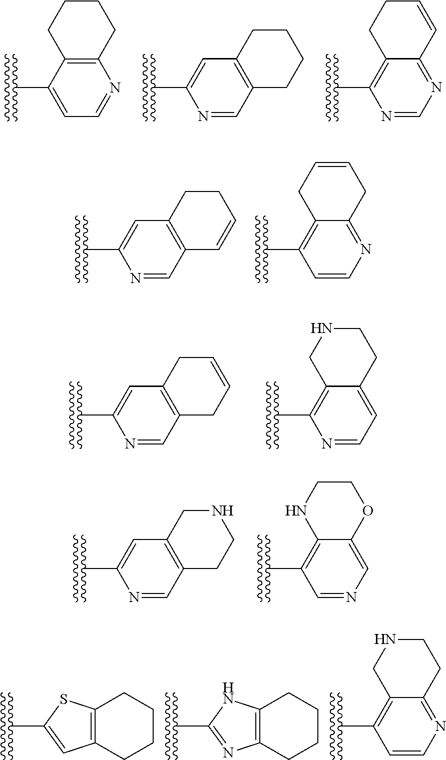

Substituted heteroaryl includes heteroaryl substituted with oxo. “Heteroaryl substituted with oxo” means that two hydrogen atoms on 3-8 membered ring fused monocyclic or polycyclic aromatic heterocycle constituting heteroaryl are substituted with ═O group. As a “heteroaryl substituted with oxo”, the following formula:

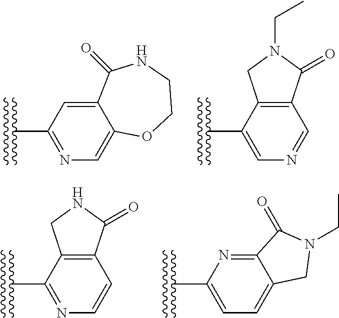

are exemplified.

“Non-aromatic heterocyclyl” means a non-aromatic heterocyclyl containing one or more heteroatom(s) arbitrarily selected from O, S and N on the ring, the non-aromatic heterocyclyl fused with one or two 3- to 8-membered cyclic group(s) (polycyclic non-aromatic heterocyclyl group(s)).

Preferable examples of “monocyclic non-aromatic heterocyclyl” are a monocyclic non-aromatic heterocyclyl group containing 1 to 4 heteroatom(s) arbitrarily selected from O, S and N on the ring. For example, dioxanyl, thiiranyl, oxiranyl, oxathiolanyl, azetidinyl, thianyl, pyrrolidinyl, pyrrolinyl, imidazolidinyl, imidazolinyl, pyrazolidinyl, pyrazolinyl, piperidyl, piperidino, piperazinyl, piperazino, morpholinyl, morpholino, oxadiazinyl, dihydropyridyl, thiomorpholinyl, thiomorpholino, tetrahydrofuryl, tetrahydropyranyl, tetrahydrothiazolyl, tetrahydroisothiazolyl, oxazolidyl, thiazolidyl, oxetanyl, thiazolidinyl, tetrahydropyridinyl, dihydrothianolyl, dihydrooxazinyl, hexahydroazepinyl, tetrahydrodiazepinyl, tetrahydropyridazinyl, hexahydropyrimidinyl, dioxolanyl, dioxazinyl, aziridinyl, dioxolinyl, oxepanyl, thiolanyl, thiazinyl and the like are exemplified.

As a monocyclic non-aromatic heterocyclyl containing 1 or more heteroatom(s) arbitrarily selected from O, S and N on the ring, for example, carbocycle (aromatic hydrocarbon ring (example: benzene ring, naphalene ring etc.), cycloalkane ring (example: cyclohexane ring, cyclopentane ring etc.), cycloalkene ring (example: cyclohexene ring, cyclopentene ring etc.)), heterocycle (aromatic heterocycle (pyridine ring, pyrimidine ring, pyrrole ring, imidazole ring etc.), non-aromatic heterocycle (example: piperidine ring, piperazine ring, morpholine ring etc.).

As a polycyclic non-aromatic heterocyclyl, for example, indolinyl, isoindolinyl, chromanyl, isochromanyl and the like are exemplified.

When “non-aromatic heterocyclyl” is polycyclic non-aromatic heterocyclyl, the bond(s) can be attached to non-aromatic heterocyclyl containing one or more heteroatom(s) arbitrarily selected from O, S and N on the ring. For example, the following groups include also non-aromatic heterocyclyl. These groups can be substituted at any arbitrary position(s). When non-aromatic heterocyclyl is substituted, the substituent(s) on the non-aromatic heterocyclyl group can be substituted on either monocycliy or polycyclyl non-aromatic heterocyclyl or 3-8 membered fused monocycliy or polycyclyl non-aromatic heterocyclyl group.

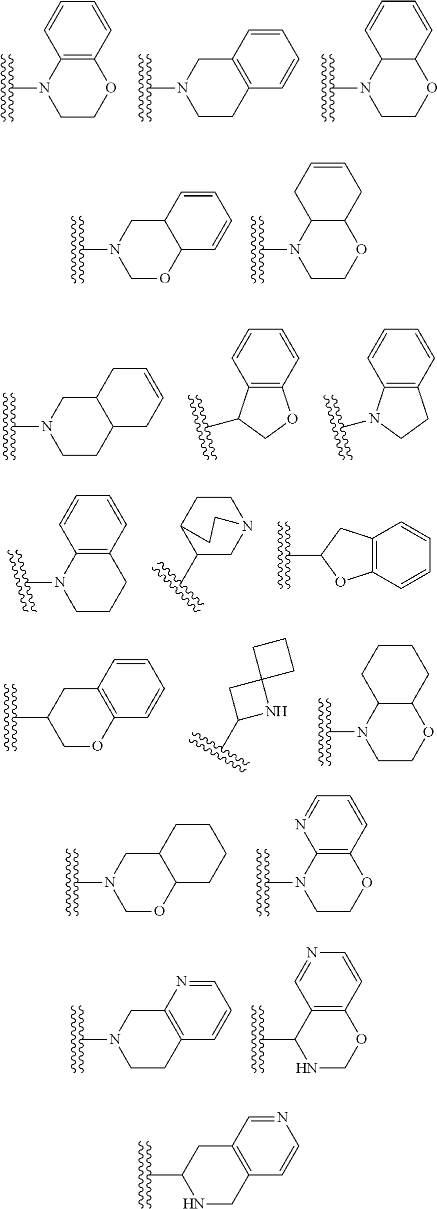

In addition, the “non-aromatic heterocyclyl” also includes a cycle having a bridge or a cycle to form a spiro ring as follows:

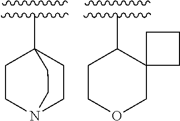

Regarding the above “cycloalkyl”, “cycloalkenyl”, “aryl” or “non-aromatic heterocyclyl”, “cycloalkane ring”, “cycloalkene ring”, “non-aromatic heterocycle”, “aromatic carbocycle”, “aromatic heterocycle”, “carbocycle” and “heterocycle” defined fusing the ring mean as follows. When the ring is substituted, the ring may have the substituent on the ring fused. “Cycloalkane ring”, “cycloalkene ring” and “non-aromatic heterocycle” may be substituted with oxo,

“Cycloalkane ring” means C3 to C8 cyclic saturated hydrocarbon group. For example, cyclohexane ring, cyclopentane ring and the like are exemplified.

“Cycloalkene ring” means C3 to C8 cyclic unsaturated hydrocarbon group. For example, cyclohexene ring, cyclopentene ring and the like are exemplified.

“Non-aromatic heterocycle” means a non-aromatic heterocycle containing one or more heteroatom(s) arbitrarily selected from O, S and N on the ring. For example, piperidine ring, piperazine ring, morpholine ring and the like are exemplified.

“Aromatic carbocycle” includes monocyclic or polycyclic aromatic carbocycle. For example, benzene ring, naphthalene ring and the like are exemplified.

“Aromatic heterocyle” means monocyclic or polycyclic aromatic heterocycle containing one or more heteroatom(s) arbitrarily selected from O, S and N on the ring or the monocyclic or polycyclic aromatic heterocycle. For example, pyridine ring, pyrimidine ring, pyrrole ring, imidazole ring and the like are exemplified.

“Carbocycle” includes the above “cycloalkane ring”, “cylcoalkene ring” and “aromatic carbocycle”.

“Heterocycle” includes the above “non-aromatic heterocycle” and “aromatic carbocycle”.

The ring that R² and R³ on the same carbon atom are taken together with the carbon atom to which they are attached to form means the above “cycloalkane ring”, “cylcoalkene ring” and “non-aromatic heterocycle ring”. Preferred is “cycloalkane ring”, cyclopropane, cyclobutane, cyclopentane and the like are exemplified. The above ring may be further substituted. As a substituents on the ring, halogen, alkyl, alkenyl, alkynyl, amino, hydroxy, alkyloxy, cyano, oxo, thioxo and the like are exemplified.

When the ring that R² and R³ on the same carbon atom are taken together with the carbon atom to which they are attached to form is “non-aromatic heterocycle”, the following formula in the compound of formula (I):

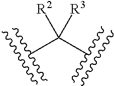

is a preferable embodiment shown by the following formula.

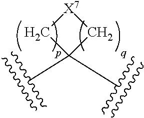

wherein p and q are each independently an integer from 0 to 3, p+q≧1, —X⁷— is bond, —O—, —S—, or —N(R¹⁵)—, R¹⁵ is hydrogen, substituted or unsubstituted alkyl, substituted or unsubstituted alkenyl or substituted or unsubstituted alkynyl.

Methylene part may be substituted with halogen, alkyl, alkenyl, alkynyl, amino, hydroxy, alkyloxy, cyano, oxo, thioxo and the like.

The ring that R⁶ and R¹³ may be taken together with the adjacent carbon atom to form means the above “cycloalkane ring”, “cycloalkene ring” and “non-aromatic heterocylcle”. Preferred is “cycloalkane ring”, and cyclopropane, cyclobutane, cyclopentane and the like are exemplified. The above ring may be further substituted. As a substituents on the ring, halogen, alkyl, alkenyl, alkynyl, amino, hydroxy, alkyloxy, cyano, oxo, thioxo and the like are exemplified.

When the ring that R⁶ and R¹³ may be taken together the adjacent carbon atom to form is “non-aromatic heterocyclyl”, the following formula in the compound of formula (I):

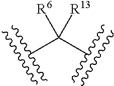

is a preferable embodiment shown by the following formula.

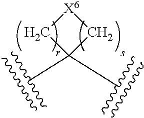

wherein r and s are each independently an integer from 0 to 3, r+s≧1, —X⁶— is bond, —O—, —S—, or —N(—R¹⁶)—, R¹⁶ is hydrogen, substituted or unsubstituted alkyl, substituted or unsubstituted alkenyl or substituted or unsubstituted alkynyl.

Methylene part may be substituted with halogen, alkyl, alkenyl, alkynyl, amino, hydroxy, alkyloxy, cyano, oxo, thioxo ane the like.

“Alkyloxy” means the above “alkyl” bonded to the oxygen atom. Examples are methoxy, ethoxy, n-propyloxy, isopropyloxy, n-butyloxy, tert-butyloxy, isobutyloxy, sec-butyloxy, pentyloxy, isopentyloxy, hexyloxy and the like. A preferable embodiment of “alkyloxy” includes methoxy, ethoxy, n-propyloxy, isopropyloxy, n-butyloxy.

“Alkenyloxy” means the above “alkenyl” bonded to the oxygen atom. Examples are vinyloxy, allyloxy, 1-propenyloxy, 2-butenyloxy, 2-pentenyloxy, 2-hexenyloxy, 2-heptenyloxy, 2-octenyloxy and the like.

“Alkynyloxy” means the above “alkynyl” bonded to the oxygen atom. Examples are ethynyloxy, 1-propynyloxy, 2-propynyloxy, 2-butynyloxy, 2-pentynyloxy, 2-hexynyloxy, 2-heptynyloxy, 2-octynyloxy and the like.

“Alkylsulfanyl” means a sulfanyl group the hydrogen atom of which is replaced by the above “alkyl”. Examples are methylsulfanyl, ethylsulfanyl, n-propylsulfanyl, isopropylsulfanyl, n-butylsulfanyl, tert-butylsulfanyl, isobutylsulfanyl, sec-butylsulfanyl, pentylsulfanyl, isopentylsulfanyl, hexylsulfanyl and the like. A preferable embodiment of “alkylsulfanyl” includes methylsulfanyl, ethylsulfanyl, n-propylsulfanyl, isopropylsulfanyl, tert-butylsulfanyl.

“Alkylsulfanylalkyl” means the above “alkyl” substituted with one or two the above “alkylsulfany”. Examples are methylsulfanylmethyl, methylsulfanylethyl, ethylsulfanylmethyl and the like.

“Alkylsulfanylalkylcarbonyl” means a carbonyl group to which the “alkylsulfanylalkyl” is bonded. Examples are methylsulfanylmethylcarbonyl, methylsulfanylethylcarbonyl, ethylsulfanylmethylcarbonyl and the like.

“Alkenylsulfanyl” means a sulfanyl group the hydrogen atom of which is replaced by the above “alkenyl”. Examples are vinylsulfanyl, allylsulfanyl, 1-propenylsulfanyl, 2-butenylsulfanyl, 2-pentenylsulfanyl, 2-hexenylsulfanyl, 2-heptenylsulfanyl, 2-octenylsulfanyl and the like.

“Alkynylsulfanyl” means a sulfanyl group the hydrogen atom of which is replaced by the above “alkynyl”. Examples are ethynylsulfanyl, 1-propynylsulfanyl, 2-propynylsulfanyl, 2-butynylsulfanyl, 2-pentynylsulfanyl, 2-hexynylsulfanyl, 2-heptynylsulfanyl, 2-octynylsulfanyl and the like.

“Alkylcarbonyl” means a carbonyl group to which the above “alkyl” is bonded. Examples are acetyl, ethylcarbonyl, propylcarbonyl, isopropylcarbonyl, tert-butylcarbonyl, isobutylcarbonyl, sec-butylcarbonyl, pentylcarbonyl, isopentylcarbonyl, hexylcarbonyl and the like. A more preferable embodiment of “alkylcarbonyl” includes acetyl, ethylcarbonyl and propylcarbonyl.

“Cyanoalkylcarbonyl” means the above “alkylcarbonyl” one or more arbitrary hydrogen(s) of which is substituted with cyano. Examples are cyanomethylcarbonyl and the like.

“Sulfamoylalkylcarbonyl” means an alkylcarbonyl substituted with sulfamoyl.

“Alkenylcarbonyl” means a carbonyl group to which the above “alkenyl” is bonded. Examples are ethylenylcarbonyl, propenylcarbonyl and the like.

“Alkynylcarbonyl” means a carbonyl group to which the above “alkynyl” is bonded. Examples are ethynylcarbonyl, propynylcarbonyl and the like.

“Alkyloxycarbonyl” means a carbonyl group to which the above “alkyloxy” is bonded. Examples are methyloxycarbonyl, ethyloxycarbonyl, propyloxycarbonyl, isopropyloxycarbonyl, tert-butyloxycarbonyl, isobutyloxycarbonyl, sec-butyloxycarbonyl, pentyloxycarbonyl, isopentyloxycarbonyl, hexyloxycarbonyl and the like. A more preferable embodiment of “alkyloxycarbonyl” includes methyloxycarbonyl, ethyloxycarbonyl, propyloxycarbonyl.

“Alkyloxycarbonylalkenyl” means the “alkenyl” the one or more arbitrary hydrogen atom(s) of which is replaced with the above “alkyloxycarbonyl”. Examples are a group of formula:

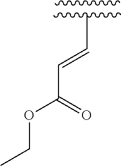

and the like.

“Alkenyloxycarbonyl” means a carbonyl group to which the above “alkenyloxy” is bonded. Examples are ethenyloxycarbonyl, propenyloxycarbonyl and the like.

“Alkynyloxycarbonyl” means a carbonyl group to which the above “alkynyloxy” is bonded. Examples are ethynyloxycarbonyl, propynyloxycarbonyl and the like.

“Arylcarbonyl” means a carbonyl group to which the above “aryl” is bonded. Examples are phenylcarbonyl, naphthylcarbonyl and the like.

“Cycloalkylcarbonyl” means a carbonyl group to which the above “cycloalkyl” is bonded. Examples are cyclopropylcarbonyl, cyclohexylcarbonyl, cyclohexenylcarbonyl and the like.

“Cycloalkylcarbonyl substituted with alkyloxycarbonyl” means the above “cycloalkylcarbonyl” substituted with one or more above “alkyloxycarbonyl”.

“Cycloalkenylcarbonyl” means a carbonyl group to which the above “cycloalkenyl” is bonded. Examples are cyclohexenylcarbonyl and the like.

“Heteroarylcarbonyl” means a carbonyl group to which the above “heteroaryl” is bonded. Examples are pyridinylcarbonyl, oxazolylcarbonyl and the like.

“Heteroarylcarbonyl substituted with alkylcarbonyl” means the above “alkylcarbonyl” substituted with one or two the above “heteroarylcarbonyl”. Examples are a group of formula:

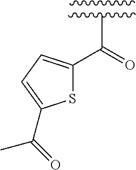

and the like.

“Non-aromatic heterocyclylcarbonyl” means a carbonyl group to which the above “non-aromatic heterocyclyl” is bonded. Examples are piperidinylcarbonyl, tetrahydrofurylcarbonyl and the like.

“Non-aromatic heterocyclylcarbonyl substituted with alkyloxycarbonyl” means the above “non-aromatic heterocyclylcarbonyl” substituted with one or two the above “alkyloxycarbonyl”. Examples are a group of formula:

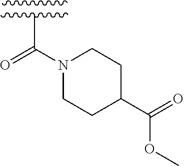

and the like.

“Alkylcarbonyloxy” means the above “alkylcarbonyl” bonded to the oxygen atom. Examples are methylcarbonyloxy, ethylcarbonyloxy, propylcarbonyloxy, isopropylcarbonyloxy, tert-butylcarbonyloxy, isobutylcarbonyloxy, sec-butylcarbonyloxy and the like. A preferable embodiment of “alkylcarbonyloxy” includes methylcarbonyloxy, ethylcarbonyloxy.

“Alkylcarbonylsulfanyl” means the above “alkylcarbonyl” bonded to the sulfur atom. Examples are methylcarbonylsulfanyl, ethylcarbonylsulfanyl, n-propylcarbonylsulfanyl, isopropylcarbonylsulfanyl, n-butylcarbonylsulfanyl, tert-butylcarbonylsulfanyl, isobutylcarbonylsulfanyl, sec-butylcarbonylsulfanyl, pentylcarbonylsulfanyl, isopentylcarbonylsulfanyl, hexylcarbonylsulfanyl and the like. A preferable embodiment of “alkylcarbonylsulfanyl” includes methylcarbonylsulfanyl, ethylcarbonylsulfanyl, propylcarbonylsulfanyl, isopropylcarbonylsulfanyl, tert-butylcarbonylsulfanyl, isobutylcarbonylsulfanyl, sec-butylcarbonylsulfanyl and the like.

“Haloalkyl” means the “alkyl” the one or more arbitrary hydrogen of which is sunstituted with the above “halogen”. Examples are monofluoromethyl, monofluoroethyl, monofluoropropyl, 2,2,3,3,3-pentafluoropropyl, monochloromethyl, trifluoromethyl, trichloromethyl, 2,2,2-trifluoroethyl, 2,2,2-trichloroethyl, 1,2-dibromoethyl, 1,1,1-trifluoropropan-2-yl and the like.

“Haloalkylcarbonyl” means a carbonyl group to which the above “haloalkyl” is bonded. Examples are monofluoromethylcarbonyl, difluoromethylcarbonyl, monofluoroethylcarbonyl, monofluoropropylcarbonyl, 2,2,3,3,3-pentafluoropropylcarbonyl, monochloromethylcarbonyl, trifluoromethylcarbonyl, trichloromethylcarbonyl, 2,2,2-trifluoroethyl, 2,2,2-trichloroethylcarbonyl, 1,2-dibromoethylcarbonyl, 1,1,1-trifluoropropan-2-ylcarbonyl and the like.

“Haloalkenyl” means the “alkenyl” the one or more arbitrary hydrogen of which is subsitutec with the above “halogen”.

“Hydroxyalkyl” means the “alkyl” the one or more arbitrary hydrogen of which is substituted with “hydroxy”.

“Trialkylsilyl” means silicon atom bonded to above three “alkyl groups”. Three alkyl groups may be same or different. Examples are trimethylsilyl, triethylsilyl, tert-butyldimethylsilyl, triisopropylsilyl and the like.

“Trialkylsilyloxy” means the above “trialkylsilyl” bonded to the oxygen atom. Examples are trimethylsilyloxy, triethylsilyloxy, tert-butyldimethylsilyloxy, triisopropylsilyloxy and the like.

“Cyanoalkyl” means the above “alkyl” the one or more arbitrary hydrogen(s) of which is substituted with cyano. Examples are cyanomethyl and the like.

“Cyanoalkyloxy” means the above “cyanoalkyl” bonded to the oxygen atom. Examples are cyanomethoxy and the like.

“Haloalkyloxy” means the above “haloalkyl” bonded to the oxygen atom. Examples are monofluoromethoxy, monofluoroethoxy, trifluoromethoxy, trichloromethoxy, trifluoroethoxy, trichloroethoxy and the like.

A preferable embodiment of “haloalkyloxy” includes trifluoromethoxy, trichloromethoxy

“Carbamoylalkylcarbonyl” means the above “alkylcarbonyl” substituted with carbamoyl. Examples are carbamoylmethylcarbonyl, carbamoylethylcarbonyl and the like.

“Monoalkylamino” means an amino group one hydrogen atom bonded to nitrogen atom of which is substituted with the above “alkyl”. Examples are methylamino, ethylamino, isopropylamino and the like.

A preferable embodiment of “monoalkylamino” includes methylamino, ethylamino.

“Mono(hydroxyalkyl)amino” means the above “monoalkylamino” the arbitrary hydrogen atoms of the alkyl of which is replaced with hydroxy. Examples are hydroxymethylamino, hydroxyethylamino and the lile.

“Dialkylamino” means an amino group two hydrogen atoms bonded to the nitrogen atom of which are replaced with the above “alkyl”. Two alkyl groups may be same or different. Examples are dimethylamino, diethylamino, N, N-diisopropylamino, N-methyl-N-ethylamino, N-isopropyl-N-ethylamino and the lile.

A preferable embodiment of “dialkylamino” includes dimethylamino, diethylamino.

“Alkylsulfonyl” means a sulfonyl group to which the above “alkyl” is bonded. Examples are methylsulfonyl, ethylsulfonyl, propylsulfonyl, isopropylsulfonyl, tert-butylsulfonyl, isobutylsulfonyl, sec-butylsulfonyl and the like.

A preferable embodiment of “alkylsulfonyl” includes methylsulfonyl, ethylsulfonyl and the like.

“Alkenylsulfonyl” means a sulfonyl group to which the above “alkenyl” is bonded. Examples are ethylenylsulfonyl, propenylsulfonyl and the like.

“Alkynylsulfonyl” means a sulfonyl group to which the above “alkynyl” is bonded. Examples are ethylnylsulfonyl, propynylsulfonyl and the like.

“Monoalkylcarbonylamino” means an amino group one hydrogen atom bonded to nitrogen atom of which is replaced with the above “alkylcarbonyl”. Examples are methylcarbonylamino, ethylcarbonylamino, propylcarbonylamino, isopropylcarbonylamino, tert-butylcarbonylamino, isobutylcarbonylamino, sec-butylcarbonylamino and the like.

A preferable embodiment of “monoalkylcarbonylamino” includes methycarbonylamino, ethycarbonylamino.

“Monoalkylcarbonylaminoalkyl” means the above “alkyl” substituted with one or more above “monoalkylcarbonylamino”. Examples are methycarbonylaminomethyl, ethycarbonylaminomethyl and the like.

“Monoalkylcarbonylaminoalkylcarbonyl” means a carbonyl group to which the above “monoalkylcarbonylaminoalkyl” is bonded. Examples are methylcarbonylaminomethylcarbonyl, ethylcarbonylaminomethylcarbonyl and the like.

“Dialkylcarbonylamino” means an amino group two hydrogen atoms bonded to nitrogen atom of which are replaced with the above “alkylcarbonyl”. Two alkylcarbonyl groups may be same or different. Examples are dimethylcarbonylamino, diethylcarbonylamino, N, N-diisopropylcarbonylamino and the like. A preferable embodiment of “dialkyloxycarbonylamino” includes dimethylcarbonylamino, diethylcarbonylamino.

“Monoalkyloxycarbonylamino” means an amino group one hydrogen atom bonded to nitrogen atom of which is replaced with the above “alkyloxycarbonyl”. A preferable embodiment of “monoalkyloxycarbonylamino” includes methyloxycarbonylamino, ethyloxycarbonylamino.

“Monoalkyloxycarbonylaminoalkyl” means the above “alkyl” substituted with one or more above “monoalkyloxycarbonylamino”. Examples are tert-butyloxycarbonylaminomethyl, tert-butyloxycarbonylaminoethyl and the like.

“Monoalkyloxycarbonylaminoalkylcarbonyl” means a carbonyl group to which the above “monoalkyloxycarbonylaminoalkyl” is bonded. Examples are tert-butyloxycarbonylaminomethylcarbonyl, tert-butyloxycarbonylaminoethylcarbonyl and the like.

“Dialkyloxycarbonylamino” means an amino group two hydrogen atoms bonded to nitrogen atom of which is replaced with the above “alkyloxycarbonyl”. Two alkyloxycarbonyl groups may be same or different. For example,

“Heteroaryl substituted with alkyloxycarbonyl” means the above “heteroaryl” substituted with one or two the above “alkyloxycarbonyl”.

“Non-aromatic heterocyclyl substituted with alkyloxycarbonyl” means the above “non-aromatic heterocyclyl” substituted with one or two the above “alkyloxycarbonyl”.

“Heteroaryl substituted with alkyl” means the above “heteroaryl” substituted with one or two the above “alkyl”.

“Monoalkylsulfonylamino” means an amino group one hydrogen atom bonded to nitrogen atom of which is replaced with the above “alkylsulfonyl” Examples are methylsulfonylamino, ethylsulfonylamino, propylsulfonylamino, isopropylsulfonylainino, tert-butylsulfonylamino, isobutylsulfonylamino, sec-butylsulfonylamino and the like.

A preferable embodiment of “monoalkylsulfonylamino” includes methylsulfonylamino, ethylsulfonylamino.

“Dialkylsulfonylamino” means aa amino group two hydrogen atoms bonded to nitrogen atom of which is replaced with the above “alkylsulfonyl”. Two alkylsulfonyl groups may be same or different. Examples are dimethylsulfonylamino, diethylsulfonylamino, N, N-diisopropylsulfonylamino and the like.

A preferable embodiment of “dialkylcarbonyllamino” includes dimethylsulfonylamino, diethylsulfonylamino.

“Alkylimino” means an imino group a hydrogen atom bonded to nitrogen atom of which is replaced with the above “alkyl”. Examples are alkylimino, ethylimino, n-propylimino, isopropylimino and the like.

“Alkenylimino” means an imino group a hydrogen atom bonded to nitrogen atom of which is replaced with the above “alkenyl”. Examples are ethylenylimino, propenylimino and the like.

“Alkynylimino” means an imino group a hydrogen atom bonded to nitrogen atom of which is replaced with the above “alkynyl”. Examples are ethynylimino, propynylimino and the like.

“Alkylcarbonylimino” means an imino group a hydrogen atom bonded to nitrogen atom of which is replaced with the above “alkylcarbonyl”. Examples are methylcarbonylimino, ethylcarbonylimino, n-propylcarbonyliinino, isopropylcarbonylimino and the like.

“Alkenylcarbonylimino” means an imino group a hydrogen atom bonded to nitrogen atom of which is replaced with the above “alkenylcarbonyl”. Examples are ethylenylcarbonylimino, propenylcarbonylimino and the like.

“Alkynylcarbonylimino” means an imino group a hydrogen atom bonded to nitrogen atom of which is replaced with the above “alkynylcarbonyl”. Examples are ethynylcarbonylimino, propynylcarbonylimino and the like.

“Alkyloxyimino” means an imino group a hydrogen atom bonded to nitrogen atom of which is replaced with the above “alkyloxy”. Examples are methyloxyimino, ethyloxyimino, n-propyloxyimino, isopropyloxyimino and the like.

“Alkenyloxyimino” means an imino group a hydrogen atom bonded to nitrogen atom of which is replaced with the above “alkenyloxy”. Examples are mthylenyloxyimino, propenyloxyimino and the like.

“Alkynyloxyimino” means an imino group a hydrogen atom bonded to nitrogen atom of which is replaced with the above “alkynyloxy”. Examples are ethynyloxyimino, propynyloxyimino and the like.

“Alkenylcarbonyloxy” means the above “alkenylcarbonyl” bonded to the oxygen atom. Examples are ethylenylcarbonyloxy, propenylcarbonyloxy and the like.

“Alkynylcarbonyloxy” means the above “alkynylcarbonyl” bonded to the oxygen atom. Examples are ethynylcarbonyloxy, propynylcarbonyloxy and the like.

“Alkylnylsulfinyl” means a sulfinyl group to which the above “alkyl” is bonded. Examples are methylsufufinyl, ethylsulfinyl, n-propylsulfinyl, isopropylsulfinyl and the like.

“Alkenylsulfinyl” means a sulfinyl group to which the above “alkenyl” is bonded. Examples are ethlenylsulfinyl, propenylsulfinyl and the like.

“Alkynylsulfinyl” means a sulfinyl group to which the above “alkynyl” is bonded. Examples are ethynylsulfinyl, propynylsulfinyl and the like.

“Monoalkylcarbamoyl” means a carbamoyl group one hydrogen atom bonded to nitrogen atom of which is replaced with the above “alkyl”. Examples are methylcarbamoyl, ethylcarbamoyl and the like.

“Monoalkylcarbamoylalkyloxy” means the above “alkyloxy” substituted with one or more above “monoalkylcarbamoyl”. Examples are methylcarbamoylmethyloxy and the like.

“Mono(hydroxyalkyl)carbamoyl” means the above “monoalkylcarbamoyl” the arbitrary hydrogen atoms of which is replaced with a hydroxy group. Examples are hydroxymethylcarbonyl, hydroxyethylcarbonyl and the lile.

“Mono(haloalkyl)carbamoyl” means the above “monoalkylcarbamoyl” the arbitrary hydrogen atoms of the alkyl of which is replaced with halogen. Examples are monochloromethylcarbamoyl, 2-chlorothylcarbamoyl and the lile.

“Dialkylcarbamoyl” means a carbamoyl group two hydrogen atoms bonded to nitrogen atom of which are replaced with the above “alkyl”. Two alkyl groups may be same or different. Examples are dimethylcarbamoyl, diethylcarbamoyl and the like.

“Alkyloxycarbonylalkyl” means the above “alkyl” substituted with one or more above “alkyloxycarbonyl”.

“Alkyloxycarbonylalkyloxy” means the above “alkyloxycarbonylalkyl” bonded to the oxygen atom. Examples are methyloxycarbonylmethyloxyl and the like.

“Mono(alkyloxycarboxyalkyl)amino” means an amino group one hydrogen atom bonded to nitrogen atom of which is replaced with the above “alkyloxycarbonylalkyl”. Examples are ethyloxycarbonylethylamino and the like.

“Alkyloxycarbonylalkylcarbonyl” means a carbonyl group to which the above “alkyloxycarbonylalkyl” is bonded. Examples are methyloxycarbonylethylcarbonyl, methyloxycarbonylmethylcarbonyl, ethyloxycarbonylethylcarbonyl, tert-butyloxycarbonylmethylcarbonyl and the like.

“Monoalkyloxycarbonylalkylcarbamoyl” means a carbamoyl group one hydrogen atom bonded to nitrogen atom of which is replaced with the above “alkyloxycarbonylalkyl”. Examples are methyloxycarbonylmethylcarbamoyl, ethyloxycarbonylmethylcarbamoyl and the like.

“Dialkyloxycarbonylalkylcarbamoyl” means a carbamoyl group two hydrogen atom bonded to nitrogen atom of which is replaced with the above “alkyloxycarbonylalkyl”.

“Carboxyalkyl” means the above “alkyl” substituted with one or more above “carboxy”.

“Carboxyalkenyl” means a group that the one or more arbitrary hydrogen of the “alkenyl” is substituted with “carboxy”. Examples are a group of formula:

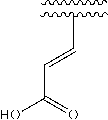

and the like.

“Carboxyalkylcarbamoyl” means a carbamoyl group one or two hydrogen atom(s) bonded to nitrogen atom of which is replaced with one or more above “carboxyalkyl”. Examples are carboxymethylcarbamoyl and the like.

“Carboxyalkyloxy” means the above “carboxyalkyl” bonded to the oxygen atom. Examples are carboxymethyloxy, carboxyethyloxy and the like.

“Monocarboxyalkylamino” means a amino group one hydrogen atom bonded to nitrogen atom of which is replaced with the above “carboxyalkyl”. Examples are carboxymethylamino, carboxyethylamino and the like.

“Dialkylaminoalkyl” means the above “alkyl” substituted with one or more above “dialkylamino”. Examples are dimethylaminomethyl, dimethylaminoethyl and the like.

“Dialkylaminocarbonyl” means a carbonyl group to which the above “dialkylamino” is bonded. Examples are dimethylaminocarbonyl and the like.

“Dialkylaminocarbonylalkylcarbonyl” means the above “alkylcarbonyl” substituted with the above “dialkylaminocarbonyl”. Examples are dimethylaminocarbonylmethylcarbonyl, dimethylaminocarbonylethylcarbonyl and the like.

“Mono(dialkylaminoalkyl)carbamoyl” means a carbamoyl group one hydrogen atom bonded to nitrogen atom of which is replaced with the above “dialkylaminoalkyl”. Examples are dimethylaminomethylcarbamoyl, dimethylaminoethylcarbamoyl and the like.

“Di(dialkylaminoalkyl)carbamoyl” means a carbamoyl group two hydrogen atoms bonded to nitrogen atom of which are replaced with the above two “dialkylaminoalkyls”. Examples are di(inethyloxycarbonylmethyl)carbamoyl, di(ethyloxycarbonylmethyl)carbamoyl and the like.

“Cycloalkylcarbamoyl” means a carbamoyl group one or two hydrogen atom(s) bonded to nitrogen atom of which is replaced with one or more above “cycloalkyl”. Examples are cyclopropylcarbamoyl and the like.

“Non-aromatic heterocyclylcarbamoyl” means a carbamoyl group the hydrogen atom bonded to nitrogen atom of which is replaced with one or more above “non-aromatic heterocyclyl”. Examples are a group of formula:

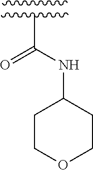

and the like.

“Monoalkyloxycarbamoyl” means a carbamoyl group one hydrogen atom bonded to nitrogen atom of which is replaced with the above “alkyloxy”. Examples are methyloxycarbamoyl and the like.

“Dialkyloxycarbamoyl” means a carbamoyl group two hydrogen atoms bonded to nitrogen atom of which is replaced with the above “alkyloxy”. Examples are di(methyloxy)carbamoyl and the like.

“Monoalkylsulfamoyl” means a sulfamoyl group one hydrogen atom bonded to nitrogen atom of which is replaced with the above “alkyl”. Examples are methylsulfamoyl, dimethylsulfamoyl and the like.

“Dialkylsulfamoyl” means a sulfamoyl group two hydrogen atoms bonded to nitrogen atom of which are replaced with the above two “alkyls”. Two alkyl groups may be same or different. Examples are dimethylcarbamoyl, dimethylcarbamoyl and the like.

“Arylalkyl” means the above “alkyl” substituted with one or more above “aryl”. Examples are benzyl, phenethyl, phenylpropenyl, benzhydryl, trityl, naphthylmethyl, a group of formula:

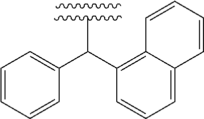

and the like.

A preferable embodiment of “arylalkyl” includes benzyl, phenethyl and, benzhydryl.

“Cycloalkylalkyl” means the above “alkyl” substituted with one or more above “cycloalkyl”. “Cycloalkylalkyl” includes “cycloalkylalkyl” which the alkyl part is further substituted with the above “aryl”. Examples are cyclopentylmethyl, cyclohexylmethyl, a group of formula:

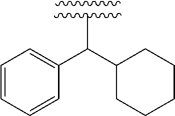

“Cycloalkenylalkyl” means the above “alkyl” substituted with one or more above “cycloalkenyl”. “Cycloalkenylalkyl” includes “cycloalkenylalkyl” which the alkyl part is further substituted with the above “aryl”. Examples are cyclopropylmethyl, cyclobutylmethyl, cyclopentylmethyl, cyclohexylmethyl and the like.

“Heteroarylalkyl” means the above “alkyl” substituted with one or more above “heteroaryl”. “Heteroarlyalkyl” includes “heteroarlyalkyl” which the alkyl part is further substituted with the above “aryl” and/or “cycloalkyl”. Examples are pyridylmethyl, furanylmethyl, imidazolymethyl, indolylmethyl, benzothiophenylmethyl, oxazolylmethyl isoxazolylmethyl, thazolylmethyl, isothiazolylmethyl, pyrrazolylmethyl, isopyrrazolylmethyl, pyrrolidinylmethyl, benzoxazolylmethyl, a group of formula:

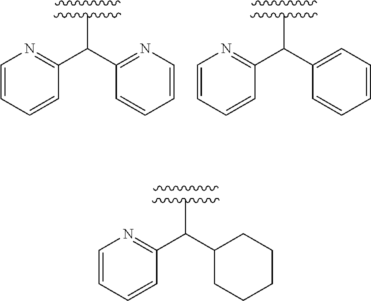

and the like.

“Heteroarylalkylcarbonyl” means a carbonyl group to which “heteroarylalkyl” is bonded. Examples are a group of formula:

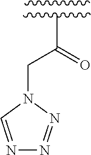

and the like.

“Non-aromatic heterocyclylalkyl” means the above “alkyl” substituted with one or more above “non-aromatic heterocyclyl”. “Non-aromatic heterocyclylalkyl” includes “non-aromatic heterocyclylalkyl” which the alkyl part is further substituted with the above “aryl”, “cycloalkyl” and/or “heteroaryl”. Examples are tetrahydropyranylmethyl, morpholinylmethyl, piperidylmethyl, piperazinylmethyl, a group of formula:

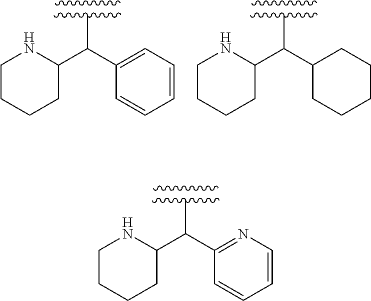

and the like.

“Non-aromatic heterocyclylalkylcarbamoyl” means a carbamoyl group one or two hydrogen atom(s) bonded to nitrogen atom of which is replaced with one or more above “non-aromatic heterocyclylalkyl”. Examples are a group of formula:

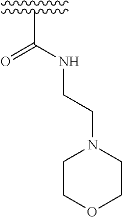

and the like.

“Non-aromatic heterocyclylalkylcarbonyl” means a carbonyl group to which one or more above ““non-aromatic heterocyclylalkyl” is bonded. Examples are a group of formula:

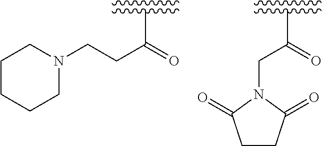

and the like.

“Arylalkyloxy” means the above “alkyloxy” substituted with one or more above “aryl”. Examples are benzyloxy, phenethyloxy, phenylpropyloxy, phenyl propynyl, benzhydryloxy, trityloxy, naphthylmethyloxy, a group of formula:

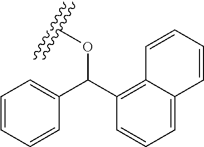

and the like.

“Cycloalkylalkyloxy” means the above “alkyloxy” substituted with one or more above “cycloalkyl”. “Cycloalkylalkyloxy” includes “cycloalkylalkyloxy” which the alkyl part is further substituted with the above “aryl”. Examples are cyclopropylmethyloxy, cyclobutylmethyloxy, cyclopentylmethyloxy, cyclohexylmethyloxy, a group of formula:

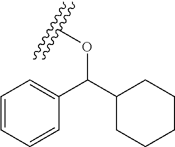

and the like.

“Cycloalkenylalkyloxy” means the above “alkyloxy” substituted with one or more above “cycloalkenyl”. “Cycloalkenylalkyloxy” includes “cycloalkenylalkyloxy” which the alkyl part is further substituted with the above “aryl”, “cycloalkyl” or both of them. Examples are cyclopropylmethyloxy, cyclobutylmethyloxy, cyclopentylmethyloxy, cyclohexylmethyloxy, a group of formula:

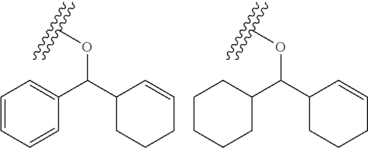

and the like.

“Heteroarylalkyloxy” means the above “alkyloxy” substituted with one or more above “heteroaryl”. “Heteroarylalkyloxy” includes “heteroarylalkyloxy” which the alkyl part is further substituted with the above “aryl” and/or “cycloalkyl”. Examples are pyridylmethyloxy, furylmethyloxy, imidazolylmethyloxy, indolylmethyloxy, benzothiophenylmethyloxy, oxazolylmethyloxy, isoxazolylmethyloxy, thiazolylmethyloxy, isothiazolylmethyloxy, pyrazolylmethyloxy, isopyrazolylmethyloxy, pyrrolidinylmethyloxy, benzoxazolylmethyloxy, a group of formula:

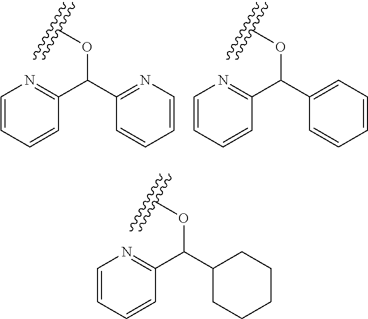

and the like.

“Non-aromatic heterocyclylalkyloxy” means the above “alkyloxy” substituted with one or more above “non-aromatic heterocyclyl”. “Non-aromatic heterocyclylalkyloxy” includes “non-aromatic heterocyclylalkyloxy” which the alkyl part is further substituted with the above “aryl”, “cycloalkyl” and/or “heteroaryl”. Examples are tetrahydropyranylmethyloxy, morpholinylethyloxy, piperidinylmethyloxy, piperazinylmethyloxy, a group of formula:

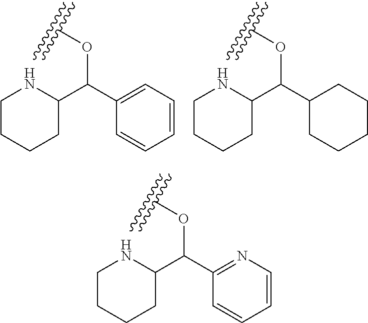

and the like.

“Arylalkyloxycarbonyl” means the above “alkyloxycarbonyl” substituted with one or more above “aryl”. Examples are benzyloxycarbonyl, phenethyloxycarbonyl, phenylpropynyloxycarbonyl, benzhydryloxycarbonyl, trityloxycarbonyl, naphthylmethyloxycarbonyl, a group of formula:

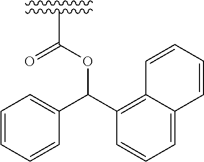

and the like.

“Cycloalkylalkyloxycarbonyl” means the above “alkyloxycarbonyl” substituted with one or more above “cycloalkyl”. “Cycloalkylalkyloxycarbonyl” includes “cycloalkylalkyloxycarbonyl” which the alkyl part is further substituted with the above “aryl”. Examples are cyclopropylmethyloxycarbonyl, cyclobutylmethyloxycarbonyl, cyclopentylmethyloxycarbonyl, cyclohexylmethyloxycarbonyl, a group of formula:

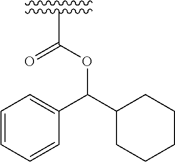

and the like.

“Cycloalkenylalkyloxycarbonyl” means the above “alkyloxycarbonyl” substituted with one or more above “cycloalkenyl”.

“Heteroarylalkyloxycarbonyl” means the above “alkyloxycarbonyl” substituted with one or more above “heteroaryl”. “Heteroarylalkyloxycarbonyl” includes “heteroarylalkyloxycarbonyl” which the alkyl part is further substituted with the above “aryl”, “cycloalkyl” and/or “cycloalkenyl”. Examples are pyridylmethyloxycarbonyl, furylmethyloxycarbonyl, imidazolylmethyloxycarbonyl, indolylmethyloxycarbonyl, benzothiophenylmethyloxycarbonyl, oxazolylmethyloxycarbonyl, isoxazolylmethyloxycarbonyl, thiazolylmethyloxycarbonyl, isothiazolylmethyloxycarbonyl, pyrazolylmethyloxycarbonyl, isopyrazolylmethyloxycarbonyl, pyrrolidinylmethyloxycarbonyl, benzoxazolylmethyloxycarbonyl, a group of formula:

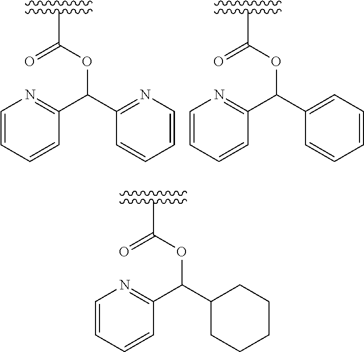

and the like.

“Non-aromatic heterocyclylalkyloxycarbonyl” means the above “alkyloxycarbonyl” substituted with one or more above “non-aromatic heterocyclyl”. “Non-aromatic heterocyclylalkyloxycarbonyl” includes “non-aromatic heterocyclylalkyloxycarbonyl” which the alkyl part is further substituted with the above “aryl”, “cycloalkyl”, “cycloalkenyl” and/or “heteroaryl”. Examples are tetrahydropyranylmethyloxy, morpholinylethyloxy, piperidinylmethyloxy, piperazinylmethyloxy, a group of formula:

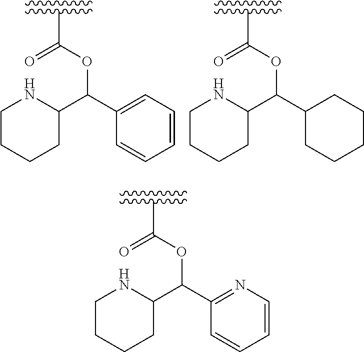

and the like.

“Arylalkylamino” means an amino group one or two hydrogen atom(s) bonded to nitrogen atom of which is replaced with the above “arylalkyl” Examples are benzylamino, phenethylamino, phenylpropynylamino, benzhydrylamino, tritylamino, naphthylmethylamino, dibenzylamino and the like.

“Cycloalkylalkylamino” means an amino group one or two hydrogen atom(s) bonded to nitrogen atom of which is replaced with the above “cycloalkylalkyl”. Examples are cyclopropylmethylamino, cyclobutylmethylamino, cyclopentylmethylamino, cyclohexylmethylamino and the like.

“Cycloalkenylalkylamino” means an amino group one or two hydrogen atom(s) bonded to nitrogen atom of which is replaced with the above “cycloalkenylalkyl”.

“Heteroarylalkylamino” means an amino group one or two hydrogen atom(s) bonded to nitrogen atom of which is replaced with the above “heteroarylalkyl”. Examples are pyridylmethylamino, furylmethylamino, imidazolylmethylamino, indolylmethylamino, benzothiophenylmethylamino, oxazolylmethylamino, isoxazolylmethylamino, thiazolylmethylamino, isothiazolylmethylamino, pyrazolylmethylamino, isopyrazolylmethylamino, pyrrolidinylmethylamino, benzoxazolylmethylamino and the like.

“Non-aromatic heterocyclylalkylamino” means an amino group one or two hydrogen atom(s) bonded to nitrogen atom of which is replaced with the above “non-aromatic heterocyclylalkyl”. Examples are tetrahydropyranylmethylamino, morpholinylethylamino, piperidinylmethylamino, piperazinylmethylamino and the like.

“Alkyloxyalkyl” means the above “alkyl” substituted with one or two the above “alkyloxy”. Examples are methyloxymethyl, methyloxyethyl, ethyloxyinethyl and the like.

“Heteroaryl substituted with alkyloxyalkyl” means the above “heteroaryl” substituted with one or two the above “alkyloxyalkyl”.

“Alkyloxyalkylcarbonyl” means a carbonyl group to which the above “alkyloxyalkylcarbonyl” is bonded. Examples are methyloxymethylcarbonyl, methyloxyethylcarbonyl, ethyloxymethylcarbonyl and the like.

“Arylalkyloxyalkyl” means the above “alkyloxyalkyl” substituted with one or more above “aryl”. Examples are benzyloxymethyl, phenethyloxymethyl, phenylpropynyloxymethyl, benzhydryloxymethyl, trityloxymethyl, naphthylmethyloxymethyl, a group of formula:

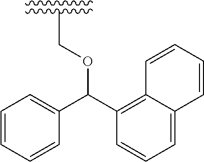

and the like.

“Cycloalkylalkyloxyalkyl” means the above “alkyloxyalkyl” substituted with one or more above “cycloalkyl”. “Cycloalkylalkyloxyalkyl” includes “cycloalkylalkyloxyalkyl” which the alkyl part bonded to cycloalkyl is further substituted with the above “aryl”. Examples are cyclopropylmethyloxymethyl, cyclobutylmethyloxymethyl, cyclopentylmethyloxy, cyclohexylmethyloxymethyl, a group of formula:

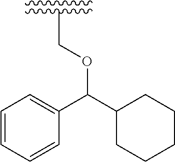

and the like.

“Cycloalkenlalkyloxyalkyl” means the above “alkyloxyalkyl” substituted with one or more above “cycloalkenyl”. “Cycloalkenylalkyloxyalkyl” includes “cycloalkenylalkyloxyalkyl” which the alkyl part bonded to cycloalkenyl is further substituted with the above “aryl”, “cycloalkyl” or both of them. Examples are a group of formula:

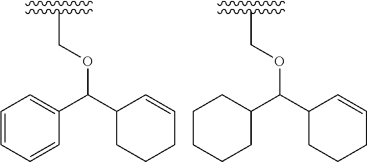

and the like.

“Heteroarylalkyloxyalkyl” means the above “alkyloxyalkyl” substituted with one or more above “heteroaryl”. “Heteroarylalkyloxyalkyl” includes “heteroarylalkyloxyalkyl” which the alkyl part bonded to aromatic heterocycle is further substituted with the above “aryl”, “cycloalkyl” and/or “cycloalkenyl”. Examples are pyridylmethyloxyalkyl, furylmethyloxyalkyl, imidazolymethyloxyalkyl, indolylmethyloxyalkyl, benzothiophenylmethyloxyalkyl, oxazolylmethyloxyalkyl, isoxazolylmethyloxyalkyl, thiazolylmethyloxyalkyl, isothiazolylmethyloxyalkyl, pyrrazolylmethyloxyalkyl, isopyrrazolylmethyloxyalkyl, pyrrolidinylmethyloxyalkyl, benzoxazolylmethyloxyalkyl, a group of formula:

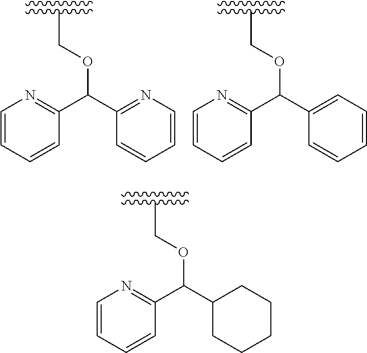

and the like.

“Non-aromatic heterocyclylalkyloxyalkyl” means the above “alkyloxyalkyl” substituted with one or more above “non-aromatic heterocyclyl”. “Non-aromatic heterocyclylalkyloxyalkyl” includes “non-aromatic heterocyclylalkyloxy” which the alkyl part bonded to non-aromatic heterocycle is further substituted with the above “aryl”, “cycloalkyl”, “cycloalkenyl”, and/or “heteroaryl”. Examples are tetrahydropyranylmethyloxymethyl, morpholinylethyloxymethyl, piperidinylmethyloxymethyl, piperazinylmethyloxymethyl, a group of formula:

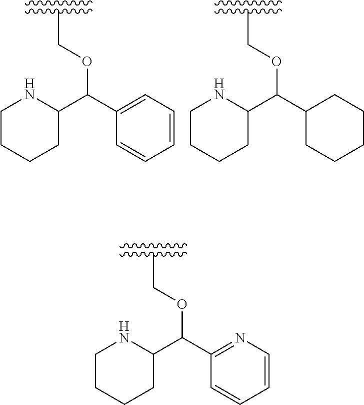

and the like.

“Aryloxy” means the above “aryl” bonded to the oxygen atom. Examples are phenyloxy, naphthyloxy and the like.

“Cycloalkyloxy” means the above “cycloalkyl” bonded to the oxygen atom. Examples are cyclopropyloxy, cyclohexyloxy, cyclohexenyloxy and the like.

“Cycloalkenyloxy” means the above “cycloalkenyl” bonded to the oxygen atom. Examples are cyclopropenyloxy, cyclobutenyloxy, cyclopentenyloxy, cyclohexenyloxy, cycloheptenyloxy, cyclohexadienyloxy and the like.

“Heteroaryloxy” means the above “heteroaryl” bonded to the oxygen atom. Examples are pyridyloxy, oxazolyloxy and the like.

“Non-aromatic heterocyclyloxy” means the above “non-aromatic heterocyclyl” bonded to the oxygen atom.

Examples of “non-aromatic heterocyclyloxy” are piperidinyloxy, tetrahydrofuryloxy and the like.

“Alkyloxyalkyloxy” means the above “alkyloxyalkyl” bonded to the oxygen atom.

“Aryloxycarbonyl” means a carbonyl group to which the above “aryloxy” is bonded. Examples are phenyloxycarbonyl, naphthyloxycarbonyl and the like.

“Cycloalkyloxycarbonyl” means a carbonyl group to which the above “cycloalkyloxy” is bonded. Examples are cyclopropyloxy carbonyl, cyclohexyloxy carbonyl, cyclohexenyloxy carbonyl and the like.

“Cycloalkenyloxycarbonyl” means a carbonyl group to which the above “cycloalkenyloxy” is bonded. Examples are cyclopropenyloxycarbonyl, cyclohexenyloxycarbonyl and the like.

“Heteroaryloxycarbonyl” means a carbonyl group to which the above “heteroaryloxy” is bonded. Examples are pyridyloxycarbonyl, oxazolyloxycarbonyl and the like.

“Non-aromatic heterocyclyloxycarbonyl” means a carbonyl group to which the above “non-aromatic heterocyclyloxy” is bonded. Examples are piperidinyloxy carbonyl, tetrahydrofuryloxycarbonyl and the like.

“Arylsulfanyl” means a sulfanyl group hydrogen atom bonded to sulfur atom of which is replaced with the above “aryl”. Examples are phenylsulfanyl, naphthysulfanyl and the like.

“Cycloalkylsulfanyl” means a sulfanyl group hydrogen atom bonded to sulfur atom of which is replaced with the above “cycloalkyl”. Examples are cyclopropylsulfanyl, cyclohexylsulfanyl, cyclohexenylsulfanyl and the like.

“Cycloalkenylsulfanyl” means a sulfanyl group hydrogen atom bonded to sulfur atom of which is replaced with the above “cycloalkenyl”. Examples are cyclopropenylsulfanyl, cyclobutenylsulfanyl, cyclohexenylsulfanyl, cyclopentenylsulfanyl, cycloheptenylsulfanyl, cyclohexadienylsulfanyl and the like.

“Heteroarylsulfanyl” means a sulfanyl group hydrogen atom bonded to sulfur atom of which is replaced with the above “heteroaryl”. Examples are pyridylsulfanyl, oxazolysulfanyl and the like.

“Non-aromatic heterocyclylsulfanyl” means a sulfanyl group hydrogen atom bonded to sulfur atom of which is replaced with the above “non-aromatic heterocyclyl”. Examples are piperidinylsulfanyl, tetrahydrofurylsulfanyl and the like.

“Arylsulfonyl” means a sulfonyl group to which the above “aryl” is bonded. Examples are phenylsulfonyl, naphthylsulfonyl and the like.

“Cycloalkylsulfonyl” means a sulfonyl group to which the above “cycloalkyl” is bonded. Examples are cyclopropylsulfonyl, cyclohexylsulfonyl, cyclohexenylsulfonyl and the like.

“Cycloalkenylsulfonyl” means a sulfonyl group to which the above “cycloalkenyl” is bonded.

“Heteroarylsulfonyl” means a sulfonyl group to which the above “heteroaryl” is bonded. Examples are pyridylsulfonyl, oxazolylsulfonyl and the like.

“Non-aromatic heterocyclylsulfonyl” means a sulfonyl group to which the above “non-aromatic heterocyclyl” is bonded. Examples are piperidinylsulfonyl, tetrahydrofurylsulfonyl and the like.

“Non-aromatic heterocyclyl substituted with alkyl” means the above “non-aromatic heterocyclyl” substituted with the one or two above “alkyl”.

“Non-aromatic heterocyclylcarbamoyl substituted with alkyloxycarbonyl” means the above “non-aromatic heterocyclylcarbamoyl” one or two hydrogen(s) bonded to the atom on the non-aromatic of which is replaced with the above “alkyloxycarbonyl”.

Examples are a group of formula:

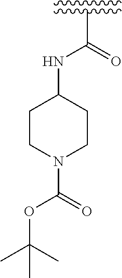

and the like

Preferable embodiments of R¹, R², R³, R⁴, R⁵, R⁶, R⁷, R⁸, R⁹, R¹³, n, m, A, X¹ and X⁵ in the compounds of formula (I′) are described below.

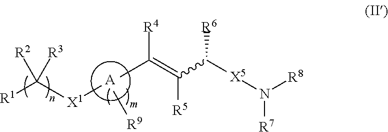

R¹ is substituted or unsubstituted aryl or substituted or unsubstituted heteroaryl. Preferred is substituted or unsubstituted aryl. Especially preferred is substituted or unsubstituted phenyl. Furthermore, preferred is substituted phenyl. As another embodiments, preferred is substituted or unsubstituted fused aryl or substituted or unsubstituted fused heteroaryl. Preferable substituted aryl or substituted heteroaryl is the followings:

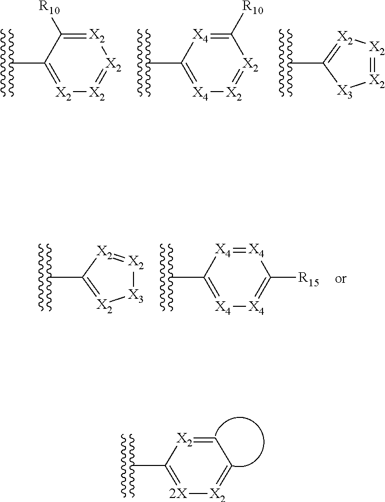

wherein X² is each independently —N═, —C(H)═ or —C(—R¹⁰)═,

### X³ is —S—, —O—, —N(H)— or —N(—R¹¹)—,

X⁴ is each independently —N═ or —C(H)═,
R¹⁰ is each independently halogen, substituted or unsubstituted alkyl, substituted or unsubstituted alkenyl, substituted or unsubstituted alkynyl, substituted or unsubstituted amino, hydroxy, substituted or unsubstituted alkyloxy, substituted or unsubstituted alkylcarbonyloxy, mercapto, substituted or unsubstituted alkylsulfanyl, substituted or unsubstituted alkylamino, substituted or unsubstituted alkylcarbonylsulfanyl, cyano, substituted or unsubstituted non-aromatic heterocyclyl, trialkylsilyloxy, substituted or unsubstituted aryloxy, substituted or unsubstituted aryl, substituted or unsubstituted heteroaryl, substituted or unsubstituted cycloalkyl, substituted or unsubstituted cycloalkeyl, substituted or unsubstituted alkylsulfonyl or substituted or unsubstituted alkylsulfonyloxy,
R¹¹ is each independently substituted or unsubstituted alkyl, substituted or unsubstituted alkeyl or substituted or unsubstituted alkynyl,
R¹⁵ is substituted or unsubstituted C2 or more alkyl, substituted or unsubstituted aryl, substituted or unsubstituted aryloxy or substituted or unsubstituted non-aromatic heterocyclyl,
Ring P is substituted or unsubstituted 5-membered aromatic heterocycle, substituted or unsubstituted 5-membered non-aromatic carbocycle, substituted or unsubstituted 5-membered non-aromatic heterocycle, substituted or unsubstituted 6-membered non-aromatic carbocycle or substituted or unsubstituted 6-membered non-aromatic heterocycle. Especially preferred is a group of formula:

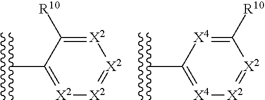

X² is each independently —N═, —C(H)═ or —C(—R¹⁰)═
X⁴ is each independently —N═ or —C(H)═,
R¹⁰ is each independently halogen, substituted or unsubstituted alkyl, substituted or unsubstituted alkenyl, substituted or unsubstituted alkynyl, substituted or unsubstituted amino, hydroxy, substituted or unsubstituted alkyloxy, substituted or unsubstituted alkylcarbonyloxy, mercapto, substituted or unsubstituted alkylsulfanyl, substituted or unsubstituted alkylamino, substituted or unsubstituted alkylcarbonylsulfanyl, cyano, substituted or unsubstituted non-aromatic heterocyclyl, trialkylsilyloxy, substituted or unsubstituted aryloxy, substituted or unsubstituted aryl, substituted or unsubstituted heteroaryl, substituted or unsubstituted cycloalkyl, substituted or unsubstituted cycloalkeyl, substituted or unsubstituted alkylsulfonyl or substituted or unsubstituted alkylsulfonyloxy.

Preferable R¹⁰ is halogen (e.g., chloro etc.), substituted or unsubstituted alkyl (e.g., haloalkyl etc.), substituted or unsubstituted amino (e.g., monoalkylamino, monoalkyloxycarbonylamino, cycloalkylalkylamino), substituted or unsubstituted alkyloxy (e.g., cycloalkylalkyloxy etc.), cyano, trialkylsilyloxy or, substituted or unsubstituted aryloxy.

Preferable examples of R⁴ are a group of formula:

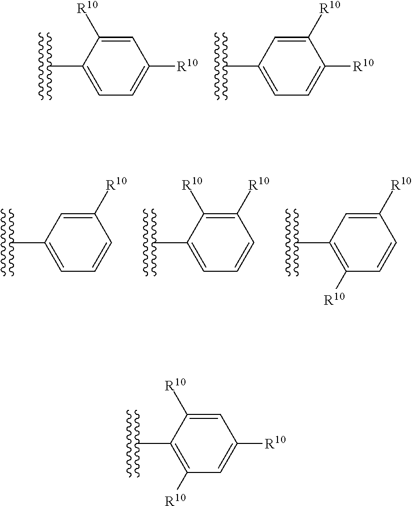

As another embodiments, preferable R¹ is substituted or unsubstituted fused aryl or substituted or unsubstituted fused heteroaryl.

Fused aryl means a group that polycyclic aromatic carbocyclyl or monocyclic or polycyclic aromatic carbocyclyl fused with one or two 3 to 8-membered cyclic group(s).

Fused heteroaryl means a group that polycyclic aromatic heterocyclyl or monocyclic or polycyclic aromatic heterocyclyl fused with one or two 3 to 8-membered cyclic group(s).

Preferred examples of substituted or unsubstituted fused aryl or substituted or unsubstituted fused heteroaryl are a group of formula:

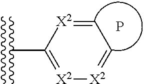

wherein X² has the same meaning as defined above.

Ring P is substituted or unsubstituted 5-membered aromatic heterocycle, substituted or unsubstituted 5-membered non-aromatic carbocycle, substituted or unsubstituted 5-membered non-aromatic heterocycle, substituted or unsubstituted 6-membered non-aromatic carbocycle or substituted or unsubstituted 6-membered non-aromatic heterocycle. Ring P and a group of formula:

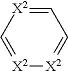

form to fuse bicyclic ring Especially preferred is substituted or unsubstituted 5-membered aromatic heterocycle, substituted or unsubstituted 5-membered non-aromatic carbocycle or substituted or unsubstituted 5-membered non-aromatic heterocycle.

A preferable embodiment of the above group of formula:

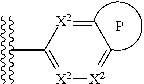

are a group of formula:

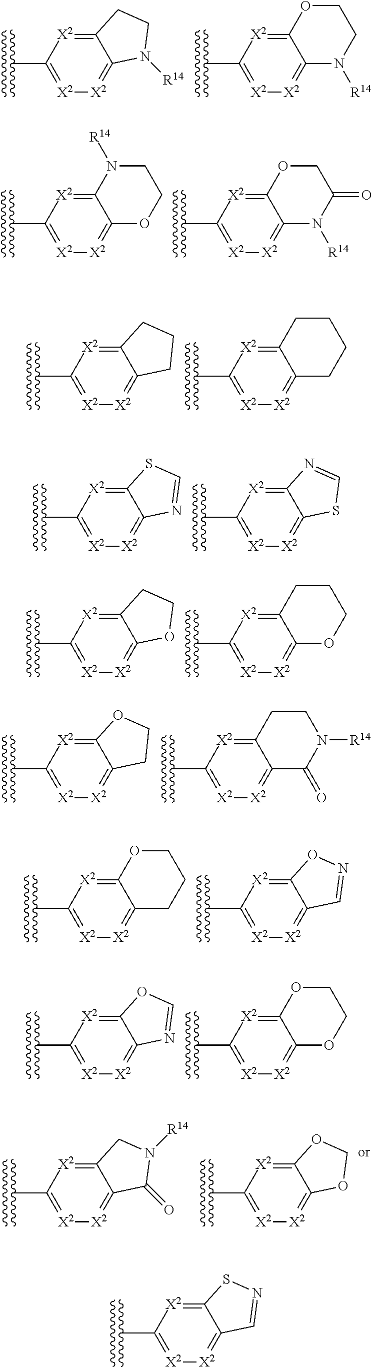

R¹⁴ is hydrogen, substituted or unsubstituted alkyl, substituted or unsubstituted alkenyl or substituted or unsubstituted alkynyl. Preferable R¹⁴ is substituted or unsubstituted alkyl (e.g., cycloalkylalkyl).

The carbon atom on Ring P may be further substituted. Examples of the substituent are halogen, substituted or unsubstituted alkyl (e.g., haloalkyl etc.) or substituted or unsubstituted cycloalkyl.

Preferable examples are a group of the following formula:

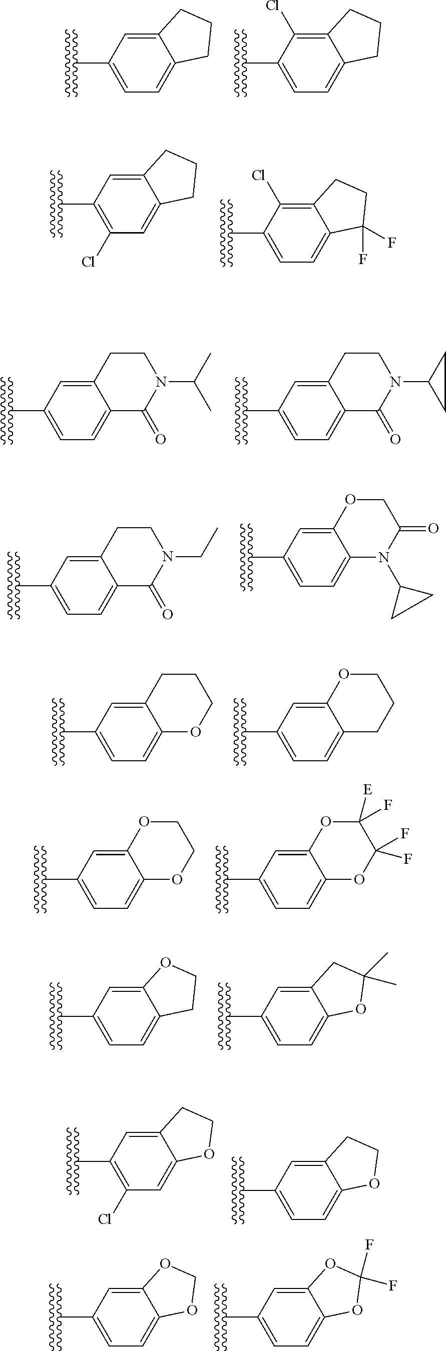

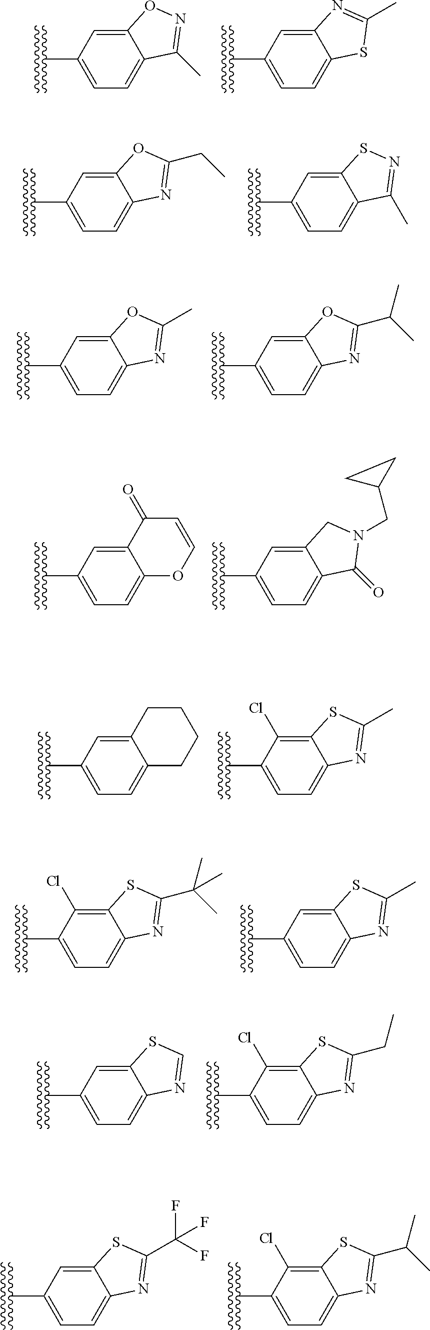

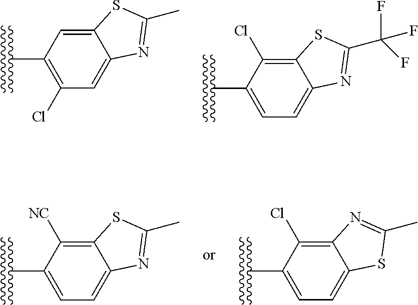

R² is each independently hydrogen, substituted or unsubstituted alkyl or halogen, R³ is each independently hydrogen, substituted or unsubstituted alkyl or halogen, or R² and R³ on the same carbon atom may be taken together with the carbon atom to which they are attached to form substituted or unsubstituted ring. Preferably R² is each independently hydrogen, substituted or unsubstituted alkyl or halogen, R³ is each independently hydrogen, substituted or unsubstituted alkyl or halogen, more preferably R² and R³ is hydrogen.

R² and R³ may be taken together with the substituent on the aryl or heteroaryl ring on R¹ and the atom and to which each is attached to form a ring. When R² is taken together with the substituent (R¹⁰) on the aryl or heteroaryl ring of R¹ and the atom to which each is attached to form a ring, a group of formula in formula (I′):

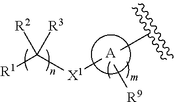

can be shown as a formula:

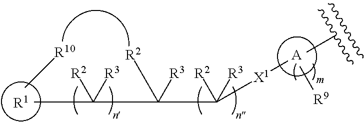

For example, a compound of formula (I) can be described as a formula (I-A):

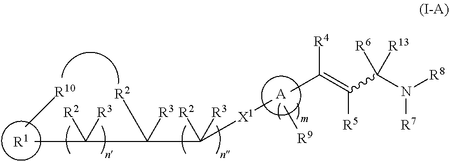

wherein each symbol has the same meaning as defined above, n is an integer from 0 to 3, n′ and n″ is 0 or more integer that satisfies a formula: n′+n″+1=n.

A preferable embodiments of a compound of the above formula (I-A) include a compound of formula (I-A1).

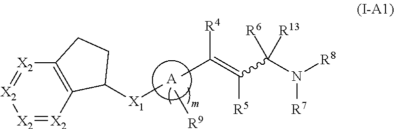

wherein each symbol has the same meaning as defined above.

When X¹ is —C(—R²)(—R³)—, —O—C(—R²)(—R³)—, —S—C(—R²)(—R³)— or —N(—R¹²)—C(—R²)(—R³)—, R² or R³ in X¹ may be taken together with the substituent on the aryl or heteroaryl ring on R¹ and the atom and to which each is attached to form a ring. In this case, a group of formula in formula (I′):

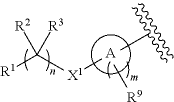

can be shown as a formula:

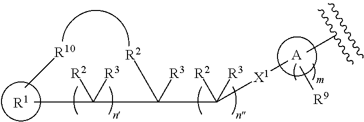

For example, a compound of formula (I) can be described as a formula (I-B):

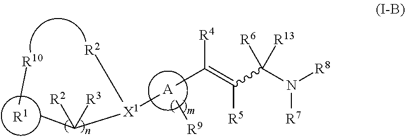

wherein each symbol has the same meaning as defined above

A preferable embodiments of a compound of the above formula (I-B) include a compound of formula (I-B1).

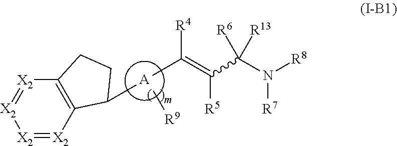

When X¹ is —N(—R¹²)— or —N(—R¹²)—C(—R²)(—R³)—, R¹² in X¹ may be taken together with the substituent on the aryl or heteroaryl ring on 10 and the atom and to which each is attached to form a ring. In this case, a group of formula in formula (I′):

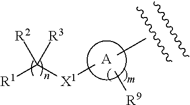

can be shown as a formula:

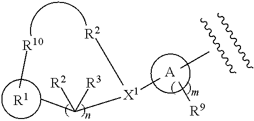

For example, a compound of formula (I) can be described as a formula (I-C):

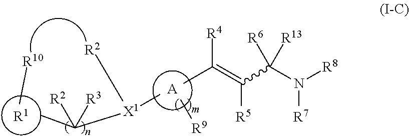

wherein each symbol has the same meaning as defined above.

Preferable embodiments of a compound of the above formula (I-C) include a compound of formula (I-C1).

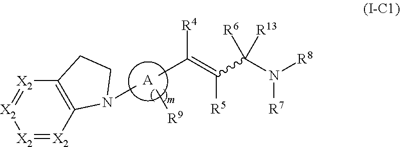

wherein each symbol has the same meaning as defined above.

R⁴ and R⁵ is each independently hydrogen, substituted or unsubstituted alkyl, substituted or unsubstituted alkenyl, substituted or unsubstituted alkynyl, halogen, substituted or unsubstituted alkyloxy or substituted or unsubstituted alkyloxycarbonyl. Preferable R⁴ is hydrogen, and preferable R⁵ is hydrogen or halogen. More preferable R⁴ and R⁵ are hydrogen.

R⁶ is substituted or unsubstituted alkyl, substituted or unsubstituted alkenyl or substituted or unsubstituted alkynyl. Preferable R⁶ is substituted or unsubstituted alkyl. Especially preferable R⁶ is methyl or ethyl. More preferable R⁶ is methyl.

R¹³ is hydrogen, substituted or unsubstituted alkyl, substituted or unsubstituted alkenyl or substituted or unsubstituted alkynyl. Preferable R¹³ is hydrogen.

R⁷ is hydrogen or substituted or unsubstituted alkyl. Preferable R⁷ is hydrogen.

R⁸ is substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted cycloalkenylcarbonyl, alkyloxycarbonyl, substituted or unsubstituted alkenyloxycarbonyl, substituted or unsubstituted alkynyloxycarbonyl, substituted or unsubstituted carbamoyl, substituted or unsubstituted sulfamoyl, substituted or unsubstituted amidino, substituted or unsubstituted arylcarbonyl, substituted or unsubstituted heteroarylcarbonyl, substituted or unsubstituted non-aromatic heterocyclylcarbonyl, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted amino, substituted or unsubstituted aryl, or substituted or unsubstituted heteroaryl, substituted or unsubstituted non-aromatic heterocyclyl, substituted or unsubstituted aryloxycarbonyl or substituted or unsubstituted sulfino.

R⁸ is preferably substituted or unsubstituted alkylcarbonyl (for example: following substituent can be substituted. halogen, alkylsulfanyl, cyano, monoalkylcarbonylamino, non-aromatic heterocyclyl, non-aromatic heterocyclyl substituted with alkyloxycarbonyl, non-aromatic heterocyclyl substituted with alkyl, non-aromatic heterocyclylalkylcarbonyl substituted with oxo, heteroaryl, heteroaryl substituted with alkyloxycarbonyl, alkyloxy, alkyloxycarbonyl, dialkylaminocarbonyl, sulfamoyl, alkyloxyalkyloxy, monoalkyloxycarbonylamino, carbamoyl, monoalkylsulfonylamino, alkylcarbonyl, hydroxy or dialkylamino), substituted or unsubstituted cycloalkylcarbonyl (for example: following substituent can be substituted. carbamoyl, alkyl, alkyloxycarbonyl, hydroxy or cyano), substituted or unsubstituted alkyloxycarbonyl (for example: following substituent can be substituted. unsubstituted alkyloxycarbonyl and the like), substituted or unsubstituted carbamoyl (for example: following substituent can be substituted. alkyl, alkyl, alkyloxy, haloalkyl, cycloalkyl, hydroxyalkyl, monoalkyloxyalkyl or cyanoalkyl), substituted or unsubstituted arylcarbonyl, (for example: following substituent can be substituted. alkyloxycarbonyl, non-aromatic heterocyclyl, heteroaryl, oxo, alkylsulfonyl, halogen, sulfamoyl, alkyl, oxo, cyano or alkyloxy), substituted or unsubstituted heteroarylcarbonyl (for example: following substituent can be substituted. hydroxylalkyl, formyl, alkyloxycarbonylalkenyl, carboxyalkenyl, alkyloxycarbonylalkyloxy, non-aromatic heterocyclylalkyloxy, mono(hydroxyalkyl)amino, carboxyalkyloxy, monocarboxyalkylamino, monoalkylcarbamoylalkyloxy, mono(hydroxyalkyl)carbamoyl, non-aromatic heterocyclylcarbamoyl, heteroarylcarbonyl optionally substituted with monoalkylcarbamoyl, carbamoyl, non-aromatic heterocyclylalkylamino, mono(alkyloxycarbonylalkyl)amino, monoalkylcarbonylamino, heteroaryl, heteroaryl substituted with alkyl, alkylheteroaryl, heteroaryl, alkyloxy, halogen, dimethylamino, amino, heteroarylcarbonyl, halogen, alkyloxycarbonyl, monoalkyloxycarbamoyl, non-aromatic heterocyclylalkylcarbamoyl, monocycloalkylcarbamoyl, non-aromatic heterocyclylcarbamoyl substituted with alkyloxycarbonyl, hydroxycarbamoyl, mono(dialkylaminoalkyl)carbamoyl, cyanocarbamoyl, monoalkyloxycarbonylalkylcarbamoyl, heteroarylcarbonyl substituted with cycloalkylcarbamoyl substituted with alkyloxycarbonyl, heteroarylcarbonyl substituted with carboxyalkylcarbamoyl, cycloalkyl substituted with carboxy, heteroaryl substituted alkylcarbonyl, dialkylamino, monoalkylcarbonylamino, non-aromatic heterocyclyl or heteroaryl substituted with alkyloxyalkyl), substituted or unsubstituted non-aromatic heterocyclylcarbonyl (for example: following substituent can be substituted. alkyloxy, alkyloxycarbonyl, hydroxyalkyl, alkyloxycarbonyl or oxo), substituted or unsubstituted alkyloxycarbonyl, substituted or unsubstituted heteroaryl (for example: following substituent can be substituted. alkyl), substituted or unsubstituted aryloxycarbonyl (for example: following substituent can be substituted. nitro.) or substituted or unsubstituted sulfino (for example: following substituent can be substituted. alkyl).

R⁸ is preferably substituted or unsubstituted alkylcarbonyl, more preferably unsubstituted alkylcarbonyl, most preferably methylcarbonyl.

R⁹ is substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted alkylsulfanyl, substituted or unsubstituted alkenylsulfanyl, substituted or unsubstituted alkynylsulfanyl, halogen, hydroxy, cyano, substituted or unsubstituted amino, substituted or unsubstituted carbamoyl, substituted or unsubstituted sulfamoyl, carboxy, substituted or unsubstituted alkylcarbonyl or substituted or unsubstituted alkyloxycarbonyl.

n is an integer from 0 to 3, preferably 0.

m is an integer from 0 to 4, preferably from 0 to 2, more preferably 0.

Ring A is aromatic carbocycle or aromatic heterocycle. Preferable aromatic carbocycle of ring A is benzene. Preferable aromatic heterocycle of ring A is 5- or 6-membered aromatic heterocycle containing 1 to 3 heteroatom(s) arbitrarily selected from O, S and N on the ring. Furthermore, pyrazole, thiazole, pyridine, pyrimidine, pyridazine or pyrazine are preferable.

X¹ is —O—, —S—, —N(—R¹²)—, —C(═O)—, —C(—R²)(—R³)—, —O—C(—R²)(—R³)—, —S—C(—R²)(—R³)— or —N(—R¹²)—C(—R²)(—R³)—, preferably —O—, —O—C(—R²)(—R³)— or —C(—R²)(—R³)—, more preferably —O—.

X⁵ is bond or —C(—R¹⁶)(—R¹⁷)—, preferably bond or methylene, more preferably bond.

“A disease associated with ACC” includes metabolic syndrome, obesity, diabetes, insulin resistance, abnormal glucose tolerance, diabetic peripheral neuropathy, diabetic nephropathy, diabetic retinal disease, diabetic macroangiopathy, hyperlipidemia, hypertension, cardiovascular illness, arterial sclerosis, atherosclerotic cardiovascular disease, cardiac arrest, cardiac infarction, infectious disease, neoplasm and the like.

The compounds of the invention are not limited to the specific isomer, include all possible isomers (For example, keto-enol isomer, imine-enamine isomer, diastereo isomer, enantiomer, rotamer and the like) and racemates or mixture thereof.

A compound of formula (I′):

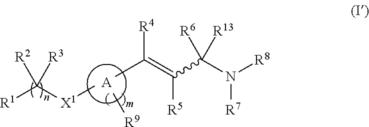

forms a double bond with a carbon atom bonded to R⁴ and a carbon atom bonded to R⁵.

The present invention includes a compound that a group of formula:

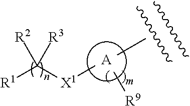

and a group of formula:

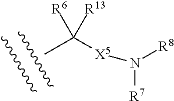

is E configuration or Z configuration to the above double bond. In the above formula (I′), the wave line means E configuration, Z configuration or the mixture of them to the above double bond.

When the wave line in the above formula (I′) is E configuration to the above double bond, the above formula (I) is described as the following formula (I′-D).

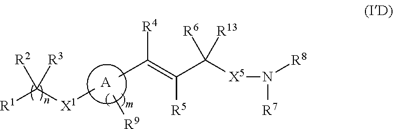

When the wave line in the above formula (I′) is Z configuration to the above double bond, the above formula (I) is described as the following formula (I′-E).

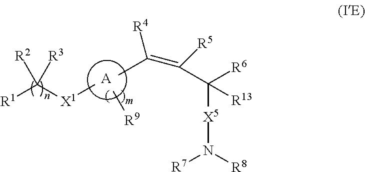

Preferably, the above each group is E configuration.

When R⁶ and R¹³ in not same substituent in the formula (I′), R isomer and S isomer exists. The present invention includes both racemate and optical isomer (R isomer and S isomer).

When R¹³ is hydrogen, a compound of formula (I′):

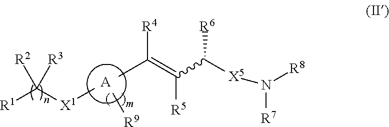

is preferably a compound of formula (II′).

A compound of formula (II′) means a compound of formula (II′-A):

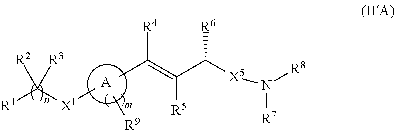

a compound of formula (II′-B):

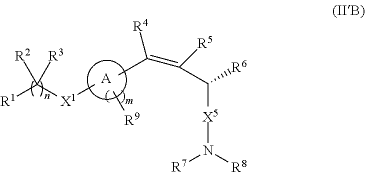

or the mixture of them. Especially preferred is a compound shown as a formula (II′-A).

When X⁵ in the formula (I′) is bond, the above formula (I′) is described as the following formula (I):

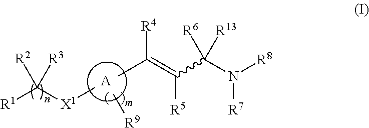

A compound of formula (I):

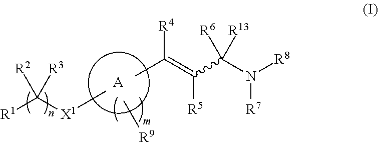

forms a double bond with a carbon atom bonded to R⁴ and a carbon atom bonded to R⁵.

The present invention includes a compound that a group of formula:

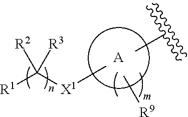

and a group of formula:

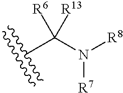

is E configuration or Z configuration to the above double bond. In the above formula (I), the wavy line means E configuration, Z configuration or the mixture of them to the above double bond.

When the wave line in the above formula (I) is E configuration to the above double bond, the above formula (I) is described as the following formula (I-D).

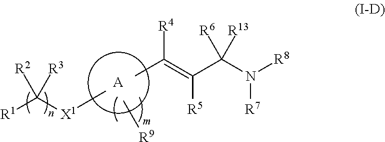

When the wave line in the above formula (I) is Z configuration to the above double bond, the above formula (I) is described as the following formula (I-E).

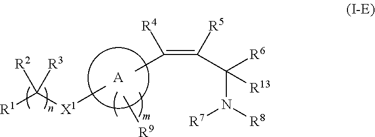

Preferably, the above each group is E configuration.

When R⁶ and R¹³ in not same substituent in the formula (I), R isomer or S isomer exists. The present invention includes both racemate and optical isomer (R isomer and S isomer).

When R¹³ is hydrogen, a compound of formula (I):

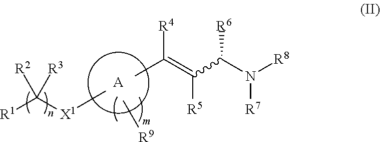

is preferably a compound of formula (II).

A compound of formula (II) means a compound of formula (II-A):

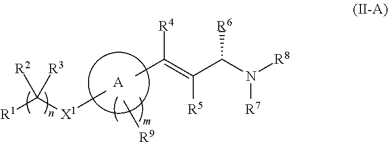

a compound of formula (II′-B):

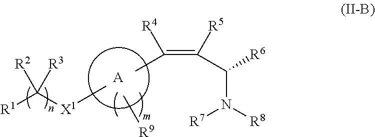

or the mixture of them. Especially preferred is a compound shown as a formula (II-A).

One or more hydrogen, carbon and/or other atoms of the compounds of formula (I′) can be replaced by an isotope of the hydrogen, carbon and/or other atoms. The examples of isotopes include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. The compounds of formula (I′) include compounds that substituted with the isotopes. And the compounds substituted with the isotopes are useful as medicine, and include radiolabeled forms of the compounds of formula (I′) “radiolabeled,” “radiolabeled form”. The process for radiolabeling the compounds of formula (I′) to prepare the “radiolabeled form” is encompassed by the invention, is useful as a research and/or diagnostic tool in metabolism pharmacokinetic studies and in binding assays.

Radiolabeled compounds of formula (I′) can be prepared by methods known in the art. For example, tritiated compounds of formula (I′) can be prepared by introducing tritium into the particular compound of formula (I′), for example, by catalytic dehalogenation with tritium. This method may include reacting a suitably halogen-substituted precursor of a compound of formula (I′) with tritium gas in the presence of a suitable catalyst such as Pd/C, in the presence or absence of a base. Other suitable methods for preparing tritiated compounds can be found in Filer, “The Preparation and Characterization of Tritiated Neurochemicals,” Chapter 6, pp. 155-192 in Isotopes in the Physical and Biomedical Sciences, Vol. 1, Labeled Compounds (Part A) (1987). 14C-labeled compounds can be prepared by employing starting materials having a 14C carbon.

Examples of “pharmaceutically acceptable salts” include salt such as a compound of formula (I′) with alkaline metals (e.g. lithium, sodium, potassium and the like), alkaline earth metals (e.g. calcium, barium and the like), magnesium, transition metals (e.g. zinc, iron and the like), ammonium, organic bases (e.g. trimethylamine, triethylamine, dicyclohexylamine, ethanolamine, diethanolamine, triethanolamine, meglumine, diethanolamine, ethylenediamine, pyridine, picoline, quinoline and the like) and amino acids, and salts with inorganic acids (e.g. hydrochloric acid, sulfuric acid, nitric acid, carbonic acid, hydrobromic acid, phosphoric acid, hydroiodic acid and the like), and organic acids (e.g. formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid, ethanesulfonic acid and the like). Specifically preferable examples are hydrochloric acid, sulfuric acid, phosphoric acid, tartaric acid, methanesulfonic acid and the like. These salts may be formed by a routine method.

The compounds of the invention of formula (I′) or its pharmaceutically acceptable salts can be prepared in a form of solvate (For example, hydrate and the like) thereof and its crystal polymorph, the present invention includes such solvate and polymorph. Any number of solvent molecules can be coordinated to form such solvate to the compounds of formula (I′). When the compounds of formula (I′) or its pharmaceutically acceptable salt are left in the atmosphere, it can absorb moisture to attach the absorbed water or to form the hydrate. Also, the compounds of formula (I′) or its pharmaceutically acceptable salt can be recrystallized to form the crystal polymorph.

The compounds of the invention of formula (I′) or its pharmaceutically acceptable salts can be formed the prodrug, the present invention includes the various prodrug. The prodrug is the derivatives of the compounds for this invention having the group decomposed by chemical or metabolic method, and are compounds that prepared by solvolysis or under condition, and are compounds having an activity in vivo. The prodrug includes compounds converted to the compounds for this invention of formula (I′) by oxidation, reduction or hydrolysis under physiological conditions in vivo and compounds hydrolyzed to the compounds for this invention of formula (I′) by gastric acid and the like.

When the compounds of the invention of formula (I′) or its pharmaceutically acceptable salt has hydroxy, for example, it is reacted with the suitable acyl halide, the suitable acid anhydride, the suitable sulfonyl chloride, the suitable sulfonyl anhydride and mixed anhydride or with condensation agent to afford the prodrug such as the acyloxy derivatives or sulfonyoxy derivatives.

Examples of the prodrug are CH₃COO—, C₂H₅COO—, t-BuCOO—, C₁₅H₃₁COO—, PhCOO—, (m-NaOOCPh) COO—, NaOOCCH₂CH₂COO—, CH₃CH(NH₂)COO—, CH₂N(CH₃)₂COO—, CH₃SO₃—, CH₃CH₂SO₃—, CF₃SO₃—, CH₂FSO₃—, CF₃CH₂SO₃—, p-CH₃—O-PhSO₃—, PhSO₃—, p-CH₃PhSO₃—.

The general procedures for the compounds of the present invention are described as follows. Any starting materials and reaction reagents are readily available or are prepared by techniques and procedures readily available to one skilled in the art.

When the compound of formula (I′) is the compound of formula (I) wherein X⁵ is bond, the compound can be prepared by the following Preparation A.

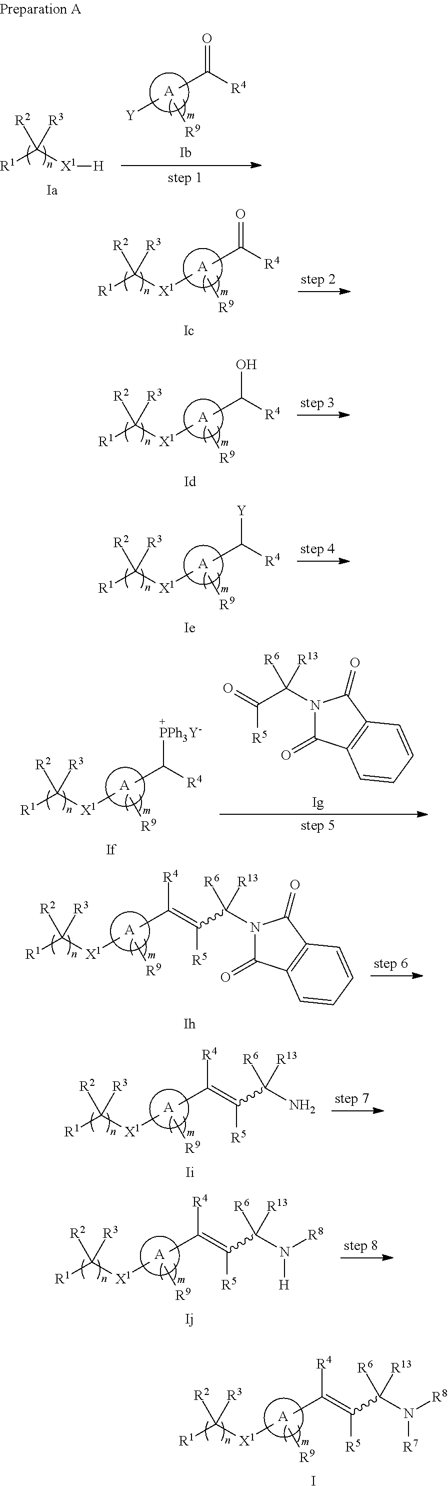

wherein Y is halogen, and other symbols are as defined in the above.

### Step 1

The compound of formula (Ic) can be prepared by reacting the compound of formula (Ia) with the compound of formula (Ib). It can be prepared in the presence of a base or a metal catalyst.

Examples of the metal catalyst include palladium acetate, bis(dibenzylideneacetone)palladium, tetrakis(triphenylphosphine)palladium, bis(triphenylphosphine)palladium(II) dichloride, bis(tri-tert-butylphosphine)palladium and the like. The amount of the metal catalyst is 0.001 to 0.5 mole equivalents to the compound of formula (Ia).

Examples of the base include lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogenphosphate, potassium phosphate, potassium hydrogenphosphate and the like. The amount of the base is 1 to 10 mole equivalent(s) to the compound of formula (Ia).

The temperature for such reaction may be 20° C. to reflux temperature of solvent, and if necessary, by a microwave irradiation.

Reaction time may be conducted for 0.1 to 48 hours and preferably for 0.5 to 12 hours.

Examples of the reaction solvent include tetrahydrofuran, toluene, DMF, dioxane, water and the like, and their mixture can be used as well as the single solvent.

### Step 2

The compound of formula (Id) can be prepared by reacting a reducing agent reagent.

Examples of the reducing agent include are sodium borohydride, lithium borohydride, lithium aluminum hydride and the like. The amount of the reducing agent is 1 to 10 mole equivalent(s) to the compound of formula (Ic).

The temperature for such reaction may be 0° C. to reflux temperature of solvent, preferably 20° C. to reflux temperature of solvent.

Examples of the reaction solvent include methanol, ethanol, propanol, isopropanol, butanol, tetrahydrofuran, diethylether, dichloromethane, water and the like, and their mixture can be used as well as the single solvent.

### Step 3

The compound of formula (Ie) can be prepared by reacting the compound of formula (Id) with a halogenation reagent.

Examples of the halogenation reagent include phosphorus bromide, phosphorus pentabromide, iodine and the like, and the amount of the halogenation reagent is 1 to 10 mole equivalent(s) to the compound of formula (Id).

The temperature for such reaction may be 0° C. to reflux temperature of solvent, and preferably 20° C. to reflux temperature of solvent.

Reaction may be conducted for 0.2 to 48 hours, and preferably for 1 to 24 hour(s).

Examples of the reaction solvent include methanol, ethanol, propanol, isopropanol, butanol, tetrahydrofuran, diethylether, dichloromethane, water and the like, and their mixture can be used as well as the single solvent.

### Step 4

The compound of formula (If) can be prepared by reacting the compound of formula (Ie) with triphenylphosphine, triethylphosphite and the like.

The temperature for such reaction may be 0° C. to reflux temperature of solvent, and preferably 20° C. to reflux temperature of solvent.

Reaction may be conducted for 0.2 to 48 hours, and preferably for 1 to 24 hour(s).

Examples of the reaction solvent include methanol, ethanol, propanol, isopropanol, butanol, tetrahydrofuran, diethylether, dichloromethane, toluene, water and the like, and their mixture can be used as well as the single solvent.

### Step 5

The compound of formula (Ih) can be prepared by reacting the compound of formula (If) with the compound of formula (Ig). It can be prepared in the presence of a base.

Examples of the base are lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogenphosphate, potassium phosphate, potassium hydrogenphosphate and the like. The amount of the base is 1 to 10 mole equivalent(s) to the compound of formula (If).

The temperature for such reaction may be 20° C. to reflux temperature of solvent, and if necessary, by a microwave irradiation.

Reaction may be conducted for 0.1 to 48 hours, and preferably for 0.5 to 12 hours.

Examples of the reaction solvent include tetrahydrofuran, toluene, DMF, dioxane, water and the like, and their mixture can be used as well as the single solvent.

### Step 6

The compound of formula (Ii) can be prepared by reacting the compound of formula (Ih) with the deprotecting reagent.

Examples of the deprotecting reagent are hydrazine, methyl hydrazine and the like. The amount of the deprotecting reagent is 1 to 10 mole equivalent(s) to the compound of formula (Ih).

The temperature for such reaction may be 20° C. to reflux temperature of solvent, and if necessary, by a microwave irradiation.

Reaction may be conducted for 0.1 to 24 hours, and preferably for 1 to 12 hour(s).

Examples of the reaction solvent include acetonitrile, tetrahydrofuran, toluene, DMF, dioxane, methanol, ethanol, water and the like, and their mixture can be used as well as the single solvent.

### Step 7

The compound of formula (Ij) can be prepared by reacting the compound of formula (Ii). The compound introduced R⁸ can be used in some conditions. The compound can be prepared by reacting isocyanate, carboxylic halide, mixed anhydride, by reacting carboxylic acid in the presence of a condensation agent, or by reacting aryl halide or heteroaryl in the presence of a metal catalyst or a base. When introduced R⁸ is aryl or heteroaryl, the reaction can be carried out in the presence of a base or a metal catalyst.

Examples of the condensation agent are dicyclohexylcarbodiimide, carbonyldiimidazole, dicyclohexylcarbodiimide-N-hydroxybenzotriazole, EDC, 4-(4,6-dimethoxy-1,3,5-triazin-2-yl)-4-methylmorpholinium chloride, HATU and the like. The amount of the condensation agent is 1 to 5 mole equivalent(s) to the compound of formula (Ii).

Examples of the metal catalyst include palladium acetate, bis(dibenzylideneacetone)palladium, tetrakis(triphenylphosphine)palladium, bis(triphenylphosphine)palladium(II) dichloride, bis(tri-tert-butylphosphine)palladium and the like. The amount of the metal catalyst is 0.001 to 0.5 mole equivalents to the compound of formula (Ii).

Examples of the base are lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogenphosphate, potassium phosphate, potassium hydrogenphosphate and the like. The amount of the base is 1 to 10 mole equivalent(s) to the compound of formula (Ii).

The temperature for such reaction may be 20° C. to reflux temperature of solvent, and if necessary, by a microwave irradiation.

Reaction may be conducted for 0.1 to 48 hours, and preferably for 0.5 to 12 hours.

Examples of the reaction solvent include tetrahydrofuran, toluene, DMF, dioxane, water and the like, and their mixture can be used as well as the single solvent.

The compound of formula (Ij) is the compound of formula (I) wherein R⁷ is hydrogen, and includes the compounds of the present invention.

### Step 8

The compound of formula (I) can be prepared by reacting the compound of formula (Ij) with the compound of formula R⁷—Y (wherein R⁷ has the same meaning as defined above, Y is halogen.). The reaction can be carried out in the presence of a base.

Examples of the base are lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogenphosphate, potassium phosphate, potassium hydrogenphosphate and the like. The amount of the base is 1 to 10 mole equivalent(s) to the compound of formula (Ij).

Examples of the compound of formula R⁷—Y (wherein R⁷ has the same meaning as defined above, Y is halogen.) are examples of alkylation agent. Examples of the alkylation agent are methyl iodide, ethyl iodide and the like. The amount of the alkylation agent is 1 to 10 mole equivalent(s) to the compound of formula (Ij).

The temperature for such reaction may be 20° C. to reflux temperature of solvent, and if necessary, by a microwave irradiation.

Reaction may be conducted for 0.1 to 48 hours, and preferably for 0.5 to 12 hours.

Examples of the reaction solvent include acetonitrile, tetrahydrofuran, toluene, DMF, dioxane, water and the like, and their mixture can be used as well as the single solvent.

When the compound of formula (I′) is the compound of formula (I-D) wherein R⁴ and R⁵ is hydrogen atom, the compound can be prepared by the following Preparation B.

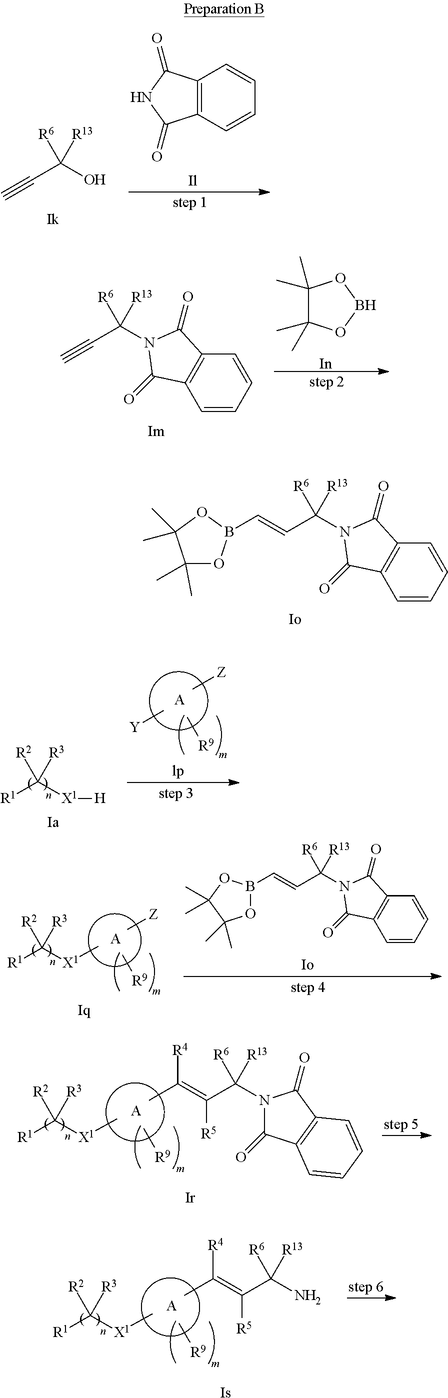

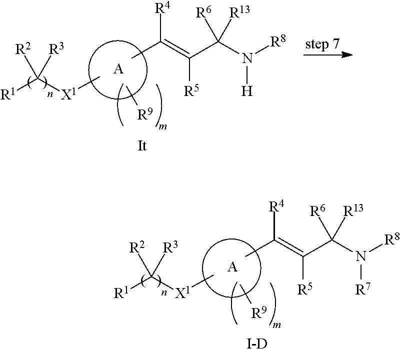

Wherein Y is halogen, Z is halogen, —O-Tf and the like, Tf is trifluoromethanesulfonyl, and other symbols are as defined in the above.

### Step 1

The compound of formula (Im) can be prepared by reacting the compound of formula (Ik) with the compound of formula (Il). The reaction can be carried out in the presence of triphenylphosphine and a condensation agent.

Examples of the condensation agent are DEAD, DIAD and the like. The amount of the condensation agent is 1 to 5 mole equivalent(s) to the compound of formula (Ik).

The temperature for such reaction may be 0° C. to 60° C., preferably 10° C. to 40° C.

Reaction may be conducted for 0.1 to 12 hours, and preferably for 0.2 to 6 hours.

Examples of the reaction solvent include tetrahydrofuran, dioxane, ethyl acetate, toluene, acetonitrile and the like, and their mixture can be used as well as the single solvent.

Step 2

The compound of formula (Io) can be prepared by reacting the compound of formula (Im) with the compound of formula (In). The reaction can be carried out in the presence of a base or a metal catalyst.

Examples of the metal catalyst are palladium acetate, bis(dibenzylideneacetone)palladium, tetrakis(triphenylphosphine)palladium, bis(triphenylphosphine)palladium(II) dichloride, bis(tri-tert-butylphosphine)palladium, bis(cyclopentadienyl)zirconium chloride hydride and the like. The amount of the metal catalyst is 0.001 to 0.5 mole equivalents to the compound of formula (Im).

Examples of the base are triethylamine, diisopropylethylamine, DBU, lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogenphosphate, potassium phosphate, potassium hydrogenphosphate and the like. The amount of the base is 1 to 10 mole equivalent(s) to the compound of formula (Im).

The temperature for such reaction may be 20° C. to reflux temperature of solvent, and if necessary, by a microwave irradiation.

Reaction may be conducted for 0.1 to 48 hours, and preferably for 0.5 to 12 hours.

Examples of the reaction solvent include tetrahydrofuran, toluene, DMF, dioxane, water and the like, and their mixture can be used as well as the single solvent.

### Step 3

The compound of formula (Iq) can be prepared by reacting the compound of formula (Ia) with the compound of formula (Ip). The reaction can be carried out in the presence of a base or a metal catalyst.

Examples of the metal catalyst are palladium acetate, bis(dibenzylideneacetone)palladium, tetrakis(triphenylphosphine)palladium, bis(triphenylphosphine)palladium(II) dichloride, bis(tri-tert-butylphosphine)palladium and the like. The amount of the metal catalyst is 0.001 to 0.5 mole equivalents to the compound of formula (Ia).

Examples of the base are lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogenphosphate, potassium phosphate, potassium hydrogenphosphate and the like. The amount of the base is 1 to 10 mole equivalent(s) to the compound of formula (Ia).

The temperature for such reaction may be 20° C. to reflux temperature of solvent, and if necessary, by a microwave irradiation.

Reaction may be conducted for 0.1 to 48 hours, and preferably for 0.5 to 12 hours.

Examples of the reaction solvent include tetrahydrofuran, toluene, DMF, dioxane, water and the like, and their mixture can be used as well as the single solvent.

### Step 4

The compound of formula (Ir) can be prepared by reacting the compound of formula (Iq) with the compound of formula (Io). The reaction can be carried out in the presence of a base or a metal catalyst.

Examples of the metal catalyst are palladium acetate, bis(dibenzylideneacetone)palladium, tetrakis(triphenylphosphine)palladium, bis(triphenylphosphine)palladium(II) dichloride, bis(tri-tert-butylphosphine)palladium, bis(cyclopentadienyl)zirconium chloride hydride and the like. The amount of the metal catalyst is 0.001 to 0.5 mole equivalents to the compound of formula (Iq).

Examples of the base are triethylamine, diisopropylethylamine, lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogenphosphate, potassium phosphate, potassium hydrogenphosphate and the like. The amount of the base is 1 to 10 mole equivalent(s) to the compound of formula (Iq).

The temperature for such reaction may be 20° C. to reflux temperature of solvent, and if necessary, by a microwave irradiation.

Reaction may be conducted for 0.1 to 48 hours, and preferably for 0.5 to 12 hours.

Examples of the reaction solvent include tetrahydrofuran, toluene, DMF, dioxane, water and the like, and their mixture can be used as well as the single solvent.

### Step 5

The of formula compound of formula (Is) can be prepared by reacting the of formula compound of formula (Ir) with deprotecting reagent.

The step can be carried out by a similar method as the above Step 6 in Step A.

### Step 6

The of formula compound of formula (It) can be prepared by reacting the of formula compound of formula (Is).

The step can be carried out by a similar method as the above Step 7 in Step A. The compound of formula of formula (It) is the compound of formula of formula (I-D) wherein R⁷ is hydrogen, and includes the compounds of the present invention.

### Step 7

The compound of formula of formula (I-D) can be prepared by reacting the compound of formula (It) with the compound of formula R⁷—Y (wherein R⁷ has the same meaning as defined above, Y is halogen.).

The step can be carried out by a similar method as the above Step 8 in Step A.

When the compound of formula (I′) is the compound of formula (II), the compound can be prepared by the following Preparation C.

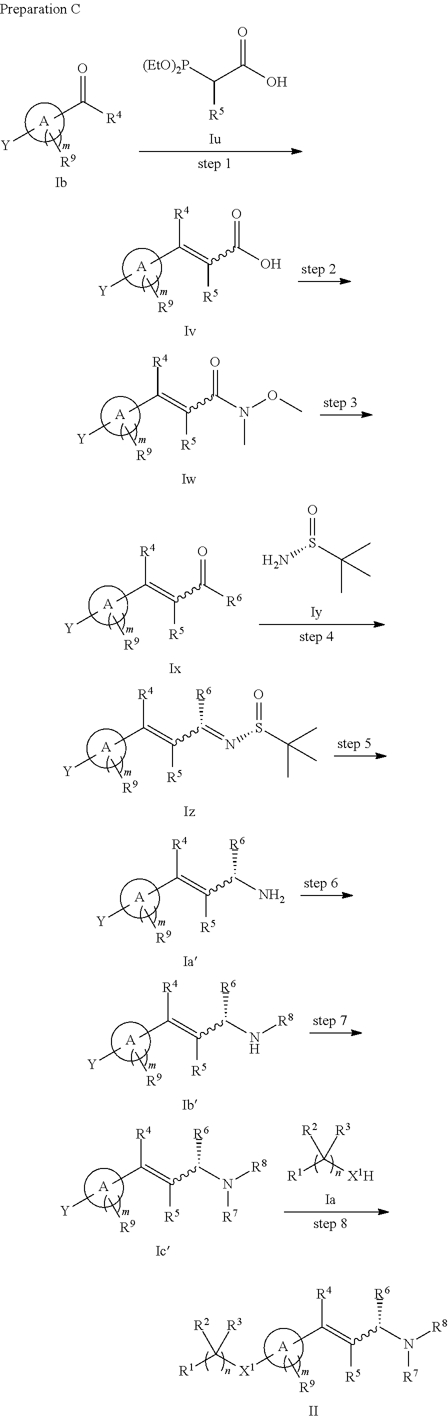

Wherein Y is halogen, —O-Tf or —O-Nf, Tf is trifluoromethanesulfonyl, Nf is nitrobenzenesulfonyl, and other symbols are as defined in the above.

### Step 1

The compound of formula (Iv) can be prepared by reacting the compound of formula (Ib) with the compound of formula (Iu). The reaction can be carried out in the presence of a base.

Examples of the base are triethylamine, diisopropylethylamine, lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogenphosphate, potassium phosphate, potassium hydrogenphosphate, Grignard reagent and the like, preferably isopropylmagnesiumbromide. The amount of the base is 1 to 10 mole equivalent(s) to the compound of formula (Ib).

The temperature for such reaction may be 0° C. to 60° C., preferably 10° C. to 40° C.

Reaction may be conducted for 0.1 to 12 hours, and preferably for 0.2 to 6 hours.

Examples of the reaction solvent include tetrahydrofuran, dioxane, ethyl acetate, toluene, acetnitrile and the like, and their mixture can be used as well as the single solvent.

### Step 2

The compound of formula (Iw) can be prepared by reacting the compound of formula (Iv) with N, O-dimethylhydroxylamine. The reaction can be carried out in the presence of a condensation agent.

Examples of the condensation agent are dicyclohexylcarbodiimide, carbonyldiimidazole, dicyclohexylcarbodiimide-N-hydroxybenzotriazole, EDC, 4-(4,6-dimethoxy-1,3,5-triazin-2-yl)-4-methylmorpholinium chloride, HATU and the like. The amount of the condensation agent is 1 to 5 mole equivalent(s) to the compound of formula (Iv).

The temperature for such reaction may be 0° C. to 60° C., preferably 0° C. to 40° C.

Reaction may be conducted for 0.1 to 12 hours, and preferably for 0.2 to 6 hours.

Examples of the reaction solvent include DMF, NMP, tetrahydrofuran, dioxane, ethyl acetate, dichloromethane, acetnitrile and the like, and their mixture can be used as well as the single solvent.

### Step 3

The compound of formula (Ix) can be prepared by reacting the compound of formula (Iw) with nucleophile.

Examples of the nucleophile are lithium reagents such as methyllithium, ethyllithium and the like, Grignard reagent such as methylmagnesium bromide, methylmagnesium chlorode, methylmagnesium iodide, ethylmagnesium bromide, ethymagnesium chlorode, ethylmagnesium iodide and the like, and their mixture reagent of them and metal salt. The amount of the nucleophile is 1 to 5 mole equivalent(s) to the compound of formula (Iw).

The temperature for such reaction may be −78° C. to reflux temperature of solvent, preferably −45° C. to 0° C.

Reaction may be conducted for 0.5 to 24 hours, and preferably for 1 to 6 hour(s).

Examples of the reaction solvent include tetrahydrofuran, hexane, diethyleter, methyl tert-butylether, toluene, dichloromethane and the like, and their mixture can be used as well as the single solvent.

### Step 4

The compound of formula (Iz) can be prepared by reacting the compound of formula (Ix) with the compound of formula (Iy). The reaction can be carried out in the presence of a Lewis acid and a reducing agent.

Examples of the Lewis acid are iodo trimethylsilane, BBr₃, AlCl₃, BF₃-(Et₂O), TiCl₄, Ti(O-iPr)₄ and the like. Preferred is Ti(O-iPr)₄. The amount of the Lewis acid is 1 to 10 mole equivalent(s) to the compound of formula (Ix).

Examples of the reducing agent are sodium boronhydride, lithium borohydride, lithium aluminum hydride, diisobutylaluminum hydride and the like. The amount of the reducing agent is 1 to 10 mole equivalent(s) to the compound of formula (Ix).

The temperature for such reaction may be −78° C. to reflux temperature of solvent.

Reaction may be conducted for 0.5 to 48 hours, and preferably for 1 to 8 hour(s).

Examples of the reaction solvent include tetrahydrofuran, dioxane, toluene, dichloromethane, chloroform and the like, and their mixture can be used as well as the single solvent.

### Step 5

The compound of formula (Ia′) can be prepared by reacting the compound of formula (Iz) with an acid.

Examples of the acid are hydrochloric acid-ethyl acetate, hydrochloric acid-methanol, hydrochloric acid-dioxane, sulfuric acid, formic acid, trifluoroacetic acid and the like. Examples of the Lewis acid are iodo trimethylsilane, BBr₃, AlCl₃, BF₃-(Et₂O) and the like. The amount of the acid is 1 to 10 mole equivalent(s) to the compound of formula (Iz).

The temperature for such reaction may be 0° C. to 60° C., and preferably 0° C. to 20° C.

Reaction may be conducted for 0.5 to 12 hours, and preferably for 1 to 6 hour(s).

Examples of the reaction solvent include methanol, ethanol, water, acetone, ace tonitrile, DMF and the like, and their mixture can be used as well as the single solvent.

### Step 6

The compound of formula (Ib′) can be prepared by reacting the compound of formula (Ia′).

The step can be carried out by a similar method as the above Step 7 in Step A.

The compound of formula (Ib′) is the compound of formula (I) wherein R⁷ is hydrogen, and includes the compounds of the present invention.

### Step 7

The compound of formula (Ic′) can be prepared by reacting the compound of formula (Ib′) with the compound of formula R⁷—Y (wherein R⁷ has the same meaning as defined above, Y is halogen.)

The step can be carried out by a similar method as the above Step 8 in Step A.

### Step 8

The compound of formula (II) can be prepared by reacting the compound of formula (Ic′) with the compound of formula (Ia). The reaction can be carried out in the presence of a base or a metal catalyst.

Examples of the metal catalyst are copper iodide, copper chloride, copper bromide, palladium acetate, bis(dibenzylideneacetone)palladium, tetrakis(triphenylphosphine)palladium, bis(triphenylphosphine)palladium(II) dichloride, bis(tri-tert-butylphosphine)palladium, bis(cyclopentadienyl)zirconium chloride hydride and the like. Preferred is copper iodide, and the amount of the metal catalyst is 0.001 to 0.5 mole equivalents to the compound of formula (Ic′).

Examples of the ligand are glycine, methylglycine, dimethylglycine, glycine ester derivatives, methylglycine ester derivatives, dimethylglycine ester derivatives and the like. Preferred is dimethylglycine, and the amount of the ligand is 1 to 10 mole equivalent(s) to the compound of formula (Ic′).

Examples of the base are triethylamine, diisopropylethylamine, lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogenphosphate, potassium phosphate, potassium hydrogenphosphate and the like. The amount of the base is 1 to 10 mole equivalent(s) to the compound of formula (Ic′).

The temperature for such reaction may be 20° C. to reflux temperature of solvent, and if necessary, by a microwave irradiation.

Reaction may be conducted for 0.1 to 48 hours, and preferably for 0.5 to 12 hours.

Examples of the reaction solvent include tetrahydrofuran, toluene, DMF, dioxane, water and the like, and their mixture can be used as well as the single solvent.

When the compound of formula (I′) is the compound wherein X⁵ is not bond, the compound can be prepared according to the method described in Example 521.

The compound of this invention has ACC2 antagonistic activity. A pharmaceutical composition comprising the compound of this invention is very useful for preventing or treating a disease associated with ACC2. Examples of the diseases associated with ACC2 means a disease induced by malonyl-CoA produced by ACC2 are metabolic syndrome, obesity, diabetes, insulin resistance, abnormal glucose tolerance, diabetic peripheral neuropathy, diabetic nephropathy, diabetic retinal disease, diabetic macroangiopathy, hyperlipidemia, hypertension, cardiovascular illness, arterial sclerosis, atherosclerotic cardiovascular disease, cardiac arrest, cardiac infarction, infectious disease, neoplasm and the like. A pharmaceutical composition comprising the compound of this invention is very useful as a medicine for preventing or treating the disedases.

Furthermore, a compound of this invention has not only ACC2 antagonistic activity but also usefulness as a medicine and any or all good characters selected from the followings:

a) weak CYP (e.g., CYP1A2, CYP2C9, CYP2C19, CYP2D6, CYP3A4 and the like) enzyme inhibition.
b) good drug disposition such as high bioavailability, appropriate clearance and the like.
c) high metabolic stability.
d) no irreversible CYP (e.g., CYP3A4) enzyme inhibition in the range of the concentration as a measuring condition described in the specification.
e) no mutagenicity
f) low cardiovascular risk.
g) high water solubility.

The pharmaceutical composition of the invention can be administered orally or parenterally as an anti-obesity agent or anorectic agent. In the case of oral administration, it may be in any usual form such as tablets, granules, powders, capsules and the like. When the compound is parenterally administered, any usual form is preferable injections and the like. Oral administration is especially preferable because the compounds of this invention show a high oral absorbability.

The pharmaceutical composition may be manufactured by mixing an effective amount of the compound of the invention with various pharmaceutical additives suitable for the administration form, such as excipients, binders, moistening agents, disintegrants, lubricants and the like

Although the dosage of the pharmaceutical composition of the invention as an anti-obesity agent or anorectic agent should be determined in consideration of the patient's age and body weight, the type and degree of diseases, the administration route and the like, a usual oral dosage for an adult is 0.05 to 100 mg/kg/day and preferable is 0.1 to 10 mg/kg/day. For parenteral administration, although the dosage highly varies with administration routes, a usual dosage is 0.005 to 10 mg/kg/day and preferably 0.01 to 1 mg/kg/day. The dosage may be administered in one to several divisions per day.

### EXAMPLES

This invention is further explained by the following Examples, which are not intended to limit the scope of this invention.

The abbreviations used in the present description stand for the following meanings.

Ac: acetyl
acac: acetyl acetone
BINAP: 2,2′-bis(diphenylphosphino)-1,1′-binaphthyl
Boc: tert-butoxycarbonyl
Boc₂O: Di-t-butyl dicarbonate
Bu: butyl
CDI: Carbonyl diimidazole
dba: dibenzylideneacetone
DEAD: diethyl azodicarboxylate
DIAD: diisopropyl azodicarboxylate
DIPEA: diisopropylethylamine
DMAP: 4-dimethyl aminopyridine

### DMF: N,N-dimethylformamide

WSCD: 1-ethyl-3-(3-dimethylaminopropyl) carbodiimide
Et: ethyl
HATU: O-(7-Azabenzotriazol-1-yl)-1,1,3,3-tetramethyl uronium hexafluorophosphate
mCPBA: m-chloroperoxybenzoic acid
Me: methyl
MEK: methylethylketone

### NBS: N-bromosuccinimide

Pd2(dba)3: Tris(dibenzylideneacetone) bispalladium
Ph: phenyl
SEM: 2-(trimethylsilyl)ethoxymethyl
TBAF: tetrabutylammonium fluoride
TBS: tert-butyldimethylsilyl
TESH: triethylsylane
Tf: trifluoromethanesulfonyl
TFA: trifluoroacetic acid
THF: tetrahydrofuran
TIPSCI: triisopropylsilyl chloride

¹H NMR spectra of the examples were measured on 300 MHz in d₆-DMSO or CDCl₃.

“RT” in the reference examples and examples or tables represents “Retention Time” by LC/MS: Liquid Chromatography/Mass Spectrometry.

LC/MS data of the compounds were measured under the following condition.

### Method 1: Column: Gemini-NX (5 μm, i.d.4.6×50 mm) (Phenomenex)

Flow rate: 3 mL/min
UV detection wavelength: 254 nm
Mobile phase: [A] is 0.1% formic acid-containing aqueous solution, and [B] is 0.1% formic acid-containing methanol solution.
Gradient: Linear gradient of 5% to 100% solvent [B] for 3.5 minutes was performed, and 100% solvent [B] was maintained for 0.5 minutes.

### Method 2: Column: Shim-pack XR-ODS (2.2 μm, i.d.50×3.0 mm) (Shimadzu)

Flow rate: 1.6 mL/min
UV detection wavelength: 254 nm
Mobile phase: [A] is 0.1% formic acid-containing aqueous solution, and [B] is 0.1% formic acid-containing acetonitrile solution.
Gradient: Linear gradient of 10% to 100% solvent [B] for 3 minutes was performed, and 100% solvent [B] was maintained for 1 minute.
Method 3: Column: ACQUITY UPLC(R) BEH C18 (1.7 μm,i.d.2.1×50 mm) (Waters) Flow rate: 0.8 mL/min UV detection wavelength: 254 nm Mobile phase: [A] is 0.1% formic acid-containing aqueous solution, and [B] is 0.1% formic acid-containing acetonitrile solution.
Gradient: Linear gradient of 10% to 100% solvent [B] for 3 minutes was performed, and 100% solvent [B] was maintained for 0.5 minutes.

### Reference Example 001

### Preparation of Compound 2

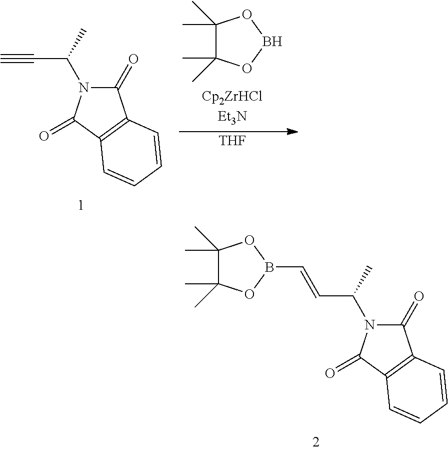

To Compound 1 (8.00 g, 40.2 mmol, described in US2006/0178400) and 4,4,5,5-tetramethyl-1,3,2-dioxabololane (7.21 mL, 48.2 mmol), bis (cyclopentadienyl) zirconium (IV) chloride hydride (1.04 g, 4.02 mmol) and triethylamine (0.557 mL, 4.02 mmol) were added. The mixture was dissolved in tetrahydrofuran (8 mL), stirred at 65° C. for 24 hours. The solvent was distilled under reduced pressure, and the residue was purified by silica gel chromatography (hexane-ethyl acetate) to afford Compound 2 (9.00 g, yield 69%).

¹H-NMR (CDCl₃) δ: 7.84-7.78 (m, 2H), 7.72-7.66 (m, 2H), 6.78 (dd, J=18.0, 5.0 Hz, 1H), 5.51 (dd, J=18.1, 1.8 Hz, 1H), 5.03-4.94 (m, 1H), 1.62 (d, J=7.3 Hz, 3H), 1.24 (s, 2H).

### Reference Example 002

### Preparation of Compound 6

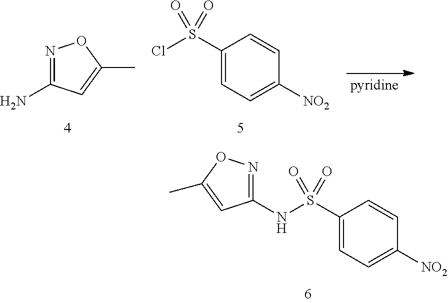

To pyridine (15 mL, 185 mmol) solution of Compound 4 (3.0 g, 30.6 mmol), Compound 5 (7.45 g, 33.6 mmol) was added, the mixture was stirred at room temperature for 5 hours. 2 mol/L hydrochloric acid (100 mL) was added to the mixture, the precipitated crystal was filtered off and dried at 60° C. under vacuum to afford Compound 6 (8.37 g, yield 97%).

¹H-NMR (DMSO-d₆) δ: 8.42 (d, J=8.5 Hz, 2H), 8.10 (d, J=8.4 Hz, 2H), 6.16 (s, 1H), 2.31 (s, 3H).

### Reference Example 003

### Preparation of Compound 10

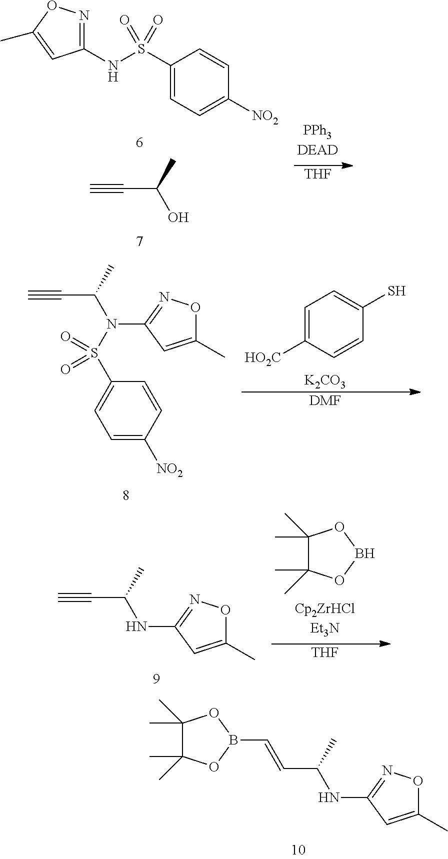

### Step 1 Preparation of Compound 8

Diethyl azodicarboxylate (2.2 mol/L toluene solution, 7.22 mL, 15.89 mmol) was added dropwise to the tetrahydrofuran solution (30 mL) of Compound 7 (1.021 mL, 12.71 mmol), Compound 6 (3.0 g, 10.59 mmol) and triphenylphosphine (4.17 g, 15.89 mmol) under a nitrogen atmosphere while cooling in ice. After the completion of addition dropwise, the mixture was stirred overnight at room temperature. The solvent was distilled under reduced pressure. Ethanol was added to the residue. The precipitated solids were filtered, washed with ethanol, and subsequently dried at 60° C. under vacuum to obtain compound 8 (2.64 g, yield 74%).

¹H-NMR (DMSO-d₆) δ: 8.44 (d, J=8.8 Hz, 2H), 8.15 (d, J=8.8 Hz, 2H), 6.40 (s, 1H), 5.32-5.23 (m, 1H), 2.44 (s, 3H), 1.35 (d, J=7.0 Hz, 3H).

### Step 2 Preparation of Compound 9

To DMF solution of the compound 8,4-mercapto benzoic acid (920 mg, 5.96 mmol) and potassium carbonate (1.65 g, 11.93 mmol). The mixture was stirred at 40° C. for 4 hours. Water was added to the reaction solution, and the mixture was extracted with ethyl acetate. The organic layer was washed with saturated brine, dried over magnesium sulfate and condensed under reduced pressure. The residue was purified by silica gel chromatography (hexane-ethyl acetate) to afford Compound 9 (386 mg, yield 86%).

¹H-NMR (CDCl₃) δ: 5.55 (t, J=0.7 Hz, 1H), 4.39-4.27 (m, 1H), 3.94 (br s, 1H), 2.30-2.28 (m, 4H), 1.52 (d, J=6.9 Hz, 3H).

### Step 3 Preparation of Compound 10

To THF suspension of the compound 9 (300 mg, 1.998 mmol), 4,4,5,5-tetramethyl-1,3,2-dioxaborolane (0.359 mL, 2.40 mmol), bis(cyclopentadienyl)zirconium(IV) chloride hydride (51.5 mg, 0.200 mmol) and triethylamine (0.028 mL, 0.200 mmol) was stirred at 60° C. for 3 hours. The mixture was purified by silica gel chromatography (hexane-ethyl acetate) to afford Compound 10 (459 mg, yield 83%).

¹H-NMR (CDCl₃) δ: 6.60 (dd, J=18.1, 4.9 Hz, 1H), 5.58 (dd, J=18.1, 1.6 Hz, 1H), 5.46 (d, J=0.8 Hz, 1H), 4.16-4.08 (m, 1H), 3.83-3.76 (m, 1H), 2.27 (s, 3H), 1.36-1.23 (m, 15H).

### Reference Example 004

### Preparation of Compound 11

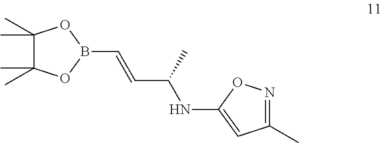

Compound 11 was obtained by using the compound obtained by the using 3-methylisoxazolyl-5-amine instead of Compound 4 in the above Reference example 002 instead of Compound 6 in Step 1 in the above Reference example 003.

¹H-NMR (CDCl₃) δ: 6.51 (dd, J=18.1, 5.3 Hz, 1H), 5.58 (dd, J=18.1, 1.4 Hz, 1H), 4.78 (s, 1H), 4.39 (d, J=6.9 Hz, 1H), 3.97-3.87 (m, 1H), 2.14 (s, 3H), 1.33 (d, J=6.7 Hz, 3H), 1.27 (s, 12H).

### Reference Example 005

### Preparation of Compound 16

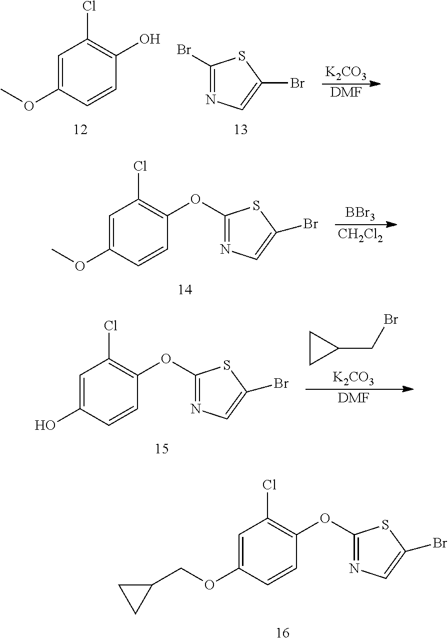

### Step 1 Preparation of Compound 14

To DMF solution of the compound 12 (7.63 g, 31.4 mmol) and compound 13 (5.98 g, 37.7 mmol), potassium carbonate (5.21 g, 37.7 mmol) was added. The mixture was stirred at 120° C. for 6 hours. Water was added to the reaction mixture, the mixture was extracted with diethylether. The organic layer was washed with saturated brine, dried over magnesium sulfate. The solvent was condensed under reduced pressure. The residue was purified by silica gel chromatography (hexane-ethyl acetate) to afford Compound 14 (9.42 g, yield 94%).

¹H-NMR (CDCl₃) δ: 7.23 (d, J=8.9 Hz, 1H), 7.11 (s, 1H), 7.00 (d, J=3.0 Hz, 1H), 6.84 (dd, J=8.9, 3.0 Hz, 1H), 3.81 (s, 3H).

### Step 2 Preparation of Compound 15

The dichloromethane solution of Compound 14 (10.68 g, 33.3 mmol) was cooled with dry ice-acetone at −78° C. in a nitrogen atmosphere. 1.0 mol/L boron tribromide (100 mL, 100 mmol) was added dropwise to the mixture, and the mixture was warmed to room temperature for 3 hours after completion of adding dropwise. The reaction mixture was added to saturated sodium bicarbonate water, and stirred. The mixture was extracted with ethyl acetate. The organic layer was washed with saturated brine, and dried over magnesium sulfate. The solvent was condensed under reduced pressure to afford Compound 15 (10.21 g, yield 100%).

¹H-NMR (DMSO-d₆) δ: 10.17 (s, 1H), 7.40 (s, 1H), 7.37 (d, J=8.9 Hz, 1H), 6.97 (d, J=2.9 Hz, 1H), 6.82 (dd, J=8.9, 2.9 Hz, 1H).

### Step 3 Preparation of Compound 16

Potassium carbonate (4.06 g, 29.4 mmol) and (bromomethyl)cyclopropane (28.7 mL, 29.4 mmol) were added to the DMF solution (15 ml) of Compound 15 (6.0 g, 19.57 mmol), and the mixture was stirred at 80° C. for 7 hours. Water was added to the mixture, and the mixture was extracted with diethylether. The organic layer was washed with saturated brine, dried over magnesium sulfate. The solvent was condensed under reduced pressure. The residue was purified by silica gel chromatography (hexane-ethyl acetate) to afford Compound 16 (6.74 g, yield 96%).

¹H-NMR (CDCl₃) δ: 7.22 (d, J=9.0 Hz, 1H), 7.11 (s, 1H), 6.99 (d, J=2.9 Hz, 1H), 6.84 (dd, J=9.0, 2.9 Hz, 1H), 3.78 (d, J=7.0 Hz, 2H), 1.33-1.20 (m, 1H), 0.70-0.63 (m, 2H), 0.38-0.32 (m, 2H).

### Reference Example 006

### Preparation of Compound 17

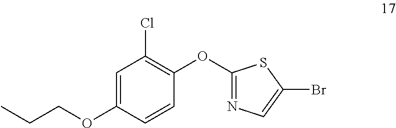 [SMILES: CCCOc1ccc(Oc2ncc(Br)s2)c(Cl)c1]

Compound 17 was obtained by using 1-bromopropane instead of (bromomethyl)cyclopropane in Step 3 in Reference example 005.

¹H-NMR (CDCl₃) δ: 7.21 (d, J=9.0 Hz, 1H), 7.11 (s, 1H), 6.99 (d, J=2.9 Hz, 1H), 6.83 (dd, J=9.0, 2.9 Hz, 1H), 3.90 (t, J=6.5 Hz, 2H), 1.79-1.83 (m, 2H), 1.03 (t, J=7.4 Hz, 3H)

### Reference Example 007

### Preparation of Compound 18

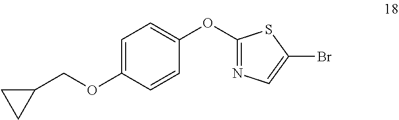 [SMILES: Brc1cnc(Oc2ccc(OCC3CC3)cc2)s1]

Compound 18 was obtained by using 4-methoxyphenol instead of Compound 12 in Step 1 in Reference example 005.

¹H-NMR (DMSO-d₆) δ: 7.39 (s, 1H), 7.28 (d, J=8.8 Hz, 2H), 7.00 (d, J=8.8 Hz, 2H), 3.82 (d, J=6.8 Hz, 2H), 1.19-1.23 (m, 1H), 0.53-0.60 (m, 2H), 0.30-0.34 (m, 2H).

### Reference Example 008

### Preparation of Compound 19

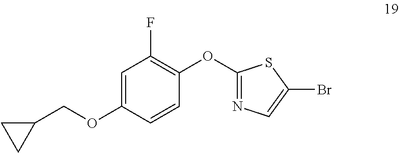 [SMILES: Fc1cc(OCC2CC2)ccc1Oc1ncc(Br)s1]

Compound 19 was obtained by using 2-fluoro-4-methoxyphenol instead of Compound 12 in Step 1 in Reference example 005.

¹H-NMR (DMSO-d₆) δ: 7.43 (t, J=8.8 Hz, 1H), 7.37 (s, 1H), 7.05 (d, J=12.4 Hz, 1H), 6.83 (d, J=8.8 Hz, 1H), 3.84 (d, J=7.1 Hz, 2H), 1.19-1.24 (m, 1H), 0.55-0.59 (m, 2H), 0.30-0.35 (m, 2H).

### Reference Example 009

### Preparation of Compound 20

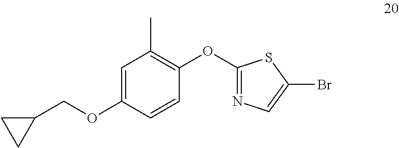 [SMILES: Cc1cc(OCC2CC2)ccc1Oc1ncc(Br)s1]

Compound 20 was obtained by using 4-methoxy-2-methylphenol instead of Compound 12 in Step 1 in Reference example 005.

¹H-NMR (CDCl₃) δ: 7.12 (s, 1H), 7.07 (d, J=8.7 Hz, 1H), 6.80-6.72 (m, 2H), 3.78 (d, J=6.9 Hz, 2H), 2.22 (s, 3H), 1.29-1.22 (m, 1H), 0.68-0.62 (m, 2H), 0.37-0.32 (m, 2H).

### Reference Example 010

### Preparation of Compound 21

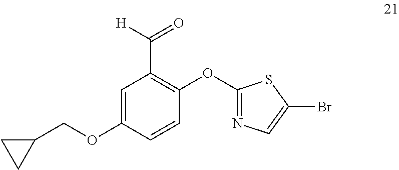 [SMILES: O=Cc1cc(OCC2CC2)ccc1Oc1ncc(Br)s1]

Compound 21 was obtained by using 2-hydroxy-5-methoxybenzaldehyde instead of Compound 12 in Step 1 in Reference example 005.

¹H-NMR (CDCl₃) δ: 10.23 (s, 1H), 7.37 (d, J=2.9 Hz, 1H), 7.28-7.18 (m, 2H), 7.12 (s, 1H), 3.85 (d, J=6.9 Hz, 2H), 1.35-1.22 (m, 1H), 0.70-0.64 (m, 2H), 0.39-0.34 (m, 2H).

### Reference Example 011

### Preparation of Compound 22

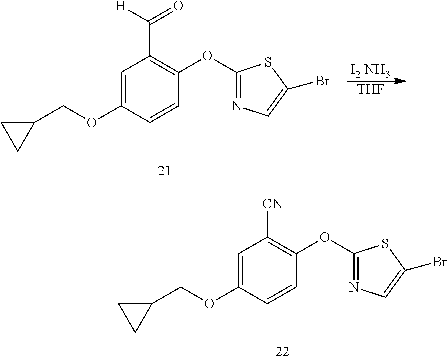

28% ammonium solution (1.2 mL, 15.5 mmol) and iodine (418 mg, 1.65 mmol) were added to the THF solution of Compound 21 (530 mg, 1.50 mmol), the mixture was stirred overnight at room temperature. Water was added to the mixture, and the mixture was extracted with chloroform. The organic layer was dried over magnesium sulfate. The solvent was condensed under reduced pressure. The residue was purified by silica gel chromatography (hexane-ethyl acetate) to afford Compound 22 (323 mg, yield 62%).

¹H-NMR (CDCl₃) δ: 7.35 (d, J=8.7 Hz, 1H), 7.18-7.11 (m, 3H), 3.81 (d, J=7.0 Hz, 2H), 1.33-1.22 (m, 1H), 0.71-0.65 (m, 2H), 0.38-0.33 (m, 2H).

### Reference Example 012

### Preparation of Compound 23

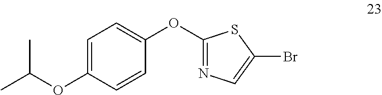 [SMILES: CC(C)Oc1ccc(Oc2ncc(Br)s2)cc1]

Compound 23 was obtained by using 4-isopropoxyphenol instead of Compound 12 in Step 1 in Reference example 005.

¹H-NMR (CDCl₃) δ: 7.18-7.13 (m, 3H), 6.92-6.87 (m, 2H), 4.57-4.45 (m, 1H), 1.34 (d, J=6.0 Hz, 6H).

### Reference Example 013

### Preparation of Compound 24

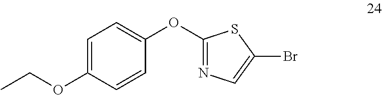 [SMILES: CCOc1ccc(Oc2ncc(Br)s2)cc1]

Compound 24 was obtained by using 4-ethoxyphenol instead of Compound 12 in Step 1 in Reference example 005.

¹H-NMR (CDCl₃) δ: 7.19-7.12 (m, 3H), 6.93-6.88 (m, 2H), 4.02 (q, J=7.0 Hz, 2H), 1.42 (t, J=6.9 Hz, 3H).

### Reference Example 014

### Preparation of Compound 27

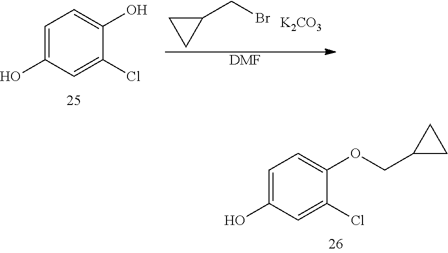

### Step 1 Preparation of Compound 26

Potassium carbonate (4.78 g, 34.6 mmol) and (bromomethyl)cyclopropane (2.03 mL, 20.8 mmol) were added to the DMF solution (10 ml) of Compound 25 (2.0 g, 13.8 mmol), and the mixture was stirred at 80° C. for 8 hours. Water was added to the mixture, and the mixture was extracted with ethyl acetate. The organic layer was washed with saturated brine, dried over magnesium sulfate. The solvent was condensed under reduced pressure. The residue was purified by silica gel chromatography (hexane-ethyl acetate) to afford Compound 26 (470 mg, yield 17%).

¹H-NMR (CDCl₃) δ: 6.89 (d, J=3.0 Hz, 1H), 6.83 (d, J=8.7 Hz, 1H), 6.66 (dd, J=8.8, 2.9 Hz, 1H), 4.56 (s, 1H), 3.81 (d, J=6.7 Hz, 2H), 1.34-1.21 (m, 1H), 0.65-0.59 (m, 2H), 0.37-0.32 (m, 2H).

### Step 2 Preparation of Compound 27

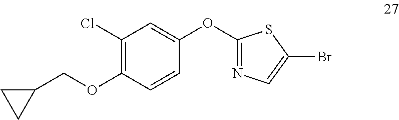 [SMILES: Clc1cc(Oc2ncc(Br)s2)ccc1OCC1CC1]

Compound 27 was obtained by using Compound 26 instead of Compound 12 in Step 1 in Reference example 005.

¹H-NMR (CDCl₃) δ: 7.31 (d, J=2.9 Hz, 1H), 7.14-7.09 (m, 2H), 6.91 (d, J=9.0 Hz, 1H), 3.88 (d, J=6.9 Hz, 2H), 1.37-1.25 (m, 1H), 0.69-0.63 (m, 2H), 0.42-0.36 (m, 2H).

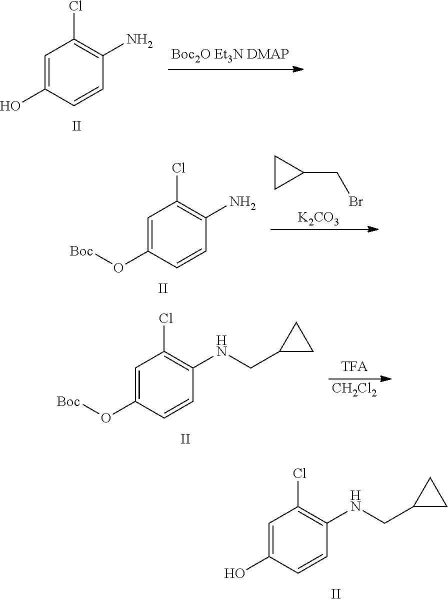

Triethylamine (4.24 mL, 30.6 mmol), Boc₂O (3.56 mL, 15.3 mmol) and DMAP (170 mg, 1.39 mmol) were added to chloroform solution of Compound 28 (2.0 g, 13.9 mmol), the mixture was stirred overnight at room temperature. Water was added to the mixture, and the mixture was extracted with chloroform. The organic layer was dried over magnesium sulfate. The solvent was condensed under reduced pressure. The residue was purified by silica gel chromatography (ethyl acetate-hexane) to afford Compound 29 (2.29 g, yield 68%).

¹H-NMR (DMSO-d₆) δ: 7.08 (s, 1H), 6.84 (m, 2H), 1.46 (s, 9H).

### Reference Example 016

### Preparation of Compound 31

### Step 1 Preparation of Compound 30

Potassium carbonate (851 mg, 6.16 mmol) and (bromomethyl)cyclopropane (0.597 mL, 6.16 mmol) were added to the DMF solution (10 ml) of Compound 29 (1.0 g, 4.10 mmol), and the mixture was stirred at 80° C. for 11 hours. Water was added to the mixture, and the mixture was extracted with ethyl acetate. The organic layer was washed with saturated brine, dried over magnesium sulfate. The solvent was condensed under reduced pressure. The residue was purified by silica gel chromatography (hexane-ethyl acetate) to afford Compound 30 (702 mg, yield 57%).

¹H-NMR (DMSO-d₆) δ: 7.14 (s, 1H), 6.95 (d, J=8.6 Hz, 1H), 6.70 (d, J=8.8 Hz, 1H), 5.12 (s, 1H), 3.00-2.95 (m, 2H), 1.46 (s, 9H), 1.10-1.07 (m, 1H), 0.42-0.47 (m, 2H), 0.21-0.25 (m, 2H).

Trifluoroacetic acid (0.906 mL, 11.75 mmol) was added to the dichloromethane solution of Compound 30 (700 mg, 2.35 mmol), the mixture was stirred at room temperature for 7 hours. The mixture was condensed under reduced pressure, and saturated sodium bicarbonate water was added to the residue. The mixture was extracted with ethyl acetate. The organic layer was washed with saturated brine, and dried over magnesium sulfate. The solvent was condensed under reduced pressure. The residue was purified by silica gel chromatography (hexane-ethyl acetate) to afford Compound 31 (472 mg, yield 100%).

[M+H]=198, Method Condition 3: retention time 1.04 min

### Reference Example 017

### Preparation of Compound 32

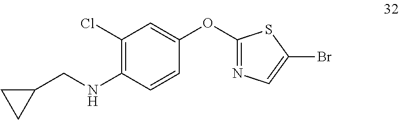 [SMILES: Clc1cc(Oc2ncc(Br)s2)ccc1NCC1CC1]

Compound 32 was obtained by using Compound 31 instead of Compound 12 in Step 1 in Reference example 005.

¹H-NMR (DMSO-d₆) δ: 10.80 (s, 1H), 7.87 (d, J=8.6 Hz, 1H), 7.69 (s, 1H), 7.51 (s, 1H), 7.37 (d, J=8.6 Hz, 1H), 3.88 (s, 2H), 1.52 (m, 1H), 0.90 (d, J=7.3 Hz, 2H), 0.61 (m, 2H).

### Reference Example 018

### Preparation of Compound 35

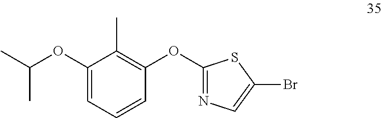 [SMILES: Cc1c(Oc2ncc(Br)s2)cccc1OC(C)C]

Compound 35 was obtained by 2-methyl-3-methoxyphenol instead of Compound 12 in Step 1 in Reference Example 005 and by 2-iodopropane instead of (bromomethyl)cyclopropane iodopropane in Step 3.

¹H-NMR (CDCl₃) δ: 7.19-7.11 (m, 2H), 6.81-6.75 (m, 2H), 4.58-4.50 (m, 1H), 2.10 (s, 3H), 1.35 (d, J=5.9 Hz, 6H).

### Reference Example 019

### Preparation of Compound 36

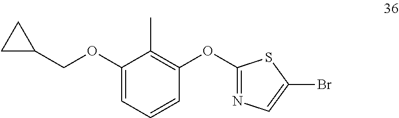 [SMILES: Cc1c(OCC2CC2)cccc1Oc1ncc(Br)s1]

Compound 36 was obtained by using 2-methyl-3-methoxyphenol instead of Compound 12 in Step 1 in Reference example 005.

¹H-NMR (CDCl₃) δ: 7.20-7.13 (m, 2H), 6.80 (d, J=8.2 Hz, 1H), 6.75 (d, J=8.5 Hz, 1H), 3.84 (dd, J=6.6, 1.9 Hz, 2H), 2.15 (s, 3H), 1.32-1.23 (m, 1H), 0.66-0.59 (m, 2H), 0.39-0.33 (m, 2H).

### Reference Example 020

### Preparation of Compound 37

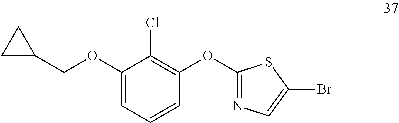 [SMILES: Clc1c(OCC2CC2)cccc1Oc1ncc(Br)s1]

Compound 37 was obtained by using 2-chloro-3-methoxyphenol instead of Compound 12 in Step 1 in Reference example 005.

¹H-NMR (CDCl₃) δ: 7.23 (t, J=8.4 Hz, 1H), 7.12 (s, 1H), 6.94 (dd, J=8.2, 1.2 Hz, 1H), 6.84 (dd, J=8.5, 1.1 Hz, 1H), 3.91 (d, J=6.9 Hz, 2H), 1.31 (m, 1H), 0.69-0.63 (m, 2H), 0.37-0.42 (m, 2H).

### Reference Example 021

### Preparation of Compound 41

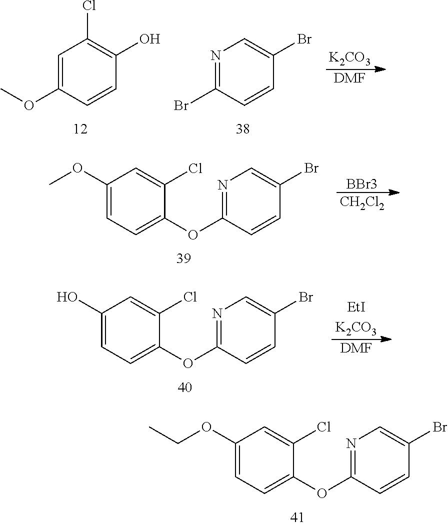

### Step 1 Preparation of Compound 39

Potassium carbonate (6.07 g, 43.9 mmol) was added to DMF solution of Compound 38 (8.00 g, 33.9 mmol) and Compound 12 (6.96 g, 43.9 mmol), and the mixture was stirred at 140° C. for 12 hours. Water was added to the mixture, and the mixture was extracted with diethylether. The organic layer was washed with saturated brine, dried over magnesium sulfate. The solvent was condensed under reduced pressure. The residue was purified by silica gel chromatography (hexane-ethyl acetate) to afford Compound 39 (9.32 g, yield 88%).

¹H-NMR (CDCl₃) δ: 8.15 (d, J=2.4 Hz, 1H), 7.76 (dd, J=8.7, 2.6 Hz, 1H), 7.10 (d, J=8.8 Hz, 1H), 7.00 (d, J=2.9 Hz, 1H), 6.87 (d, J=8.8 Hz, 1H), 6.84 (dd, J=8.9, 3.0 Hz, 1H), 3.81 (s, 3H).

### Step 2 Preparation of Compound 400

The dichloromethane solution of Compound 39 (9.0 g, 28.6 mmol) was cooled with dry ice-acetone at −78° C. in a nitrogen atmosphere. 1.0 mol/L boron tribromide (65 mL, 65.0 mmol) was added dropwise to the mixture, and the mixture was warmed to room temperature for 3 hours after completion of adding dropwise. The reaction mixture was added to saturated sodium bicarbonate water, and stirred. The mixture was extracted with ethyl acetate. The organic layer was washed with saturated brine, and dried over magnesium sulfate. The solvent was condensed under reduced pressure to afford Compound 40 (7.53 g, yield 88%).

¹H-NMR (DMSO-d₆) δ: 9.87 (s, 1H), 8.22 (d, J=2.6 Hz, 1H), 8.03 (dd, J=8.7, 2.6 Hz, 1H), 7.12 (d, J=8.7 Hz, 1H), 7.04 (d, J=8.7 Hz, 1H), 6.90 (d, J=2.7 Hz, 1H), 6.77 (dd, J=8.7, 2.8 Hz, 1H).

### Step 3 Preparation of Compound 41

Potassium carbonate (1.38 g, 9.98 mmol) and iodoethane (0.807 mL, 9.98 mmol) were added to DMF solution of Compound 40 (2.0 g, 6.65 mmol), and the mixture was stirred at 50° C. for 3 hours. Water was added to the mixture, and the mixture was extracted with diethylether. The organic layer was washed with saturated brine, dried over magnesium sulfate. The solvent was condensed under reduced pressure. The residue was purified by silica gel chromatography (hexane-ethyl acetate) to afford Compound 41 (2.05 g, yield 94%).

¹H-NMR (CDCl₃) δ: 8.16 (d, J=2.4 Hz, 1H), 7.76 (dd, J=8.7, 2.4 Hz, 1H), 7.09 (d, J=8.8 Hz, 1H), 6.98 (d, J=2.9 Hz, 1H), 6.86 (d, J=8.7 Hz, 1H), 6.83 (dd, J=8.8, 2.8 Hz, 1H), 4.01 (q, J=7.0 Hz, 2H), 1.42 (t, J=6.9 Hz, 3H).

### Reference Example 022

### Preparation of Compound 42

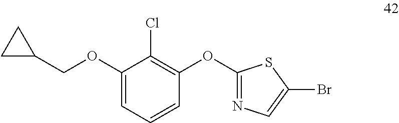 [SMILES: Clc1c(OCC2CC2)cccc1Oc1ncc(Br)s1]

Compound 42 was obtained by using (bromomethyl)cyclopropane instead of iodoethane in Step 3 in Reference example 021.

¹H-NMR (CDCl₃) δ: 7.35 (d, J=8.7 Hz, 1H), 7.18-7.11 (m, 3H), 3.81 (d, J=7.0 Hz, 2H), 1.33-1.22 (m, 1H), 0.71-0.65 (m, 2H), 0.38-0.33 (m, 2H).

### Reference Example 023

### Preparation of Compound 43

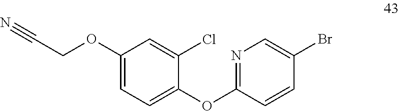 [SMILES: N#CCOc1ccc(Oc2ccc(Br)cn2)c(Cl)c1]

Compound 43 was obtained by using bromoacetonitrile instead of iodoethane in Step 3 in Reference example 021.

¹H-NMR (CDCl₃) δ: 8.14 (dd, J=2.4, 0.6 Hz, 1H), 7.79 (dd, J=8.7, 2.6 Hz, 1H), 7.18 (d, J=9.0 Hz, 1H), 7.11 (d, J=2.9 Hz, 1H), 6.97-6.89 (m, 2H), 4.77 (s, 2H).

### Reference Example 024

### 1 Preparation of Compound 44

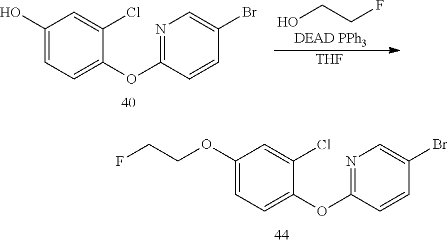

The tetrahydrofuran solution (30 mL) of Compound 40 (500 mg. 1.66 mmol) obtained in Step 2 in Reference Example 021, 2-fluoroethanol (0.145 mL, 2.50 mmol) and triphenylphosphine (655 mg, 2.50 mmol) was cooled with ice in a cool bath in a nitrogen atmosphere. Diethyl azodicarboxylate (2.2 mol/L toluene solution, 1.13 mL, 2.50 mmol) was added dropwise to the mixture. After the completion of addition dropwise, the mixture was stirred overnight at room temperature. The solvent was condensed under reduced pressure. The residue was purified by silica gel chromatography (hexane-ethyl acetate) to afford Compound 44 (479 mg, yield 86%).

¹H-NMR (CDCl₃) δ: 8.15 (d, J=2.6 Hz, 1H), 7.77 (ddd, J=8.7, 2.4, 0.9 Hz, 1H), 7.12 (d, J=8.8 Hz, 1H), 7.03 (d, J=2.9 Hz, 1H), 6.90-6.86 (m, 2H), 4.75 (dt, J=47.3, 4.2 Hz, 2H), 4.21 (dt, J=27.7, 4.2 Hz, 2H).

### Reference Example 025

### Preparation of Compound 45

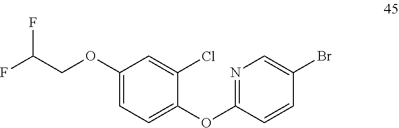 [SMILES: FC(F)COc1ccc(Oc2ccc(Br)cn2)c(Cl)c1]

Compound 45 was obtained by using 2,2-difluoroethanol instead of 2-fluoroethanol in Step 1 in Reference example 024.

¹H-NMR (CDCl₃) δ: 8.14 (d, J=2.4 Hz, 1H), 7.78 (dd, J=8.8, 2.5 Hz, 1H), 7.14 (d, J=8.8 Hz, 1H), 7.04 (d, J=2.9 Hz, 1H), 6.91-6.86 (m, 2H), 6.08 (tt, J=54.9, 4.0 Hz, 1H), 4.18 (td, J=12.9, 4.1 Hz, 2H).

### Reference Example 026

### Preparation of Compound 46

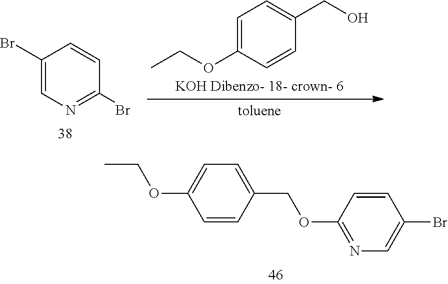 [SMILES: Brc1ccc(Br)nc1 ; CCOc1ccc(CO)cc1 ; CCOc1ccc(COc2ccc(Br)cn2)cc1]

Dibenzo-18-crown-6 (0.152 mg, 11.8 mmol) and potassium hydroxide (1.14 g, 20.3 mmol) were added to the toluene solution (30 mL) of Compound 38 (2.0 g, 8.44 mmol) and 4-ethoxybenzylalcohol (1.80 g, 11.8 mmol), and the mixture was stirred at 120° C. for 2 hours. Water was added to the mixture, and the mixture was extracted with chloroform. The organic layer was dried over magnesium sulfate. The solvent was condensed under reduced pressure. The residue was purified by silica gel chromatography (ethyl acetate-hexane) to afford Compound 46 (2.42 g, yield 93%).

¹H-NMR (CDCl₃) δ: 8.20 (d, J=2.4 Hz, 1H), 7.62 (dd, J=8.8, 2.5 Hz, 1H), 7.38-7.32 (m, 2H), 6.91-6.86 (m, 2H), 6.68 (d, J=8.7 Hz, 1H), 5.25 (s, 2H), 4.03 (q, J=7.0 Hz, 2H), 1.41 (t, J=7.0 Hz, 3H).

### Reference Example 027

### Preparation of Compound 47

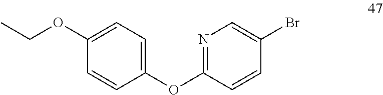 [SMILES: CCOc1ccc(Oc2ccc(Br)cn2)cc1]

Compound 47 was obtained by using 4-ethoxyphenol instead of Compound 12 in Step 1 in Reference example 021.

¹H-NMR (CDCl₃) δ: 8.20 (d, J=2.5 Hz, 1H), 7.73 (ddd, J=8.7, 2.6, 0.6 Hz, 1H), 7.06-7.00 (m, 2H), 6.93-6.88 (m, 2H), 6.78 (d, J=8.7 Hz, 1H), 4.02 (q, J=7.0 Hz, 2H), 1.42 (t, J=7.0 Hz, 3H).

### Reference Example 028

### Preparation of Compound 48

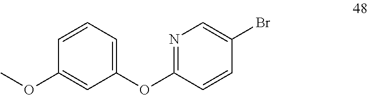 [SMILES: COc1cccc(Oc2ccc(Br)cn2)c1]

Compound 48 was obtained by using 3-methoxyphenol instead of Compound 12 in Step 1 in Reference example 021.

¹H-NMR (CDCl₃) δ: 8.24 (d, J=2.5 Hz, 1H), 7.76 (dd, J=8.6, 2.6 Hz, 1H), 7.29 (t, J=8.1 Hz, 1H), 6.84-6.67 (m, 4H), 3.80 (s, 3H).

### Reference Example 029

### Preparation of Compound 49

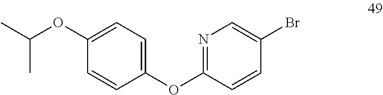 [SMILES: CC(C)Oc1ccc(Oc2ccc(Br)cn2)cc1]

Compound 49 was obtained by using 4-isopropoxyphenol instead of Compound 12 in Step 1 in Reference example 021.

¹H-NMR (CDCl₃) δ: 8.20 (d, J=2.4 Hz, 1H), 7.72 (dd, J=8.7, 2.6 Hz, 1H), 7.04-6.99 (m, 2H), 6.92-6.86 (m, 2H), 6.78 (d, J=8.7 Hz, 1H), 4.56-4.44 (m, 1H), 1.34 (d, J=5.9 Hz, 6H).

### Reference Example 030

### Preparation of Compound 50

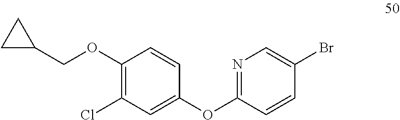 [SMILES: Clc1cc(Oc2ccc(Br)cn2)ccc1OCC1CC1]

Compound 50 was obtained by using Compound 26 instead of Compound 12 in Step 1 in Reference example 021.

¹H-NMR (CDCl₃) δ: 8.19 (d, J=2.6 Hz, 1H), 7.75 (dd, J=8.9, 2.2 Hz, 1H), 7.17 (d, J=2.6 Hz, 1H), 6.97 (dd, J=8.9, 2.5 Hz, 1H), 6.91 (d, J=8.8 Hz, 1H), 6.81 (d, J=8.7 Hz, 1H), 3.88 (d, J=6.7 Hz, 2H), 1.39-1.23 (m, 1H), 0.69-0.63 (m, 2H), 0.41-0.36 (m, 2H).

### Reference Example 031

### Preparation of Compound 51

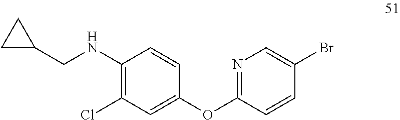 [SMILES: Clc1cc(Oc2ccc(Br)cn2)ccc1NCC1CC1]

Compound 51 was obtained by using Compound 31 instead of Compound 12 in Step 1 in Reference example 021.

¹H-NMR (CDCl₃) δ: 8.19 (d, J=2.6 Hz, 1H), 7.72 (dd, J=8.8, 2.5 Hz, 1H), 7.08 (d, J=2.6 Hz, 1H), 6.92 (dd, J=8.8, 2.7 Hz, 1H), 6.77 (d, J=8.7 Hz, 1H), 6.63 (d, J=8.8 Hz, 1H), 4.34 (br s, 1H), 3.00 (d, J=4.7 Hz, 2H), 1.22-1.10 (m, 1H), 0.63-0.57 (m, 2H), 0.30-0.25 (m, 2H).

### Reference Example 032

### Preparation of Compound 52

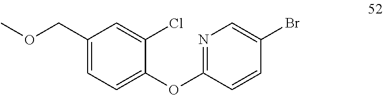 [SMILES: COCc1ccc(Oc2ccc(Br)cn2)c(Cl)c1]

Compound 52 was obtained by using 2-chloro-4-(methoxymethyl)phenol (The preparation for method is described in Heterocycles, 1985, vol. 23, #6 p1483-1491) instead of Compound 12 in Step 1 in Reference example 021.

¹H-NMR (CDCl₃) δ: 8.20 (d, J=2.0 Hz, 1H), 7.65 (dd, J=8.6, 2.5 Hz, 1H), 7.47 (d, J=2.0 Hz, 1H), 7.30 (dd, J=8.1, 2.0 Hz, 1H), 6.92 (d, J=8.6 Hz, 1H), 6.70 (d, J=8.6 Hz, 1H), 5.25 (s, 2H), 3.90 (s, 3H).

### Reference Example 033

### Preparation of Compound 53

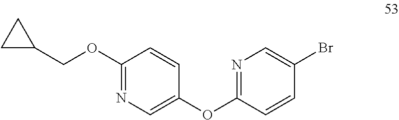 [SMILES: Brc1ccc(Oc2ccc(OCC3CC3)nc2)nc1]

Compound 53 was obtained by using 6-(cyclopropylmethoxy)pyridin-3-ol (The preparation for method is described in WO2010/05445) instead of Compound 12 in Step 1 in Reference example 021.

¹H-NMR (CDCl₃) δ: 8.17 (d, J=2.5 Hz, 1H), 7.98 (d, J=3.0 Hz, 1H), 7.77 (dd, J=8.9, 2.3 Hz, 1H), 7.39 (dd, J=8.6, 3.0 Hz, 1H), 6.86 (d, J=8.6 Hz, 1H), 6.81 (d, J=8.6 Hz, 1H), 4.12 (d, J=7.1 Hz, 2H), 1.29 (m, 1H), 0.62 (m, 2H), 0.35 (m, 2H). [M+H]=322, Method Condition 2: retention time 2.55 min

### Reference Example 034

### Preparation of Compound 54

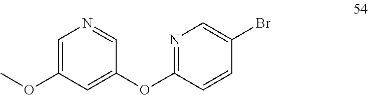 [SMILES: COc1cncc(Oc2ccc(Br)cn2)c1]

Compound 54 was obtained by using 5-methoxypyridin-3-ol instead of Compound 12 in Step 1 in Reference example 021.

¹H-NMR (CDCl₃) δ: 8.20 (dd, J=5.6, 2.5 Hz, 2H), 8.11 (d, J=2.03 Hz, 1H), 7.82 (dd, J=8.6, 2.5 Hz, 1H), 7.04 (t, J=2.3 Hz, 1H), 6.91 (d, J=8.6, 1H), 3.86 (s, 3H). [M+H]=282, Method Condition 2: retention time 1.74 min

### Reference Example 035

### Preparation of Compound 55

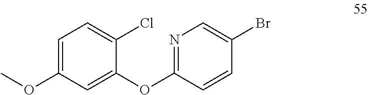 [SMILES: COc1ccc(Cl)c(Oc2ccc(Br)cn2)c1]

Compound 55 was obtained by using 2-chloro-5-methoxyphenol instead of Compound 12 in Step 1 in Reference example 021.

¹H-NMR (CDCl₃) δ: 8.18 (d, J=2.4 Hz, 1H), 7.76-7.80 (m, 1H), 7.36-7.32 (m, 1H), 6.89 (d, J=8.7 Hz, 1H), 6.78-6.74 (m, 2H), 3.79 (s, 3H).

### Reference Example 036

### Preparation of Compound 56

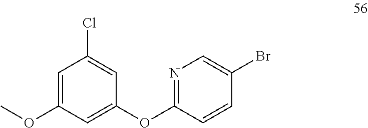 [SMILES: COc1cc(Cl)cc(Oc2ccc(Br)cn2)c1]

Compound 56 was obtained by using 3-chloro-5-methoxyphenol instead of Compound 12 in Step 1 in Reference example 021.

¹H-NMR (CDCl₃) δ: 8.23 (d, J=2.4 Hz, 1H), 7.76-7.80 (m, 1H), 6.85 (d, J=8.7 Hz, 1H), 6.76-6.71 (m, 2H), 6.58-6.56 (m, 1H), 3.78 (s, 3H).

### Reference Example 037

### Preparation of Compound 57

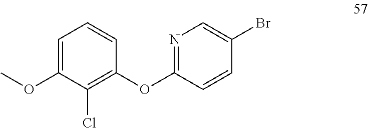 [SMILES: COc1cccc(Oc2ccc(Br)cn2)c1Cl]

Compound 57 was obtained by using 2-chloro-3-methoxyphenol instead of Compound 12 in Step 1 in Reference example 021.

¹H-NMR (CDCl₃) δ: 8.16 (d, J=2.4 Hz, 1H), 7.77 (dd, J=8.7, 2.4 Hz, 1H), 7.27 (t, J=8.3 Hz, 1H), 6.90-6.81 (m, 3H), 3.93 (s, 3H).

### Reference Example 038

### Preparation of Compound 59

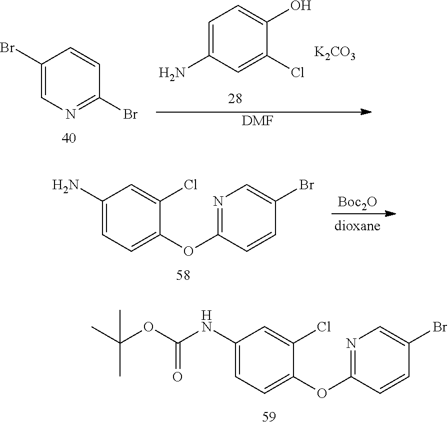

### Step 1 Preparation of Compound 58

Potassium carbonate (2.10 g, 15.2 mmol) was added to DMF solution (10 mL) of Compound 38 (3.00 g, 12.7 mmol) and Compound 28 (2.00 g, 13.9 mmol), and the mixture was stirred at 160° C. for 3 hours. The mixture wad diluted with ethyl acetate, the insoluble matter was filtered. The filtrate was condensed under reduced pressure. The residue was purified by silica gel chromatography (hexane-ethyl acetate) to afford Compound 58 (1.67 g, yield 44%).

¹H-NMR (CDCl₃) δ: 8.16 (d, J=2.4 Hz, 1H), 7.74 (dd, J=8.7, 2.6 Hz, 1H), 6.97 (d, J=8.5 Hz, 1H), 6.83 (d, J=8.7 Hz, 1H), 6.77 (d, J=2.7 Hz, 1H), 6.59 (dd, J=8.5, 2.7 Hz, 1H), 3.69 (br s, 2H).

Boc₂O (0.930 mL, 4.01 mmol) was added to the dioxane solution of Compound 58 (1.00 g, 3.34 mmol), the mixture was stirred at 60° C. for 7 hours. The solvent was condensed under reduced pressure. The residue was purified by silica gel chromatography (ethyl acetate-hexane) to afford Compound 59 (1.21 g, yield 91%).

¹H-NMR (CDCl₃) δ: 8.14 (d, J=2.6 Hz, 1H), 7.76 (dd, J=8.3, 2.1 Hz, 1H), 7.63 (d, J=1.7 Hz, 1H), 7.21 (dd, J=8.9, 2.4 Hz, 1H), 7.10 (d, J=8.4 Hz, 1H), 6.86 (d, J=8.8 Hz, 1H), 6.48 (br s, 1H), 1.52 (s, 9H).

### Reference Example 039

### Step 1 Preparation of Compound 64

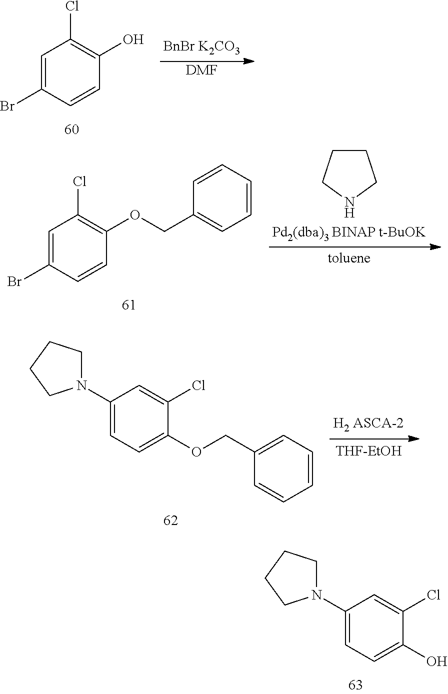

### Step 1 Preparation of Compound 61

Benzyl bromide (1.57 mL, 13.3 mmol) and potassium carbonate (2.17 g, 15.7 mmol) were added to DMF solution (25 mL) of Compound 60 (2.50 g, 12.1 mmol), and the mixture was stirred at room temperature for 3 hours. Water was added to the mixture, and the mixture was extracted with ethyl acetate. The organic layer was washed with water, dried over magnesium sulfate. The solvent was condensed under reduced pressure. The residue was purified by silica gel chromatography (ethyl acetate-hexane) to afford Compound 61 (3.56 g, yield 99%).

¹H-NMR (CDCl₃) δ: 7.52 (d, J=2.0 Hz, 1H), 7.45-7.25 (m, 6H), 6.83 (d, J=8.6 Hz, 1H), 5.14 (s, 2H).

[M+H]=298, Method Condition 2: retention time 2.79 min

### Step 2 Preparation of Compound 62

Potassium t-butoxide (0.388 g, 4.03 mmol), Pd₂(dba)₃ (31.0 mg, 0.0336 mmol) and BINAP (63.0 mg, 0.101 mmol) were added to the toluene solution of Compound 61 (1.00 g, 3.36 mmol) and pyrrolidine (0.281 mL, 3.36 mmol). The atmosphere was replaced with nitrogen, and the mixture was stirred at 100° C. for 4 hours. Water was added to the mixture, and the mixture was extracted with ethyl acetate. The organic layer was washed with 2 mol/L hydrochloric acid, saturated sodium bicarbonate water and saturated brine, and dried over magnesium sulfate. The solvent was condensed under reduced pressure. The residue was purified by silica gel chromatography (ethyl acetate-hexane) to afford Compound 62 (1.00 g, yield 69%).

¹H-NMR (CDCl₃) δ: 7.50-7.25 (m, 5H), 6.88 (d, J=8.6 Hz, 1H), 6.60 (d, J=3.0 Hz, 1H), 6.35 (dd, J=9.1, 3.0 Hz, 1H), 5.04 (s, 2H), 3.21 (t, J=6.3 Hz, 4H), 1.96 (m, 4H).

[M+H]=288, Method Condition 2: retention time 2.86 min

### Step 3 Preparation of Compound 63

Platinum-palladium/carbon (trade name: ASCA-2, NEM cat, 96.0 mg) was added to the mixed solution of tetrahydrofuran (5 mL) and ethanol (10 mL) of Compound 62 (0.960 mg, 3.36 mmol) and the mixture was stirred for 7 hours in a hydrogen atmosphere. Catalyst was filtered, and the filtrate was condensed under reduced pressure. The residue was purified by silica gel chromatography (ethyl acetate-hexane) to afford Compound 63 (154 mg, yield 23%).

¹H-NMR (CDCl₃) δ: 6.90 (d, J=8.6 Hz, 1H), 6.51 (s, 1H), 6.42 (d, J=8.6 Hz, 1H), 4.89 (s, 1H), 3.20 (m, 4H), 1.99 (m, 4H).

[M+H]=198, Method Condition 2: retention time 1.25 min Step 4 Preparation of Compound 57

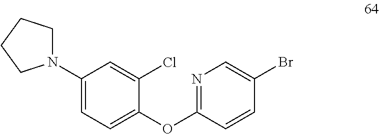 [SMILES: Clc1cc(N2CCCC2)ccc1Oc1ccc(Br)cn1]

Compound 64 was obtained by using Compound 63 obtained in step 3 instead of Compound 12 in Step 1 in Reference example 021.

¹H-NMR (CDCl₃) δ: 8.18 (d, J=2.5, 1H), 7.73 (dd, J=8.6, 2.5 Hz, 1H), 7.03 (d, J=8.6 Hz, 1H), 6.82 (d, J=8.6 Hz, 1H), 6.60 (d, J=3.0 Hz, 1H), 6.46 (dd, J=8.9, 2.8 Hz, 1H), 3.27 (m, 4H), 2.01 (m, 4H).

[M+H]=354, Method Condition 2: retention time 2.87 min

### Reference Example 040

### Preparation of Compound 65

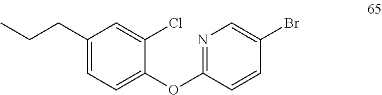 [SMILES: CCCc1ccc(Oc2ccc(Br)cn2)c(Cl)c1]

Compound 65 was obtained by using 2-chloro-4-propylphenol (The preparation for method is described in U.S. Pat. No. 1,980,966) instead of Compound 12 in Step 1 in Reference example 021.

¹H-NMR (CDCl₃) δ: 8.17 (d, J=2.5, 1H), 7.77 (dd, J=8.6, 2.5 Hz, 1H), 7.28 (s, 1H), 7.13-7.08 (m, 2H), 6.88 (d, J=8.6 Hz, 1H), 2.58 (t, J=7.6 Hz, 2H), 1.66 (td, J=15.0, 7.8 Hz, 2H), 0.97 (t, J=7.8 Hz, 3H).

[M+H]=327, Method Condition 2: retention time 2.91 min

### Reference Example 041

### Preparation of Compound 68

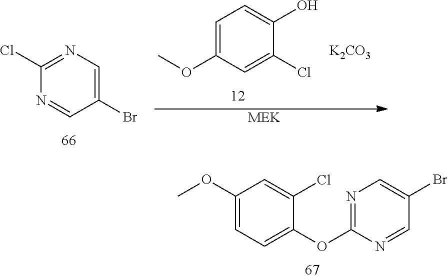

### Step 1 Preparation of Compound 67

Potassium carbonate (2.57 g, 18.6 mmol) was added to 2-butanone solution (50 mL) of Compound 66 (3.00 g, 15.5 mmol) and Compound 12 (3.20 g, 20.2 mmol), and the mixture was stirred at 100° C. for 5 hours. Water was added to the mixture, and the mixture was extracted with ethyl acetate. The solvent was condensed under reduced pressure. 5% aqueous sodium hydroxide was added to the residue, and the precipitated crystal was filtered off and dried to afford Compound 67 (4.90 g, yield 100%).

¹H-NMR (DMSO-d₆) δ: 8.81 (s, 2H), 7.32 (d, J=8.9 Hz, 1H), 7.17 (d, J=3.0 Hz, 1H), 6.98 (dd, J=9.0, 2.9 Hz, 1H), 3.80 (s, 3H).

### Step 2 Preparation of Compound 68

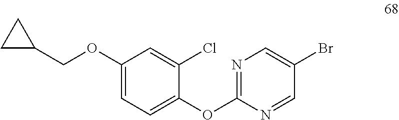 [SMILES: Clc1cc(OCC2CC2)ccc1Oc1ncc(Br)cn1]

Compound 68 was obtained by Compound 67 instead of Compound 39 in Step 2 in Reference Example 021 and by (bromomethyl)cyclopropane instead of iodoethane in Step 3.

¹H-NMR (CDCl₃) δ: 8.55 (d, J=0.8 Hz, 2H), 7.13 (d, J=9.0 Hz, 1H), 7.00 (d, J=2.9 Hz, 1H), 6.85 (dd, J=8.8, 2.9 Hz, 1H), 3.79 (d, J=6.9 Hz, 2H), 1.34-1.20 (m, 1H), 0.69-0.63 (m, 2H), 0.38-0.32 (m, 2H).

### Reference Example 042

### Preparation of Compound 70

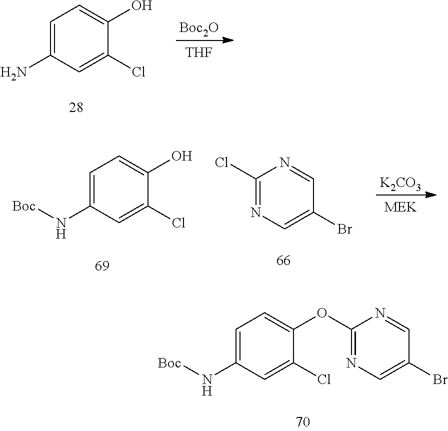

### Step 1 Preparation of Compound 69

Boc₂O (5.82 mL, 25.1 mmol) was added to the dioxane solution (30 mL) of Compound 28 (3.0 g, 20.9 mmol), the mixture was stirred overnight at room temperature. The solvent was condensed under reduced pressure. The residue was purified by silica gel chromatography (ethyl acetate-hexane) to afford Compound 69 (6.10 g, yield 99%).

¹H-NMR (DMSO-d₆) δ: 9.71 (s, 1H), 9.17 (s, 1H), 7.46 (d, J=1.8 Hz, 1H), 7.14 (dd, J=8.7, 2.4 Hz, 1H), 6.83 (d, J=8.7 Hz, 1H), 1.45 (s, 911).

### Step 2 Preparation of Compound 70

Potassium carbonate (1.32 g, 9.55 mmol) was added to 2-butanone solution (20 mL) of Compound 69 (1.54 g, 7.95 mmol) and Compound 66 (3.00 g, 10.34 mmol), and the mixture was stirred at 100° C. for 4 hours. The solvent was condensed under reduced pressure, and water was added to the residue. The mixture was extracted with ethyl acetate. The organic layer was washed with saturated brine, dried over magnesium sulfate, and filtered. The solvent was condensed under reduced pressure. The residue was purified by silica gel chromatography (ethyl acetate-hexane) to afford Compound 70 (3.15 g, yield 97%).

¹H-NMR (CDCl₃) δ: 8.55 (s, 2H), 7.66 (d, J=2.4 Hz, 1H), 7.23 (dd, J=8.7, 2.4 Hz, 1H), 7.13 (d, J=8.7 Hz, 1H), 6.63 (s, 1H), 1.52 (s, 9H).

### Reference Example 043

### Preparation of Compound 74

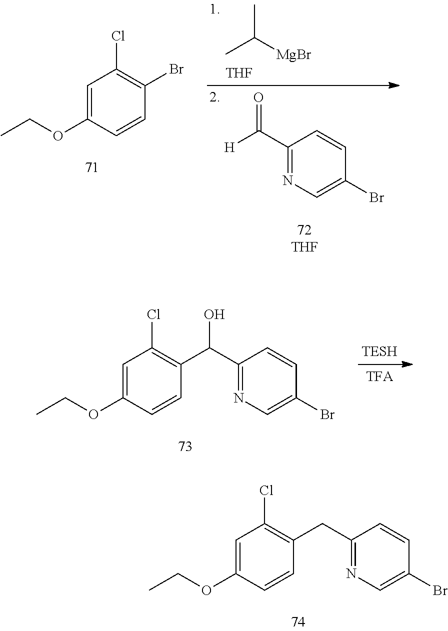

### Step 1 Preparation of Compound 73

Isopropyl magnesium bromide (15% tetrahydrofuran solution, 1 mol/L, 2.34 mmol) was added to the tetrahydrofuran solution (5 mL) of Compound 71 (500 mg, 2.12 mmol), and the mixture was stirred for 2.5 hours. The mixture was cooled to −30° C. The tetrahydrofuran solution of Compound 72 (395 mg, 2.12 mmol) was added dropwise to the mixture, and the mixture was stirred and warmed to −10° C. for 1 hour after completion of adding dropwise. Saturated ammonium chloride solution was added to the mixture, and the mixture was extracted with ethyl acetate. The organic layer was washed with saturated brine, dried over magnesium sulfate. The solvent was condensed under reduced pressure. The residue was purified by silica gel chromatography (hexane-ethyl acetate) to afford Compound 73 (552 mg, yield 88%).

¹H-NMR (CDCl₃) δ: 8.63 (d, J=2.0 Hz, 1H), 7.75 (dd, J=8.6, 2.0 Hz, 1H), 7.22 (d, J=8.6 Hz, 1H), 7.14 (d, J=8.6 Hz, 1H), 6.91 (d, J=2.5 Hz, 1H), 6.78 (dd, J=8.6, 2.5 Hz, 1H), 6.15 (d, J=4.1 Hz, 1), 4.81 (d, J=4.5 Hz, 1H), 4.00 (q, J=7.1 Hz, 2H), 1.39 (t, J=7.1 Hz, 3H).

[M+H]=342, Method Condition 2: retention time 2.18 min

### Step 2 Preparation of Compound 74

Triethylsilane (0.106 mL, 0.654 mmol) was added to the trifluoroacetic acid solution (2 mL) of Compound 73 (112 mg, 0.327 mmol), and the mixture was stirred at 60° C. for 6.5 hours. The mixture was added to Saturated sodium bicarbonate water, and the mixture was extracted with ethyl acetate. The organic layer was washed with saturated brine, and dried over magnesium sulfate. The solvent was condensed under reduced pressure. The residue was purified by silica gel chromatography (hexane-ethyl acetate) to afford Compound 74 (68 mg, yield 64%).

¹H-NMR (CDCl₃) δ: 8.59 (d, J=2.0 Hz, 1H), 7.68 (dd, J=8.1, 2.5 Hz, 1H), 7.16 (d, J=8.6 Hz, 1H), 6.98 (d, J=8.6 Hz, 1H), 6.93 (d, J=2.5 Hz, 1H), 6.77 (dd, J=8.6, 2.5 Hz, 1H), 4.17 (d, J=8.6 Hz, 2H), 4.00 (q, J=7.1 Hz, 2H), 1.40 (t, J=7.1 Hz, 3H).

[M+H]=328, Method Condition 2: retention time 2.60 min

### Reference Example 044

### Preparation of Compound 76

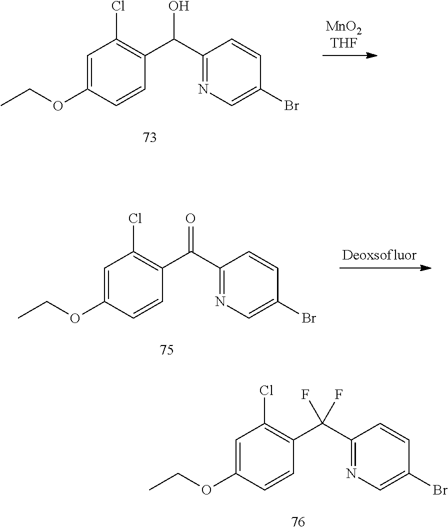 [SMILES: CCOc1ccc(C(O)c2ccc(Br)cn2)c(Cl)c1 ; CCOc1ccc(C(=O)c2ccc(Br)cn2)c(Cl)c1 ; CCOc1ccc(C(F)(F)c2ccc(Br)cn2)c(Cl)c1]

### Step 1 Preparation of Compound 75

Manganese dioxide (1.69 g, 19.4 mmol) was added to the tetrahydrofuran solution of Compound 73 (665 mg, 1.94 mmol), and the mixture was stirred at room temperature for 2.5 hours. The insoluble matter was filtered, and the filtrate was condensed under reduced pressure. The residue was purified by silica gel chromatography (hexane-ethyl acetate) to afford Compound 75 (528 mg, yield 80%).

¹H-NMR (CDCl₃) δ: 8.71 (d, J=2.0 Hz, 1H), 8.02 (dd, J=8.1, 2.0 Hz, 1H), 7.96 (d, J=8.6 Hz, 1H), 7.52 (d, J=8.6 Hz, 1H), 6.95 (d, J=2.5 Hz, 1H), 6.88 (dd, J=8.6, 2.5 Hz, 1H), 4.09 (q, J=7.1 Hz, 2H), 1.44 (t, J=7.1 Hz, 3H).

[M+H]=341, Method Condition 2: retention time 2.44 min

### Step 2 Preparation of Compound 76

Deoxo-fluor(TM) (0.411 mL, 2.23 mmol) was added to Compound 75 (152 mg, 0.446 mmol), and the mixture was stirred at 90° C. for 10 hours. Saturated sodium bicarbonate water was added to the mixture, the mixture was extracted with ethyl acetate. The organic layer was washed with saturated brine, and dried over magnesium sulfate. The solvent was condensed under reduced pressure. The residue was purified by silica gel chromatography (hexane-ethyl acetate) to afford Compound 76 (131 mg, yield 81%).

¹H-NMR (CDCl₃) δ: 8.62 (s, 1H), 7.96 (dd, J=8.3, 2.3 Hz, 1H), 7.77 (m, 2H), 6.90 (m, 2H), 4.05 (q, J=7.1 Hz, 2H), 1.42 (t, J=7.1 Hz, 3H).

[M+H]=362, Method Condition 2: retention time 2.66 min

### Reference Example 045

### Preparation of Compound 80

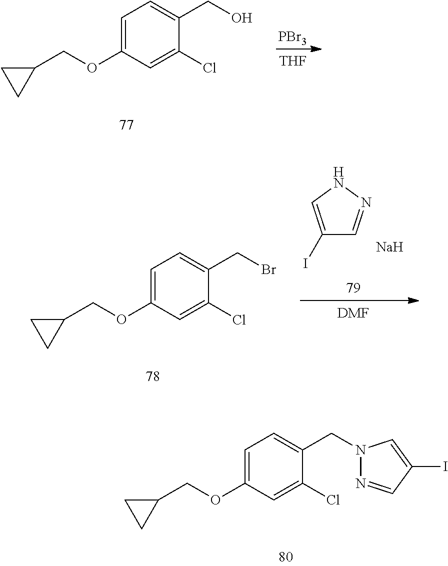

### Step 1 Preparation of Compound 78

The tetrahydrofuran solution of Compound 77 (3.35 g, 15.75 mmol. The preparation for method is described in WO2010/05445.) was cooled with ice in a cool bath in a nitrogen atmosphere. Phosphorus(III) Bromide (6.30 ml, 6.30 mmol, 1 mol/L dichloromethane solution) was added dropwise to the mixture, and the mixture was stirred for 30 minutes while cooling in a ice. The reaction mixture was added to saturated sodium bicarbonate water. The mixture was extracted with diethylether. The organic layer was washed with saturated brine, and dried over magnesium sulfate. The organic layer was filtrated, and the filtrate was condensed under reduced pressure. The obtained residue was used to the next step. Compound 78 (4.23 g, yield 93%).

### Step 2 Preparation of Compound 80

The DMF suspension (4 mL) of sodium hydride (0.217 g, 5.41 mmol) was cooled with ice in a cool bath in a nitrogen atmosphere. Compound 79 (700 mg, 3.61 mmol) was added to the mixture, and the mixture was stirred at room temperature for 30 minutes. The mixture was also cooled with ice in a cool bath, the DMF solution (2.000 ml) of Compound 78 (1.193 g, 4.33 mmol) was added to the mixture. The mixture was stirred at room temperature. Water was added to the mixture, and the mixture was extracted with diethylether. The organic layer was washed with saturated brine, and dried over magnesium sulfate. The organic layer was filtrated, and the filtrate was condensed under reduced pressure. The residue was purified by silica gel chromatography (hexane-ethyl acetate) to afford Compound 80 (1.31 g, yield 93%).

¹H-NMR (CDCl₃) δ: 7.52 (s, 1H), 7.43 (s, 1H), 7.10 (d, J=8.6 Hz, 1H), 6.94 (d, J=2.5 Hz, 1H), 6.79 (dd, J=8.6, 2.5 Hz, 1H), 5.34 (s, 2H), 3.78 (d, J=6.9 Hz, 2H), 1.32-1.18 (m, 1H), 0.68-0.62 (m, 2H), 0.37-0.31 (m, 2H).

### Reference Example 046

### Preparation of Compound 81

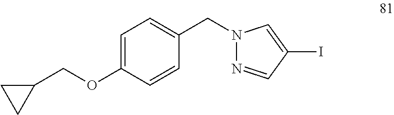 [SMILES: Ic1cnn(Cc2ccc(OCC3CC3)cc2)c1]

Compound 81 was obtained by using 1-(bromomethyl)-4-(cyclopropylmethoxy)benzene (The preparation for method is described in WO2010/127212.) instead of Compound 78 in Step 2 in Reference example 045.

¹H-NMR (CDCl₃) δ: 7.52 (s, 1H), 7.34 (s, 1H), 7.20-7.14 (m, 2H), 6.91-6.85 (m, 2H), 5.22 (s, 2H), 3.79 (d, J=6.9 Hz, 2H), 1.33-1.20 (m, 1H), 0.67-0.61 (m, 2H), 0.37-0.31 (m, 2H).

### Reference Example 047

### Preparation of Compound 85

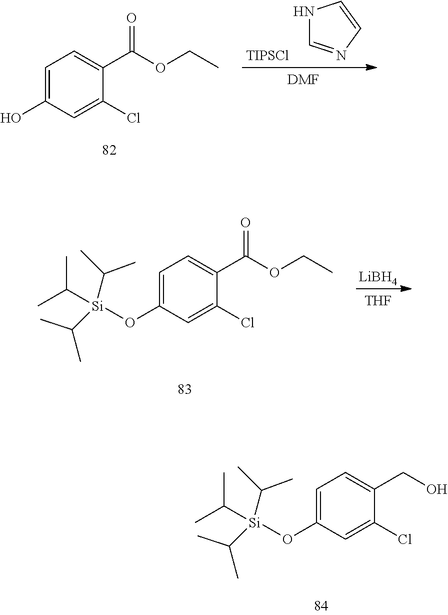

### Step 1 Preparation of Compound 83

The DMF solution (60 mL) of Compound 82 (6.10 g, 30.4 mmol) was cooled in ice under a nitrogen atmosphere. Imidazole (8.18 g, 121 mmol) and triisopropylsilyl chloride (14.3 mL, 66.8 mmol) were added to the mixture, and the mixture was stirred at 60° C. for 10 hours. Water was added to the reaction mixture, the reaction mixture was extracted with diethylether. The organic layer was washed with saturated brine, dried over magnesium sulfate, and filtrated. The solvent was condensed under reduced pressure. The residue was purified by silica gel chromatography (hexane-ethyl acetate) to afford Compound 83 (10.2 g, yield 94%).

¹H-NMR (CDCl₃) δ: 7.80 (d, J=8.7 Hz, 1H), 6.94 (d, J=2.4 Hz, 1H), 6.77 (dd, J=8.6, 2.4 Hz, 1H), 4.35 (q, J=7.1 Hz, 2H), 1.38 (t, J=7.1 Hz, 3H), 1.32-1.21 (m, 3H), 1.10 (d, J=7.0 Hz, 18H).

### Step 2 Preparation of Compound 84

The tetrahydrofuran solution (20 mL) of Compound 83 (7.00 g, 19.6 mmol) was cooled in ice under a nitrogen atmosphere. Lithium borohydride (1.28 g, 58.8 mmol) was added to the mixture, and the mixture was stirred at 80° C. for 4 hours under a nitrogen atmosphere. Water was added to the reaction mixture, and the mixture was extracted with ethyl acetate. The organic layer was washed with saturated brine, and dried over magnesium sulfate, and filtered. The solvent was condensed under reduced pressure. The residue was purified by silica gel chromatography (hexane-ethyl acetate) to afford Compound 84 (5.33 g, yield 86%).

¹H-NMR (CDCl₃) δ: 7.27 (d, J=8.4 Hz, 1H), 6.90 (d, J=2.4 Hz, 1H), 6.77 (dd, J=8.4, 2.4 Hz, 1H), 4.69 (d, J=6.4 Hz, 2H), 1.84 (t, J=6.3 Hz, 1H), 1.31-1.18 (m, 3H), 1.11 (d, J=7.0 Hz, 18H).

### Step 3 Preparation of Compound 85

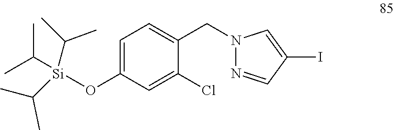 [SMILES: CC(C)[Si](Oc1ccc(Cn2cc(I)cn2)c(Cl)c1)(C(C)C)C(C)C]

Compound 85 was obtained by using Compound 84 instead of Compound 77 in Step 1 in Reference example 045.

¹H-NMR (CDCl₃) δ: 7.53 (s, 1H), 7.43 (s, 1H), 6.99 (d, J=8.5 Hz, 1H), 6.92 (d, J=2.4 Hz, 1H), 6.74 (dd, J=8.5, 2.5 Hz, 1H), 5.33 (s, 2H), 1.33-1.18 (m, 3H), 1.09 (d, J=7.0 Hz, 18H)

### Reference Example 048

### Preparation of Compound 86

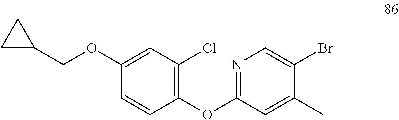 [SMILES: Cc1cc(Oc2ccc(OCC3CC3)cc2Cl)ncc1Br]

Compound 86 was obtained by 2,5-dibromo-3-picoline instead of Compound 38 in Step 1 in Reference Example 021 and by (bromomethyl)cyclopropane instead of iodoethane in Step 3.

¹H-NMR (CDCl₃) δ: 7.94 (d, J=2.1 Hz, 1H), 7.61 (d, J=1.7 Hz, 1H), 7.09 (d, J=9.0 Hz, 1H), 6.98 (d, J=2.9 Hz, 1H), 6.84 (dd, J=8.8, 3.0 Hz, 1H), 3.78 (d, J=6.9 Hz, 2H), 2.37 (s, 3H), 1.33-1.20 (m, 1H), 0.69-0.63 (m, 2H), 0.37-0.32 (m, 2H).

### Reference Example 049

### Preparation of Compound 87

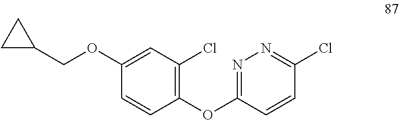 [SMILES: Clc1ccc(Oc2ccc(OCC3CC3)cc2Cl)nn1]

Compound 87 was obtained by 3,6-dichloropyridazine instead of Compound 38 in Step 1 in Reference Example 021 and by (bromomethyl)cyclopropane instead of iodoethane in Step 3.

¹H-NMR (DMSO-d₆) δ: 7.96 (d, J=9.3 Hz, 1H), 7.65 (d, J=9.3 Hz, 1H), 7.33 (d, J=8.8 Hz, 1H), 7.16 (s, 1H), 6.99 (d, J=8.8 Hz, 1H), 3.85 (d, J=6.8 Hz, 2H), 1.20-1.24 (m, 1H), 0.55-0.59 (m, 2H), 0.30-0.34 (m, 2H).

### Reference Example 050

### Preparation of Compound 89

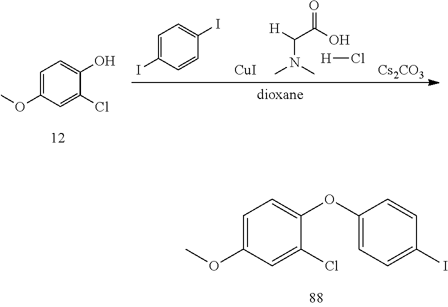

### Step 1 Preparation of Compound 88

Cesium carbonate (8.22 g, 25.2 mmol), copper iodide (0.240 g, 1.26 mg) and N, N-dimethylglycine hydrochloride (0.176 g, 1.26 mmol) were added to the dioxane solution (20 mL) of Compound 12 (2.00 g, 12.6 mmol) and 1,4-diiodobenzene (8.32 g, 1.26 mmol), and the mixture was stirred at 100° C. for 12 hours. The mixture wad diluted with chloroform, and the insoluble matter was filtered. The filtrate was condensed under reduced pressure. The residue was purified by silica gel chromatography (hexane-ethyl acetate) to afford Compound 88 (3.16 g, yield 70%).

¹H-NMR (CDCl₃) δ: 7.58-7.53 (m, 2H), 7.02-6.97 (m, 2H), 6.80 (dd, J=9.0, 2.9 Hz, 1H), 6.66-6.61 (m, 2H), 3.81 (s, 3H).

### Step 2 Preparation of Compound 89

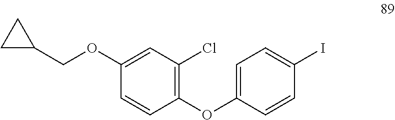 [SMILES: Clc1cc(OCC2CC2)ccc1Oc1ccc(I)cc1]

Compound 89 was obtained by Compound 88 instead of Compound 39 in Step 2 in Reference Example 021 and by (bromomethyl)cyclopropane instead of Iodoethane in Step 3.

¹H-NMR (CDCl₃) δ: 7.59-7.53 (m, 2H), 7.00-6.96 (m, 2H), 6.80 (dd, J=8.8, 2.9 Hz, 1H), 6.66-6.61 (m, 2H), 3.78 (d, J=7.0 Hz, 2H), 1.32-1.22 (m, 1H), 0.70-0.63 (m, 2H), 0.38-0.33 (m, 2H).

### Reference Example 051

### Preparation of Compound 90

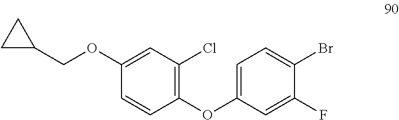 [SMILES: Fc1cc(Oc2ccc(OCC3CC3)cc2Cl)ccc1Br]

Compound 90 was obtained by using 1-bromo-2-fluoro-4-iodobenzene instead of 1,4-diiodobenzene in Step 1 in Reference example 050.

¹H-NMR (CDCl₃) δ: 7.41 (t, J=8.2 Hz, 1H), 7.04-6.99 (m, 2H), 6.82 (dd, J=8.8, 2.9 Hz, 1H), 6.65-6.55 (m, 2H), 3.79 (d, J=7.0 Hz, 2H), 1.34-1.21 (m, 1H), 0.71-0.63 (m, 2H), 0.40-0.33 (m, 2H).

### Reference Example 052

### Preparation of Compound 99

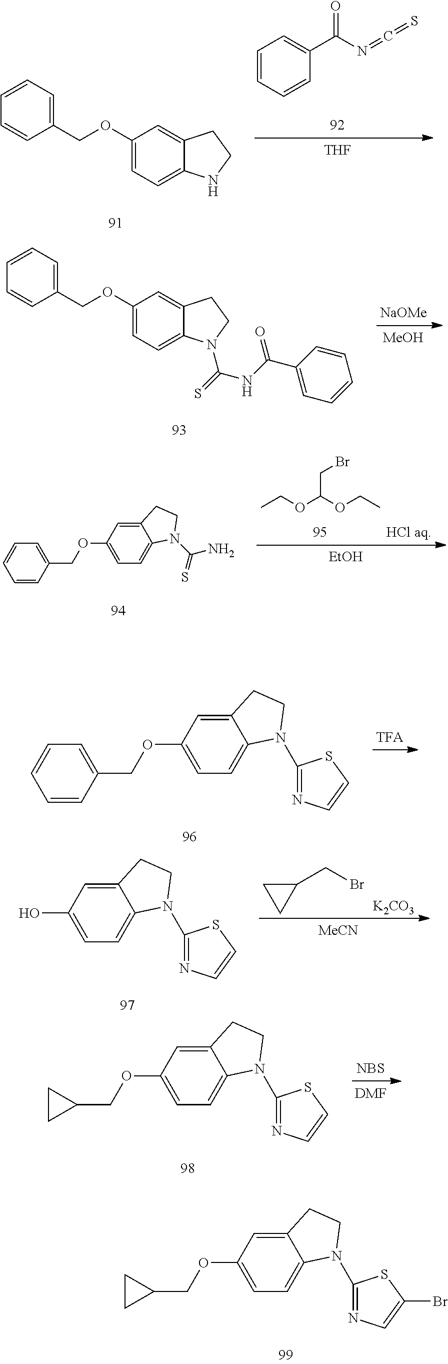

### Step 1 Preparation of Compound 93

The tetrahydrofuran solution (20 mL) of Compound 91 (2.06 g, 8.87 mmol) was cooled in ice under a nitrogen atmosphere. Compound 92 (1.32 mL, 9.76 mmol) was added to the mixture, and the mixture was stirred at room temperature for 30 minutes. The solvent was condensed under reduced pressure. The residue was suspended in diisopropylether, and the precipitated solid was filtrated. The obtained solid was advanced to the next step without purification.

Step 2 Preparation of Compound 94

1 mol/L sodium methoxide solution (methanol solution) was added to the methanol suspension (30 mL) of Compound 93 (3.43 g, 8.83 mmol), and the mixture was stirred at 80° C. for 48 hours under a nitrogen atmosphere. The reaction mixture was added to the saturated ammonium chloride aqueous solution, and the reaction mixture was extracted with ethyl acetate. The organic layer was washed with saturated brine, dried over magnesium sulfate, and filtered. The solvent was condensed under reduced pressure. The obtained residue was filtered off with ethyl acetate/diisopropylether. The obtained solid was advanced to the next step without purification.

### Step 3 Preparation of Compound 96

Compound 95 (1.54 mL, 10.0 mmol) and 2 mol/L hydrochloric acid (0.334 mL, 0.668 mmol) were added to the ethanol suspension (20 mL) of Compound 94, and the mixture was stirred at 100° C. for 4 hours. Saturated sodium bicarbonate water was added to the mixture, and the mixture was extracted with ethyl acetate. The organic layer was washed with saturated brine, dried over magnesium sulfate, and filtered. The solvent was condensed under reduced pressure. The residue was purified by silica gel chromatography (hexane-ethyl acetate) to afford Compound 96 (1.12 g, yield 54%).

¹H-NMR (CDCl₃) δ: 7.82 (d, J=8.5 Hz, 1H), 7.46-7.29 (m, 6H), 6.88-6.82 (m, 2H), 6.66 (d, J=3.5 Hz, 1H), 5.04 (s, 2H), 4.10 (t, J=8.5 Hz, 2H), 3.26 (t, J=8.5 Hz, 2H).

### Step 4 Preparation of Compound 97

Trifluoroacetic acid (3 mL, 38.9 mmol) of Compound 96 (880 mg, 2.85 mmol) was stirred at 80° C. for 30 hours. The solvent was condensed under reduced pressure. The residue was purified by silica gel chromatography (chloroform-methanol) to afford Compound 97 (490 mg, yield 79%).

¹H-NMR (CDCl₃) δ: 7.47 (d, J=4.0 Hz, 1H), 7.41 (d, J=7.9 Hz, 1H), 6.79-6.70 (m, 3H), 4.31 (t, J=8.5 Hz, 2H), 3.27 (t, J=8.4 Hz, 2H).

### Step 5 Preparation of Compound 98

Potassium carbonate (608 mg, 4.40 mmol) and (bromomethyl)cyclopropane (0.323 mL, 3.30 mmol) were added to the acetonitrile solution (5 ml) of Compound 97 (480 mg, 2.20 mmol), and the mixture was stirred at 100° C. for 16 hours. Water was added to the mixture, and the mixture was extracted with ethyl acetate. The organic layer was washed with saturated brine, dried over magnesium sulfate. The solvent was condensed under reduced pressure. The residue was purified by silica gel chromatography (hexane-ethyl acetate) to afford Compound 98 (344 mg, yield 57%).

¹H-NMR (CDCl₃) δ: 7.80 (d, J=8.5 Hz, 1H), 7.37 (d, J=3.7 Hz, 1H), 6.82-6.74 (m, 2H), 6.65 (d, J=3.7 Hz, 1H), 4.10 (t, J=8.6 Hz, 2H), 3.77 (d, J=6.9 Hz, 2H), 3.25 (t, J=8.5 Hz, 2H), 1.33-1.20 (m, 1H), 0.67-0.61 (m, 2H), 0.37-0.32 (m, 2H).

### Step 6 Preparation of Compound 99

N-bromosuccinimide (247 mg, 1.39 mmol) was added to the DMF solution (2 mL) of Compound 98 (343 mg, 1.26 mmol), and the mixture was stirred at room temperature for 3 hours. Water was added to the reaction mixture, and the mixture was extracted with diethylether. The organic layer was washed with saturated brine, and dried over magnesium sulfate. The solvent was condensed under reduced pressure. The residue was purified by silica gel chromatography (hexane-ethyl acetate) to afford Compound 99 (270 mg, yield 61%).

¹H-NMR (CDCl₃) δ: 7.67 (d, J=8.7 Hz, 1H), 7.23 (s, 1H), 6.81 (d, J=2.6 Hz, 1H), 6.74 (dd, J=8.7, 2.6 Hz, 1H), 4.03 (t, J=8.5 Hz, 2H), 3.77 (d, J=7.0 Hz, 2H), 3.25 (t, J=8.5 Hz, 2H), 1.32-1.19 (m, 1H), 0.67-0.61 (m, 2H), 0.38-0.30 (m, 2H).

### Reference Example 053

### Preparation of Compound 102

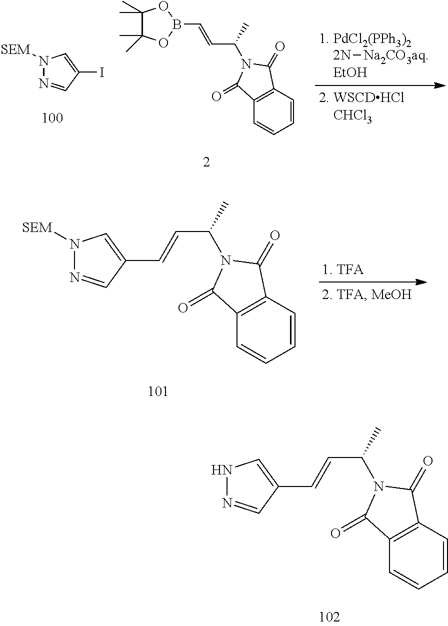

### Step 1 Preparation of Compound 101

2 mol/L sodium carbonate aqueous solution (12.2 mL, 24.4 mmol) was added to the ethanol solution (13 mL) of Compound 100 (4.00 g, 12.2 mmol. The preparation for method is described in WO2007/107346.) and Compound 2 (3.96 g, 12.2 mmol). The atmosphere was replaced with nitrogen, bis(triphenylphosphine) palladium(II) dichloride (0.858 g, 1.22 mmol) was added to the mixture. The mixture was subjected to microwave irradiation and stirred at 80° C. for 20 minuets. The mixture wad diluted with chloroform (26 mL), and WSCD (3.52 g, 18.3 mmol) was added to the mixture. The mixture was stirred at room temperature for 1 hour. Water was added to the mixture, and the mixture was extracted with chloroform. The organic layer was dried over magnesium sulfate. The solvent was condensed under reduced pressure. The residue was purified by silica gel chromatography (hexane-ethyl acetate) to afford Compound 101 (3.78 g, yield 78%).

¹H-NMR (CDCl₃) δ: 7.86 (dd, J=5.5, 3.0 Hz, 2H), 7.74 (dd, J=5.0, 3.0 Hz, 2H), 7.63 (s, 1H), 7.56 (s, 1H), 6.49 (d, J=15.7 Hz, 1H), 6.41 (dd, J=15.9, 7.3 Hz, 1H), 5.39 (s, 2H), 5.07 (m, 1H), 3.55 (m, 2H), 1.68 (d, J=7.1 Hz, 3H), 0.92 (t, J=8.3 Hz, 2H), 0.03 (s, 9H).

[M+H]=398, Method Condition 2: retention time 2.59 min

### Step 2 Preparation of Compound 102

Trifluoroacetic acid (20 mL) was added to Compound 101 (3.78 g, 9.51 mmol), and the mixture was stirred at room temperature for 1 hour. The solvent was condensed under reduced pressure. Saturated sodium bicarbonate water was added to the residue, and the mixture was extracted with chloroform. The organic layer was dried over magnesium sulfate. The solvent was condensed under reduced pressure. Methanol (10 mL) and trifluoroacetic acid (20 mL) were added to the residue obtained by condensing the solvent under reduced pressure. The mixture was stirred at 50° C. for 3.5 hours. The solvent was condensed under reduced pressure. Saturated sodium bicarbonate water was added to the residue, and the mixture was extracted with chloroform. The organic layer was dried over magnesium sulfate. The solvent was condensed under reduced pressure to afford Compound 102 (2.06 g, yield 81%).

¹H-NMR (DMSO-d₆) δ: 12.75 (s, 1H), 7.61-7.88 (m, 6H), 6.42 (d, J=16.2 Hz, 1.0H), 6.24 (dd, J=16.0, 6.3 Hz, 1H), 4.94 (m, 1H), 1.57 (d, J=7.1 Hz, 3H).

### Reference Example 054

### Preparation of Compound 104

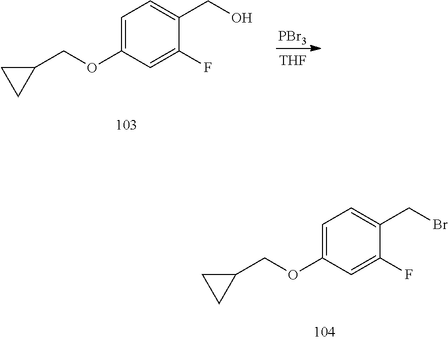

Compound 104 was obtained by using Compound 103 (The preparation for method is described in WO2010/050445.) instead of Compound 77 in Step 1 in Reference example 045.

¹H-NMR (CDCl₃) δ: 7.29-7.22 (m, 1H), 6.69-6.56 (m, 2H), 4.50 (s, 2H), 3.77 (d, J=7.0 Hz, 211), 1.32-1.19 (m, 1H), 0.70-0.61 (m, 2H), 0.39-0.31 (m, 2H).

### Reference Example 055

### Preparation of Compound 107

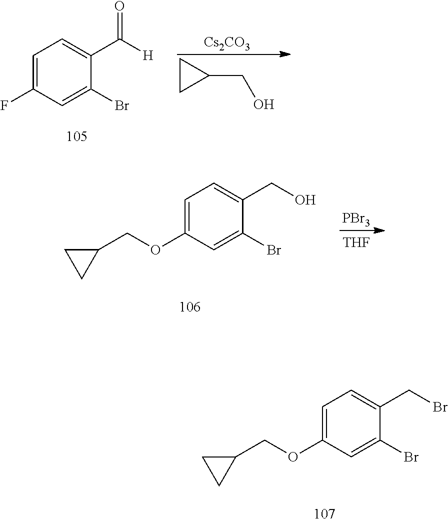

### Step 1 Preparation of Compound 106

Compound 105 (1.00 g, 4.93 mmol) was dissolved in cyclopropanecarbinol (3.00 mL, 37.0 mmol). Cesium carbonate (3.21 g, 9.85 mmol) was added to the mixture. The mixture was subjected to microwave irradiation and stirred at 180° C. for 80 minutes. Water was added to the mixture, and the mixture was extracted with ethyl acetate. The organic layer was washed with saturated brine, and dried over magnesium sulfate. The solvent was condensed under reduced pressure. The residue was purified by silica gel chromatography (hexane-ethyl acetate) to afford Compound 106 (496 mg, yield 39%).

¹H-NMR (CDCl₃) δ: 7.33 (d, J=8.5 Hz, 1H), 7.10 (d, J=2.4 Hz, 1H), 6.85 (dd, J=8.5, 2.5 Hz, 1H), 4.67 (d, J=6.3 Hz, 2H), 3.78 (d, J=7.0 Hz, 2H), 1.91 (t, J=6.3 Hz, 1H), 1.33-1.19 (m, 1H), 0.68-0.62 (m, 2H), 0.40-0.29 (m, 2H).

### Step 2 Preparation of Compound 107

Compound 107 was obtained by using Compound 106 instead of Compound 77 in Step 1 in Reference example 045.

¹H-NMR (CDCl₃) δ: 7.33 (d, J=8.5 Hz, 1H), 7.10 (d, J=2.6 Hz, 1H), 6.82 (dd, J=8.5, 2.6 Hz, 1H), 4.59 (s, 2H), 3.78 (d, J=6.9 Hz, 2H), 1.32-1.17 (m, 1H), 0.71-0.61 (m, 2H), 0.38-0.30 (m, 2H).

### Reference Example 056

### Preparation of Compound 111

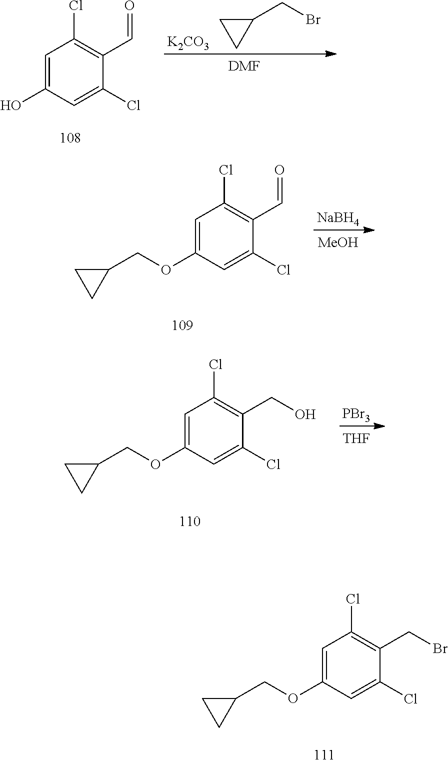

### Step 1 Preparation of Compound 109

Potassium carbonate (724 mg, 5.24 mmol) and (bromomethyl)cyclopropane (0.384 mL, 3.393 mmol) were added to the DMF solution (5 ml) of Compound 108 (500 mg, 2.62 mmol), and the mixture was stirred at 80° C. for 2 hours. Water was added to the mixture, and the mixture was extracted with diethylether. The organic layer was washed with saturated brine, dried over magnesium sulfate. The solvent was condensed under reduced pressure. The residue was purified by silica gel chromatography (hexane-ethyl acetate) to afford Compound 109 (647 mg, yield 100%).

¹H-NMR (CDCl₃) δ: 10.41 (s, 1H), 6.90 (s, 2H), 3.87 (d, J=7.1 Hz, 2H), 1.27 (s, 1H), 0.69 (q, J=6.4 Hz, 2H), 0.37 (q, J=5.1 Hz, 2H).

[M+H]=245.15, Method Condition 2: retention time 2.40 min

### Step 2 Preparation of Compound 110

Sodium borohydride (149 mg, 3.95 mmol) was added to the methanol solution (5 mL) of Compound 109 (645 mg, 2.63 mmol), and the mixture was stirred at room temperature for 2 hours. Saturated ammonium chloride aqueous solution was added to the reaction mixture, and the reaction mixture was extracted with ethyl acetate. The organic layer was washed with saturated sodium bicarbonate water and saturated brine, and dried over magnesium sulfate. The solvent was condensed under reduced pressure to afford Compound 110 (629 mg, yield 97%).

¹H-NMR (CDCl₃) δ: 6.88 (s, 2H), 4.88 (d, J=4.1 Hz, 2H), 3.78 (d, J=7.1 Hz, 2H), 1.21-1.28 (m, 1H), 0.66 (q, J=6.3 Hz, 2H), 0.35 (q, J=5.1 Hz, 2H).

### Step 3 Preparation of Compound 111

Compound 111 was obtained by using Compound 106 instead of Compound 77 in Step 1 in Reference example 045.

¹H-NMR (CDCl₃) δ: 6.88 (s, 2H), 4.73 (s, 2H), 3.78 (d, J=7.1 Hz, 2H), 1.21-1.27 (m, 1H), 0.66 (q, J=6.3 Hz, 2H), 0.34 (q, J=5.1 Hz, 2H).

### Reference Example 057

### Preparation of Compound 114

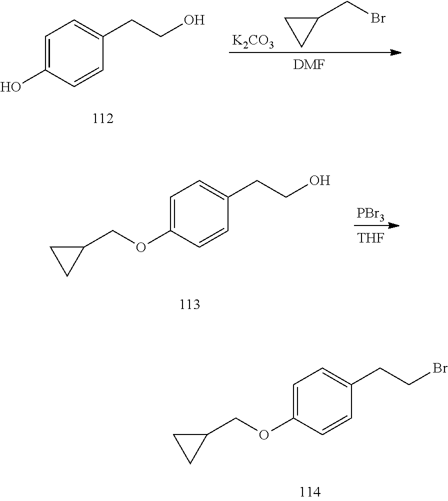

### Step 1 Preparation of Compound 113

Potassium carbonate (2.00 g, 14.5 mmol) and (bromomethyl)cyclopropane (1.06 mL, 10.9 mmol) were added to the DMF solution (10 ml) of Compound 112 (1.00 g, 7.24 mmol), and the mixture was stirred at 80° C. for 5.5 hours. Water was added to the mixture, and the mixture was extracted with diethylether. The organic layer was washed with saturated brine, dried over magnesium sulfate. The solvent was condensed under reduced pressure. The residue was purified by silica gel chromatography (hexane-ethyl acetate) to afford Compound 113 (874 mg, yield 63%).

¹H-NMR (CDCl₃) δ: 7.1 (d, J=8.6 Hz, 2H), 6.86 (d, J=8.1 Hz, 2H), 3.83-3.77 (m, 4H), 2.80 (t, J=6.3 Hz, 2H), 1.21-1.31 (m, 1H), 0.64 (q, J=6.3 Hz, 2H), 0.34 (q, J=5.1 Hz, 2H).

### Step 2 Preparation of Compound 114

Compound 114 was obtained by using Compound 113 instead of Compound 77 in Step 1 in Reference example 045.

¹H-NMR (CDCl₃) δ: 7.11 (d, J=8.6 Hz, 2.H), 6.85 (d, J=8.1 Hz, 2H), 3.78 (d, J=7.1 Hz, 2H), 3.52 (t, J=7.6 Hz, 2H), 3.09 (t, J=7.6 Hz, 2H), 1.21-1.31 (s, 1H), 0.64 (q, J=6.2 Hz, 2H), 0.34 (q, J=5.1 Hz, 2H).

### Reference Example 044

### Preparation of Compound 119

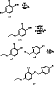

### Step 1 Preparation of Compound 116

Potassium carbonate (1.77 g, 12.8 mmol) and iodoethane (0.542 mL, 6.71 mmol) were added to DMF solution of Compound 115 (1.00 g, 6.39 mmol), and the mixture was stirred at 80° C. for 2.5 hours. Water was added to the reaction mixture, and the reaction mixture was extracted with diethylether. The organic layer was washed with saturated brine, and dried over magnesium sulfate. The solvent was condensed under reduced pressure. The residue was purified by silica gel chromatography (hexane-ethyl acetate) to afford Compound 116 (1.10 g, yield 93%).

¹H-NMR (DMSO-d₆) δ: 10.2 (s, 1H), 7.82 (d, J=8.6 Hz, 1H), 7.15 (d, J=2.5 Hz, 1H), 7.07 (t, J=4.3 Hz, 1H), 4.18 (q, J=7.1 Hz, 2H), 1.35 (t, J=6.8 Hz, 3H).

### Step 2 Preparation of Compound 117

Meta-chloroperbenzoic acid (2.27 g, 8.94 mmol) was added to the dichloromethane solution (10 mL) of Compound 116 (1.10 g, 5.96 mmol), and the mixture was stirred at room temperature for 18 hours. Saturated sodium hydrogen carbonate solution was added to the reaction mixture, and the reaction mixture was extracted with chloroform. The organic layer was washed with saturated brine, and dried over magnesium sulfate. The solvent was condensed under reduced pressure. 2N aqueous sodium hydroxide (8.94 mL, 17.9 mmol) was added to the methanol solution (10 mL) of the residue, and the mixture was stirred at room temperature for 1 hour. Water was added to the mixture, and the mixture was extracted with diethylether. The organic layer was washed with saturated brine, and dried over magnesium sulfate. The solvent was condensed under reduced pressure. The residue was purified by silica gel chromatography (hexane-ethyl acetate) to afford Compound 117 (0.867 g, yield 84%).

¹H-NMR (CDCl₃) δ: 6.90 (dd, J=19.3, 5.6 Hz, 2H), 6.74 (dd, J=8.9, 2.8 Hz, 1H), 5.20 (s, 1H), 3.95 (q, J=6.9 Hz, 2H), 1.38 (t, J=7.1 Hz, 3H).

Step Preparation of Compound 119

Compound 118 (0.282 g, 1.12 mmol) and potassium carbonate (0.202 g, 1.46 mmol) were added to DMF solution of Compound 117 (0.194 g, 1.12 mmol), and the mixture was stirred overnight at room temperature. Water was added to the reaction mixture, and the reaction mixture was extracted with ethyl acetate. The organic layer was washed with water, and dried over magnesium sulfate. The solvent was condensed under reduced pressure. The residue was purified by silica gel chromatography (hexane-ethyl acetate) to afford Compound 119 (0.338 g, yield 88%).

¹H-NMR (CDCl₃) δ: 8.63 (d, J=2.03 Hz, 1H), 7.86 (dd, J=8.36, 2.28 Hz, 1H), 7.56 (d, J=8.11 Hz, 1H), 6.97 (d, J=3.04 Hz, 1H), 6.88 (d, J=9.12 Hz, 1H), 6.73 (dd, J=9.12, 3.04 Hz, 1H), 5.15 (s, 2H), 3.97 (q, J=6.93 Hz, 2H), 1.39 (t, J=7.10 Hz, 3H).

[M+H]=343, Method Condition 2: retention time 2.65 min

### Example 001

### Preparation of Compound I-1

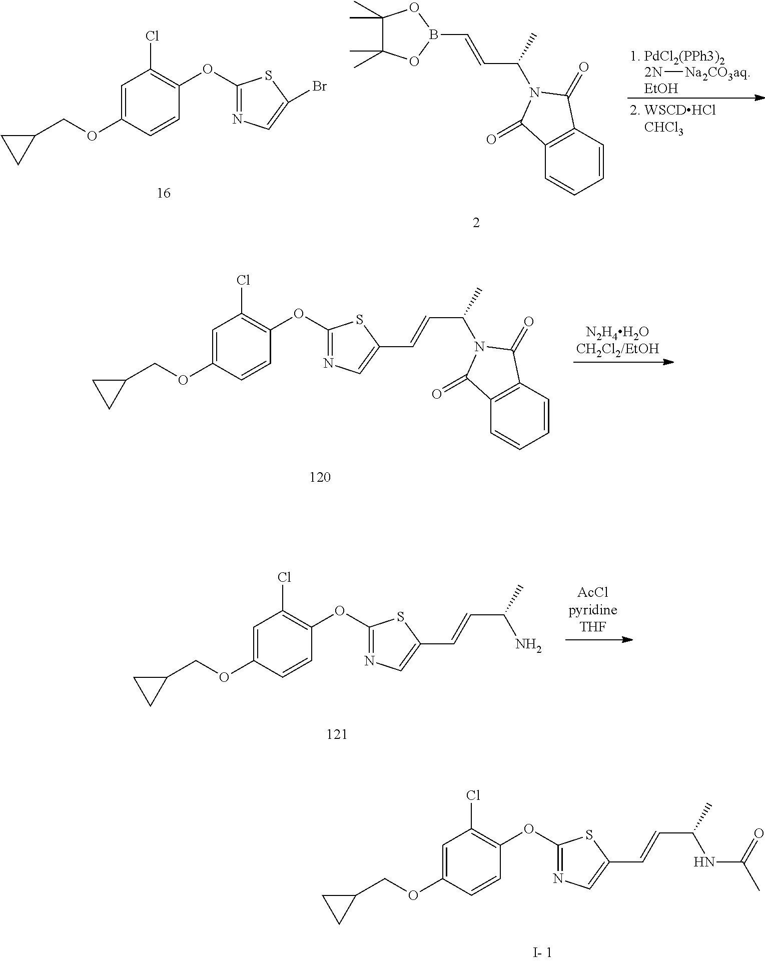

### Step 1 Preparation of Compound 120

2 mol/L sodium carbonate aqueous solution (27.7 mL, 55.5 mmol) was added to the ethanol solution (80 mL) of Compound 16 (10.0 g, 27.76 mmol) in Reference Example 005 and Compound 2 (1.79 g, 5.48 mmol) in Reference Example 001. The atmosphere was replaced with nitrogen, and bis(triphenylphosphine) palladium(II) dichloride (1.95 g, 2.77 mmol) was added to the mixture. The mixture was subjected to microwave irradiation and stirred at 80° C. for 1.5 hours. The mixture was diluted with Chroloform (160 mL), and WSCD (7.97 g, 41.6 mmol) was added to the mixture. The mixture was stirred at room temperature for 1 hour. Water was added to the mixture, and the mixture was extracted with chloroform. The organic layer was dried over magnesium sulfate. The solvent was condensed under reduced pressure. The residue was purified by silica gel chromatography (hexane-ethyl acetate) to afford Compound 120 (12.0 g, yield 87%).

¹H-NMR (CDCl₃) δ: 7.83-7.79 (m, 2H), 7.73-7.68 (m, 2H), 7.21 (d, J=9.0 Hz, 1H), 7.01 (s, 1H), 6.98 (d, J=2.9 Hz, 1H), 6.83 (dd, J=9.0, 2.9 Hz, 1H), 6.57 (d, J=15.7 Hz, 1H), 6.15 (dd, J=15.7, 7.6 Hz, 1H), 5.05-4.96 (m, 1H), 3.78 (d, J=6.9 Hz, 2H), 1.62 (d, J=7.2 Hz, 3H), 1.29-1.23 (m, 1H), 0.70-0.62 (m, 2H), 0.38-0.32 (m, 2H).

### Step 2 Preparation of Compound 121

Hydrazine monohydrate (11.76 mL, 242 mmol) and ethanol (15 mL) were added to the dichloromethane solution (90 mL) of Compound 120 (12.0 g, 24.20 mmol), and the mixture was stirred at 60° C. for 1.5 hours. The mixture was cooled to room temperature, and saturated sodium bicarbonate water was added to the mixture. The mixture was stirred, and the mixture was extracted with chloroform. The organic layer was dried over magnesium sulfate, and filered. The filtrate was condensed under reduced pressure. The residue was dried under vacuum to afford Compound 121 (8.49 g, yield 100%).

¹H-NMR (CDCl₃) δ: 7.23 (d, J=8.9 Hz, 1H), 6.99 (d, J=2.9 Hz, 1H), 6.98 (s, 1H), 6.84 (dd, J=9.0, 2.9 Hz, 1H), 6.45 (d, J=15.6 Hz, 1H), 5.80 (dd, J=15.6, 6.5 Hz, 1H), 3.79 (d, J=6.9 Hz, 2H), 3.64-3.55 (m, 1H), 1.32-1.21 (m, 1H), 1.21 (d, J=6.4 Hz, 3H), 0.69-0.63 (m, 2H), 0.38-0.33 (m, 2H).

### Step 3 Preparation of Compound I-1

The tetrahydrofuran solution (50 mL) of Compound 121 (5.0 g, 14.25 mmol) was cooled in ice under a nitrogen atmosphere. Pyridine (1.73 mL, 21.4 mmol) and acetyl chloride (1.53 mL, 21.4 mmol) were added to the mixture, and the mixture was stirred for 10 minutes. Methanol (20 mL) was added to the mixture, and the solvent was removed under reduced pressure. 0.2 mol/L hydrochloric acid aqueous solution was added to the residue, the mixture was extracted with ethyl acetate. The organic layer was washed with saturated brine, and dried over magnesium sulfate. The solvent was condensed under reduced pressure. The residue was purified by silica gel chromatography (hexane-ethyl acetate) to afford Compound I-1 (5.12 g, yield 91%).

¹H-NMR (DMSO-d₆) δ: 7.93 (d, J=7.9 Hz, 1H), 7.45 (d, J=9.0 Hz, 1H), 7.20 (d, J=2.7 Hz, 1H), 7.18 (s, 1H), 6.99 (dd, J=8.9, 3.0 Hz, 1H), 6.49 (d, J=15.9 Hz, 1H), 5.78 (dd, J=15.8, 5.4 Hz, 1H), 4.46-4.35 (m, 1H), 3.86 (d, J=7.0 Hz, 2H), 1.81 (s, 3H),

### Example 002

### Preparation of Compound I-2

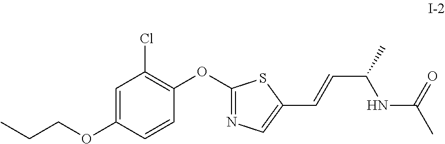 [SMILES: CCCOc1ccc(Oc2ncc(/C=C/[C@H](C)NC(C)=O)s2)c(Cl)c1]

Compound I-2 was obtained by using Compound 17 instead of Compound 16 in Step 1 in Example 001.

[M+H]=381, Method Condition 2: retention time 2.30 min

### Example 003

### Preparation of Compound I-3

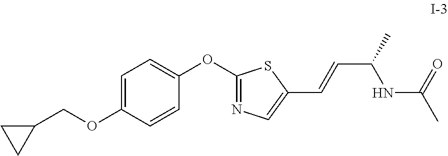 [SMILES: CC(=O)N[C@@H](C)/C=C/c1cnc(Oc2ccc(OCC3CC3)cc2)s1]

Compound I-3 was obtained by using Compound 18 instead of Compound 16 in Step 1 in Example 001.

### Example 004

### Preparation of Compound I-4

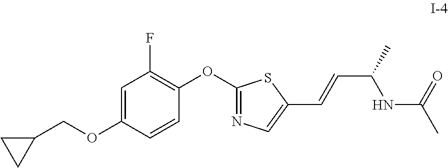 [SMILES: CC(=O)N[C@@H](C)/C=C/c1cnc(Oc2ccc(OCC3CC3)cc2F)s1]

Compound I-4 was obtained by using Compound 19 instead of Compound 16 in Step 1 in Example 001.

[M+H]=377, Method Condition 2: retention time 2.16 min

### Example 005

### Preparation of Compound I-5

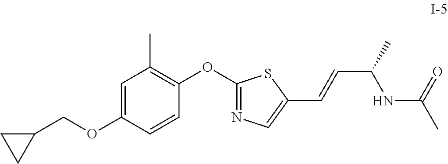 [SMILES: CC(=O)N[C@@H](C)/C=C/c1cnc(Oc2ccc(OCC3CC3)cc2C)s1]

Compound I-5 was obtained by using Compound 20 instead of Compound 16 in Step 1 in Example 001.

[M+H]=373, Method Condition 2: retention time 2.17 min

### Example 006

### Preparation of Compound I-6

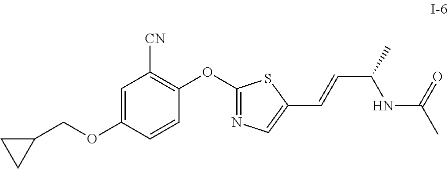 [SMILES: CC(=O)N[C@@H](C)/C=C/c1cnc(Oc2ccc(OCC3CC3)cc2C#N)s1]

Compound I-6 was obtained by using Compound 22 instead of Compound 16 in Step 1 in Example 001.

[M+H]=384, Method Condition 2: retention time 2.08 min

### Example 007

### Preparation of Compound I-7

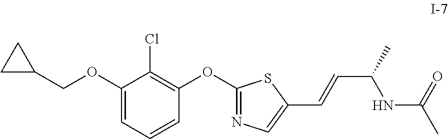 [SMILES: CC(=O)N[C@@H](C)/C=C/c1cnc(Oc2cccc(OCC3CC3)c2Cl)s1]

Compound I-7 was obtained by using Compound 37 instead of Compound 16 in Step 1 in Example 001.

### Example 008

### Preparation of Compound I-8

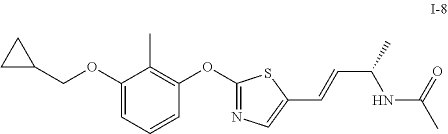 [SMILES: CC(=O)N[C@@H](C)/C=C/c1cnc(Oc2cccc(OCC3CC3)c2C)s1]

Compound I-8 was obtained by using Compound 36 instead of Compound 16 in Step 1 in Example 001.

[M+H]=373, Method Condition 2: retention time 2.27 min

### Example 009

### Preparation of Compound I-9

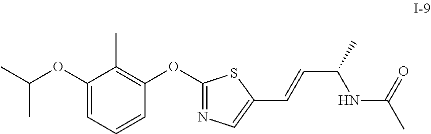 [SMILES: CC(=O)N[C@@H](C)/C=C/c1cnc(Oc2cccc(OC(C)C)c2C)s1]

Compound I-9 was obtained by using Compound 35 instead of Compound 16 in Step 1 in Example 001.

[M+H]=361, Method Condition 2: retention time 2.23 min

### Example 010

### Preparation of Compound I-10

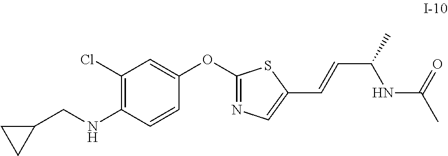 [SMILES: CC(=O)N[C@@H](C)/C=C/c1cnc(Oc2ccc(NCC3CC3)c(Cl)c2)s1]

Compound I-10 was obtained by using Compound 32 instead of Compound 16 in Step 1 in Example 001.

[M+H]=392, Method Condition 2: retention time 2.30 min

### Example 011

### Preparation of Compound I-11

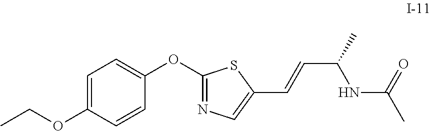 [SMILES: CCOc1ccc(Oc2ncc(/C=C/[C@H](C)NC(C)=O)s2)cc1]

Compound I-11 was obtained by using Compound 24 instead of Compound 16 in Step 1 in Example 001.

[M+H]=333, Method Condition 2: retention time 1.90 min

### Example 012

### Preparation of Compound I-12

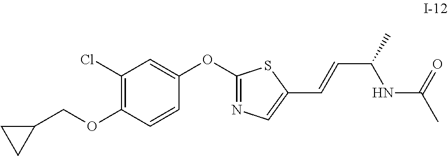 [SMILES: CC(=O)N[C@@H](C)/C=C/c1cnc(Oc2ccc(OCC3CC3)c(Cl)c2)s1]

Compound I-12 was obtained by using Compound 27 instead of Compound 16 in Step 1 in Example 001.

[M+H]=393, Method Condition 2: retention time 2.26 min

### Example 013

### Preparation of Compound I-13

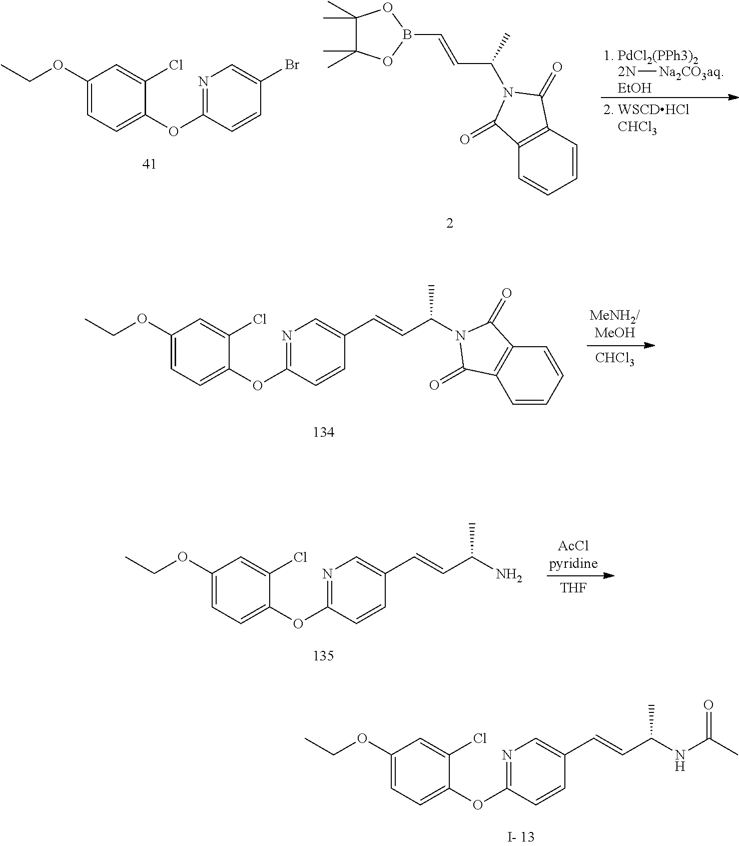

### Step 1 Preparation of Compound 134

2 mol/L sodium carbonate aqueous solution (4.56 mL, 9.13 mmol) was added to the ethanol solution (12 mL) of Compound 41 (1.50 g, 4.56 mmol) and Compound 2 (1.79 g, 5.48 mmol). The atmosphere was replaced with nitrogen, and bis(triphenylphosphine) palladium(II) dichloride (0.320 g, 0.456 mmol) was added to the mixture. The mixture was subjected to microwave irradiation and stirred at 80° C. for 20 minutes. The mixture was diluted with Chroloform (24 mL), and WSCD (1.31 g, 6.85 mmol) was added to the mixture. The mixture was stirred at room temperature for 1 hour. Water was added to the mixture, and the mixture was extracted with chloroform. The organic layer was dried over magnesium sulfate. The solvent was condensed under reduced pressure. The residue was purified by silica gel chromatography (hexane-ethyl acetate) to afford Compound 134 (2.23 g, yield 98%).

¹H-NMR (CDCl₃) δ: 8.05 (d, J=2.4 Hz, 1H), 7.84-7.81 (m, 2H), 7.76 (dd, J=8.3, 2.4 Hz, 1H), 7.71-7.68 (m, 2H), 7.08 (d, J=8.8 Hz, 1H), 6.97 (d, J=2.9 Hz, 1H), 6.87 (d, J=8.5 Hz, 1H), 6.82 (dd, J=8.9, 3.0 Hz, 1H), 6.54-6.53 (m, 2H), 5.12-5.03 (m, 1H), 4.01 (q, J=6.8 Hz, 2H), 1.66 (d, J=7.0 Hz, 3H), 1.41 (t, J=7.0 Hz, 3H).

### Step 2 Preparation of Compound 135

40% methylamine-methanol solution (100 mL) was added to the chloroform solution (20 mL) of Compound 134 (2.2 g, 4.41 mmol), and the mixture was stirred overnight at room temperature. The mixture was condensed under reduced pressure. The residue was suspended in ethyl acetate-hexane, and the insoluble matter was filtered. The filtrate was condensed under reduced pressure. The residue was advanced to the next step.

¹H-NMR (CDCl₃) δ: 8.06 (d, J=2.1 Hz, 1H), 7.73 (dd, J=8.7, 2.3 Hz, 1H), 7.10 (d, J=8.8 Hz, 1H), 6.98 (d, J=2.7 Hz, 1H), 6.88 (d, J=8.7 Hz, 1H), 6.83 (dd, J=9.1, 2.8 Hz, 1H), 6.40 (d, J=15.9 Hz, 1H), 6.13 (dd, J=15.9, 6.6 Hz, 1H), 4.01 (q, J=6.9 Hz, 2H), 3.71-3.62 (m, 1H), 1.42 (t, J=7.0 Hz, 3H), 1.25 (d, J=6.9 Hz, 3H).

### Step 3 Preparation of Compound I-13

The tetrahydrofuran solution (15 mL) of Compound 135 (1.41 g, 4.41 mmol) was cooled in ice under a nitrogen atmosphere. Pyridine (0.535 mL, 6.62 mmol) and acetyl chloride (0.472 mL, 6.62 mmol) were added to the mixture, and the mixture was stirred for 10 minutes. Methanol (20 mL) was added to the mixture, and the solvent was removed under reduced pressure. 0.2 mol/L hydrochloric acid aqueous solution was added to the residue, the mixture was extracted with ethyl acetate. The organic layer was wased with saturated brine, and dried over magnesium sulfate. The solvent was condensed under reduced pressure. The residue was purified by silica gel chromatography (hexane-ethyl acetate) to afford Compound I-13 (1.08 g, yield 68%).

¹H-NMR (DMSO-d₆) δ: 8.06 (d, J=2.1 Hz, 1H), 8.00-7.92 (m, 2H), 7.20 (d, J=9.0 Hz, 1H), 7.11 (d, J=2.9 Hz, 1H), 7.00 (d, J=8.5 Hz, 1H), 6.94 (dd, J=9.0, 2.9 Hz, 1H), 6.40 (d, J=16.2 Hz, 1H), 6.23 (dd, J=16.1, 5.4 Hz, 1H), 4.55-4.43 (m, 1H), 4.05 (q, J=7.0 Hz, 2H), 1.83 (s, 3H), 1.33 (t, J=7.0 Hz, 3H), 1.20 (d, J=6.9 Hz, 3H).

### Example 014

### Preparation of Compound I-14

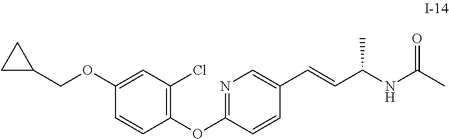 [SMILES: CC(=O)N[C@@H](C)/C=C/c1ccc(Oc2ccc(OCC3CC3)cc2Cl)nc1]

Compound I-14 was obtained by using Compound 42 instead of Compound 41 in Step 1 in Example 013.

¹H-NMR (DMSO-d₆) δ: 8.06 (d, J=2.1 Hz, 1H), 7.99-7.93 (m, 2H), 7.19 (d, J=9.0 Hz, 1H), 7.11 (d, J=2.7 Hz, 1H), 6.99 (d, J=8.5 Hz, 1H), 6.94 (dd, J=9.0, 3.0 Hz, 1H), 6.40 (d, J=16.2 Hz, 1H), 6.23 (dd, J=16.2, 5.1 Hz, 1H), 4.53-4.44 (m, 1H), 3.84 (d, J=7.0 Hz, 2H), 1.83 (s, 3H), 1.27-1.18 (m, 1H), 1.19 (d, J=7.0 Hz, 3H), 0.61-0.55 (m, 2H), 0.35-0.30 (m, 2H).

### Example 015

### Preparation of Compound I-15

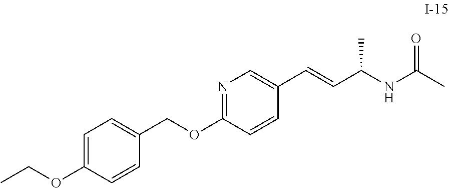 [SMILES: CCOc1ccc(COc2ccc(/C=C/[C@H](C)NC(C)=O)cn2)cc1]

Compound I-15 was obtained by using Compound 46 instead of Compound 41 in Step 1 in Example 013.

¹H-NMR (DMSO-d₆) δ: 8.15 (d, J=2.4 Hz, 1H), 7.97 (d, J=7.9 Hz, 1H), 7.84 (dd, J=8.6, 2.4 Hz, 1H), 7.35 (d, J=8.4 Hz, 2H), 6.90 (d, J=8.4 Hz, 2H), 6.80 (d, J=8.6 Hz, 1H), 6.40 (d, J=16.3 Hz, 1H), 6.19 (dd, J=16.1, 5.5 Hz, 1H), 5.24 (s, 2H), 4.55-4.44 (m, 1H), 4.01 (q, J=7.0 Hz, 2H), 1.83 (s, 3H), 1.31 (t, J=7.0 Hz, 3H), 1.20 (d, J=6.9 Hz, 3H).

### Example 016

### Preparation of Compound I-16

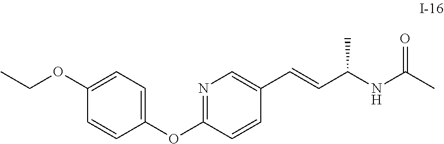 [SMILES: CCOc1ccc(Oc2ccc(/C=C/[C@H](C)NC(C)=O)cn2)cc1]

Compound I-16 was obtained by using Compound 47 instead of Compound 41 in Step 1 in Example 013.

[M+H]=327, Method Condition 2: retention time 1.93 min

### Example 017

### Preparation of Compound I-17

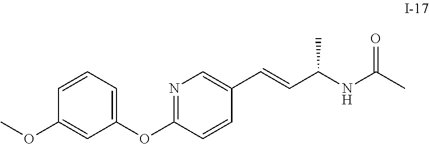 [SMILES: COc1cccc(Oc2ccc(/C=C/[C@H](C)NC(C)=O)cn2)c1]

Compound I-17 was obtained by using Compound 48 instead of Compound 41 in Step 1 in Example 013.

[M+H]=313, Method Condition 2: retention time 1.79 min

### Example 018

### Preparation of Compound I-18

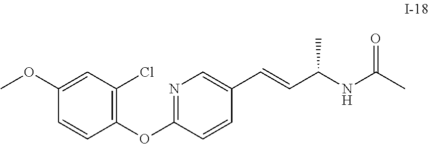 [SMILES: COc1ccc(Oc2ccc(/C=C/[C@H](C)NC(C)=O)cn2)c(Cl)c1]

Compound I-18 was obtained by using Compound 39 instead of Compound 41 in Step 1 in Example 013.

¹H-NMR (DMSO-d₆) δ: 8.06 (d, J=2.3 Hz, 1H), 8.00-7.94 (m, 2H), 7.22 (d, J=8.8 Hz, 1H), 7.14 (d, J=2.9 Hz, 1H), 7.01 (d, J=8.5 Hz, 2H), 6.95 (dd, J=8.8, 2.9 Hz, 2H), 6.40 (d, J=16.2 Hz, 1H), 6.23 (dd, J=16.2, 5.4 Hz, 1H), 4.54-4.43 (m, 1H), 3.79 (s, 3H), 1.83 (s, 3H), 1.20 (d, J=6.9 Hz, 3H).

### Example 019

### Preparation of Compound I-19

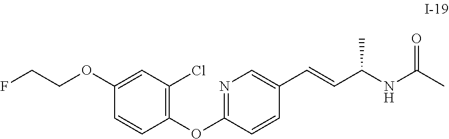 [SMILES: CC(=O)N[C@@H](C)/C=C/c1ccc(Oc2ccc(OCCF)cc2Cl)nc1]

Compound I-19 was obtained by using Compound 44 instead of Compound 41 in Step 1 in Example 013.

[M+H]=379, Method Condition 2: retention time 1.90 min

### Example 020

### Preparation of Compound I-20

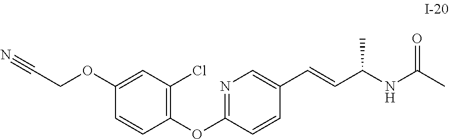 [SMILES: CC(=O)N[C@@H](C)/C=C/c1ccc(Oc2ccc(OCC#N)cc2Cl)nc1]

Compound I-20 was obtained by using Compound 43 instead of Compound 41 in Step 1 in Example 013.

[M+H]=372, Method Condition 2: retention time 1.80 min

### Example 021

### Preparation of Compound I-21

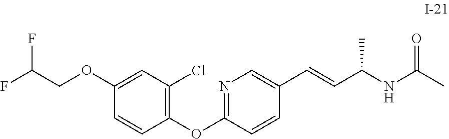 [SMILES: CC(=O)N[C@@H](C)/C=C/c1ccc(Oc2ccc(OCC(F)F)cc2Cl)nc1]

Compound I-21 was obtained by using Compound 45 instead of Compound 41 in Step 1 in Example 013.

[M+H]=397, Method Condition 2: retention time 1.97 min

### Example 022

### Preparation of Compound I-22

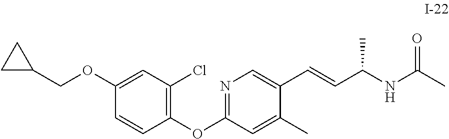 [SMILES: CC(=O)N[C@@H](C)/C=C/c1cnc(Oc2ccc(OCC3CC3)cc2Cl)cc1C]

Compound I-22 was obtained by using Compound 86 instead of Compound 41 in Step 1 in Example 013.

[M+H]=401, Method Condition 2: retention time 2.43 min

### Example 023

### Preparation of Compound I-23

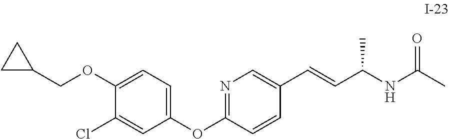 [SMILES: CC(=O)N[C@@H](C)/C=C/c1ccc(Oc2ccc(OCC3CC3)c(Cl)c2)nc1]

Compound I-23 was obtained by using Compound 50 instead of Compound 41 in Step 1 in Example 013.

[M+H]=387, Method Condition 2: retention time 2.24 min

### Example 024

### Preparation of Compound I-24

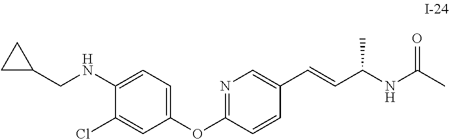 [SMILES: CC(=O)N[C@@H](C)/C=C/c1ccc(Oc2ccc(NCC3CC3)c(Cl)c2)nc1]

Compound I-24 was obtained by using Compound 51 instead of Compound 41 in Step 1 in Example 013.

¹H-NMR (DMSO-d₆) δ: 8.09 (d, J=2.0 Hz, 1H), 7.98 (d, J=7.8 Hz, 1H), 7.91 (dd, J=8.7, 2.1 Hz, 1H), 7.11 (d, J=2.4 Hz, 1H), 6.95-6.89 (m, 2H), 6.75 (d, J=8.8 Hz, 1H), 6.40 (d, J=16.2 Hz, 1H), 6.22 (dd, J=16.0, 5.3 Hz, 1H), 5.12 (t, J=5.6 Hz, 1H), 4.55-4.42 (m, 1H), 3.01 (t, J=6.1 Hz, 2H), 1.83 (s, 3H), 1.23-1.07 (m, 4H), 0.51-0.44 (m, 2H), 0.29-0.22 (m, 2H).

### Example 025

### Preparation of Compound I-25

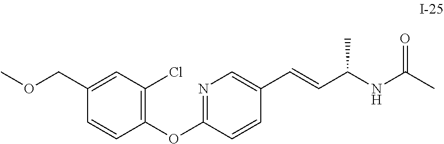 [SMILES: COCc1ccc(Oc2ccc(/C=C/[C@H](C)NC(C)=O)cn2)c(Cl)c1]

Compound I-25 was obtained by using Compound 52 instead of Compound 41 in Step 1 in Example 013.

¹H-NMR (CDCl₃) δ: 8.09 (d, J=1.5 Hz, 1H), 7.64 (dd, J=8.6, 2.0 Hz, 1H), 7.48 (s, 1H), 7.31 (d, J=8.1 Hz, 1H), 6.91 (d, J=8.6 Hz, 1H), 6.74 (d, J=8.6 Hz, 1H), 6.44 (d, J=16.2 Hz, 1H), 6.06 (dd, J=16.2, 5.6 Hz, 1H), 5.43 (d, J=7.6 Hz, 1H), 5.28 (s, 2H), 4.74 (dd, J=13.4, 6.3 Hz, 1H), 3.90 (s, 3H), 2.02 (s, 3H), 1.34 (d, J=6.6 Hz, 3H).

[M+H]=361, Method Condition 2: retention time 2.00 min

### Example 026

### Preparation of Compound I-26

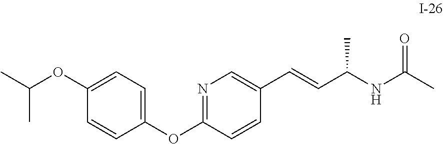 [SMILES: CC(=O)N[C@@H](C)/C=C/c1ccc(Oc2ccc(OC(C)C)cc2)nc1]

Compound I-26 was obtained by using Compound 49 instead of Compound 41 in Step 1 in Example 013.

[M+H]=341, Method Condition 2: retention time 2.01 min

### Example 027

### Preparation of Compound I-27

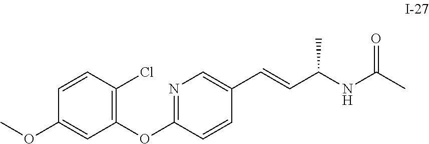 [SMILES: COc1ccc(Cl)c(Oc2ccc(/C=C/[C@H](C)NC(C)=O)cn2)c1]

Compound I-27 was obtained by using Compound 55 instead of Compound 41 in Step 1 in Example 013.

[M+H]=347, Method Condition 2: retention time 1.91 min

### Example 028

### Preparation of Compound I-28

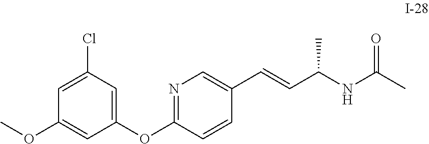 [SMILES: COc1cc(Cl)cc(Oc2ccc(/C=C/[C@H](C)NC(C)=O)cn2)c1]

Compound I-28 was obtained by using Compound 56 instead of Compound 41 in Step 1 in Example 013.

[M+H]=347, Method Condition 2: retention time 2.04 min

### Example 029

### Preparation of Compound I-29

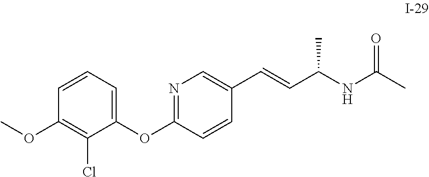 [SMILES: COc1cccc(Oc2ccc(/C=C/[C@H](C)NC(C)=O)cn2)c1Cl]

Compound I-29 was obtained by using Compound 57 instead of Compound 41 in Step 1 in Example 013.

[M+H]=347, Method Condition 2: retention time 1.84 min

### Example 030

### Preparation of Compound I-30

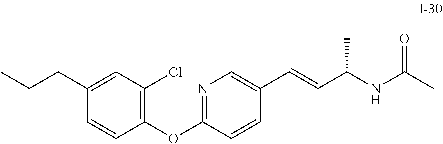 [SMILES: CCCc1ccc(Oc2ccc(/C=C/[C@H](C)NC(C)=O)cn2)c(Cl)c1]

Compound I-30 was obtained by using Compound 65 instead of Compound 41 in Step 1 in Example 013.

¹H-NMR (CDCl₃) δ: 8.07 (d, J=2.0 Hz, 1H), 7.72 (dd, J=8.4, 2.3 Hz, 1H), 7.28 (s, 1H), 7.10 (d, J=1.0 Hz, 2H), 6.89 (d, J=8.6 Hz, 1H), 6.44 (d, J=16.2 Hz, 1H), 6.09 (dd, J=16.2, 5.6 Hz, 1H), 5.44 (d, J=7.6 Hz, 1H), 4.76-4.69 (m, 1H), 2.58 (t, J=7.6 Hz, 2H), 2.01 (s, 3H), 1.66 (td, J=15.0, 7.3 Hz, 2H), 1.33 (d, J=6.6 Hz, 3H), 0.97 (t, J=7.3 Hz, 3H).

[M+H]=359, Method Condition 2: retention time 2.37 min

### Example 031

### Preparation of Compound I-31

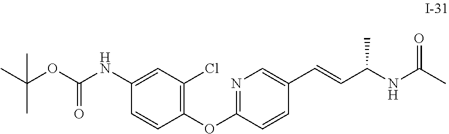 [SMILES: CC(=O)N[C@@H](C)/C=C/c1ccc(Oc2ccc(NC(=O)OC(C)(C)C)cc2Cl)nc1]

Compound I-31 was obtained by using Compound 59 instead of Compound 41 in Step 1 in Example 013.

[M+H]=432, Method Condition 2: retention time 2.17 min

### Example 032

### Preparation of Compound I-32

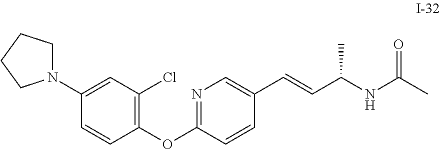 [SMILES: CC(=O)N[C@@H](C)/C=C/c1ccc(Oc2ccc(N3CCCC3)cc2Cl)nc1]

Compound I-32 was obtained by using Compound 64 instead of Compound 41 in Step 1 in Example 013.

¹H-NMR (CDCl₃) δ: 8.08 (d, J=2.0 Hz, 1H), 7.69 (dd, J=8.6, 2.5 Hz, 1H), 7.05 (d, J=9.1 Hz, 1H), 6.83 (d, J=8.6 Hz, 1H), 6.60 (d, J=3.0 Hz, 1H), 6.50-6.40 (m, 2H), 6.07 (dd, J=16.0, 5.8 Hz, 1H), 5.45 (d, J=7.6 Hz, 1H), 4.73 (m, 1H), 3.27 (t, J=6.3 Hz, 4H), 2.01 (t, J=6.3 Hz, 4H), 2.01 (s, 3H), 1.32 (d, J=7.1 Hz, 3H).

[M+H]=386, Method Condition 2: retention time 2.27 min

### Example 033

### Preparation of Compound I-33

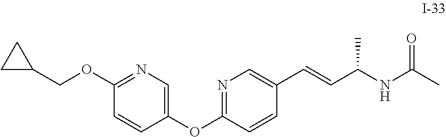 [SMILES: CC(=O)N[C@@H](C)/C=C/c1ccc(Oc2ccc(OCC3CC3)nc2)nc1]

Compound I-33 was obtained by using Compound 53 instead of Compound 41 in Step 1 in Example 013.

¹H-NMR (CDCl₃) δ: 8.07 (d, J=2.0 Hz, 1H), 7.99 (d, J=3.0 Hz, 1H), 7.72 (dd, J=8.3, 2.4 Hz, 1H), 7.41 (dd, J=8.9, 2.8 Hz, 1H), 6.88 (d, J=8.6 Hz, 1H), 6.81 (d, J=8.6 Hz, 1H), 6.44 (d, J=16.2 Hz, 1H), 6.10 (dd, J=16.2, 5.6 Hz, 1H), 5.46 (d, J=8.1 Hz, 1H), 4.74 (m, 1H), 4.12 (d, J=7.1 Hz, 2H), 2.02 (s, 3H), 1.29 (m, 1H), 1.34 (d, J=7.1 Hz, 3H), 0.62 (m, 2H), 0.35 (m, 2H).

[M+H]=354, Method Condition 2: retention time 1.96 min

### Example 034

### Preparation of Compound I-34

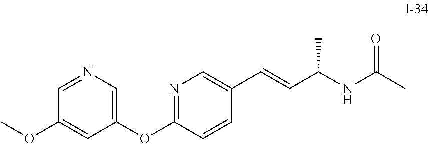 [SMILES: COc1cncc(Oc2ccc(/C=C/[C@H](C)NC(C)=O)cn2)c1]

Compound I-34 was obtained by using Compound 54 instead of Compound 41 in Step 1 in Example 013.

¹H-NMR (CDCl₃) δ: 8.17 (d, J=2.0 Hz, 1H), 8.11 (s, 2H), 7.76 (dd, J=8.6, 2.0 Hz, 1H), 7.05 (t, J=2.3 Hz, 1H), 6.93 (d, J=8.6 Hz, 1H), 6.46 (d, J=15.7 Hz, 1H), 6.13 (dd, J=16.2, 5.6 Hz, 1H), 5.51 (d, J=7.6 Hz, 1H), 4.75 (m, 1H), 3.86 (s, 3H), 2.02 (s, 3H), 1.34 (d, J=6.6 Hz, 3H).

[M+H]=314, Method Condition 2: retention time 1.25 min

### Example 035

### Preparation of Compound I-35

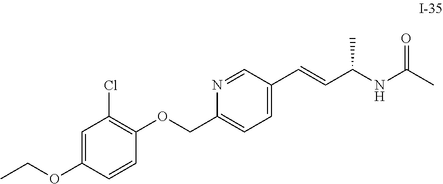 [SMILES: CCOc1ccc(OCc2ccc(/C=C/[C@H](C)NC(C)=O)cn2)c(Cl)c1]

Compound I-35 was obtained by using Compound 119 instead of Compound 41 in Step 1 in Example 013.

¹H-NMR (CDCl₃) δ: 8.53 (s, 1H), 7.72 (d, J=8.1 Hz, 1H), 7.57 (d, J=8.1 Hz, 1H), 6.97 (d, J=3.0 Hz, 1H), 6.88 (d, J=8.6 Hz, 1H), 6.71 (dd, J=9.1, 2.5 Hz, 1H), 6.50 (d, J=16.2 Hz, 1H), 6.24 (dd, J=16.2, 5.6 Hz, 1H), 5.46 (d, J=7.6 Hz, 1H), 5.19 (s, 2H), 4.77 (m, 1H), 3.96 (q, J=7.1 Hz, 2H), 2.03 (s, 3H), 1.39 (d, J=7.1 Hz, 3H), 1.36 (t, J=7.1 Hz, 3H).

[M+H]=375, Method Condition 2: retention time 1.94 min

### Example 036

### Preparation of Compound I-36

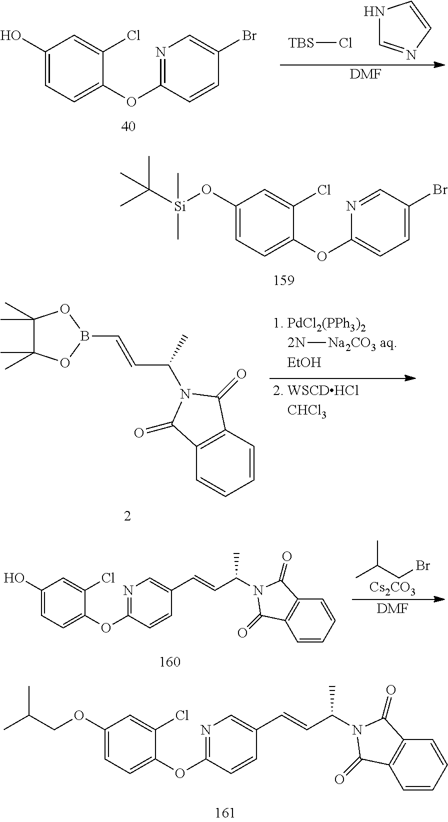

### Step 1 Preparation of Compound 159

Imidazole (0.453 g, 0.65 mmol) and TBS-C1 (0.620 g, 3.99 mmol) were added to the DMF solution (1 mL) of the Compound 40 (1.00 g, 3.33 mmol) obtained in Reference Example 021, and the mixture was stirred overnight at room temperature. Water was added to the reaction mixture, the reaction mixture was extracted with diethylether. The organic layer was washed with saturated brine, and dried over magnesium sulfate. The solvent was condensed under reduced pressure. The residue was purified by silica gel chromatography (hexane-ethyl acetate) to afford Compound 159 (1.27 g, yield 92%).

¹H-NMR (CDCl₃) δ: 8.17 (d, J=2.5 Hz, 1H), 7.78-7.74 (m, 1H), 7.04 (d, J=8.9 Hz, 1H), 6.94 (d, J=2.9 Hz, 1H), 6.84 (d, J=8.7 Hz, 1H), 6.76 (dd, J=8.8, 2.8 Hz, 1H), 0.99 (d, J=0.8 Hz, 9H), 0.23 (d, J=0.8 Hz, 6H).

### Step 2 Preparation of Compound 160

2 mol/L sodium carbonate aqueous solution (2.00 mL, 2.40 mmol) was added to the ethanol solution (6.0 mL) of Compound 159 (830 mg, 2.00 mmol) and Compound 2 (786 mg, 2.40 mmol). The atmosphere was replaced with nitrogen, and bis(triphenylphosphine) palladium(II) dichloride (140 mg, 0.200 mmol) was added to the mixture. The mixture was subjected to microwave irradiation and stirred at 80° C. for 20 minutes. The mixture was diluted with Chroloform (12 mL), and WSCD (575 mg, 3.00 mmol) was added to the mixture. The mixture was stirred at room temperature for 1 hour. Water was added to the mixture, and the mixture was extracted with chloroform. The organic layer was dried over magnesium sulfate. The solvent was condensed under reduced pressure. The residue was purified by silica gel chromatography (hexane-ethyl acetate) to afford Compound 160 (530 mg, yield 63%).

¹H-NMR (CDCl₃) δ: 8.05 (d, J=2.4 Hz, 1H), 7.86-7.79 (m, 3H), 7.74-7.67 (m, 2H), 7.00 (d, J=8.7 Hz, 1H), 6.95 (d, J=8.7 Hz, 1H), 6.77 (d, J=2.7 Hz, 1H), 6.66 (dd, J=8.8, 3.0 Hz, 1H), 6.62-6.49 (m, 2H), 5.13-5.04 (m, 1H), 1.67 (d, J=7.2 Hz, 3H).

### Step 3 Preparation of Compound 161

Cesium carbonate (88.0 mg, 0.269 mmol) and 1-bromo-2-methylpropane (0.0370 mL, 0.337 mmol) were added to the DMF solution (2 ml) of Compound 160 (105 mg, 0.225 mmol), and the mixture was stirred at 50° C. for 3 hours. Saturated ammonium chloride was added to the mixture, and the mixture was extracted with diethylether. The organic layer was washed with saturated brine, and dried over magnesium sulfate. The solvent was condensed under reduced pressure. The residue was purified by silica gel chromatography (hexane-ethyl acetate) to afford Compound 161 (54.4 mg, yield 51%).

¹H-NMR (CDCl₃) δ: 8.05 (d, J=2.4 Hz, 1H), 7.84-7.68 (m, 5H), 7.08 (d, J=8.8 Hz, 1H), 6.98 (d, J=2.9 Hz, 1H), 6.88 (s, 1H), 6.82 (dd, J=8.8, 2.9 Hz, 1H), 6.59-6.48 (m, 2H), 5.12-5.03 (m, 1H), 3.69 (d, J=6.4 Hz, 2H), 2.12-2.03 (m, 1H), 1.66 (d, J=7.2 Hz, 3H), 1.02 (d, J=6.7 Hz, 7H).

### Step 4 Preparation of Compound I-36

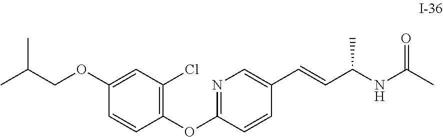 [SMILES: CC(=O)N[C@@H](C)/C=C/c1ccc(Oc2ccc(OCC(C)C)cc2Cl)nc1]

Compound I-36 was obtained by using Compound 161 instead of Compound 134 in Step 2 in Example 013.

¹H-NMR (DMSO-d₆) δ: 8.06 (d, J=2.1 Hz, 1H), 7.99-7.93 (m, 2H), 7.20 (d, J=8.8 Hz, 1H), 7.12 (d, J=2.7 Hz, 1H), 7.00 (d, J=8.5 Hz, 1H), 6.94 (dd, J=8.9, 2.8 Hz, 1H), 6.40 (d, J=16.2 Hz, 1H), 6.23 (dd, J=16.0, 5.5 Hz, 1H), 4.54-4.43 (m, 1H), 3.77 (d, J=6.6 Hz, 2H), 2.06-1.97 (m, 1H), 1.83 (s, 3H), 1.19 (d, J=6.9 Hz, 3H), 0.98 (d, J=6.6 Hz, 6H).

### Example 037

### Preparation of Compound I-37

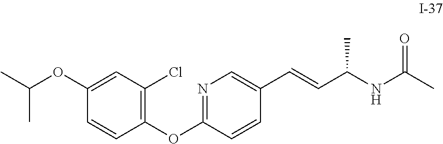 [SMILES: CC(=O)N[C@@H](C)/C=C/c1ccc(Oc2ccc(OC(C)C)cc2Cl)nc1]

Compound I-37 was obtained by using 2-iodopropane instead of 1-bromo-2-methylpropane in Step 3 in Example 036.

¹H-NMR (DMSO-d₆) δ: 8.06 (d, J=2.1 Hz, 1H), 7.99-7.93 (m, 2H), 7.19 (d, J=8.8 Hz, 1H), 7.10 (d, J=2.7 Hz, 1H), 7.00 (d, J=8.5 Hz, 1H), 6.92 (dd, J=8.8, 2.7 Hz, 1H), 6.40 (d, J=16.2 Hz, 1H), 6.23 (dd, J=16.2, 5.4 Hz, 1H), 4.67-4.59 (m, 1H), 4.52-4.45 (m, 1H), 1.83 (s, 3H), 1.28 (d, J=5.9 Hz, 6H), 1.19 (d, J=6.9 Hz, 3H).

### Example 038

### Preparation of Compound I-38

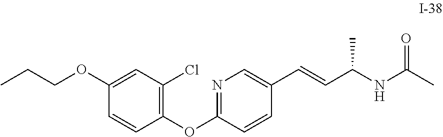 [SMILES: CCCOc1ccc(Oc2ccc(/C=C/[C@H](C)NC(C)=O)cn2)c(Cl)c1]

Compound I-38 was obtained by using 1-bromopropane instead of 1-bromo-2-methylpropane in Step 3 in Example 036.

[M+H]=375, Method Condition 2: retention time 2.28 min

### Example 039

### Preparation of Compound I-39

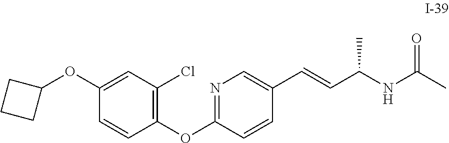 [SMILES: CC(=O)N[C@@H](C)/C=C/c1ccc(Oc2ccc(OC3CCC3)cc2Cl)nc1]

Compound I-39 was obtained by using bromocyclobutane instead of 1-bromo-2-methylpropane in Step 3 in Example 036.

[M+H]=387, Method Condition 2: retention time 2.33 min

### Example 040

### Preparation of Compound I-40

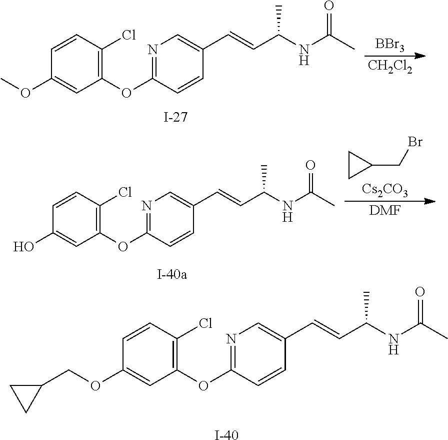

### Step 1 Preparation of Compound I-40a

The dichloromethane solution (6 mL) of Compound I-27 (500 mg, 1.44 mmol) was cooled with dry ice-acetone at −78° C. in a nitrogen atmosphere. 1.0 mol/L boron tribromide (3.00 mL, 3.00 mmol) was added dropwise to the mixture, and the mixture was warmed to room temperature for 3 hours after completion of adding dropwise. The reaction mixture was added to saturated sodium bicarbonate water, and stirred. The mixture was extracted with ethyl acetate. The organic layer was washed with saturated brine, and dried over magnesium sulfate. The solvent was condensed under reduced. The residue was purified by silica gel chromatography (chloroform-methanol) to afford Compound I-40a (355 mg, yield 74%).

¹H-NMR (DMSO-d₆) δ: 9.89 (s, 1H), 8.11 (d, J=2.2 Hz, 1H), 8.00-7.95 (m, 2H), 7.31 (d, J=8.7 Hz, 1H), 7.01 (d, J=8.6 Hz, 1H), 6.67 (dd, J=8.6, 2.8 Hz, 1H), 6.62 (d, J=2.7 Hz, 1H), 6.42 (d, J=16.1 Hz, 1H), 6.25 (dd, J=16.1, 5.4 Hz, 1H), 4.55-4.43 (m, 1H), 1.83 (s, 3H), 1.20 (d, J=6.9 Hz, 3H).

### Step 2 Preparation of Compound I-40

Cesium carbonate (206 mg, 0.631 mmol) and (bromomethyl)cyclopropane (0.0820 mL, 0.841 mmol) were added to the DMF solution (2 ml) of Compound I-40a (140 mg, 0.421 mmol), and the mixture was stirred at 65° C. for 1.5 hours. Water was added to the mixture, and the mixture was extracted with chloroform. The organic layer was dried over magnesium sulfate. The solvent was condensed under reduced pressure. The residue was purified by silica gel chromatography (chloroform-methanol) to afford Compound I-40 (140 mg, yield 86%).

¹H-NMR (DMSO-d₆) δ: 8.09 (d, J=2.1 Hz, 1H), 8.00-7.95 (m, 2H), 7.42 (d, J=8.4 Hz, 1H), 7.03 (d, J=8.5 Hz, 1H), 6.88-6.82 (m, 2H), 6.41 (d, J=16.2 Hz, 1H), 6.25 (dd, J=16.1, 5.4 Hz, 1H), 4.55-4.44 (m, 1H), 3.80 (d, J=7.0 Hz, 2H), 1.83 (s, 3H), 1.25-1.14 (m, 4H), 0.59-0.53 (m, 2H), 0.34-0.27 (m, 2H).

### Example 041

### Preparation of Compound I-41

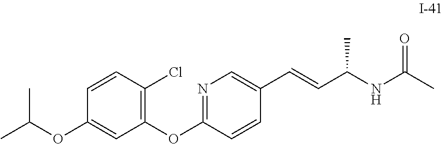 [SMILES: CC(=O)N[C@@H](C)/C=C/c1ccc(Oc2cc(OC(C)C)ccc2Cl)nc1]

Compound I-41 was obtained by using 2-iodopropane instead of (bromomethyl)cyclopropane in Step 2 in Example 040.

[M+H]=375, Method Condition 2: retention time 2.20 min

### Example 042

### Preparation of Compound I-42

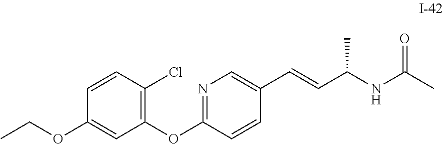 [SMILES: CCOc1ccc(Cl)c(Oc2ccc(/C=C/[C@H](C)NC(C)=O)cn2)c1]

Compound I-42 was obtained by using iodoethane instead of (bromomethyl)cyclopropane in Step 2 in Example 040.

[M+H]=361, Method Condition 2: retention time 2.08 min

### Example 043

### Preparation of Compound I-43

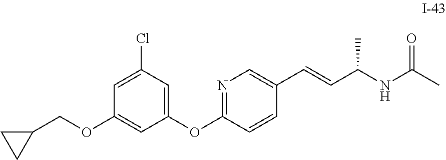 [SMILES: CC(=O)N[C@@H](C)/C=C/c1ccc(Oc2cc(Cl)cc(OCC3CC3)c2)nc1]

Compound I-43 was obtained by Compound I-28 instead of Compound I-27 in Step 1 in Example 040.

[M+H]=387, Method Condition 2: retention time 2.35 min

### Example 044

### Preparation of Compound I-44

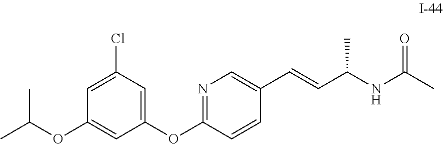 [SMILES: CC(=O)N[C@@H](C)/C=C/c1ccc(Oc2cc(Cl)cc(OC(C)C)c2)nc1]

Compound I-44 was obtained by using Compound I-28 instead of Compound I-27 in Step 1 in Example 040 and by using 2-iodopropane instead of (bromomethyl)cyclopropane in Step 2.

[M+H]=361, Method Condition 2: retention time 2.22 min

### Example 045

### Preparation of Compound I-45

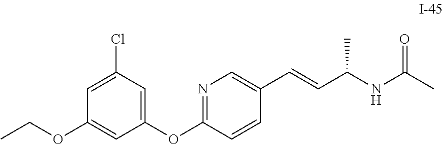 [SMILES: CCOc1cc(Cl)cc(Oc2ccc(/C=C/[C@H](C)NC(C)=O)cn2)c1]

Compound I-45 was obtained by using Compound I-28 instead of Compound I-27 in Step 1 in Example 040 and by using iodoethane instead of (bromomethyl)cyclopropane in Step 2.

[M+H]=361, Method Condition 2: retention time 2.22 min

### Example 046

### Preparation of Compound I-46

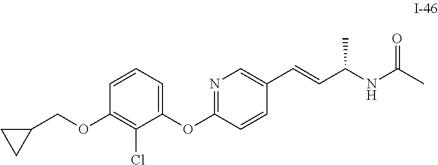 [SMILES: CC(=O)N[C@@H](C)/C=C/c1ccc(Oc2cccc(OCC3CC3)c2Cl)nc1]

Compound I-46 was obtained by using Compound I-29 instead of Compound I-27 in Step 1 in Example 040.

[M+H]=387, Method Condition 2: retention time 2.18 min

### Example 047

### Preparation of Compound I-47

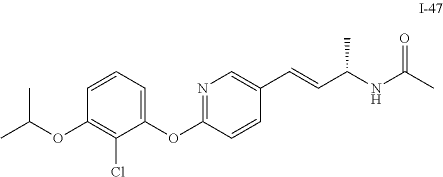 [SMILES: CC(=O)N[C@@H](C)/C=C/c1ccc(Oc2cccc(OC(C)C)c2Cl)nc1]

Compound I-47 was obtained by using Compound I-29 instead of Compound I-27 in Step 1 in Example 040 and by using 2-iodopropane instead of (bromomethyl)cyclopropane in Step 2.

[M+H]=375, Method Condition 2: retention time 2.15 min

### Example 048

### Preparation of Compound I-48

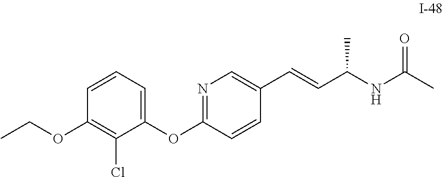 [SMILES: CCOc1cccc(Oc2ccc(/C=C/[C@H](C)NC(C)=O)cn2)c1Cl]

Compound I-48 was obtained by using Compound I-29 instead of Compound I-27 in Step 1 in Example 040 and by using iodoethane instead of (bromomethyl)cyclopropane in Step 2.

[M+H]=361, Method Condition 2: retention time 2.02 min

### Example 049

### Preparation of Compound I-49

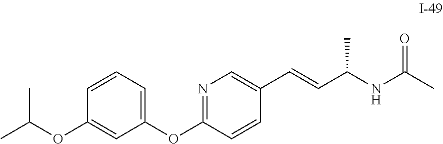 [SMILES: CC(=O)N[C@@H](C)/C=C/c1ccc(Oc2cccc(OC(C)C)c2)nc1]

Compound I-49 was obtained by using Compound I-17 instead of Compound I-27 in Step 1 in Example 040 and by using 2-iodopropane instead of (bromomethyl)cyclopropane.

[M+H]=341, Method Condition 2: retention time 2.03 min

### Example 050

### Preparation of Compound I-50

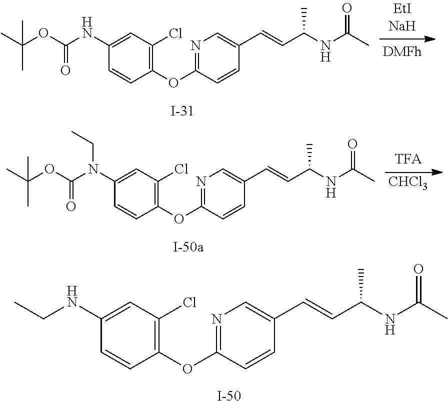

### Step 1 Preparation of Compound I-50a

The DMF solution (2 mL) of Compound I-31 (80.0 mg, 0.185 mmol) was cooled with ice in a cool bath in a nitrogen atmosphere. Sodium hydride (22.2 mg, 0.556 mmol) was added to the mixture, and the mixture was stirred for 10 minutes. Iodoethane (0.030 mL, 0.370 mmol) was added to the mixture, and the mixture was stirred for 30 minutes while cooling in ice. Water was added to the mixture, and the mixture was extracted with diethylether. The organic layer was washed with saturated brine, dried over magnesium sulfate. The solvent was condensed under reduced pressure. The residue was purified by silica gel chromatography (hexane-ethyl acetate) to afford Compound I-50a (12.5 mg, yield 15%).

[M+H]=460, Method Condition 2: retention time 2.39 min

### Step 2 Preparation of Compound I-50

Trifluoroacetic acid (1 mL, 13.0 mmol) was added to the chloroform solution (2 mL) of Compound I-50a (12.5 mg, 0.027 mmol), and the mixture was stirred overnight at room temperature. The solvent was condensed under reduced pressure. Saturated sodium bicarbonate water was added to the residue, and the mixture was extracted with chloroform. The organic layer was dried over magnesium sulfate. The solvent was condensed under reduced pressure. The residue was purified by silica gel chromatography (chloroform-methanol) to afford Compound I-50 (9.20 mg, yield 94%).

¹H-NMR (DMSO-d₆) δ: 8.06 (d, J=2.3 Hz, 1H), 7.97 (d, J=7.9 Hz, 1H), 7.91 (dd, J=8.8, 2.1 Hz, 1H), 6.94 (dd, J=24.7, 9.0 Hz, 2H), 6.62 (d, J=2.4 Hz, 1H), 6.53 (dd, J=8.5, 2.2 Hz, 1H), 6.39 (d, J=15.7 Hz, 1H), 6.21 (dd, J=16.2, 5.6 Hz, 1H), 5.82-5.76 (m, 1H), 4.52-4.44 (m, 1H), 3.06-2.97 (m, 2H), 1.83 (s, 3H), 1.19 (d, J=6.1 Hz, 3H), 1.15 (d, J=6.7 Hz, 3H).

### Example 051

### Preparation of Compound I-51

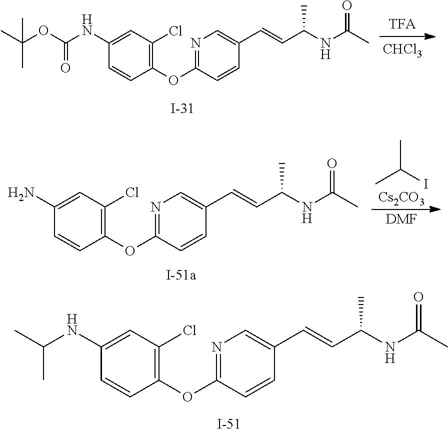

### Step 1 Preparation of Compound I-51a

Trifluoroacetic acid (1 mL, 13.0 mmol) was added to the chloroform solution (2 mL) of Compound I-31 (80.0 mg, 0.185 mmol), and the mixture was stirred overnight at room temperature. The solvent was condensed under reduced pressure. Saturated sodium bicarbonate water was added to the residue, and the mixture was extracted with chloroform. The organic layer was dried over magnesium sulfate. The solvent was condensed under reduced pressure. The residue was purified by silica gel chromatography (chloroform-methanol) to afford Compound I-51a (61.6 mg, yield 100%).

¹H-NMR (CDCl₃) δ: 8.07 (d, J=2.4 Hz, 1H), 7.70 (dd, J=8.6, 2.5 Hz, 1H), 6.98 (d, J=8.7 Hz, 1H), 6.85 (d, J=8.5 Hz, 1H), 6.77 (d, J=2.7 Hz, 1H), 6.60 (dd, J=8.7, 2.7 Hz, 1H), 6.42 (d, J=16.6 Hz, 1H), 6.07 (dd, J=16.1, 5.7 Hz, 1H), 5.40 (d, J=7.3 Hz, 1H), 4.80-4.68 (m, 1H), 3.67 (br s, 2H), 2.01 (s, 3H), 1.33 (d, J=6.9 Hz, 3H).

### Step 2 Preparation of Compound I-51

Cesium carbonate (68.3 mg, 0.210 mmol) and 2-iodopropane (0.021 mL, 0.210 mmol) were added to the DMF solution (2 mL) of Compound I-51a (58.0 mg, 0.175 mmol), and the mixture was stirred at 100° C. for 9 hours. Water was added to the mixture, and the mixture was extracted with ethyl acetate. The organic layer was washed with saturated brine, and dried over magnesium sulfate. The solvent was condensed under reduced pressure. The residue was purified by silica gel chromatography (hexane-ethyl acetate) to afford Compound I-51 (25.0 mg, yield 36%).

¹H-NMR (DMSO-d₆) δ: 8.07 (d, J=2.2 Hz, 1H), 7.98 (d, J=8.0 Hz, 1H), 7.92 (dd, J=8.7, 2.3 Hz, 1H), 6.98 (d, J=8.8 Hz, 1H), 6.91 (d, J=8.5 Hz, 1H), 6.64 (d, J=2.5 Hz, 1H), 6.53 (dd, J=8.8, 2.5 Hz, 1H), 6.41 (d, J=15.9 Hz, 1H), 6.22 (dd, J=16.3, 5.4 Hz, 1H), 5.66 (d, J=8.0 Hz, 1H), 4.54-4.45 (m, 1H), 3.57-3.45 (m, 1H), 1.83 (s, 3H), 1.20 (d, J=7.1 Hz, 3H), 1.13 (d, J=6.3 Hz, 6H)

### Example 052

### Preparation of Compound I-52

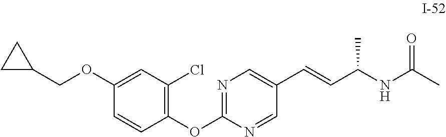 [SMILES: CC(=O)N[C@@H](C)/C=C/c1cnc(Oc2ccc(OCC3CC3)cc2Cl)nc1]

Compound I-52 was obtained by using Compound 68 instead of Compound 41 in Step 1 in Example 013.

¹H-NMR (DMSO-d₆) δ: 8.70 (s, 2H), 8.01 (d, J=8.2 Hz, 1H), 7.27 (d, J=8.4 Hz, 1H), 7.13 (d, J=2.6 Hz, 1H), 6.96 (dd, J=9.0, 2.3 Hz, 1H), 6.39 (s, 2H), 4.55-4.45 (m, 1H), 3.85 (d, J=7.0 Hz, 2H), 1.84 (s, 3H), 1.29-1.17 (m, 4H), 0.62-0.56 (m, 2H), 0.37-0.30 (m, 2H).

### Example 053

### Preparation of Compound I-53

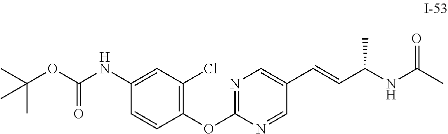 [SMILES: CC(=O)N[C@@H](C)/C=C/c1cnc(Oc2ccc(NC(=O)OC(C)(C)C)cc2Cl)nc1]

Compound I-53 was obtained by using Compound 68 instead of Compound 41 in Step 1 in Example 013.

[M+H]=433, Method Condition 2: retention time 1.98 min

### Example 054

### Preparation of Compound I-54

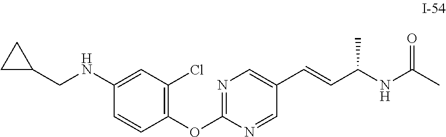 [SMILES: CC(=O)N[C@@H](C)/C=C/c1cnc(Oc2ccc(NCC3CC3)cc2Cl)nc1]

Compound I-54 was obtained by using Compound I-53 instead of Compound I-31 in Step 1 in Example 013 and by using (bromomethyl)cyclopropane instead of iodoethane.

¹H-NMR (DMSO-δ₆) δ: 8.67 (s, 2H), 8.00 (d, J=7.8 Hz, 1H), 7.02 (d, J=8.7 Hz, 1H), 6.66 (d, J=2.6 Hz, 1H), 6.56 (dd, J=8.8, 2.6 Hz, 1H), 6.41-6.35 (m, 2H), 6.01-5.90 (m, 1H), 4.54-4.45 (m, 1H), 2.88 (d, J=5.2 Hz, 2H), 1.84 (s, 3H), 1.20 (d, J=6.9 Hz, 3H), 1.11-0.97 (m, 1H), 0.52-0.46 (m, 2H), 0.26-0.18 (m, 2H).

### Example 055

### Preparation of Compound I-55

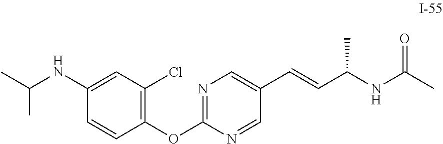 [SMILES: CC(=O)N[C@@H](C)/C=C/c1cnc(Oc2ccc(NC(C)C)cc2Cl)nc1]

Compound I-55 was obtained by using Compound I-53 instead of Compound I-31 in Step 1 in Example 013.

[M+H]=375, Method Condition 2: retention time 1.66 min

### Example 056

### Preparation of Compound I-56

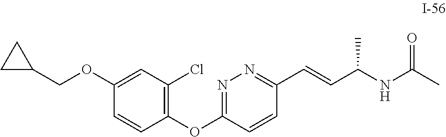 [SMILES: CC(=O)N[C@@H](C)/C=C/c1ccc(Oc2ccc(OCC3CC3)cc2Cl)nn1]

Compound I-56 was obtained by using Compound 87 instead of Compound 41 in Step 1 in Example 013.

[M+H]=388, Method Condition 2: retention time 2.00 min

### Example 057

### Preparation of Compound I-57

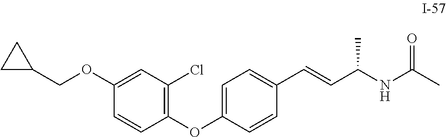 [SMILES: CC(=O)N[C@@H](C)/C=C/c1ccc(Oc2ccc(OCC3CC3)cc2Cl)cc1]

Compound I-57 was obtained by using Compound 89 instead of Compound 41 in Step 1 in Example 013.

[M+H]=386, Method Condition 2: retention time 2.47 min

### Example 057

### Preparation of Compound I-57

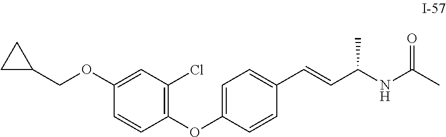 [SMILES: CC(=O)N[C@@H](C)/C=C/c1ccc(Oc2ccc(OCC3CC3)cc2Cl)cc1]

Compound I-58 was obtained by using Compound 90 instead of Compound 41 in Step 1 in Example 013.

[M+H]=404, Method Condition 2: retention time 2.54 min

### Example 059

### Preparation of Compound I-59

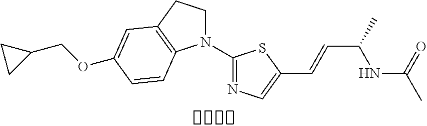 [SMILES: CC(=O)N[C@@H](C)/C=C/c1cnc(N2CCc3cc(OCC4CC4)ccc32)s1]

Compound I-59 was obtained by using Compound 99 instead of Compound 41 in Step 1 in Example 013.

[M+H]=384, Method Condition 2: retention time 2.16 min

### Example 060

### Preparation of Compound L60

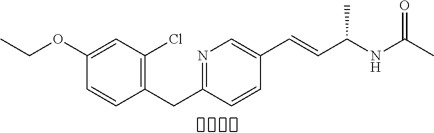 [SMILES: CCOc1ccc(Cc2ccc(/C=C/[C@H](C)NC(C)=O)cn2)c(Cl)c1]

Compound I-60 was obtained by using Compound 74 instead of Compound 16 in Step 1 in Example 001.

¹H-NMR (CDCl₃) δ: 8.47 (d, J=2.0 Hz, 1H), 7.99 (d, J=8.1 Hz, 1H), 7.77 (dd, J=8.1, 2.0 Hz, 1H), 7.23 (d, J=8.6 Hz, 1H), 7.07 (d, J=8.1 Hz, 1H), 6.99 (d, J=2.5 Hz, 1H), 6.87 (dd, J=8.6, 2.5 Hz, 1H), 6.42 (d, J=16.7 Hz, 1H), 6.31 (dd, J=16.0, 5.3 Hz, 1H), 4.45-4.54 (m, 1H), 4.10 (s, 2H), 4.02 (q, J=6.9 Hz, 2H), 1.83 (s, 3H), 1.30 (t, J=6.8 Hz, 3H), 1.20 (d, J=6.6 Hz, 3H).

[M+H]=359, Method Condition 2: retention time 1.52 min

### Example 061

### Preparation of Compound I-61

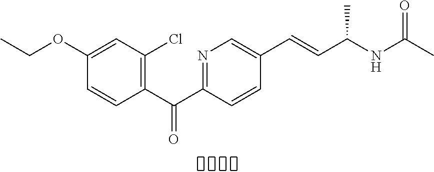 [SMILES: CCOc1ccc(C(=O)c2ccc(/C=C/[C@H](C)NC(C)=O)cn2)c(Cl)c1]

Compound I-61 was obtained by using Compound 75 instead of Compound 16 in Step 1 in Example 001.

¹H-NMR (CDCl₃) δ: 8.61 (s, 1H), 8.02 (d, J=8.1 Hz, 1H), 7.85 (dd, J=8.1, 2.0 Hz, 1H), 7.52 (d, J=8.6 Hz, 1H), 6.95 (d, J=2.5 Hz, 1H), 6.87 (dd, J=8.4, 2.3 Hz, 1H), 6.56 (d, J=15.7 Hz, 1H), 6.37 (dd, J=15.7, 5.6 Hz, 1H), 5.48 (d, J=7.6 Hz, 1H), 4.77-4.82 (m, 1H), 4.09 (q, J=7.1 Hz, 2H), 2.04 (s, 3H), 1.44 (t, J=7.1 Hz, 3H), 1.37 (d, J=7.1 Hz, 3H).

[M+H]=373, Method Condition 2: retention time 1.87 min

### Example 062

### Preparation of Compound I-62

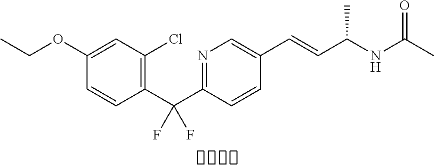 [SMILES: CCOc1ccc(C(F)(F)c2ccc(/C=C/[C@H](C)NC(C)=O)cn2)c(Cl)c1]

Compound I-62 was obtained by using Compound 76 instead of Compound 16 in Step 1 in Example 001.

¹H-NMR (CDCl₃) δ: 8.52 (s, 1H), 7.75-7.78 (m, 3H), 6.89-6.91 (m, 2H), 6.51 (d, J=16.2 Hz, 1H), 6.28 (dd, J=15.7, 5.6 Hz, 1H), 5.45 (d, J=7.6 Hz, 1H), 4.77 (dd, J=13.7, 6.6 Hz, 1H), 4.05 (q, J=6.9 Hz, 2H), 2.02 (s, 3H), 1.44-1.34 (m, 6H).

[M+H]=395, Method Condition 2: retention time 2.11 min

### Example 063

### Preparation of Compound I-63

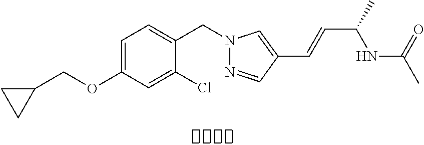 [SMILES: CC(=O)N[C@@H](C)/C=C/c1cnn(Cc2ccc(OCC3CC3)cc2Cl)c1]

Compound I-63 was obtained by using Compound 80 instead of Compound 16 in Step 1 in Example 001.

¹H-NMR (DMSO-d₆) δ: 7.90 (d, J=8.1 Hz, 1H), 7.76 (s, 1H), 7.57 (s, 1H), 7.04-6.98 (m, 2H), 6.89 (dd, J=8.5, 2.4 Hz, 1H), 6.23 (d, J=15.6 Hz, 1H), 5.88 (dd, J=16.1, 5.6 Hz, 1H), 5.27 (s, 2H), 4.46-4.34 (m, 1H), 3.82 (d, J=7.0 Hz, 2H), 1.80 (s, 3H), 1.23-1.14 (m, 4H), 0.59-0.53 (m, 2H), 0.33-0.27 (m, 2H).

### Example 064

### Preparation of Compound I-64

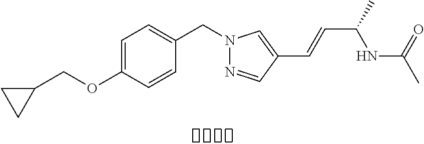 [SMILES: CC(=O)N[C@@H](C)/C=C/c1cnn(Cc2ccc(OCC3CC3)cc2)c1]

Compound I-64 was obtained by using Compound 81 instead of Compound 16 in Step 1 in Example 001.

[M+H]=340, Method Condition 2: retention time 1.82 min

### Example 065

### Preparation of Compound I-65

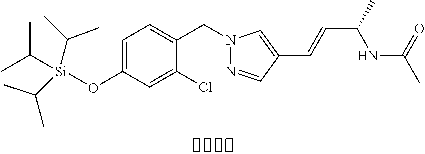 [SMILES: CC(=O)N[C@@H](C)/C=C/c1cnn(Cc2ccc(O[Si](C(C)C)(C(C)C)C(C)C)cc2Cl)c1]

Compound I-65 was obtained by using Compound 85 instead of Compound 16 in Step 1 in Example 001.

[M+H]=476, Method Condition 2: retention time 3.06 min

### Example 066

### Preparation of Compound I-66

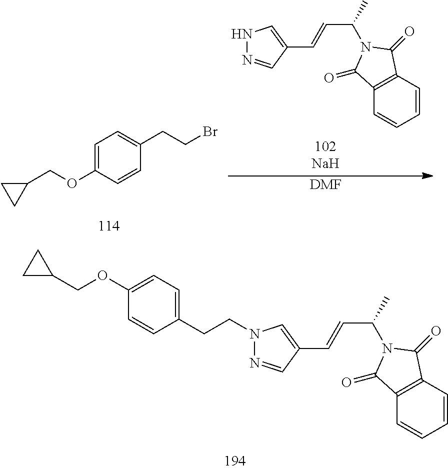

### Step 1 Preparation of Compound 194

The DMF solution (2 mL) of Compound 102 (120 mg, 0.449 mmol) was cooled with ice in a cool bath in a nitrogen atmosphere. Sodium hydride (35.9 mg, 0.898 mmol) was added to the mixture, the DMF solution (1 mL) of Compound 114 was added to the mixture. The mixture was stirred at room temperature for 1 hour. The reaction mixture was added to 10% aqueous solution of citric acid, and the mixture was extracted with ethyl acetate. The organic layer was washed with saturated brine, dried over magnesium sulfate, and filtered. The solvent was condensed under reduced pressure. The residue was purified by silica gel chromatography (hexane-ethyl acetate) to afford Compound 194 (52.1 mg, yield 26%).

¹H-NMR (CDCl₃) δ: 7.82 (dd, J=5.3, 3.3 Hz, 2H), 7.70 (dd, J=5.1, 3.0 Hz, 2H), 7.56 (s, 1H), 7.17 (s, 1H), 6.97 (d, J=8.6 Hz, 2H), 6.80 (d, J=8.6 Hz, 2H), 6.38 (d, J=16.2 Hz, 1H), 6.29 (dd, J=16.0, 7.4 Hz, 1H), 4.96-5.03 (m, 1H), 4.22 (t, J=7.4 Hz, 2H), 3.76 (d, J=6.6 Hz, 2H), 3.05 (t, J=7.1 Hz, 2H), 1.62 (d, J=6.6 Hz, 3H), 1.21-1.29 (m, 1H), 0.60-0.65 (m, 2H), 0.31-0.35 (m, 2H).

[M+H]=442, Method Condition 2: retention time 2.52 min

### Step 2 Preparation of Compound I-66

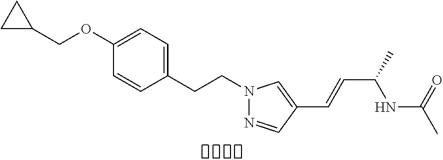 [SMILES: CC(=O)N[C@@H](C)/C=C/c1cnn(CCc2ccc(OCC3CC3)cc2)c1]

Compound I-66 was obtained by using Compound 194 instead of Compound 120 in Step 1 in Example 001.

¹H-NMR (CDCl₃) δ: 7.55 (s, 1H), 7.14 (s, 1H), 6.97 (d, J=8.6 Hz, 2H), 6.81 (d, J=8.1 Hz, 2H), 6.28 (d, J=16.2 Hz, 1H), 5.84 (dd, J=16.0, 5.8 Hz, 1H), 5.37 (d, J=7.6 Hz, 1H), 4.63-4.68 (m, 1H), 4.24 (t, J=7.1 Hz, 2H), 3.77 (d, J=7.1 Hz, 2H), 3.07 (t, J=7.4 Hz, 2H), 1.99 (s, 3H), 1.23-1.29 (m, 4H), 0.61-0.66 (m, 2H), 0.32-0.35 (m, 2H).

[M+H]=354, Method Condition 2: retention time 1.86 min

### Example 067

### Preparation of Compound I-67

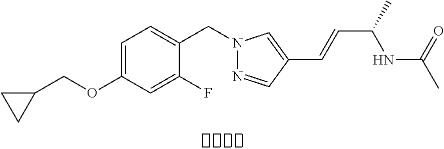 [SMILES: CC(=O)N[C@@H](C)/C=C/c1cnn(Cc2ccc(OCC3CC3)cc2F)c1]

Compound I-67 was obtained by using Compound 104 instead of Compound 114 in Step 1 in Example 066.

¹H-NMR (DMSO-d₆) δ: 7.88 (d, J=8.1 Hz, 1H), 7.76 (s, 1H), 7.53 (s, 1H), 7.14 (t, J=8.6 Hz, 1H), 6.73-6.78 (m, 2H), 6.23 (d, J=15.7 Hz, 1H), 5.88 (dd, J=16.2, 5.6 Hz, 1H), 5.21 (s, 2.0H), 4.36-4.45 (m, 1H), 3.81 (d, J=7.1 Hz, 2H), 1.81 (s, 3H), 1.14-1.19 (m, 4H), 0.53-0.58 (m, 2H), 0.28-0.32 (m, 2H).

[M+H]=358, Method Condition 2: retention time 1.86 min

### Example 068

### Preparation of Compound I-68

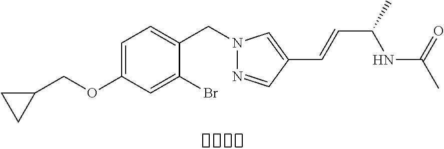 [SMILES: CC(=O)N[C@@H](C)/C=C/c1cnn(Cc2ccc(OCC3CC3)cc2Br)c1]

Compound I-68 was obtained by using Compound 107 instead of Compound 114 in Step 1 in Example 066.

¹H-NMR (DMSO-d₆) δ: 7.88 (d, J=8.1 Hz, 1H), 7.76 (s, 1H), 7.58 (s, 1H), 7.19 (d, J=2.0 Hz, 1H), 6.98-6.92 (m, 2H), 6.24 (d, J=16.2 Hz, 1H), 5.89 (dd, J=16.2, 5.6 Hz, 1H), 5.26 (s, 2H), 4.37-4.45 (m, 1H), 3.82 (d, J=7.1 Hz, 2H), 1.81 (s, 3H), 1.14-1.19 (m, 4H), 0.53-0.58 (m, 2H), 0.30 (m, 2H).

[M+H]=420, Method Condition 2: retention time 2.03 min

### Example 069

### Preparation of Compound I-69

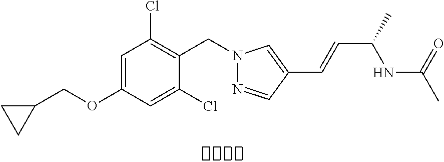 [SMILES: CC(=O)N[C@@H](C)/C=C/c1cnn(Cc2c(Cl)cc(OCC3CC3)cc2Cl)c1]

Compound I-69 was obtained by using Compound 111 instead of Compound 114 in Step 1 in Example 066.

¹H-NMR (CDCl₃) δ: 7.55 (s, 1H), 7.28 (s, 1H), 6.93 (s, 2H), 6.29 (d, J=17.2 Hz, 1H), 5.85 (dd, J=16.0, 5.8 Hz, 1H), 5.49 (s, 2H), 5.35 (d, J=8.1 Hz, 1H), 4.62-4.67 (m, 1H), 3.79 (d, J=6.6 Hz, 2H), 1.97 (s, 3H), 1.28-1.22 (m, 4H), 0.64-0.69 (m, 2H), 0.35 (m, 2H).

[M+H]=408, Method Condition 2: retention time 2.17 min

### Example 070

### Preparation of Compound I-70

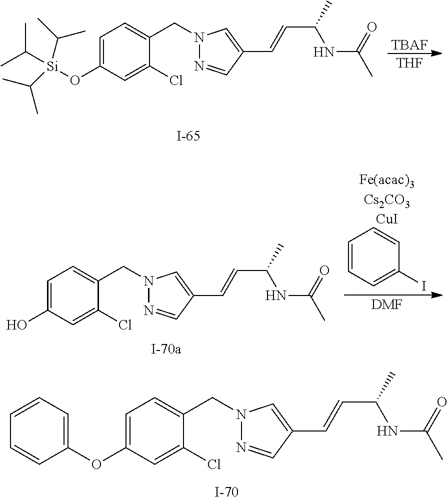

### Step 1 Preparation of Compound I-70a

Tetrabuthylammonium fluoride (1 mol/L tetrahydrofuran solution, 3.65 mL, 3.65 mmol) was added to the tetrahydrofuran solution of Compound I-65 (348 mg, 0.731 mmol), and the mixture was stirred at 80° C. for 2 hours. Water was added to the mixture, and the mixture was extracted with ethyl acetate. The organic layer was washed with saturadried brine, and dried over magnesium sulfate. The solvent was condensed under reduced pressure. The residue was purified by silica gel chromatography (chloroform-methanol) to afford Compound I-70a (197 mg, yield 84%). ¹H-NMR (DMSO-d₆) δ: 9.95 (s, 1H), 7.87 (d, J=8.1 Hz, 1H), 7.74 (s, 1H), 7.56 (s, 1H), 6.97 (d, J=8.6 Hz, 1H), 6.8 (d, J=2.0 Hz, 1H), 6.71 (dd, J=8.6, 2.5 Hz, 1H), 6.23 (d, J=15.7 Hz, 1H), 5.89 (dd, J=16.2, 5.6 Hz, 1H), 5.23 (s, 2H), 4.28-4.43 (m, 1H), 1.80 (s, 3H), 1.15 (t, J=6.6 Hz, 3H).

[M+H]=320, Method Condition 2: retention time 1.33 min

### Step 2 Preparation of Compound I-70

Cesium carbonate (71.2 mg, 0.219 mmol), iodobenzene (44.6 mg, 0.219 mmol), Copper iodide (2.78 mg, 0.015 mmol) and acetylacetone iron (III) were added to the DMF solution (2 mL) of Compound I-70a (46.6 mg, 0.146 mmol). mixture, and the mixture was stirred at 135° C. for 7 hours. Water was added to the mixture, and the mixture was extracted with diethylether. The organic layer was washed with saturated brine, and dried over magnesium sulfate. The solvent was condensed under reduced pressure. The residue was purified by prep. HPCL (acetonitrile-water) to afford Compound I-70 (3.30 mg, yield 5.7%).

¹H-NMR (CDCl₃) δ: 7.59 (s, 1H), 7.34-7.41 (m, 3H), 7.16 (t, J=7.6 Hz, 1H), 7.00-7.05 (m, 4H), 6.85 (dd, J=8.4, 2.3 Hz, 1H), 6.33 (d, J=15.7 Hz, 1H), 5.89 (dd, J=16.2, 5.6 Hz, 1H), 5.33-5.38 (m, 3H), 4.62-4.70 (m, 1H), 1.99 (s, 3H), 1.29 (d, J=7.1 Hz, 3H).

[M+H]=396, Method Condition 2: retention time 2.14 min

### Example 071-160

HATU (32.5 mg, 0.086 mmol), N-ethyldiisopropylamine (19.91 μl, 0.114 mmol) were added to the DMF solution (0.5 mL) of each carboxylic acid (0.086 mmol), and the mixture was stirred for 10 minutes. The DMF solution (0.5 mL) of Compound 121 obtained in Step 2 in Example 001 was added to the mixture, the mixture was stirred for 3 hours. Saturated sodium bicarbonate water (1 mL) was added to the mixture, and the mixture was extracted with CHCl₃ (1 ml). The solvent was condensed under reduced pressure by centrifugal evaporator. The residue was dissolved in DMSO (1 mL), and the solution purified by prep. LC/MS to afford the following compounds.

TABLE 1

| | | LC/MS | | |
|---|---|---|---|---|
| | | | reten- | |
| | | | tion | |
| | | | time | condi- |
| No. | Structure | [M + H] | (min) | tion |
| I-71 | 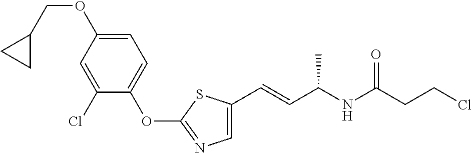 [SMILES: C[C@@H](/C=C/c1cnc(Oc2ccc(OCC3CC3)cc2Cl)s1)NC(=O)CCCl] | 441 | 2.45 | 1 |
| I-72 | 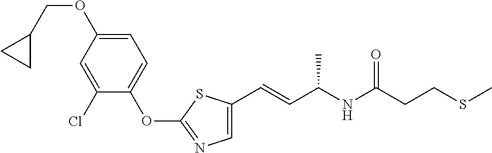 [SMILES: CSCCC(=O)N[C@@H](C)/C=C/c1cnc(Oc2ccc(OCC3CC3)cc2Cl)s1] | 453 | 2.46 | 1 |
| I-73 | 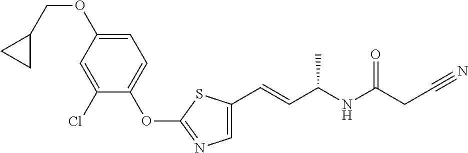 [SMILES: C[C@@H](/C=C/c1cnc(Oc2ccc(OCC3CC3)cc2Cl)s1)NC(=O)CC#N] | 418 | 2.32 | 1 |
| I-74 | 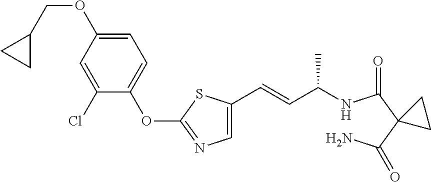 [SMILES: C[C@@H](/C=C/c1cnc(Oc2ccc(OCC3CC3)cc2Cl)s1)NC(=O)C1(C(N)=O)CC1] | 462 | 2.22 | 1 |
| I-75 | 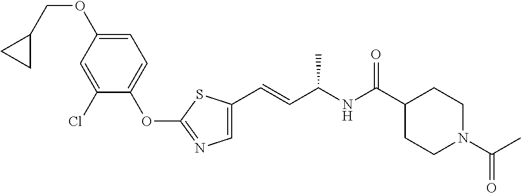 [SMILES: CC(=O)N1CCC(C(=O)N[C@@H](C)/C=C/c2cnc(Oc3ccc(OCC4CC4)cc3Cl)s2)CC1] | 504 | 2.17 | 1 |
| I-76 | 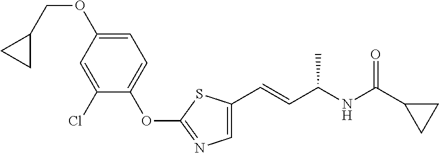 [SMILES: C[C@@H](/C=C/c1cnc(Oc2ccc(OCC3CC3)cc2Cl)s1)NC(=O)C1CC1] | 419 | 2.42 | 1 |

TABLE 2

| | | LC/MS | | |
|---|---|---|---|---|
| | | | reten- | |
| | | | tion | |
| | | | time | condi- |
| No. | Structure | [M + H] | (min) | tion |
| I-77 | 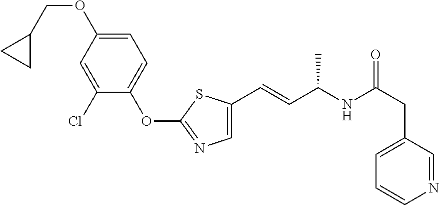 [SMILES: C[C@@H](/C=C/c1cnc(Oc2ccc(OCC3CC3)cc2Cl)s1)NC(=O)Cc1cccnc1] | 470 | 1.87 | 1 |
| I-78 | 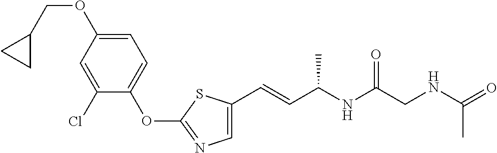 [SMILES: CC(=O)NCC(=O)N[C@@H](C)/C=C/c1cnc(Oc2ccc(OCC3CC3)cc2Cl)s1] | 450 | 2.05 | 1 |
| I-79 | 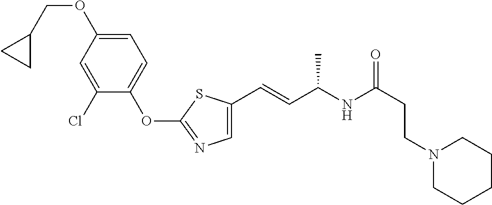 [SMILES: C[C@@H](/C=C/c1cnc(Oc2ccc(OCC3CC3)cc2Cl)s1)NC(=O)CCN1CCCCC1] | 490 | 1.92 | 1 |
| I-80 | 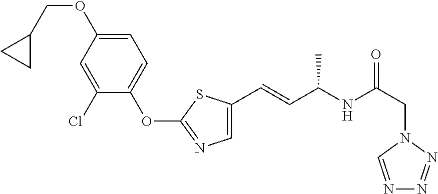 [SMILES: C[C@@H](/C=C/c1cnc(Oc2ccc(OCC3CC3)cc2Cl)s1)NC(=O)Cn1cnnn1] | 461 | 2.23 | 1 |
| I-81 | 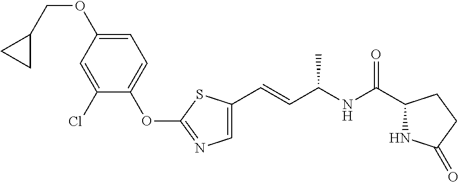 [SMILES: C[C@@H](/C=C/c1cnc(Oc2ccc(OCC3CC3)cc2Cl)s1)NC(=O)[C@@H]1CCC(=O)N1] | 462 | 2.05 | 1 |
| I-82 | 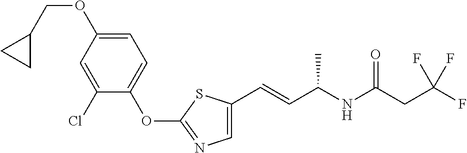 [SMILES: C[C@@H](/C=C/c1cnc(Oc2ccc(OCC3CC3)cc2Cl)s1)NC(=O)CC(F)(F)F] | 461 | 2.52 | 1 |

TABLE 3

| | | LC/MS | | |
|---|---|---|---|---|
| | | | reten- | |
| | | | tion | |
| | | | time | condi- |
| No. | Structure | [M + H] | (min) | tion |
| I-83 | 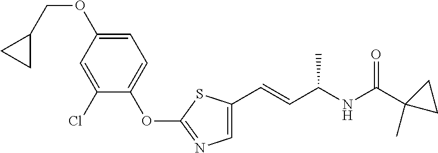 [SMILES: C[C@@H](/C=C/c1cnc(Oc2ccc(OCC3CC3)cc2Cl)s1)NC(=O)C1(C)CC1] | 433 | 2.58 | 1 |
| I-84 | 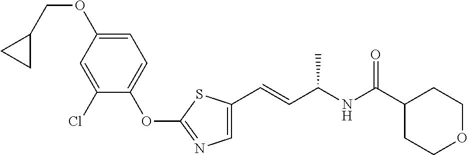 [SMILES: C[C@@H](/C=C/c1cnc(Oc2ccc(OCC3CC3)cc2Cl)s1)NC(=O)C1CCOCC1] | 463 | 2.3 | 1 |
| I-85 | 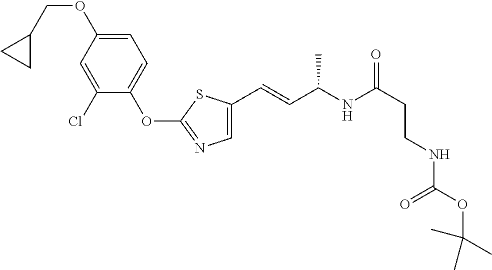 [SMILES: C[C@@H](/C=C/c1cnc(Oc2ccc(OCC3CC3)cc2Cl)s1)NC(=O)CCNC(=O)OC(C)(C)C] | 522 | 2.52 | 1 |
| I-86 | 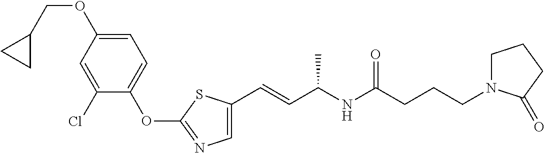 [SMILES: C[C@@H](/C=C/c1cnc(Oc2ccc(OCC3CC3)cc2Cl)s1)NC(=O)CCCN1CCCC1=O] | 504 | 2.17 | 1 |
| I-87 | 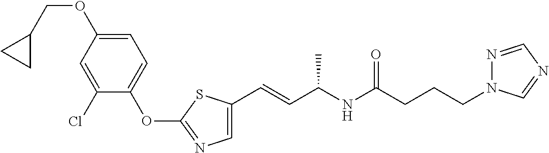 [SMILES: C[C@@H](/C=C/c1cnc(Oc2ccc(OCC3CC3)cc2Cl)s1)NC(=O)CCCn1cncn1] | 488 | 2.11 | 1 |

TABLE 4

| | | LC/MS | | |
|---|---|---|---|---|
| | | | reten- | |
| | | | tion | |
| | | | time | condi- |
| No. | Structure | [M + H] | (min) | tion |
| I-88 | 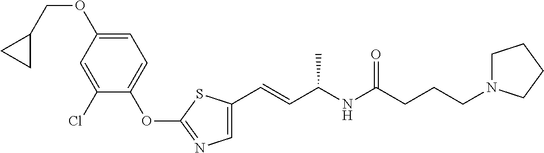 [SMILES: C[C@@H](/C=C/c1cnc(Oc2ccc(OCC3CC3)cc2Cl)s1)NC(=O)CCCN1CCCC1] | 490 | 1.85 | 1 |
| I-89 | 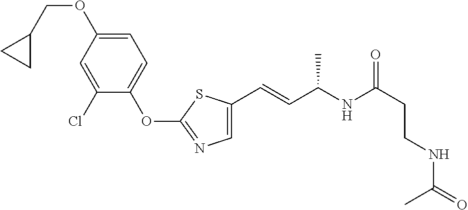 [SMILES: CC(=O)NCCC(=O)N[C@@H](C)/C=C/c1cnc(Oc2ccc(OCC3CC3)cc2Cl)s1] | 464 | 2.04 | 1 |
| I-90 | 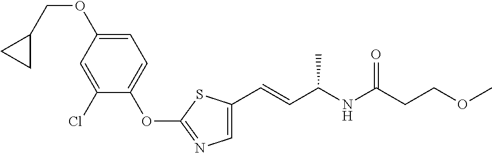 [SMILES: COCCC(=O)N[C@@H](C)/C=C/c1cnc(Oc2ccc(OCC3CC3)cc2Cl)s1] | 437 | 2.29 | 1 |
| I-91 | 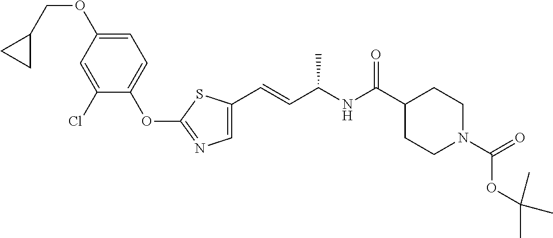 [SMILES: C[C@@H](/C=C/c1cnc(Oc2ccc(OCC3CC3)cc2Cl)s1)NC(=O)C1CCN(C(=O)OC(C)(C)C)CC1] | 562 | 2.71 | 1 |
| I-92 | 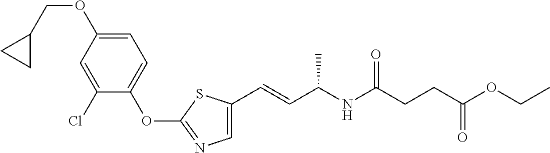 [SMILES: CCOC(=O)CCC(=O)N[C@@H](C)/C=C/c1cnc(Oc2ccc(OCC3CC3)cc2Cl)s1] | 479 | 2.45 | 1 |
| I-93 | 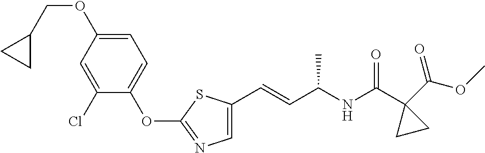 [SMILES: COC(=O)C1(C(=O)N[C@@H](C)/C=C/c2cnc(Oc3ccc(OCC4CC4)cc3Cl)s2)CC1] | 477 | 2.69 | 1 |

TABLE 5

| | | LC/MS | | |
|---|---|---|---|---|
| | | | retention | |
| | | | time | condi- |
| No. | Structure | [M + H] | (min) | tion |
| I-94 | 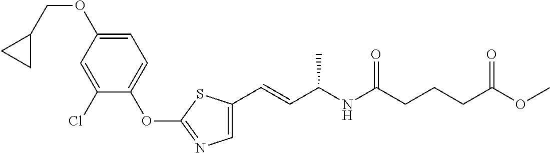 [SMILES: COC(=O)CCCC(=O)N[C@@H](C)/C=C/c1cnc(Oc2ccc(OCC3CC3)cc2Cl)s1] | 479 | 2.38 | 1 |
| I-95 | 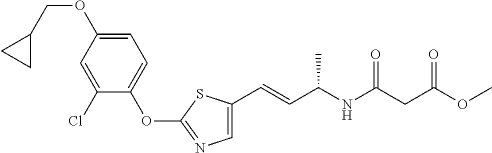 [SMILES: COC(=O)CC(=O)N[C@@H](C)/C=C/c1cnc(Oc2ccc(OCC3CC3)cc2Cl)s1] | 451 | 2.32 | 1 |
| I-96 | 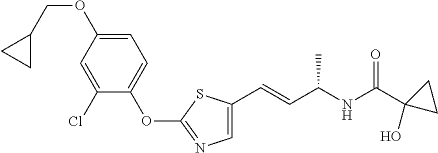 [SMILES: C[C@@H](/C=C/c1cnc(Oc2ccc(OCC3CC3)cc2Cl)s1)NC(=O)C1(O)CC1] | 435 | 2.25 | 1 |
| I-97 | 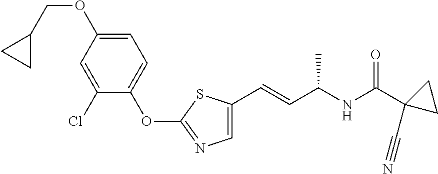 [SMILES: C[C@@H](/C=C/c1cnc(Oc2ccc(OCC3CC3)cc2Cl)s1)NC(=O)C1(C#N)CC1] | 444 | 2.55 | 1 |
| I-98 | 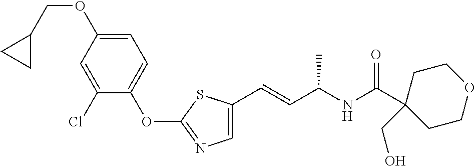 [SMILES: C[C@@H](/C=C/c1cnc(Oc2ccc(OCC3CC3)cc2Cl)s1)NC(=O)C1(CO)CCOCC1] | 493 | 2.16 | 1 |
| I-99 | 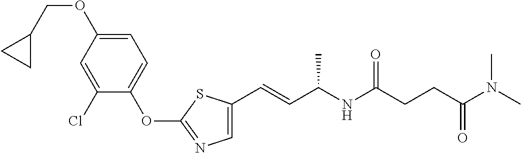 [SMILES: C[C@@H](/C=C/c1cnc(Oc2ccc(OCC3CC3)cc2Cl)s1)NC(=O)CCC(=O)N(C)C] | 487 | 2.16 | 1 |

TABLE 6

| | | LC/MS | | |
|---|---|---|---|---|
| | | | reten- | |
| | | | tion | |
| | | | time | condi- |
| No. | Structure | [M + H] | (min) | tion |
| I-100 | 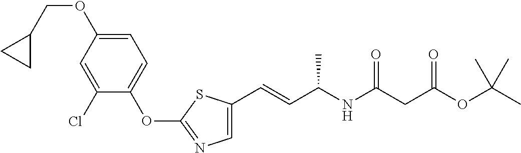 [SMILES: C[C@@H](/C=C/c1cnc(Oc2ccc(OCC3CC3)cc2Cl)s1)NC(=O)CC(=O)OC(C)(C)C] | 493 | 2.64 | 1 |
| I-101 | 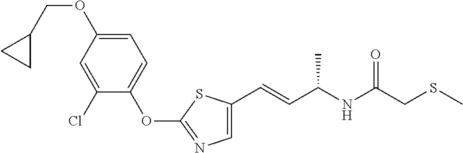 [SMILES: CSCC(=O)N[C@@H](C)/C=C/c1cnc(Oc2ccc(OCC3CC3)cc2Cl)s1] | 439 | 2.44 | 1 |
| I-102 | 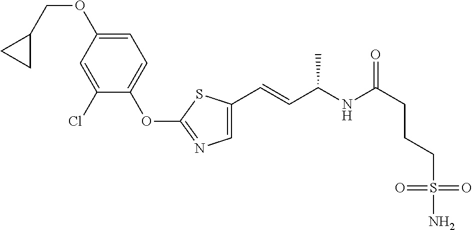 [SMILES: C[C@@H](/C=C/c1cnc(Oc2ccc(OCC3CC3)cc2Cl)s1)NC(=O)CCCS(N)(=O)=O] | 500 | 2.12 | 1 |
| I-103 | 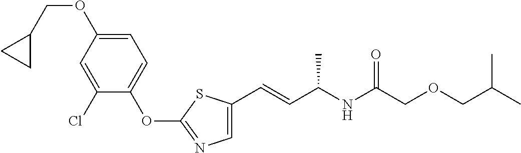 [SMILES: CC(C)COCC(=O)N[C@@H](C)/C=C/c1cnc(Oc2ccc(OCC3CC3)cc2Cl)s1] | 465 | 2.82 | 1 |
| I-104 | 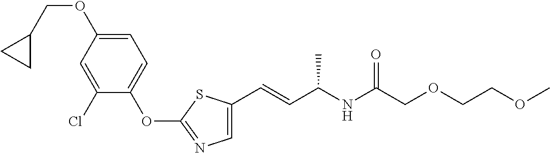 [SMILES: COCCOCC(=O)N[C@@H](C)/C=C/c1cnc(Oc2ccc(OCC3CC3)cc2Cl)s1] | 467 | 2.37 | 1 |
| I-105 | 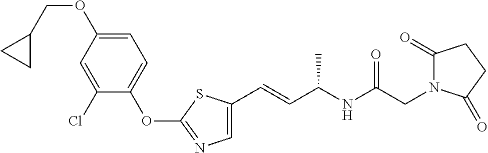 [SMILES: C[C@@H](/C=C/c1cnc(Oc2ccc(OCC3CC3)cc2Cl)s1)NC(=O)CN1C(=O)CCC1=O] | 490 | 2.21 | 1 |

TABLE 7

| | | LC/MS | | |
|---|---|---|---|---|
| | | | reten- | |
| | | | tion | |
| | | | time | condi- |
| No. | Structure | [M + H] | (min) | tion |
| I-106 | 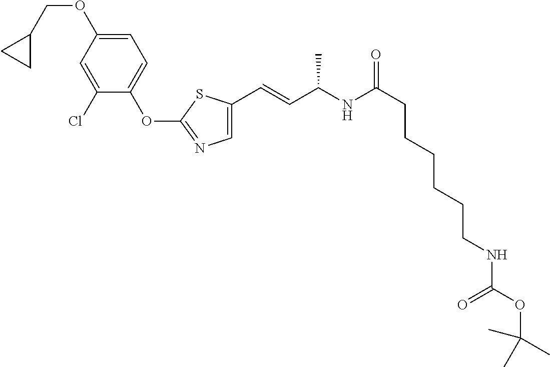 [SMILES: C[C@@H](/C=C/c1cnc(Oc2ccc(OCC3CC3)cc2Cl)s1)NC(=O)CCCCCCNC(=O)OC(C)(C)C] | 578 | 2.75 | 1 |
| I-107 | 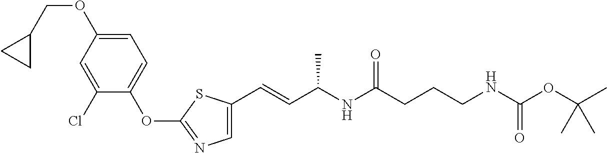 [SMILES: C[C@@H](/C=C/c1cnc(Oc2ccc(OCC3CC3)cc2Cl)s1)NC(=O)CCCNC(=O)OC(C)(C)C] | 536 | 2.56 | 1 |
| I-108 | 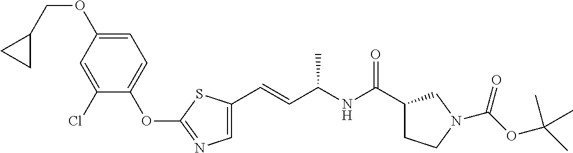 [SMILES: C[C@@H](/C=C/c1cnc(Oc2ccc(OCC3CC3)cc2Cl)s1)NC(=O)[C@H]1CCN(C(=O)OC(C)(C)C)C1] | 548 | 2.68 | 1 |
| I-109 | 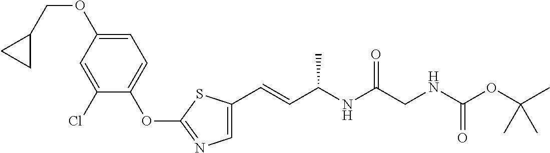 [SMILES: C[C@@H](/C=C/c1cnc(Oc2ccc(OCC3CC3)cc2Cl)s1)NC(=O)CNC(=O)OC(C)(C)C] | 508 | 2.51 | 1 |
| I-110 | 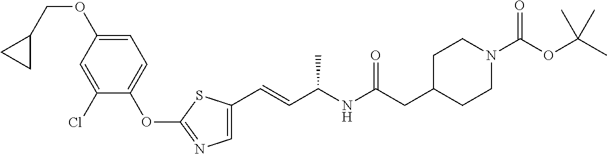 [SMILES: C[C@@H](/C=C/c1cnc(Oc2ccc(OCC3CC3)cc2Cl)s1)NC(=O)CC1CCN(C(=O)OC(C)(C)C)CC1] | 576 | 2.75 | 1 |

TABLE 8

| | | LC/MS | | |
|---|---|---|---|---|
| | | | reten- | |
| | | | tion | |
| | | | time | condi- |
| No. | Structure | [M + H] | (min) | tion |
| I-111 | 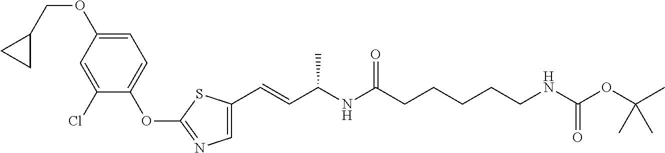 [SMILES: C[C@@H](/C=C/c1cnc(Oc2ccc(OCC3CC3)cc2Cl)s1)NC(=O)CCCCCNC(=O)OC(C)(C)C] | 564 | 2.66 | 1 |
| I-112 | 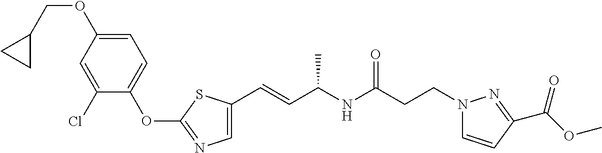 [SMILES: COC(=O)c1ccn(CCC(=O)N[C@@H](C)/C=C/c2cnc(Oc3ccc(OCC4CC4)cc3Cl)s2)n1] | 531 | 2.31 | 1 |
| I-113 | 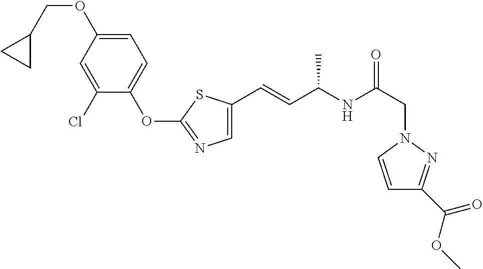 [SMILES: COC(=O)c1ccn(CC(=O)N[C@@H](C)/C=C/c2cnc(Oc3ccc(OCC4CC4)cc3Cl)s2)n1] | 517 | 2.35 | 1 |
| I-114 | 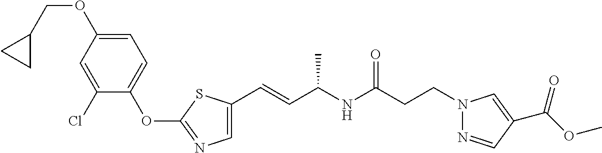 [SMILES: COC(=O)c1cnn(CCC(=O)N[C@@H](C)/C=C/c2cnc(Oc3ccc(OCC4CC4)cc3Cl)s2)c1] | 531 | 2.32 | 1 |
| I-115 | 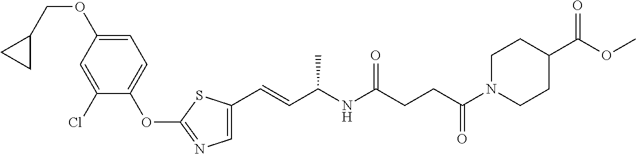 [SMILES: COC(=O)C1CCN(C(=O)CCC(=O)N[C@@H](C)/C=C/c2cnc(Oc3ccc(OCC4CC4)cc3Cl)s2)CC1] | 576 | 2.31 | 1 |

TABLE 9

| | | LC/MS | | |
|---|---|---|---|---|
| | | | reten- | |
| | | | tion | |
| | | | time | condi- |
| No. | Structure | [M + H] | (min) | tion |
| I-116 | 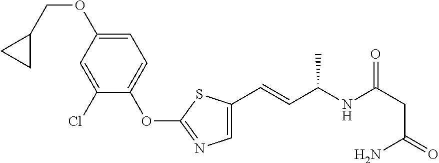 [SMILES: C[C@@H](/C=C/c1cnc(Oc2ccc(OCC3CC3)cc2Cl)s1)NC(=O)CC(N)=O] | 436 | 2.03 | 1 |
| I-117 | 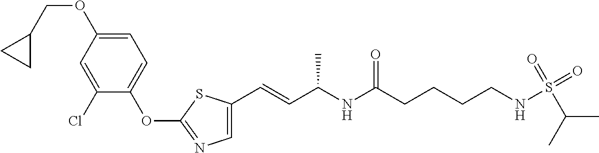 [SMILES: CC(C)S(=O)(=O)NCCCCC(=O)N[C@@H](C)/C=C/c1cnc(Oc2ccc(OCC3CC3)cc2Cl)s1] | 556 | 2.37 | 1 |
| I-118 | 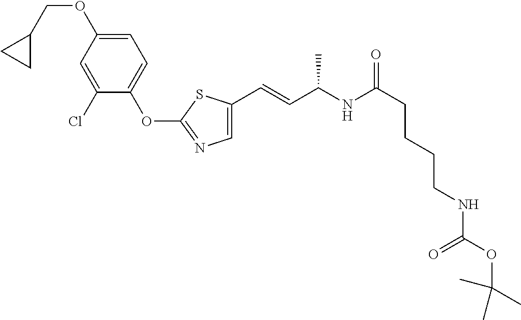 [SMILES: C[C@@H](/C=C/c1cnc(Oc2ccc(OCC3CC3)cc2Cl)s1)NC(=O)CCCCNC(=O)OC(C)(C)C] | 550 | 2.59 | 1 |
| I-119 | 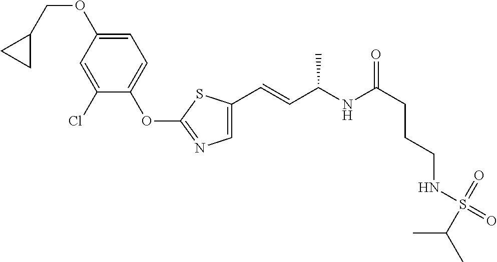 [SMILES: CC(C)S(=O)(=O)NCCCC(=O)N[C@@H](C)/C=C/c1cnc(Oc2ccc(OCC3CC3)cc2Cl)s1] | 542 | 2.33 | 1 |
| I-120 | 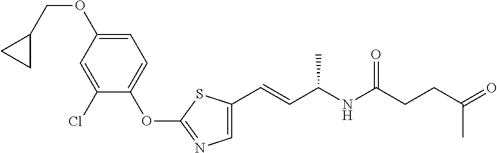 [SMILES: CC(=O)CCC(=O)N[C@@H](C)/C=C/c1cnc(Oc2ccc(OCC3CC3)cc2Cl)s1] | 449 | 2.28 | 1 |

TABLE 10

| | | LC/MS | | |
|---|---|---|---|---|
| | | | reten- | |
| | | | tion | |
| | | | time | condi- |
| No. | Structure | [M + H] | (min) | tion |
| I-121 | 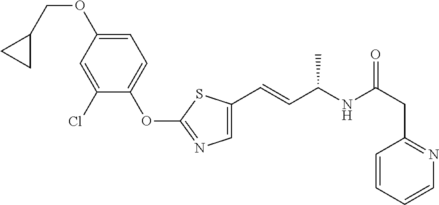 [SMILES: C[C@@H](/C=C/c1cnc(Oc2ccc(OCC3CC3)cc2Cl)s1)NC(=O)Cc1ccccn1] | 470 | 1.98 | 1 |
| I-122 | 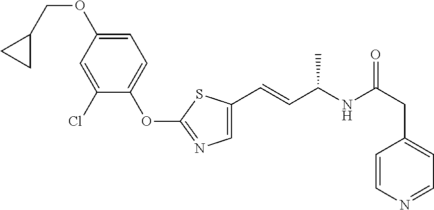 [SMILES: C[C@@H](/C=C/c1cnc(Oc2ccc(OCC3CC3)cc2Cl)s1)NC(=O)Cc1ccncc1] | 470 | 1.84 | 1 |
| I-123 | 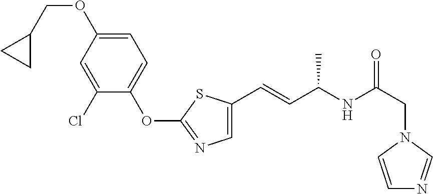 [SMILES: C[C@@H](/C=C/c1cnc(Oc2ccc(OCC3CC3)cc2Cl)s1)NC(=O)Cn1ccnc1] | 459 | 1.86 | 1 |
| I-124 | 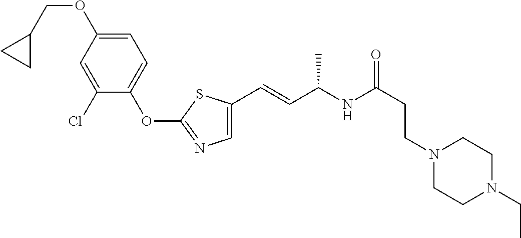 [SMILES: CCN1CCN(CCC(=O)N[C@@H](C)/C=C/c2cnc(Oc3ccc(OCC4CC4)cc3Cl)s2)CC1] | 519 | 1.69 | 1 |
| I-125 | 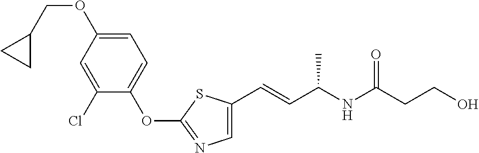 [SMILES: C[C@@H](/C=C/c1cnc(Oc2ccc(OCC3CC3)cc2Cl)s1)NC(=O)CCO] | 423 | 2.07 | 1 |
| I-126 | 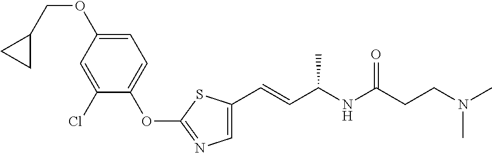 [SMILES: C[C@@H](/C=C/c1cnc(Oc2ccc(OCC3CC3)cc2Cl)s1)NC(=O)CCN(C)C] | 450 | 1.79 | 1 |

TABLE 11

| | | LC/MS | | |
|---|---|---|---|---|
| | | | reten- | |
| | | | tion | |
| | | | time | condi- |
| No. | Structure | [M + H] | (min) | tion |
| I-127 | 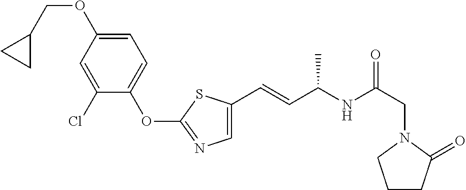 [SMILES: C[C@@H](/C=C/c1cnc(Oc2ccc(OCC3CC3)cc2Cl)s1)NC(=O)CN1CCCC1=O] | 476 | 2.15 | 1 |
| I-128 | 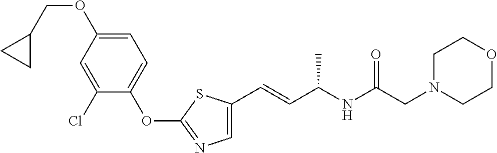 [SMILES: C[C@@H](/C=C/c1cnc(Oc2ccc(OCC3CC3)cc2Cl)s1)NC(=O)CN1CCOCC1] | 478 | 1.8 | 1 |
| I-129 | 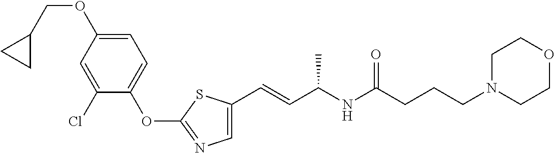 [SMILES: C[C@@H](/C=C/c1cnc(Oc2ccc(OCC3CC3)cc2Cl)s1)NC(=O)CCCN1CCOCC1] | 506 | 1.78 | 1 |
| I-130 | 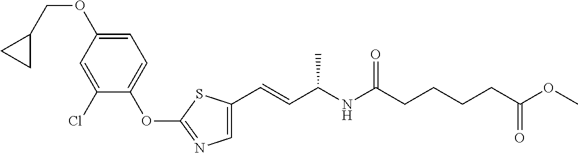 [SMILES: COC(=O)CCCCC(=O)N[C@@H](C)/C=C/c1cnc(Oc2ccc(OCC3CC3)cc2Cl)s1] | 493 | 2.44 | 1 |
| I-131 | 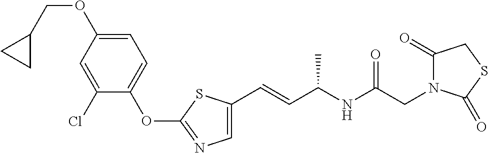 [SMILES: C[C@@H](/C=C/c1cnc(Oc2ccc(OCC3CC3)cc2Cl)s1)NC(=O)CN1C(=O)CSC1=O] | 508 | 2.36 | 1 |
| I-132 | 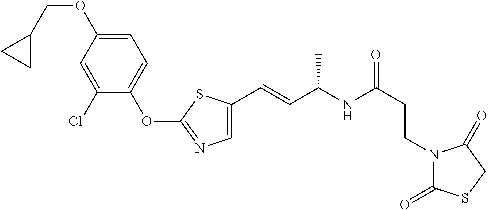 [SMILES: C[C@@H](/C=C/c1cnc(Oc2ccc(OCC3CC3)cc2Cl)s1)NC(=O)CCN1C(=O)CSC1=O] | 522 | 2.33 | 1 |

TABLE 12

| | | LC/MS | | |
|---|---|---|---|---|
| | | | reten- | |
| | | | tion | |
| | | | time | condi- |
| No. | Structure | [M + H] | (min) | tion |
| I-133 | 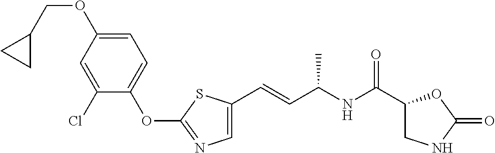 [SMILES: C[C@@H](/C=C/c1cnc(Oc2ccc(OCC3CC3)cc2Cl)s1)NC(=O)[C@H]1CNC(=O)O1] | 464 | 2.12 | 1 |
| I-134 | 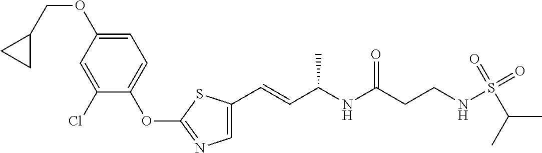 [SMILES: CC(C)S(=O)(=O)NCCC(=O)N[C@@H](C)/C=C/c1cnc(Oc2ccc(OCC3CC3)cc2Cl)s1] | 528 | 2.31 | 1 |
| I-135 | 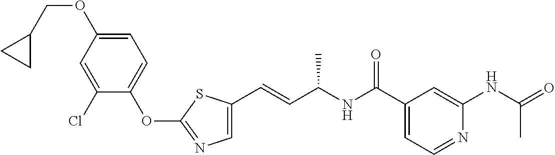 [SMILES: CC(=O)Nc1cc(C(=O)N[C@@H](C)/C=C/c2cnc(Oc3ccc(OCC4CC4)cc3Cl)s2)ccn1] | 513 | 2.3 | 2 |
| I-136 | 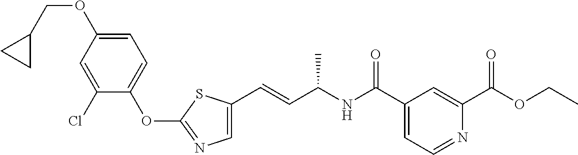 [SMILES: CCOC(=O)c1cc(C(=O)N[C@@H](C)/C=C/c2cnc(Oc3ccc(OCC4CC4)cc3Cl)s2)ccn1] | 528 | 2.5 | 2 |
| I-137 | 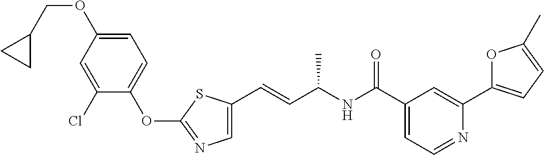 [SMILES: Cc1ccc(-c2cc(C(=O)N[C@@H](C)/C=C/c3cnc(Oc4ccc(OCC5CC5)cc4Cl)s3)ccn2)o1] | 536 | 2.69 | 2 |
| I-138 | 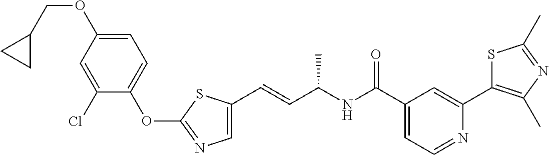 [SMILES: Cc1nc(C)c(-c2cc(C(=O)N[C@@H](C)/C=C/c3cnc(Oc4ccc(OCC5CC5)cc4Cl)s3)ccn2)s1] | 567 | 2.6 | 2 |

TABLE 13

| | | LC/MS | | |
|---|---|---|---|---|
| | | | retention | |
| | | | time | |
| No. | Structure | [M + H] | (min) | condition |
| I-139 | 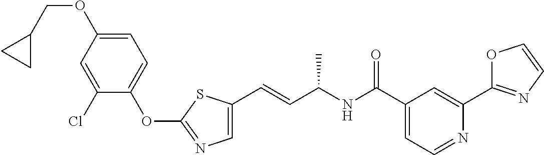 [SMILES: C[C@@H](/C=C/c1cnc(Oc2ccc(OCC3CC3)cc2Cl)s1)NC(=O)c1ccnc(-c2ncco2)c1] | 523 | 2.41 | 2 |
| I-140 | 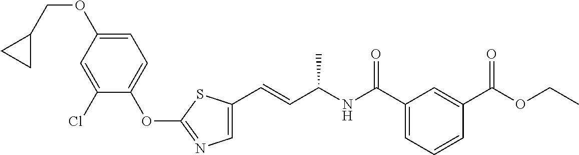 [SMILES: CCOC(=O)c1cccc(C(=O)N[C@@H](C)/C=C/c2cnc(Oc3ccc(OCC4CC4)cc3Cl)s2)c1] | 527 | 2.75 | 2 |
| I-141 | 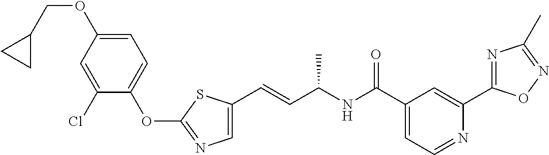 [SMILES: Cc1noc(-c2cc(C(=O)N[C@@H](C)/C=C/c3cnc(Oc4ccc(OCC5CC5)cc4Cl)s3)ccn2)n1] | 538 | 2.52 | 2 |
| I-142 | 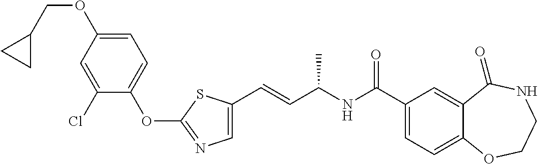 [SMILES: C[C@@H](/C=C/c1cnc(Oc2ccc(OCC3CC3)cc2Cl)s1)NC(=O)c1ccc2c(c1)C(=O)NCCO2] | 540 | 2.3 | 2 |
| I-143 | 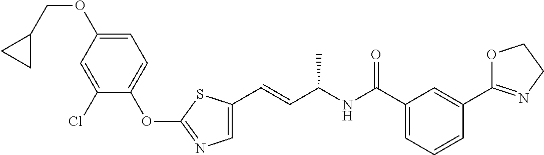 [SMILES: C[C@@H](/C=C/c1cnc(Oc2ccc(OCC3CC3)cc2Cl)s1)NC(=O)c1cccc(C2=NCCO2)c1] | 524 | 2.36 | 2 |
| I-144 | 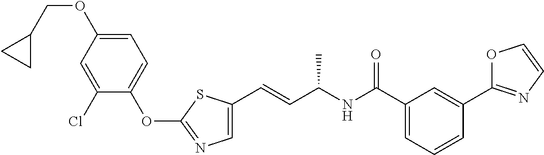 [SMILES: C[C@@H](/C=C/c1cnc(Oc2ccc(OCC3CC3)cc2Cl)s1)NC(=O)c1cccc(-c2ncco2)c1] | 522 | 2.61 | 2 |

TABLE 14

| | | LC/MS | | |
|---|---|---|---|---|
| | | | retention | |
| | | | time | |
| No. | Structure | [M + H] | (min) | condition |
| I-145 | 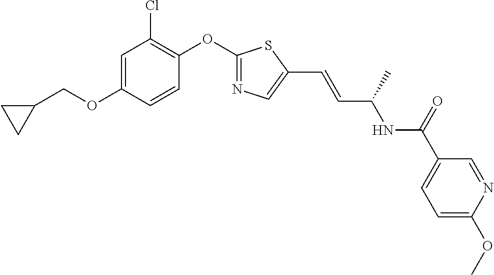 [SMILES: COc1ccc(C(=O)N[C@@H](C)/C=C/c2cnc(Oc3ccc(OCC4CC4)cc3Cl)s2)cn1] | 486 | 2.56 | 3 |
| I-146 | 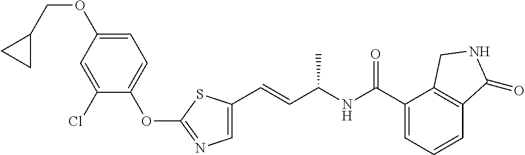 [SMILES: C[C@@H](/C=C/c1cnc(Oc2ccc(OCC3CC3)cc2Cl)s1)NC(=O)c1cccc2c1CNC2=O] | 510 | 2.26 | 2 |
| I-147 | 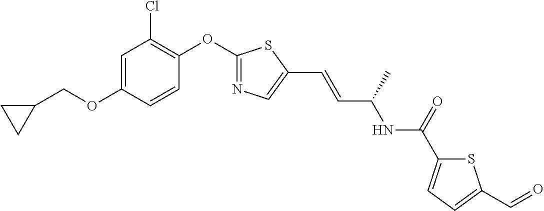 [SMILES: C[C@@H](/C=C/c1cnc(Oc2ccc(OCC3CC3)cc2Cl)s1)NC(=O)c1ccc(C=O)s1] | 489 | 2.57 | 3 |
| I-148 | 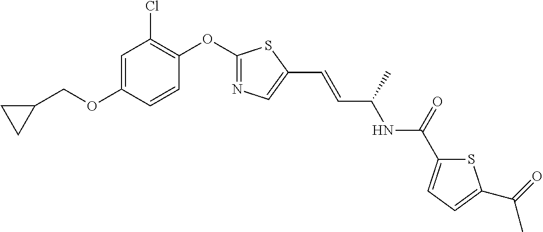 [SMILES: CC(=O)c1ccc(C(=O)N[C@@H](C)/C=C/c2cnc(Oc3ccc(OCC4CC4)cc3Cl)s2)s1] | 503 | 2.58 | 3 |
| I-149 | 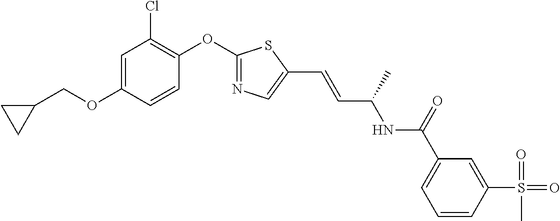 [SMILES: C[C@@H](/C=C/c1cnc(Oc2ccc(OCC3CC3)cc2Cl)s1)NC(=O)c1cccc(S(C)(=O)=O)c1] | 533 | 2.46 | 3 |
| I-150 | 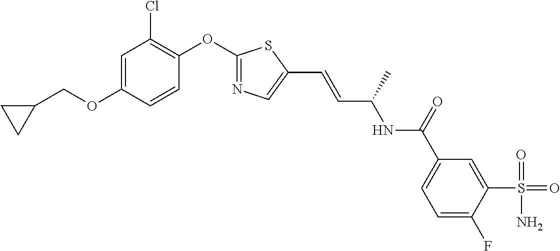 [SMILES: C[C@@H](/C=C/c1cnc(Oc2ccc(OCC3CC3)cc2Cl)s1)NC(=O)c1ccc(F)c(S(N)(=O)=O)c1] | 552 | 2.38 | 3 |

TABLE 15

| | | LC/MS | | |
|---|---|---|---|---|
| | | | retention | |
| | | | time | |
| No. | Structure | [M + H] | (min) | condition |
| I-151 | 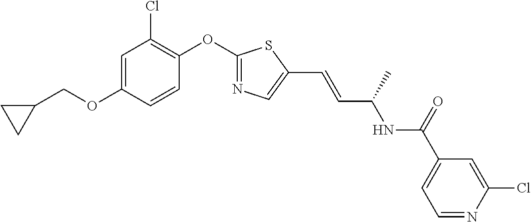 [SMILES: C[C@@H](/C=C/c1cnc(Oc2ccc(OCC3CC3)cc2Cl)s1)NC(=O)c1ccnc(Cl)c1] | 491 | 2.63 | 3 |
| I-152 | 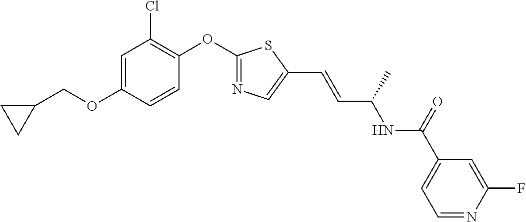 [SMILES: C[C@@H](/C=C/c1cnc(Oc2ccc(OCC3CC3)cc2Cl)s1)NC(=O)c1ccnc(F)c1] | 474 | 2.55 | 3 |
| I-153 | 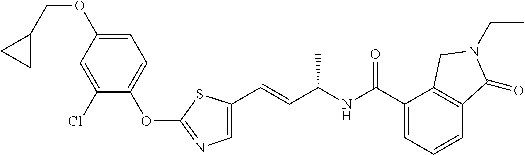 [SMILES: CCN1Cc2c(C(=O)N[C@@H](C)/C=C/c3cnc(Oc4ccc(OCC5CC5)cc4Cl)s3)cccc2C1=O] | 538 | 2.52 | 2 |
| I-154 | 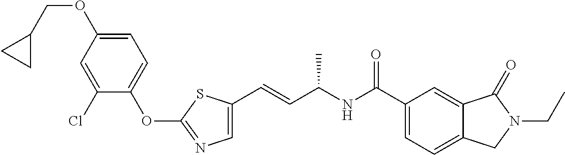 [SMILES: CCN1Cc2ccc(C(=O)N[C@@H](C)/C=C/c3cnc(Oc4ccc(OCC5CC5)cc4Cl)s3)cc2C1=O] | 538 | 2.44 | 2 |
| I-155 | 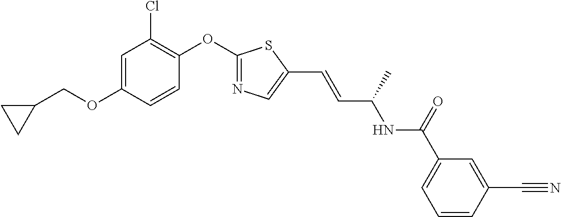 [SMILES: C[C@@H](/C=C/c1cnc(Oc2ccc(OCC3CC3)cc2Cl)s1)NC(=O)c1cccc(C#N)c1] | 480 | 2.62 | 3 |
| I-156 | 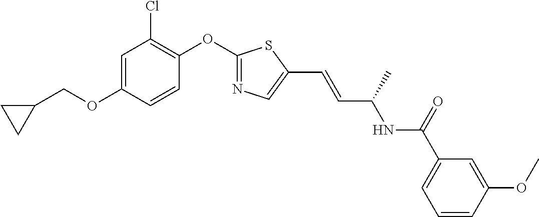 [SMILES: COc1cccc(C(=O)N[C@@H](C)/C=C/c2cnc(Oc3ccc(OCC4CC4)cc3Cl)s2)c1] | 485 | 2.66 | 3 |

TABLE 16

| | | LC/MS | | |
|---|---|---|---|---|
| | | | retention | |
| | | | time | |
| No. | Structure | [M + H] | (min) | condition |
| I-157 | 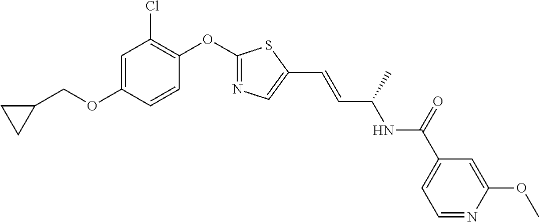 [SMILES: COc1cc(C(=O)N[C@@H](C)/C=C/c2cnc(Oc3ccc(OCC4CC4)cc3Cl)s2)ccn1] | 486 | 2.54 | 3 |
| I-158 | 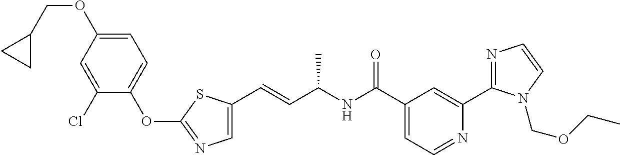 [SMILES: CCOCn1ccnc1-c1cc(C(=O)N[C@@H](C)/C=C/c2cnc(Oc3ccc(OCC4CC4)cc3Cl)s2)ccn1] | 580 | 2.18 | 2 |
| I-159 | 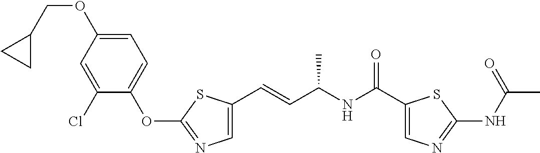 [SMILES: CC(=O)Nc1ncc(C(=O)N[C@@H](C)/C=C/c2cnc(Oc3ccc(OCC4CC4)cc3Cl)s2)s1] | 519 | 2.31 | 2 |
| I-160 | 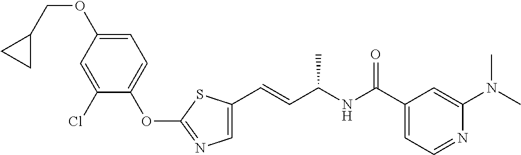 [SMILES: C[C@@H](/C=C/c1cnc(Oc2ccc(OCC3CC3)cc2Cl)s1)NC(=O)c1ccnc(N(C)C)c1] | 499 | 1.81 | 2 |

### Examples 161-170

The following Compounds were obtained by using the intermediate in Example 002 in the same manner

TABLE 17

| | | LC/MS | | |
|---|---|---|---|---|
| | | | retention | |
| | | | time | |
| No. | Structure | [M + H] | (min) | condition |
| I-161 | 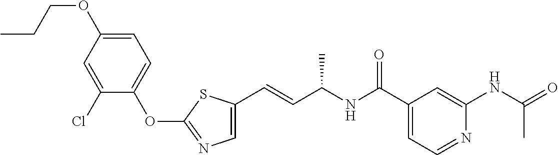 [SMILES: CCCOc1ccc(Oc2ncc(/C=C/[C@H](C)NC(=O)c3ccnc(NC(C)=O)c3)s2)c(Cl)c1] | 501 | 2.35 | 2 |
| I-162 | 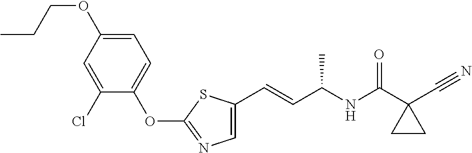 [SMILES: CCCOc1ccc(Oc2ncc(/C=C/[C@H](C)NC(=O)C3(C#N)CC3)s2)c(Cl)c1] | 432 | 2.58 | 2 |
| I-163 | 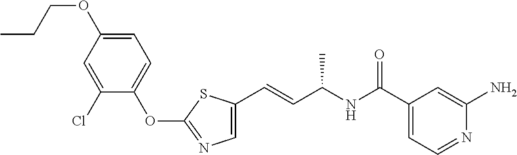 [SMILES: CCCOc1ccc(Oc2ncc(/C=C/[C@H](C)NC(=O)c3ccnc(N)c3)s2)c(Cl)c1] | 459 | 1.67 | 2 |
| I-164 | 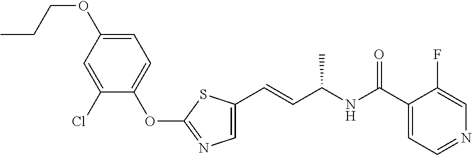 [SMILES: CCCOc1ccc(Oc2ncc(/C=C/[C@H](C)NC(=O)c3ccncc3F)s2)c(Cl)c1] | 462 | 2.5 | 2 |
| I-165 | 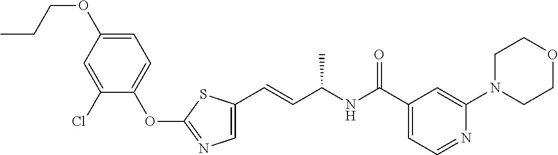 [SMILES: CCCOc1ccc(Oc2ncc(/C=C/[C@H](C)NC(=O)c3ccnc(N4CCOCC4)c3)s2)c(Cl)c1] | 529 | 2.39 | 2 |
| I-166 | 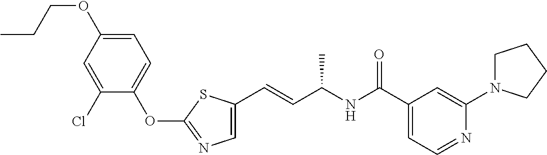 [SMILES: CCCOc1ccc(Oc2ncc(/C=C/[C@H](C)NC(=O)c3ccnc(N4CCCC4)c3)s2)c(Cl)c1] | 513 | 1.81 | 2 |

TABLE 18

| | | LC/MS | | |
|---|---|---|---|---|
| | | | retention | |
| | | | time | |
| No. | Structure | [M + H] | (min) | condition |
| I-167 | 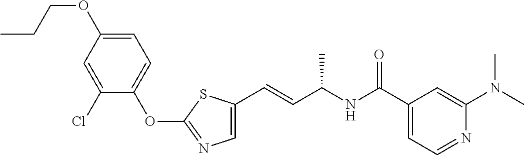 [SMILES: CCCOc1ccc(Oc2ncc(/C=C/[C@H](C)NC(=O)c3ccnc(N(C)C)c3)s2)c(Cl)c1] | 487 | 1.84 | 2 |
| I-168 | 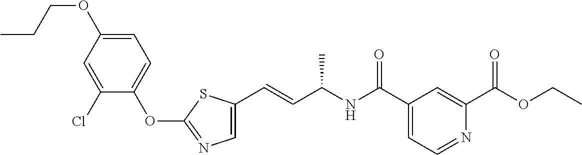 [SMILES: CCCOc1ccc(Oc2ncc(/C=C/[C@H](C)NC(=O)c3ccnc(C(=O)OCC)c3)s2)c(Cl)c1] | 516 | 2.55 | 2 |
| I-169 | 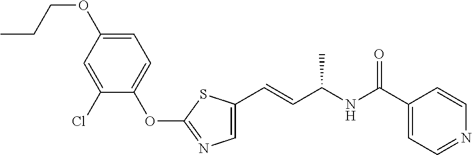 [SMILES: CCCOc1ccc(Oc2ncc(/C=C/[C@H](C)NC(=O)c3ccncc3)s2)c(Cl)c1] | 444 | 2.28 | 2 |
| I-170 | 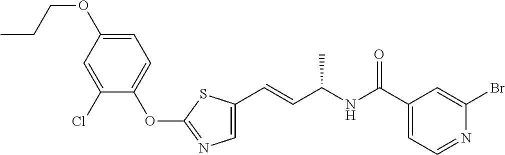 [SMILES: CCCOc1ccc(Oc2ncc(/C=C/[C@H](C)NC(=O)c3ccnc(Br)c3)s2)c(Cl)c1] | 524 | 2.69 | 2 |

### Examples 171-176

The following Compounds were obtained by using the intermediate in Example 63 in the same manner.

TABLE 19

| | | LC/MS | | |
|---|---|---|---|---|
| | | | retention | |
| | | | time | |
| No. | Structure | [M + H] | (min) | condition |
| I-171 | 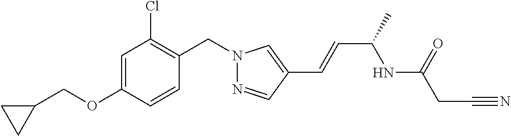 [SMILES: C[C@@H](/C=C/c1cnn(Cc2ccc(OCC3CC3)cc2Cl)c1)NC(=O)CC#N] | 399 | 2.07 | 2 |
| I-172 | 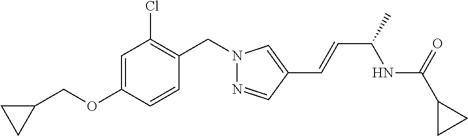 [SMILES: C[C@@H](/C=C/c1cnn(Cc2ccc(OCC3CC3)cc2Cl)c1)NC(=O)C1CC1] | 400 | 2.18 | 2 |
| I-173 | 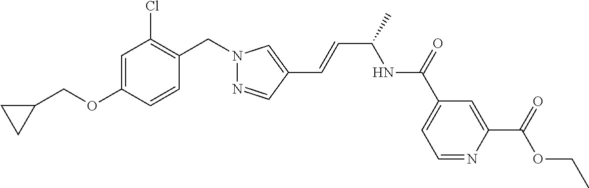 [SMILES: CCOC(=O)c1cc(C(=O)N[C@@H](C)/C=C/c2cnn(Cc3ccc(OCC4CC4)cc3Cl)c2)ccn1] | 509 | 2.25 | 2 |
| I-174 | 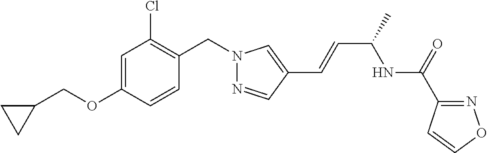 [SMILES: C[C@@H](/C=C/c1cnn(Cc2ccc(OCC3CC3)cc2Cl)c1)NC(=O)c1ccon1] | 427 | 2.25 | 2 |
| I-175 | 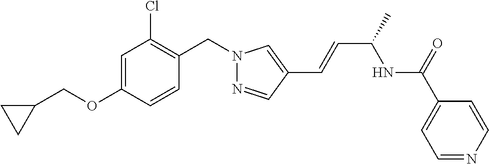 [SMILES: C[C@@H](/C=C/c1cnn(Cc2ccc(OCC3CC3)cc2Cl)c1)NC(=O)c1ccncc1] | 437 | 1.99 | 2 |
| I-176 | 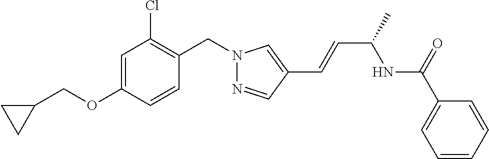 [SMILES: C[C@@H](/C=C/c1cnn(Cc2ccc(OCC3CC3)cc2Cl)c1)NC(=O)c1ccccc1] | 436 | 2.38 | 2 |

### Example 177

### Preparation of Compound I-177

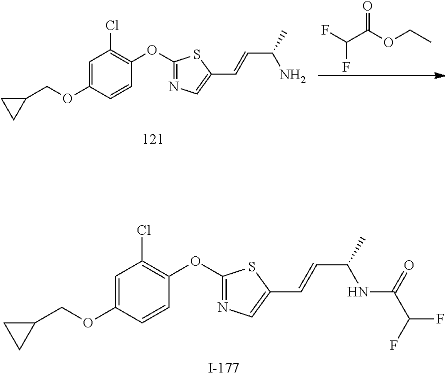

The ethyl difluoroacetate (1 mL, 10.3 mmol) solution of Compound 121 (62.0 mg, 0.177 mmol) was stirred under microwave irradiation at 150° C. for 20 minutes. The mixture was purified by silica gel chromatography (hexane-ethyl acetate) to afford Compound I-177 (55.4 mg, yield 73%).

¹H-NMR (DMSO-d₆) δ: 8.90 (d, J=7.9 Hz, 1H), 7.46 (d, J=9.1 Hz, 1H), 7.24-7.18 (m, 2H), 7.00 (dd, J=9.0, 2.9 Hz, 1H), 6.56 (d, J=15.8 Hz, 1H), 6.19 (t, J=53.7 Hz, 1H), 5.82 (dd, J=15.8, 6.0 Hz, 1H), 4.56-4.42 (m, 1H), 3.87 (d, J=7.1 Hz, 2H), 1.30-1.19 (m, 4H), 0.62-0.55 (m, 2H), 0.37-0.30 (m, 2H).

### Example 178

### Preparation of Compound I-178

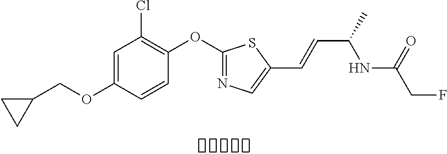 [SMILES: C[C@@H](/C=C/c1cnc(Oc2ccc(OCC3CC3)cc2Cl)s1)NC(=O)CF]

Compound I-178 was obtained by using ethyl fluoroacetate instead of ethyl difluoroacetate in Example 177.

[M+H]=411, Method Condition 2: retention time 2.37 min

### Example 179

### Preparation of Compound I-179

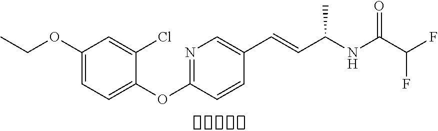 [SMILES: CCOc1ccc(Oc2ccc(/C=C/[C@H](C)NC(=O)C(F)F)cn2)c(Cl)c1]

Compound I-179 was obtained by using Compound 135 instead of Compound 121 in Example 177.

[M+H]=397, Method Condition 2: retention time 2.28 min

### Example 180

### Preparation of Compound I-180

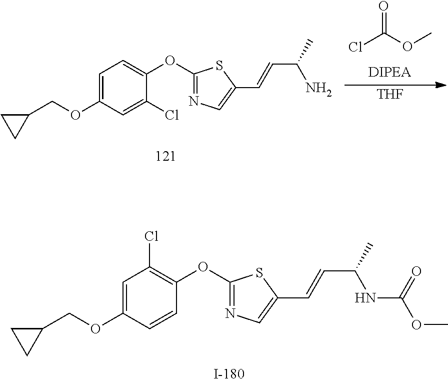

The tetrahydrofuran solution (2 mL) of Compound 121 (150 mg, 0.428 mmol) was cooled with ice in a cool bath in a nitrogen atmosphere. N-ethyldiisopropylamine (0.224 mL, 1.28 mmol) and methyl chloroformate (0.050 mL, 0.641 mmol) were added to the mixture, and the mixture was stirred 10 minutes. Methanol was added to the mixture, and the solvent was condensed under reduced pressure. The residue was purified by silica gel chromatography (hexane-ethyl acetate) to afford Compound I-180 (123 mg, yield 70%).

¹H-NMR (DMSO-d₆) δ: 7.45 (d, J=9.0 Hz, 1H), 7.30 (d, J=7.5 Hz, 1H), 7.21-7.18 (m, 2H), 6.99 (dd, J=9.0, 2.9 Hz, 1H), 6.50 (d, J=15.7 Hz, 1H), 5.78 (dd, J=15.7, 5.8 Hz, 1H), 4.23-4.10 (m, 1H), 3.86 (d, J=7.0 Hz, 2H), 3.52 (s, 3H), 1.27-1.14 (m, 4H), 0.62-0.54 (m, 2H), 0.37-0.30 (m, 2H).

### Example 181

### Preparation of Compound I-181

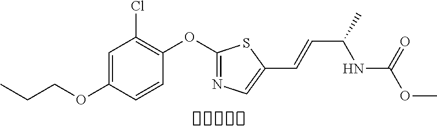 [SMILES: CCCOc1ccc(Oc2ncc(/C=C/[C@H](C)NC(=O)OC)s2)c(Cl)c1]

Compound I-181 was obtained by using Intermediate of Example 002 instead of Compound 121 in Example 180.

[M+H]=397, Method Condition 2: retention time 2.55 min

### Example 182

### Preparation of Compound I-182

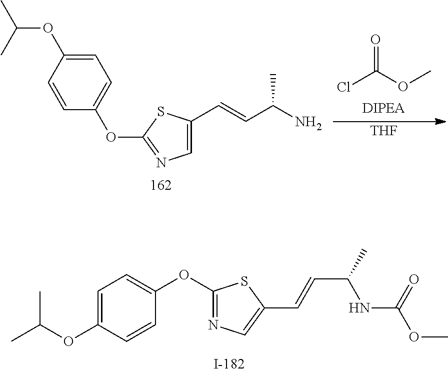

Compound I-182 was obtained by using Compound 162 instead of Compound 121 in Example 180.

[M+H]=363, Method Condition 2: retention time 2.31 min

### Example 183

### Preparation of Compound I-183

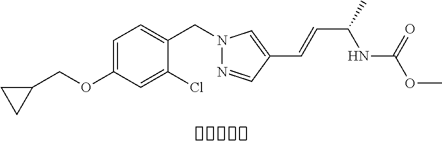 [SMILES: COC(=O)N[C@@H](C)/C=C/c1cnn(Cc2ccc(OCC3CC3)cc2Cl)c1]

Compound I-183 was obtained by using Intermediate of Example 063 instead of Compound 121 in Example 180.

¹H-NMR (CDCl₃) δ: 7.56 (s, 1H), 7.36 (s, 1H), 7.05 (d, J=8.6 Hz, 1H), 6.94 (d, J=2.5 Hz, 1H), 6.77 (dd, J=8.6, 2.5 Hz, 1H), 6.32 (d, J=15.7 Hz, 1.H), 5.86 (dd, J=16.0, 5.8 Hz, 1H), 5.29 (s, 2H), 4.36 (s, 1H), 3.77 (d, J=7.1 Hz, 2H), 3.67 (s, 3H), 1.22-1.29 (t, J=8.62 Hz, 4H), 0.62-0.67 (m, 2H), 0.32-0.36 (m, 2H).

[M+H]=390, Method Condition 2: retention time 2.24 min

### Example 184

### Preparation of Compound I-184

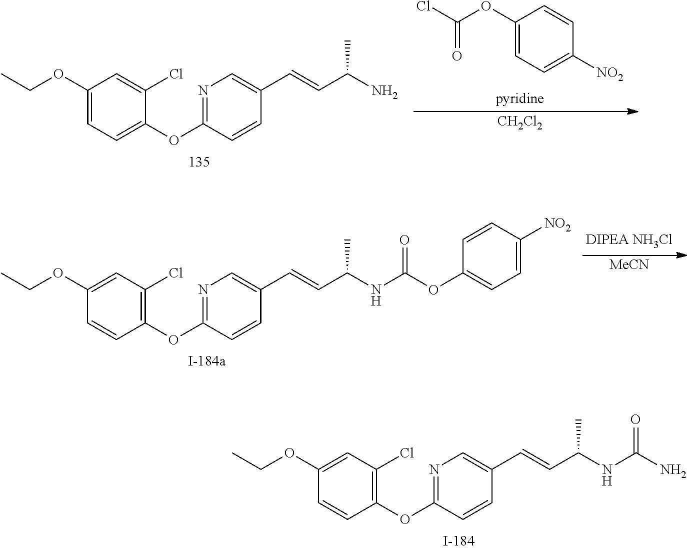 [SMILES: "MeCN" ; CCOc1ccc(Oc2ccc(/C=C/[C@H](C)N)cn2)c(Cl)c1 ; O=C(Cl)Oc1ccc([N+](=O)[O-])cc1 ; "CH2Cl2" ; CCOc1ccc(Oc2ccc(/C=C/[C@H](C)NC(=O)Oc3ccc([N+](=O)[O-])cc3)cn2)c(Cl)c1 ; CCOc1ccc(Oc2ccc(/C=C/[C@H](C)NC(N)=O)cn2)c(Cl)c1]

Step 1 Preparation of Compound I-184a

Pyridine (0.225 mL, 2.78 mmol) was added to the dichloromethane solution of Compound 135 (295 mg, 0.925 mmol), and the mixture was cooled in ice under a nitrogen atmosphere. 4-Nitrophenyl Chloroformate (205 mg, 1.018 mmol) was added to the mixture, and the mixture was stirred at room temperature for 10 hours. The solvent was condensed under reduced pressure. 1 mol/L hydrochloric acid was added to the mixture, the mixture was extracted with ethyl acetate. The organic layer was washed with saturated brine, and dried over magnesium sulfate. The solvent was condensed under reduced pressure to afford Compound I-184a (405 mg, 0.753 mmol, purity 90%, yield 81.4%). The obtained Compound I-184a was followed to the next step without purification.

### Step 2 Preparation of Compound I-184

Ammonium chloride (105 mg, 1.96 mmol) and diisopropylethylamine (0.343 mL, 1.96 mmol) were added to acetonitrile suspension of the compound 1-184a (190 mg, 0.393 mmol), and the mixture was stirred at 60° C. for 1 hour. 2 mol/L aqueous sodium hydroxide was added to the mixture, the mixture was extracted with chloroform. The organic layer was dried over magnesium sulfate. The solvent was condensed under reduced pressure. The residue was purified by silica gel chromatography (chloroform-methanol) to afford Compound I-184 (84.7 mg, yield 61%).

¹H-NMR (DMSO-d₆) δ: 8.05 (d, J=1.8 Hz, 1H), 7.95 (dd, J=8.6, 2.4 Hz, 1H), 7.20 (d, J=8.7 Hz, 1H), 7.11 (d, J=2.9 Hz, 1H), 7.00 (d, J=8.4 Hz, 1H), 6.93 (dd, J=8.9, 2.7 Hz, 1H), 6.39 (d, J=16.2 Hz, 1H), 6.26 (dd, J=16.2, 4.8 Hz, 1H), 6.06 (d, J=8.4 Hz, 1H), 5.42 (s, 2H), 4.36-4.24 (m, 1H), 4.05 (q, J=6.9 Hz, 2H), 1.33 (t, J=6.9 Hz, 3H), 1.17 (d, J=6.9 Hz, 3H).

### Example 185

### Preparation of Compound I-185

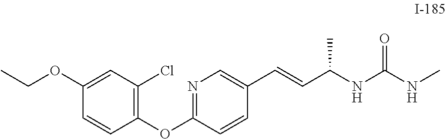 [SMILES: CCOc1ccc(Oc2ccc(/C=C/[C@H](C)NC(=O)NC)cn2)c(Cl)c1]

Compound I-185 was obtained by using methylamine chloride instead of ammonium chloride in step 2 in Example 184.

[M+H]=376, Method Condition 2: retention time 2.03 min

### Example 186

### Preparation of Compound I-186

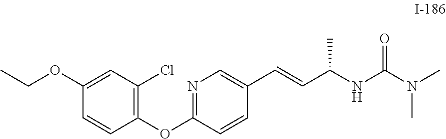 [SMILES: CCOc1ccc(Oc2ccc(/C=C/[C@H](C)NC(=O)N(C)C)cn2)c(Cl)c1]

Compound I-186 was obtained by using methylamine chloride instead of ammonium chloride in step 2 in Example 184.

[M+H]=390, Method Condition 2: retention time 2.16 min

### Example 187

### Preparation of Compound I-187

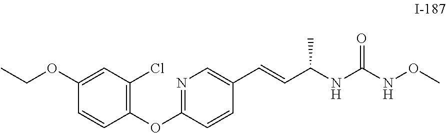 [SMILES: CCOc1ccc(Oc2ccc(/C=C/[C@H](C)NC(=O)NOC)cn2)c(Cl)c1]

Compound I-187 was obtained by using O-methylhydroxylamine hydrochloride instead of ammonium chloride in step 2 in Example 184.

[M+H]=392, Method Condition 2: retention time 2.12 min

### Example 188

### Preparation of Compound I-188

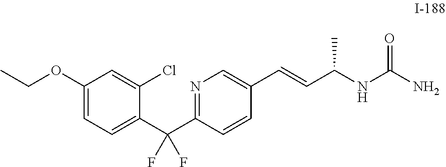 [SMILES: CCOc1ccc(C(F)(F)c2ccc(/C=C/[C@H](C)NC(N)=O)cn2)c(Cl)c1]

Compound I-188 was obtained by using Intermediate of Example 062 instead of Compound 135 in step 1 in Example 184.

¹H-NMR (CDCl₃) δ: 8.53 (s, 1H), 7.76-7.78 (m, 3H), 6.89-6.91 (m, 2H), 6.54 (d, J=16.2 Hz, 1H), 6.31 (dd, J=15.7, 5.1 Hz, 1H), 4.49 (br-s, 2H), 4.36 (br-s, 2H), 4.05 (q, J=6.9 Hz, 2H), 1.42 (t, J=7.1 Hz, 3H), 1.35 (d, J=6.6 Hz, 3H)

[M+H]=396, Method Condition 2: retention time 1.98 min

### Examples 189-194

Following Compounds were obtained by using Compound 121 instead of Compound 135 in step 1 in Example 184 and by using an amine instead of ammonium chloride in step 2.

TABLE 20

| | | LC/MS | | |
|---|---|---|---|---|
| | | | retention | |
| | | | time | |
| No. | Structure | [M + H] | (min) | condition |
| I-189 | 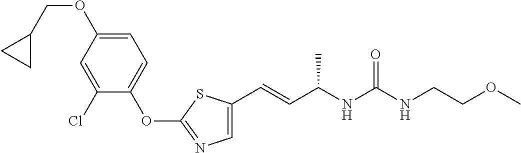 [SMILES: COCCNC(=O)N[C@@H](C)/C=C/c1cnc(Oc2ccc(OCC3CC3)cc2Cl)s1] | 452 | 2.25 | 2 |
| I-190 | 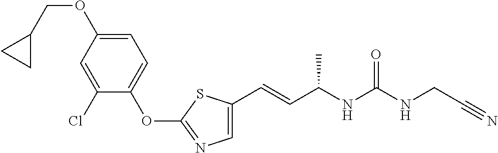 [SMILES: C[C@@H](/C=C/c1cnc(Oc2ccc(OCC3CC3)cc2Cl)s1)NC(=O)NCC#N] | 443 | 2.24 | 2 |
| I-191 | 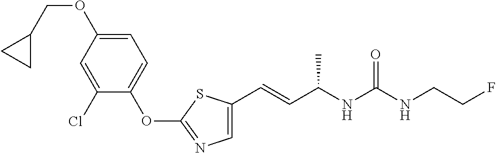 [SMILES: C[C@@H](/C=C/c1cnc(Oc2ccc(OCC3CC3)cc2Cl)s1)NC(=O)NCCF] | 440 | 2.28 | 2 |
| I-192 | 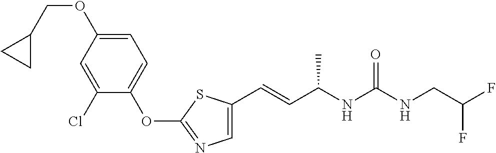 [SMILES: C[C@@H](/C=C/c1cnc(Oc2ccc(OCC3CC3)cc2Cl)s1)NC(=O)NCC(F)F] | 458 | 2.36 | 2 |
| I-193 | 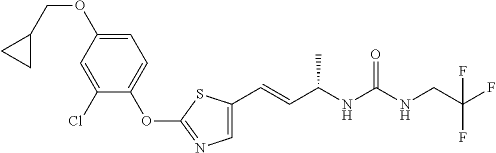 [SMILES: C[C@@H](/C=C/c1cnc(Oc2ccc(OCC3CC3)cc2Cl)s1)NC(=O)NCC(F)(F)F] | 476 | 2.45 | 2 |
| I-194 | 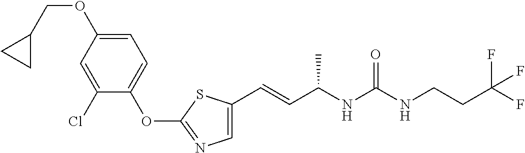 [SMILES: C[C@@H](/C=C/c1cnc(Oc2ccc(OCC3CC3)cc2Cl)s1)NC(=O)NCCC(F)(F)F] | 490 | 2.47 | 2 |

### Example 195

### Preparation of Compound I-195

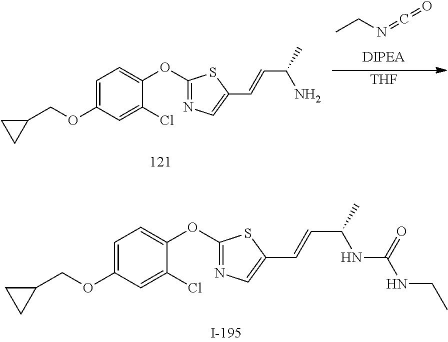

The tetrahydrofuran solution (2 mL) of Compound 121 (64 mg, 0.182 mmol) was cooled with ice in a cool bath in a nitrogen atmosphere. N-ethyldiisopropylamine (0.048 mL, 0.274 mmol), ethyl isocyanate (0.022 mL, 0.274 mmol) were added to the mixture, and the mixture was stirred at room temperature for 1 hour. Methanol was added to the mixture, and the solvent was condensed under reduced pressure. The residue was purified by silica gel chromatography (hexane-ethyl acetate) to afford Compound I-195 (65.7 mg, yield 85%).

¹H-NMR (DMSO-d₆) δ: 7.45 (d, J=9.2 Hz, 1H), 7.20 (d, J=2.7 Hz, 1H), 7.17 (s, 1H), 6.99 (dd, J=8.8, 2.7 Hz, 1H), 6.46 (d, J=15.7 Hz, 1H), 5.89 (d, J=8.2 Hz, 1H), 5.81 (dd, J=15.8, 5.4 Hz, 1H), 5.72 (t, J=5.6 Hz, 1H), 4.31-4.19 (m, 1H), 3.86 (d, J=7.0 Hz, 2H), 3.04-2.95 (m, 2H), 1.28-1.17 (m, 1H), 1.12 (d, J=6.9 Hz, 3H), 0.97 (t, J=7.2 Hz, 3H), 0.62-0.54 (m, 2H), 0.37-0.30 (m, 2H).

### Example 196

### Preparation of Compound I-196

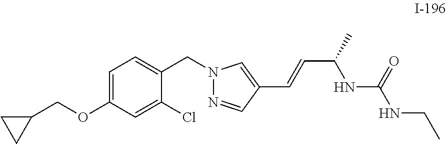 [SMILES: CCNC(=O)N[C@@H](C)/C=C/c1cnn(Cc2ccc(OCC3CC3)cc2Cl)c1]

Compound I-196 was obtained by using Intermediate of Example 063 instead of Compound 121 in Example 195.

¹H-NMR (DMSO-d₆) δ: 7.76 (s, 1H), 7.56 (s, 1H), 7.01-7.03 (d, J=8.6 Hz, 2H), 6.89 (br-d, J=8.6 Hz, 1H), 6.21 (d, J=16.2 Hz, 1H), 5.91 (dd, J=16.2, 5.6 Hz, 1H), 5.81 (d, J=8.6 Hz, 1H), 5.68 (t, J=5.6 Hz, 1H), 5.27 (s, 2H), 4.22-4.27 (d, J=6.1 Hz, 1H), 3.82 (d, J=7.1 Hz, 2H), 2.97-3.03 (m, 2H), 1.12-1.19 (m, 4H), 0.98 (t, J=7.1 Hz, 3H), 0.56 (br-d, J=8.1 Hz, 2H), 0.30 (br-d, J=4.6 Hz, 2H).

[M+H]=403, Method Condition 2: retention time 2.06 min

### Example 197

### Preparation of Compound I-197

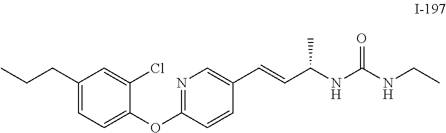 [SMILES: CCCc1ccc(Oc2ccc(/C=C/[C@H](C)NC(=O)NCC)cn2)c(Cl)c1]

Compound I-197 was obtained by using Intermediate of Example 030 instead of Compound 121 in Example 195.

¹H NMR (CDCl₃) δ: 8.07 (d, J=2.0 Hz, 1H), 7.72 (dd, J=8.6, 2.0 Hz, 1H), 7.27 (s, 1H), 7.10 (s, 2H), 6.89 (d, J=8.6 Hz, 1H), 6.45 (d, J=16.2 Hz, 1H), 6.11 (dd, J=16.2, 5.6 Hz, 1H), 4.45 (m, 1H), 4.31 (m, 2H), 3.21 (m, 2H), 2.58 (t, J=7.6 Hz, 2H), 1.66 (m, 2H), 1.31 (d, J=7.1 Hz, 3H), 1.12 (t, J=7.4 Hz, 3H), 0.97 (t, J=7.4 Hz, 3H).

[M+H]=388, Method Condition 2: retention time 2.40 min

### Example 198

### Preparation of Compound I-198

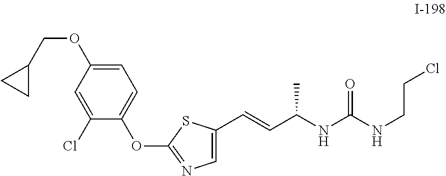 [SMILES: C[C@@H](/C=C/c1cnc(Oc2ccc(OCC3CC3)cc2Cl)s1)NC(=O)NCCCl]

Compound I-198 was obtained by using isocyanic acid 2-chloroethyl ester instead of ethyl isocyanate in Example 195.

[M+H]=456, Method Condition 2: retention time 2.37 min

### Example 199

### Preparation of Compound I-199

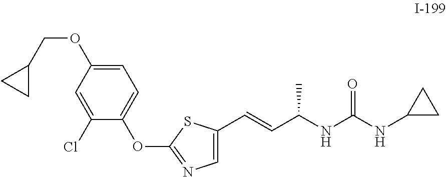 [SMILES: C[C@@H](/C=C/c1cnc(Oc2ccc(OCC3CC3)cc2Cl)s1)NC(=O)NC1CC1]

Compound I-199 was obtained by using cyclopropyl isocyanate instead of ethyl isocyanate in Example 195.

[M+H]=434, Method Condition 2: retention time 2.34 min

### Example 200

### Preparation of Compound I-200

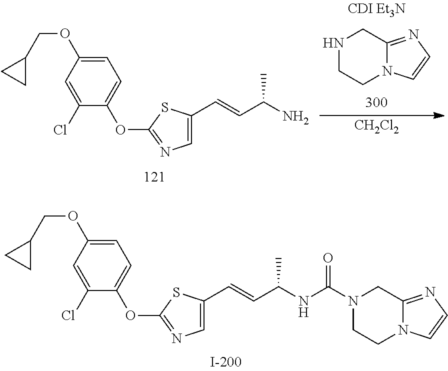 [SMILES: C[C@H](N)/C=C/c1cnc(Oc2ccc(OCC3CC3)cc2Cl)s1 ; "CH2Cl2" ; c1cn2c(n1)CNCC2 ; C[C@@H](/C=C/c1cnc(Oc2ccc(OCC3CC3)cc2Cl)s1)NC(=O)N1CCn2ccnc2C1]

The tetrahydrofuran solution (2 mL) of CDI (27.7 mg, 0.171 mmol) was cooled with ice in a cool bath in a nitrogen atmosphere. Compound 121 (50 mg, 0.143 mmol) and triethylamine (0.040 mL, 0.285 mmol) were added to the mixture, and the mixture was stirred at room temperature for 5 hours. Water was added to the mixture, and the mixture was extracted with chloroform. The organic layer was dried over magnesium sulfate. The solvent was condensed under reduced pressure. The residue was purified by silica gel chromatography (chloroform-methanol) to afford Compound I-200 (44.0 mg, yield 62%).

¹H-NMR (DMSO-d₆) δ: 7.45 (d, J=8.9 Hz, 1H), 7.20 (d, J=3.0 Hz, 1H), 7.17 (s, 1H), 7.07 (s, 1H), 7.00 (dd, J=9.0, 2.9 Hz, 1H), 6.86 (s, 1H), 6.76 (d, J=7.7 Hz, 1H), 6.50 (d, J=15.8 Hz, 1H), 5.84 (dd, J=15.7, 5.6 Hz, 1H), 4.54 (s, 2H), 4.43-4.32 (m, 1H), 3.95 (t, J=5.2 Hz, 2H), 3.86 (d, J=7.1 Hz, 2H), 3.76 (t, J=5.2 Hz, 2H), 1.28-1.17 (m, OH), 0.61-0.55 (m, 2H), 0.37-0.30 (m, 2H).

### Example 201

### Preparation of Compound I-201

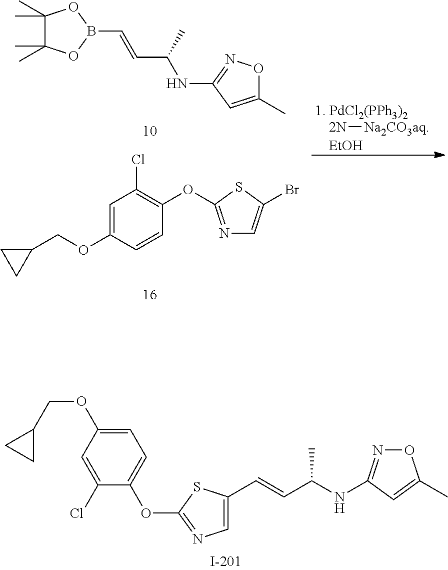

2 mol/L sodium carbonate aqueous solution (0.277 mL, 0.555 mmol) was added to the ethanol solution (2.0 mL) of Compound 16 (100 mg, 0.277 mmol) and Compound 10 (93 mg, 0.333 mmol), and bis(triphenylphosphine) palladium(II) dichloride (19.46 mg, 0.028 mmol) was added to the mixture. The mixture was subjected to microwave irradiation and stirred at 80° C. for 20 minutes. Water was added to the mixture, and the mixture was extracted with chloroform. The organic layer was washed dried over magnesium sulfate. The solvent was condensed under reduced pressure. The residue was purified by silica gel chromatography (hexane-ethyl acetate) to afford Compound I-201 (96.2 mg, yield 80%).

¹H-NMR (DMSO-d₆) δ: 7.44 (d, J=9.3 Hz, 1H), 7.21-7.15 (m, 2H), 6.99 (dd, J=9.0, 2.6 Hz, 1H), 6.55 (d, J=15.7 Hz, 1H), 6.13 (d, J=7.8 Hz, 1H), 5.80 (dd, J=15.7, 5.9 Hz, 1H), 5.61 (s, 1H), 4.08-3.96 (m, 1H), 3.86 (d, J=7.2 Hz, 2H), 2.20 (s, 3H), 1.25-1.15 (m, 4H), 0.61-0.55 (m, 2H), 0.37-0.28 (m, 2H).

### Example 202

### Preparation of Compound I-202

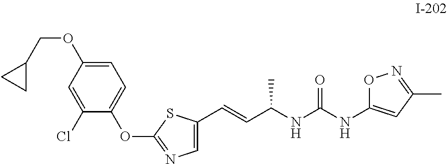 [SMILES: Cc1cc(NC(=O)N[C@@H](C)/C=C/c2cnc(Oc3ccc(OCC4CC4)cc3Cl)s2)on1]

Compound I-202 was obtained by using Compound 11 instead of Compound 10 in Example 013.

[M+1-1]=432, Method Condition 2: retention time 2.59 min

### Example 203

### Preparation of Compound I-203

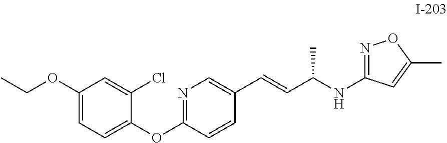 [SMILES: CCOc1ccc(Oc2ccc(/C=C/[C@H](C)Nc3cc(C)on3)cn2)c(Cl)c1]

Compound I-203 was obtained by using Compound 41 instead of Compound 16 in Example 201.

[M+H]=400, Method Condition 2: retention time 2.44 min

### Example 204

### Preparation of Compound I-204

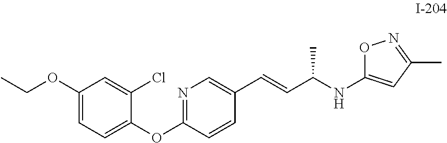 [SMILES: CCOc1ccc(Oc2ccc(/C=C/[C@H](C)Nc3cc(C)no3)cn2)c(Cl)c1]

Compound I-204 was obtained by using Compound 11 instead of Compound 10 and Compound 41 instead of Compound 16 in Example 201.

[M+H]=400, Method Condition 2: retention time 2.46 min

### Example 205

### Preparation of Compound I-205

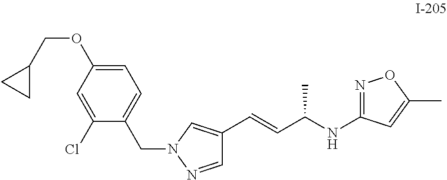 [SMILES: Cc1cc(N[C@@H](C)/C=C/c2cnn(Cc3ccc(OCC4CC4)cc3Cl)c2)no1]

Compound I-205 was obtained by using Compound 80 instead of Compound 16 in Example 201.

[M+H]=413, Method Condition 2: retention time 2.36 min

### Example 206

### Preparation of Compound I-206

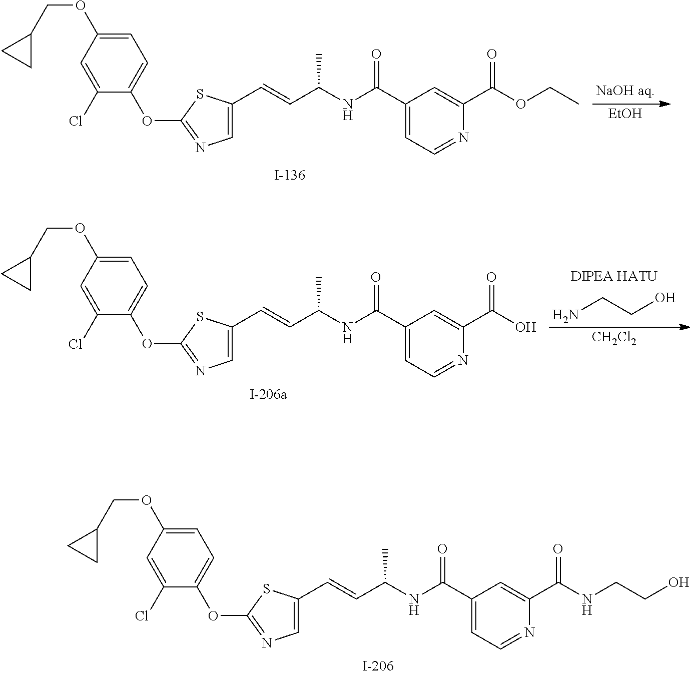

### Step 1 Preparation of Compound I-206a

2 mol/L aqueous sodium hydroxide (1.0 mL, 2.00 mmol) was added to the ethanol solution (3 mL) of Compound I-136 (305 mg, 0.578 mmol), and the mixture was stirred at room temperature for 1 hour. 10% aqueous solution of citric acid was added to the mixture, and the mixture was neutralized. The precipitated crystal was filtered off and dried at 80° C. under vacuum to afford Compound I-206a (288 mg, 0.576 mmol, yield 100%)

¹H-NMR (DMSO-d₆) δ: 8.98 (d, J=7.8 Hz, 1H), 8.83 (d, J=4.9 Hz, 1H), 8.45 (s, 1H), 7.99 (dd, J=4.9, 1.2 Hz, 1H), 7.45 (d, J=9.0 Hz, 1H), 7.21 (s, 1H), 7.19 (d, J=2.9 Hz, 1H), 6.99 (dd, J=9.0, 2.9 Hz, 1H), 6.61 (d, J=15.9 Hz, 1H), 5.91 (dd, J=15.9, 5.7 Hz, 1H), 4.77-4.64 (m, 1H), 3.86 (d, J=7.2 Hz, 2H), 1.31 (d, J=6.7 Hz, 3H), 1.27-1.16 (m, 1H), 0.61-0.55 (m, 2H), 0.35-0.30 (m, 2H).

### Step 2 Preparation of Compound I-206

N-ethyldiisopropyl amine (0.044 mL, 0.252 mmol) and HATU (83 mg, 0.218 mmol) were added to the dichloromethane suspension (2 mL) of Compound I-206a (84 mg, 0.168 mmol) and ethanolamine (0.015 mL, 0.252 mmol), and the mixture was stirred at room temperature for 3 hours. Saturated sodium bicarbonate water was added to the mixture, and the mixture was extracted with chloroform. The organic layer was dried over magnesium sulfate. The solvent was condensed under reduced pressure. The residue was purified by silica gel chromatography (hexane-ethyl acetate) to afford Compound I-206.

¹H-NMR (DMSO-d₆) δ: 9.01 (d, J=7.3 Hz, 1H), 8.80-8.71 (m, 2H), 8.46 (s, 1H), 7.97 (d, J=4.3 Hz, 1H), 7.45 (d, J=9.0 Hz, 1H), 7.24-7.17 (m, 2H), 6.99 (dd, J=9.0, 2.7 Hz, 1H), 6.60 (d, J=15.4 Hz, 1H), 5.92 (dd, J=15.4, 5.6 Hz, 1H), 4.85-4.65 (m, 2H), 3.86 (d, J=6.4 Hz, 2H), 3.57-3.49 (m, 2H), 3.44-3.36 (m, 2H), 1.31 (d, J=6.4 Hz, 3H), 1.28-1.15 (m, 1H), 0.62-0.54 (m, 2H), 0.37-0.29 (m, 2H).

### Examples 207-223

Following Compounds were obtained by using corresponding amine or hydroxyl amine in Step 2 in Example 206.

TABLE 21

| | | LC/MS | | |
|---|---|---|---|---|
| | | | re- | |
| | | | tention | |
| | | [M + | time | con- |
| No. | Structure | H] | (min) | dition |
| I-207 | 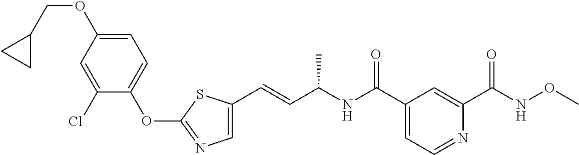 [SMILES: CONC(=O)c1cc(C(=O)N[C@@H](C)/C=C/c2cnc(Oc3ccc(OCC4CC4)cc3Cl)s2)ccn1] | 529 | 2.31 | 2 |
| I-208 | 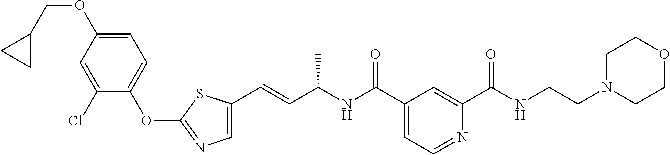 [SMILES: C[C@@H](/C=C/c1cnc(Oc2ccc(OCC3CC3)cc2Cl)s1)NC(=O)c1ccnc(C(=O)NCCN2CCOCC2)c1] | 612 | 1.68 | 2 |
| I-209 | 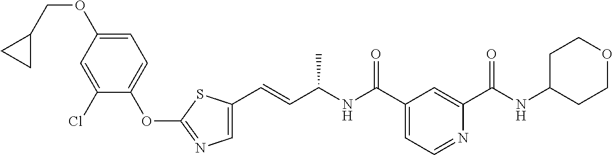 [SMILES: C[C@@H](/C=C/c1cnc(Oc2ccc(OCC3CC3)cc2Cl)s1)NC(=O)c1ccnc(C(=O)NC2CCOCC2)c1] | 583 | 2.43 | 2 |
| I-210 | 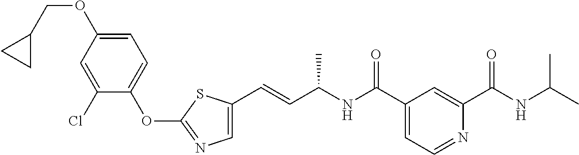 [SMILES: CC(C)NC(=O)c1cc(C(=O)N[C@@H](C)/C=C/c2cnc(Oc3ccc(OCC4CC4)cc3Cl)s2)ccn1] | 541 | 2.59 | 2 |
| I-211 | 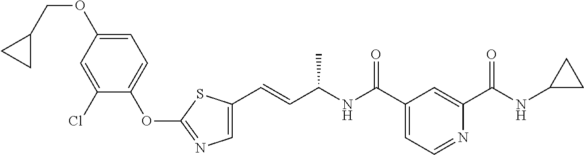 [SMILES: C[C@@H](/C=C/c1cnc(Oc2ccc(OCC3CC3)cc2Cl)s1)NC(=O)c1ccnc(C(=O)NC2CC2)c1] | 539 | 2.49 | 2 |
| I-212 | 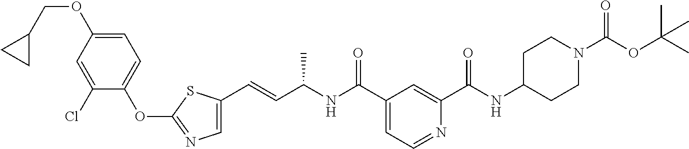 [SMILES: C[C@@H](/C=C/c1cnc(Oc2ccc(OCC3CC3)cc2Cl)s1)NC(=O)c1ccnc(C(=O)NC2CCN(C(=O)OC(C)(C)C)CC2)c1] | 682 | 2.76 | 2 |

TABLE 22

| | | LC/MS | | |
|---|---|---|---|---|
| | | | retention | |
| | | | time | |
| No. | Structure | [M + H] | (min) | condition |
| I-213 | 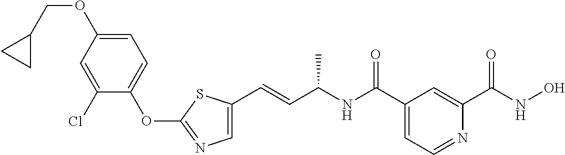 [SMILES: C[C@@H](/C=C/c1cnc(Oc2ccc(OCC3CC3)cc2Cl)s1)NC(=O)c1ccnc(C(=O)NO)c1] | 515 | 2.15 | 2 |
| I-214 | 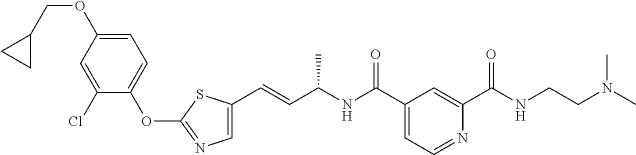 [SMILES: C[C@@H](/C=C/c1cnc(Oc2ccc(OCC3CC3)cc2Cl)s1)NC(=O)c1ccnc(C(=O)NCCN(C)C)c1] | 570 | 1.65 | 2 |
| I-215 | 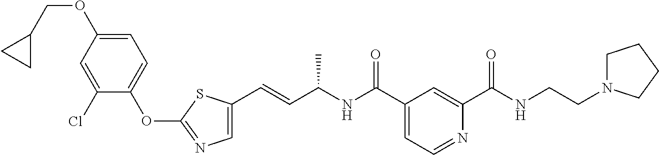 [SMILES: C[C@@H](/C=C/c1cnc(Oc2ccc(OCC3CC3)cc2Cl)s1)NC(=O)c1ccnc(C(=O)NCCN2CCCC2)c1] | 596 | 1.69 | 2 |
| I-216 | 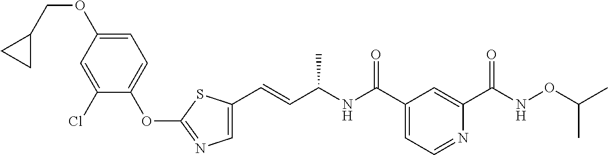 [SMILES: CC(C)ONC(=O)c1cc(C(=O)N[C@@H](C)/C=C/c2cnc(Oc3ccc(OCC4CC4)cc3Cl)s2)ccn1] | 557 | 2.49 | 2 |
| I-217 | 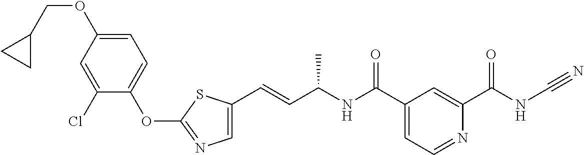 [SMILES: C[C@@H](/C=C/c1cnc(Oc2ccc(OCC3CC3)cc2Cl)s1)NC(=O)c1ccnc(C(=O)NC#N)c1] | 524 | 2.54 | 2 |
| I-218 | 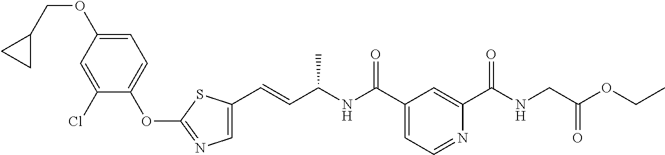 [SMILES: CCOC(=O)CNC(=O)c1cc(C(=O)N[C@@H](C)/C=C/c2cnc(Oc3ccc(OCC4CC4)cc3Cl)s2)ccn1] | 585 | 2.51 | 2 |

TABLE 23

| | | LC/MS | | |
|---|---|---|---|---|
| | | | re- | |
| | | | tention | |
| | | [M + | time | con- |
| No. | Structure | H] | (min) | dition |
| I-219 | 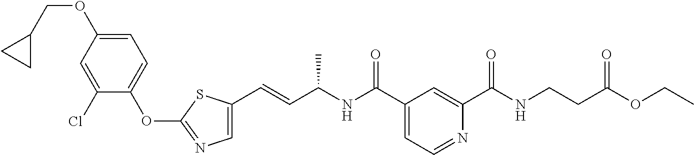 [SMILES: CCOC(=O)CCNC(=O)c1cc(C(=O)N[C@@H](C)/C=C/c2cnc(Oc3ccc(OCC4CC4)cc3Cl)s2)ccn1] | 599 | 2.54 | 2 |
| I-220 | 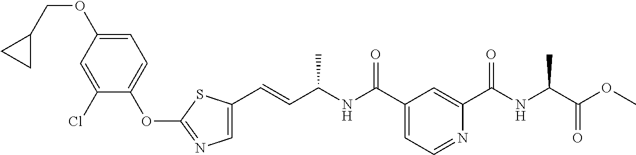 [SMILES: COC(=O)[C@H](C)NC(=O)c1cc(C(=O)N[C@@H](C)/C=C/c2cnc(Oc3ccc(OCC4CC4)cc3Cl)s2)ccn1] | 585 | 2.53 | 2 |
| I-221 | 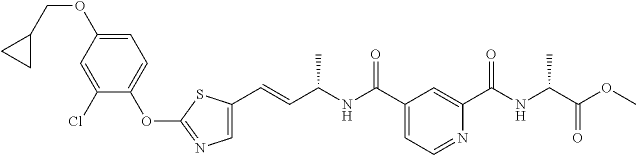 [SMILES: COC(=O)[C@@H](C)NC(=O)c1cc(C(=O)N[C@@H](C)/C=C/c2cnc(Oc3ccc(OCC4CC4)cc3Cl)s2)ccn1] | 585 | 2.53 | 2 |
| I-222 | 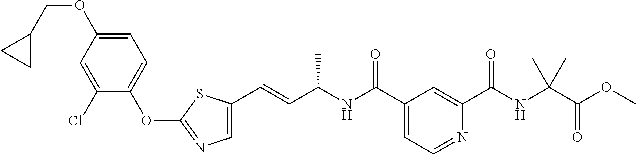 [SMILES: COC(=O)C(C)(C)NC(=O)c1cc(C(=O)N[C@@H](C)/C=C/c2cnc(Oc3ccc(OCC4CC4)cc3Cl)s2)ccn1] | 599 | 2.61 | 2 |
| I-223 | 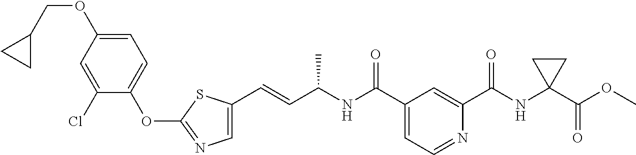 [SMILES: COC(=O)C1(NC(=O)c2cc(C(=O)N[C@@H](C)/C=C/c3cnc(Oc4ccc(OCC5CC5)cc4Cl)s3)ccn2)CC1] | 597 | 2.48 | 2 |

### Examples 224-229

The following compounds were synthesized by hydrolyzing Compounds I-218-I-223 by the similar operation in Step 1 in Example 206.

TABLE 24

| | | LC/MS | | |
|---|---|---|---|---|
| | | | retention | |
| | | | time | condi- |
| No. | Structure | [M + H] | (min) | tion |
| I-224 | 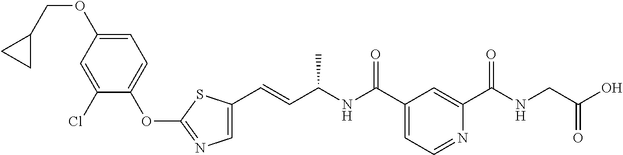 [SMILES: C[C@@H](/C=C/c1cnc(Oc2ccc(OCC3CC3)cc2Cl)s1)NC(=O)c1ccnc(C(=O)NCC(=O)O)c1] | 557 | 2.26 | 2 |
| I-225 | 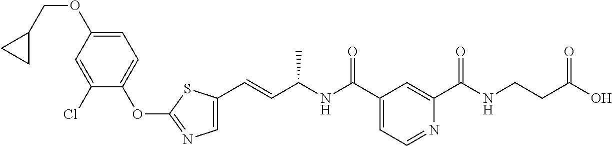 [SMILES: C[C@@H](/C=C/c1cnc(Oc2ccc(OCC3CC3)cc2Cl)s1)NC(=O)c1ccnc(C(=O)NCCC(=O)O)c1] | 571 | 2.25 | 2 |
| I-226 | 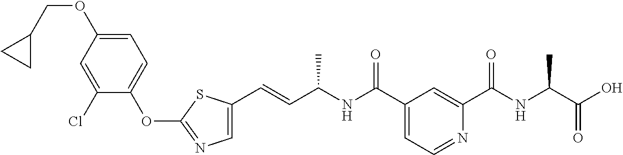 [SMILES: C[C@H](NC(=O)c1cc(C(=O)N[C@@H](C)/C=C/c2cnc(Oc3ccc(OCC4CC4)cc3Cl)s2)ccn1)C(=O)O] | 571 | 2.36 | 2 |
| I-227 | 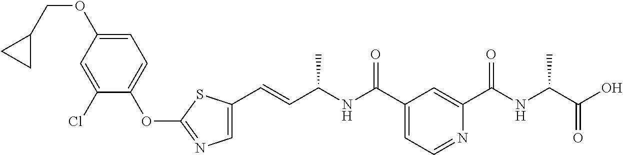 [SMILES: C[C@@H](/C=C/c1cnc(Oc2ccc(OCC3CC3)cc2Cl)s1)NC(=O)c1ccnc(C(=O)N[C@H](C)C(=O)O)c1] | 571 | 2.36 | 2 |
| I-228 | 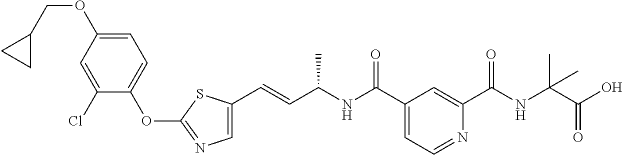 [SMILES: C[C@@H](/C=C/c1cnc(Oc2ccc(OCC3CC3)cc2Cl)s1)NC(=O)c1ccnc(C(=O)NC(C)(C)C(=O)O)c1] | 585 | 2.41 | 2 |
| I-229 | 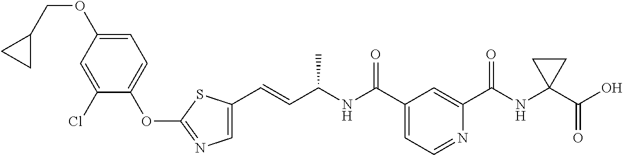 [SMILES: C[C@@H](/C=C/c1cnc(Oc2ccc(OCC3CC3)cc2Cl)s1)NC(=O)c1ccnc(C(=O)NC2(C(=O)O)CC2)c1] | 583 | 2.31 | 2 |

Following compounds were obtained by using Compound I-173 instead of Compound I-136 in Step 1 in Example 206 and by using corresponding amines in Step 2.

TABLE 25

| | | LC/MS | | |
|---|---|---|---|---|
| | | | retention | |
| | | | time | |
| No. | Structure | [M + H] | (min) | condition |
| I-230 | 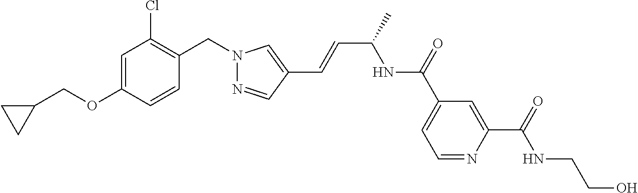 [SMILES: C[C@@H](/C=C/c1cnn(Cc2ccc(OCC3CC3)cc2Cl)c1)NC(=O)c1ccnc(C(=O)NCCO)c1] | 524 | 1.97 | 2 |
| I-231 | 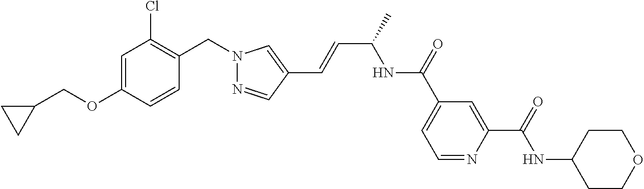 [SMILES: C[C@@H](/C=C/c1cnn(Cc2ccc(OCC3CC3)cc2Cl)c1)NC(=O)c1ccnc(C(=O)NC2CCOCC2)c1] | 564 | 2.18 | 2 |
| I-232 | 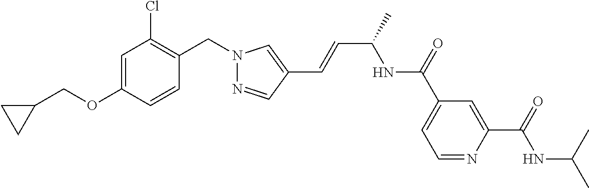 [SMILES: CC(C)NC(=O)c1cc(C(=O)N[C@@H](C)/C=C/c2cnn(Cc3ccc(OCC4CC4)cc3Cl)c2)ccn1] | 522 | 2.35 | 2 |
| I-233 | 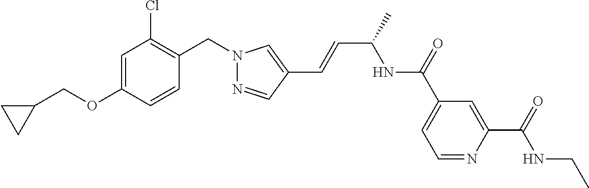 [SMILES: CCNC(=O)c1cc(C(=O)N[C@@H](C)/C=C/c2cnn(Cc3ccc(OCC4CC4)cc3Cl)c2)ccn1] | 508 | 2.21 | 2 |
| I-234 | 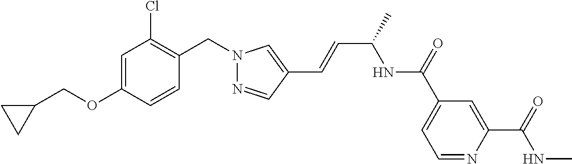 [SMILES: CNC(=O)c1cc(C(=O)N[C@@H](C)/C=C/c2cnn(Cc3ccc(OCC4CC4)cc3Cl)c2)ccn1] | 494 | 2.09 | 2 |
| I-235 | 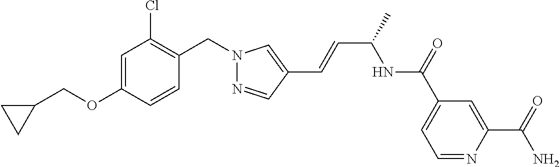 [SMILES: C[C@@H](/C=C/c1cnn(Cc2ccc(OCC3CC3)cc2Cl)c1)NC(=O)c1ccnc(C(N)=O)c1] | 480 | 2 | 2 |

TABLE 26

| | | LC/MS | | |
|---|---|---|---|---|
| | | | retention | |
| | | | time | |
| No. | Structure | [M + H] | (min) | condition |
| I-236 | 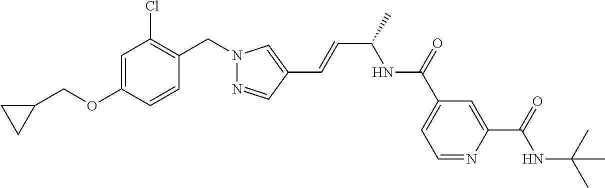 [SMILES: C[C@@H](/C=C/c1cnn(Cc2ccc(OCC3CC3)cc2Cl)c1)NC(=O)c1ccnc(C(=O)NC(C)(C)C)c1] | 536 | 2.51 | 2 |

### Example 237

### Preparation of Compound I-237

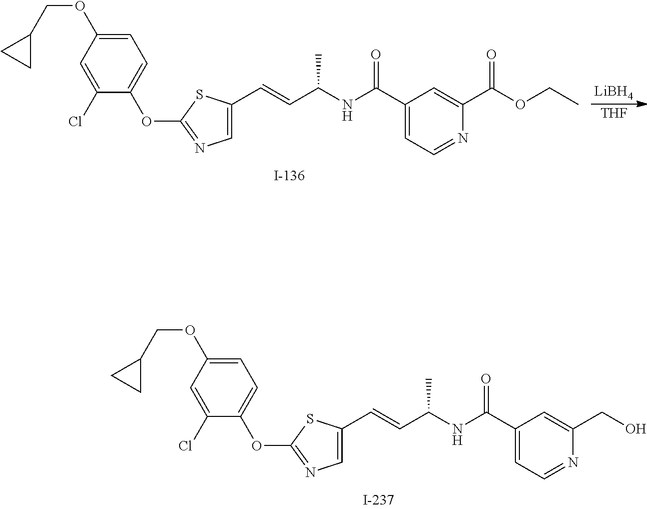

Lithium borohydride (0.58 mg, 0.440 mmol) was added to the tetrahydrofuran solution (2 ml) of Compound I-136 (86 mg, 0.147 mmol), and the mixture was stirred at room temperature for 1.5 hours. Water (15 ml) was added to the mixture, and the mixture was extracted with ethyl acetate. The organic layer was washed with saturated brine, and dried over magnesium sulfate. The solvent was condensed under reduced pressure. The residue was purified by silica gel chromatography (chloroform-methol) to afford Compound I-237 (47.9 mg, yield 67%).

¹H-NMR (DMSO-d₆) δ: 8.81 (d, J=8.2 Hz, 1H), 8.59 (d, J=5.0 Hz, 1H), 7.88 (s, 1H), 7.63 (d, J=4.3 Hz, 1H), 7.45 (d, J=9.0 Hz, 1H), 7.21-7.18 (m, 2H), 6.99 (dd, J=8.9, 3.0 Hz, 1H), 6.59 (d, J=15.7 Hz, 1H), 5.91 (dd, J=15.7, 5.8 Hz, 1H), 5.56-5.48 (m, OH), 4.75-4.64 (m, 1H), 4.61 (d, J=5.6 Hz, 2H), 3.86 (d, J=7.0 Hz, 2H), 1.29 (d, J=6.6 Hz, 3H), 1.26-1.16 (m, 1H), 0.62-0.54 (m, 2H), 0.37-0.29 (m, 2H).

### Example 238

### Preparation of Compound I-238

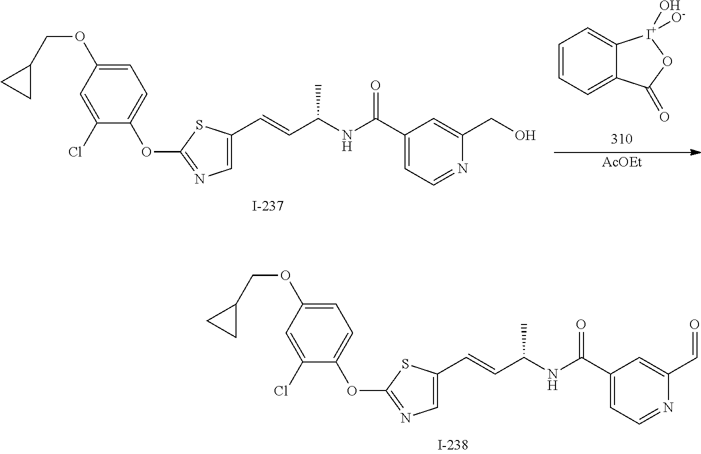 [SMILES: "AcOEt" ; C[C@@H](/C=C/c1cnc(Oc2ccc(OCC3CC3)cc2Cl)s1)NC(=O)c1ccnc(CO)c1 ; O=C1O[I+]([O-])(O)c2ccccc21 ; C[C@@H](/C=C/c1cnc(Oc2ccc(OCC3CC3)cc2Cl)s1)NC(=O)c1ccnc(C=O)c1]

Compound 310 (26.4 mg, 0.094 mmol) was added to the ethyl acetate solution (2 mL) of Compound I-237 (23.0 mg, 0.047 mmol), and the mixture was stirred at 80° C. for 6 hours. The precipitation was filtered, and the filtrate was condensed under reduced pressure. The residue was purified by silica gel chromatography (hexane-ethyl acetate) to afford Compound I-238 (20.8 mg, yield 91.0%).

¹H-NMR (DMSO-d₆) δ: 10.04 (s, 1H), 9.03 (d, J=7.5 Hz, 1H), 8.96 (d, J=4.9 Hz, 1H), 8.34 (s, 1H), 8.08 (d, J=4.1 Hz, 1H), 7.46 (d, J=9.0 Hz, 1H), 7.23-7.17 (m, 2H), 6.99 (dd, J=9.1, 2.8 Hz, 1H), 6.61 (d, J=15.6 Hz, 1H), 5.91 (dd, J=15.8, 5.7 Hz, 1H), 4.77-4.66 (m, 1H), 3.86 (d, J=6.9 Hz, 2H), 1.31 (d, J=6.7 Hz, 3H), 1.28-1.14 (m, 1H), 0.62-0.55 (m, 2H), 0.36-0.30 (m, 2H).

### Example 239

### Preparation of Compound I-239

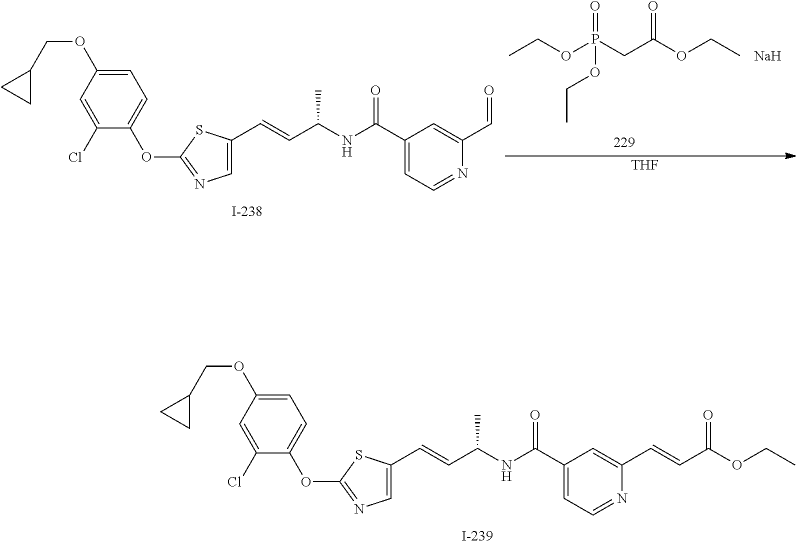

The tetrahydrofuran suspension (2 mL) of sodium hydride (6.69 mg, 0.167 mmol) was cooled with ice in a cool bath in a nitrogen atmosphere. Compound 229 (0.033 mL, 0.167 mmol) was added to the mixture, and the mixture was stirred at room temperature for 10 minutes. Compound I-238 (54 mg, 0.112 mmol) was added to the mixture, and the mixture was stirred at room temperature for 10 minutes. The mixture was added to the saturated ammonium chrolide solution (10 mL), and extracted with chloroform. The organic layer was dried over magnesium sulfate. The solvent was condensed under reduced pressure. The residue was purified by silica gel chromatography (hexane-ethyl acetate) to afford Compound I-239 (47 mg, yield 76%).

¹H-NMR (DMSO-d₆) δ: 8.80-8.75 (m, 2H), 8.15 (s, 1H), 7.77 (dd, J=5.0, 1.6 Hz, 1H), 7.69 (d, J=15.9 Hz, 1H), 7.46 (d, J=9.0 Hz, 1H), 7.22 (s, 1H), 7.20 (d, J=2.9 Hz, 1H), 7.00 (dd, J=9.0, 3.1 Hz, 1H), 6.94 (d, J=15.9 Hz, 1H), 6.62 (d, J=15.7 Hz, 1H), 5.91 (dd, J=15.9, 5.6 Hz, 1H), 4.75-4.65 (m, 1H), 4.22 (q, J=7.1 Hz, 2H), 3.86 (d, J=7.0 Hz, 2H), 1.32-1.22 (m, 7H), 0.61-0.55 (m, 2H), 0.36-0.30 (m, 2H).

### Example 240

### Preparation of Compound I-240

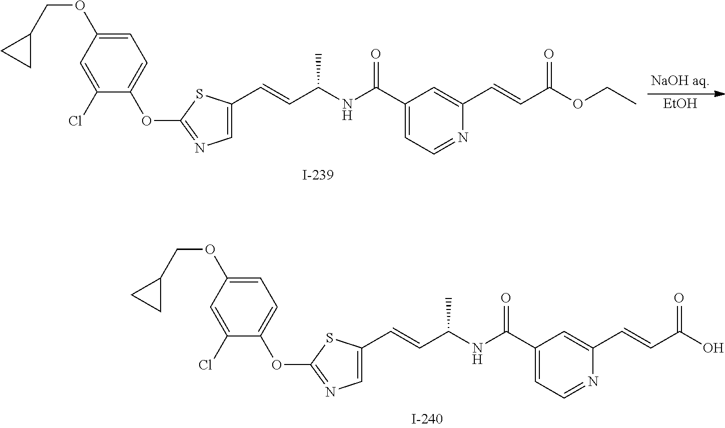 [SMILES: "EtOH" ; CCOC(=O)/C=C/c1cc(C(=O)N[C@@H](C)/C=C/c2cnc(Oc3ccc(OCC4CC4)cc3Cl)s2)ccn1 ; C[C@@H](/C=C/c1cnc(Oc2ccc(OCC3CC3)cc2Cl)s1)NC(=O)c1ccnc(/C=C/C(=O)O)c1]

2 mol/L aqueous sodium hydroxide (0.10 mL, 0.200 mmol) was added to the ethanol solution (1 mL) of Compound I-239 (38 mg, 0.069 mmol), and the mixture was stirred at room temperature for 1 hours. 2 mol/L hydrochloric acid was added to the mixture and the mixture was neutralized. The mixture was extracted with chloroform. The organic layer was dried over magnesium sulfate. The solvent was condensed under reduced pressure. The residue was purified by silica gel chromatography (chloroform-methanol) to afford Compound I-240 (29.3 mg, yield 81%).

¹H-NMR (DMSO-d₆) δ: 8.79 (d, J=8.1 Hz, 1H), 8.75 (d, J=5.2 Hz, 1H), 8.11 (s, 1H), 7.77-7.73 (m, 1H), 7.61 (d, J=15.6 Hz, 1H), 7.46 (d, J=9.0 Hz, 1H), 7.22-7.19 (m, 2H), 6.99 (dd, J=9.0, 3.0 Hz, 1H), 6.88 (d, J=15.6 Hz, 1H), 6.61 (d, J=15.6 Hz, 1H), 5.91 (dd, J=15.6, 5.6 Hz, 1H), 4.75-4.65 (m, 1H), 3.86 (d, J=6.9 Hz, 2H), 1.31 (d, J=6.9 Hz, 3H), 1.26-1.18 (m, 1H), 0.61-0.55 (m, 2H), 0.36-0.30 (m, 2H).

### Example 241

### Preparation of Compound I-241

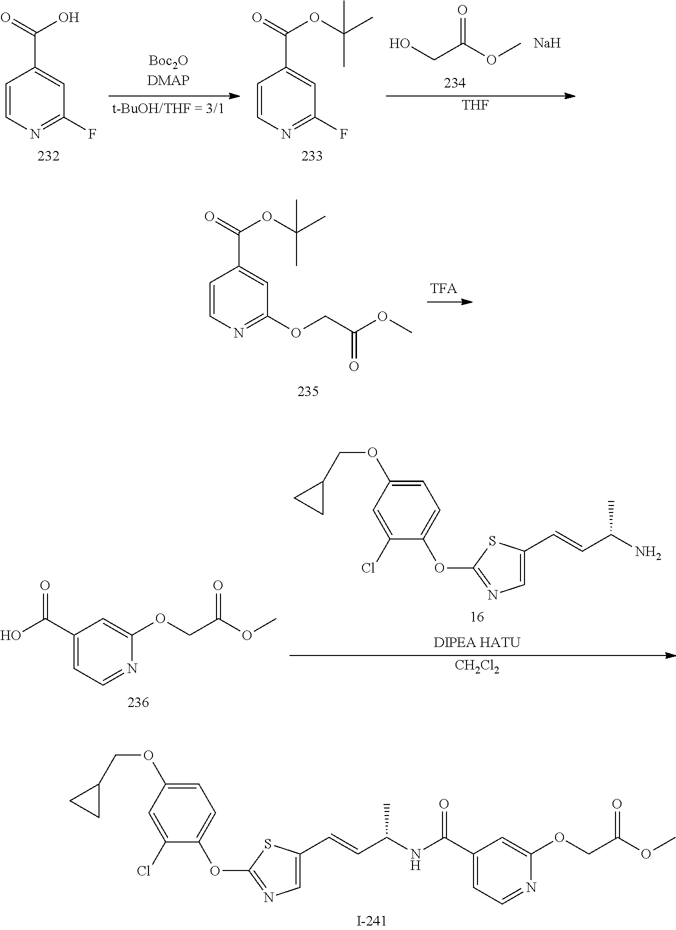

Boc₂O (3.29 mL, 14.17 mmol) and DMAP (170 mg, 1.39 mmol) were added to the suspension of Compound 232 (1.0 g, 7.09 mmol) in 2-methyl-propanol (12 mL) and tetrahydrofuran (4 mL), the mixture was stirred overnight at room temperature. The solvent was condensed under reduced pressure. The residue was purified by silica gel chromatography (hexane-ethyl acetate) to afford Compound 233 (1.26 g, yield 90%).

¹H-NMR (CDCl₃) δ: 8.32 (dd, J=5.1, 0.5 Hz, 1H), 7.68 (dt, J=5.0, 1.4 Hz, 1H), 7.42-7.41 (m, 1H), 1.61 (s, 9H).

### Step 2 Preparation of Compound 235

The tetrahydrofuran suspension (4 mL) of sodium hydride (122 mg, 3.04 mmol) was cooled with ice in a cool bath in a nitrogen atmosphere. Compound 234 (0.231 mL, 3.04 mmol) was added to the mixture, and the mixture was stirred at room temperature for 15 minutes. The tetrahydrofuran solution (2 mL) of Compound 233 (400 mg, 2.03 mmol) was added to the mixture, and the mixture was stirred at 60° C. for 2 hours. The mixture was added to the saturated ammonium chrolide, and extracted with chloroform. The organic layer was dried over magnesium sulfate. The solvent was condensed under reduced pressure. The residue was purified by silica gel chromatography (hexane-ethyl acetate) to afford Compound 235 (201 mg, yield 35%).

¹H-NMR (CDCl₃) δ: 8.18 (d, J=5.2 Hz, 1H), 7.41-7.38 (m, 2H), 4.93 (s, 2H), 3.77 (s, 3H), 1.58 (s, 9H).

### Step 3 Preparation of Compound 236

Trifluorocetic acid (1 mL, 12.98 mmol) was added to the Compound 235 (58 mg, 0.206 mmol), and the mixture was stirred at room temperature for 3 hours. The mixture was condensed under reduced pressure. The residue was followed as such to the next step.

### Step 4 Preparation of Compound I-241

N-ethyldiisopropyl amine (0.182 mL, 1.04 mmol) and HATU (119 mg, 0.313 mmol) were added to the dichloromethane suspension of Compound 16 (73.1 mg, 0.208 mmol) and Compound 236 (44 mg, 0.208), and the mixture was stirred at room temperature for 3 hours. Saturated sodium bicarbonate water was added to the mixture, and the mixture was extracted with chloroform. The organic layer was dried over magnesium sulfate. The solvent was condensed under reduced pressure. The residue was purified by silica gel chromatography (hexane-ethyl acetate) to afford Compound I-241 (90.6 mg, yield 80%).

¹H-NMR (DMSO-d₆) δ: 8.75 (d, J=7.8 Hz, 1H), 8.22 (d, J=5.5 Hz, 1H), 7.45 (d, J=9.2 Hz, 1H), 7.39 (d, J=5.3 Hz, 1H), 7.32 (s, 1H), 7.23-7.17 (m, 2H), 6.99 (dd, J=9.2, 2.9 Hz, 1H), 6.58 (d, J=15.6 Hz, 1H), 5.90 (dd, J=15.9, 5.7 Hz, 1H), 4.96 (s, 2H), 4.74-4.61 (m, 1H), 3.86 (d, J=7.0 Hz, 2H), 3.66 (s, 3H), 1.28 (d, J=6.9 Hz, 3H), 1.26-1.15 (m, 1H), 0.62-0.54 (m, 2H), 0.36-0.30 (m, 2H).

### Example 242

### Preparation of Compound I-242

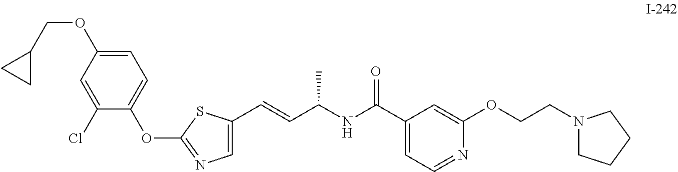 [SMILES: C[C@@H](/C=C/c1cnc(Oc2ccc(OCC3CC3)cc2Cl)s1)NC(=O)c1ccnc(OCCN2CCCC2)c1]

Compound I-242 was obtained by using 2-(pyrrolidine-1-yl) ethanol instead of Compound 234 in Step 2 in Example 241.

[M+H]=569, Method Condition 2: retention time 1.79 min

### Example 243

### Preparation of Compound I-243

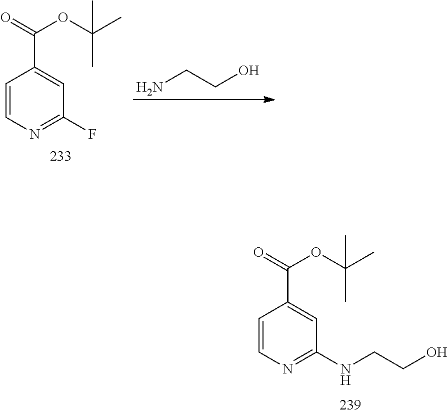

Compound 233 (200 mg, 1.01 mmol) was dissolved in aminoethanol (1 mL, 16.5 mmol), and the mixture was stirred at 80° C. for 1 hour. Water was added to the mixture, and the mixture was extracted with chloroform. The organic layer was dried over magnesium sulfate. The solvent was condensed under reduced pressure. The residue was purified by silica gel chromatography (hexane-ethyl acetate) to afford Compound 239 (107 mg, yield 44%).

¹H-NMR (CDCl₃) δ: 8.12 (d, J=5.4 Hz, 1H), 7.05 (dd, J=5.4, 1.2 Hz, 1H), 6.98 (d, J=1.2 Hz, 1H), 5.01-4.93 (m, 1H), 3.82 (t, J=4.7 Hz, 2H), 3.57-3.54 (m, 2H), 1.58 (s, 9H).

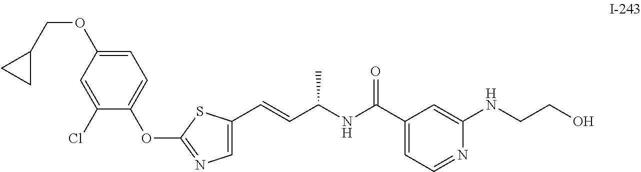 [SMILES: C[C@@H](/C=C/c1cnc(Oc2ccc(OCC3CC3)cc2Cl)s1)NC(=O)c1ccnc(NCCO)c1]

Compound I-243 was obtained by operation similar to that Steps 3 and 4 in Example 106.

¹H-NMR (DMSO-d₆) δ: 8.50 (d, J=8.1 Hz, 1H), 8.01 (d, J=5.0 Hz, 1H), 7.45 (d, J=9.0 Hz, 1H), 7.22-7.17 (m, 2H), 7.03-6.96 (m, 1H), 6.89-6.80 (m, 2H), 6.68 (t, J=5.5 Hz, 1H), 6.55 (d, J=15.8 Hz, 1H), 5.88 (dd, J=15.8, 5.9 Hz, 1H), 4.73-4.59 (m, 2H), 3.86 (d, J=7.0 Hz, 2H), 3.55-3.46 (m, 1H), 1.30-1.17 (m, 4H), 0.61-0.55 (m, 2H), 0.37-0.29 (m, 2H).

The following Compounds were obtained by using the corresponding amine in Step 1 in Example 243.

TABLE 27

| | | LC/MS | | |
|---|---|---|---|---|
| | | | retention | |
| | | | time | |
| No. | Structure | [M + H] | (min) | condition |
| I-244 | 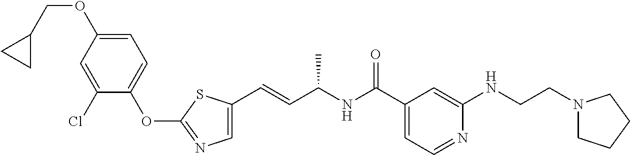 [SMILES: C[C@@H](/C=C/c1cnc(Oc2ccc(OCC3CC3)cc2Cl)s1)NC(=O)c1ccnc(NCCN2CCCC2)c1] | 568 | 1.68 | 2 |
| I-245 | 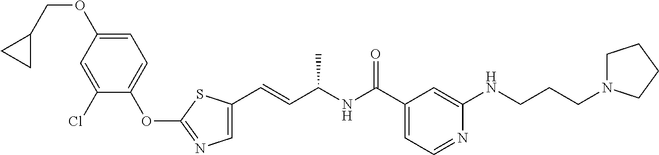 [SMILES: C[C@@H](/C=C/c1cnc(Oc2ccc(OCC3CC3)cc2Cl)s1)NC(=O)c1ccnc(NCCCN2CCCC2)c1] | 582 | 1.6 | 2 |
| I-246 | 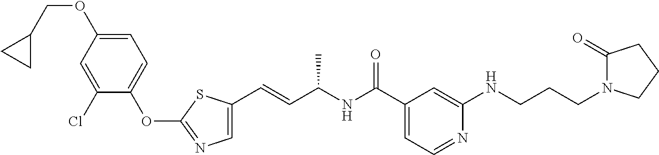 [SMILES: C[C@@H](/C=C/c1cnc(Oc2ccc(OCC3CC3)cc2Cl)s1)NC(=O)c1ccnc(NCCCN2CCCC2=O)c1] | 596 | 1.74 | 2 |
| I-247 | 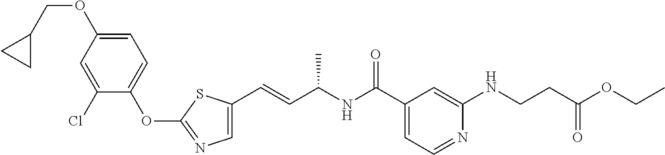 [SMILES: CCOC(=O)CCNc1cc(C(=O)N[C@@H](C)/C=C/c2cnc(Oc3ccc(OCC4CC4)cc3Cl)s2)ccn1] | 571 | 1.99 | 2 |

### Example 248

### Preparation of Compound I-248

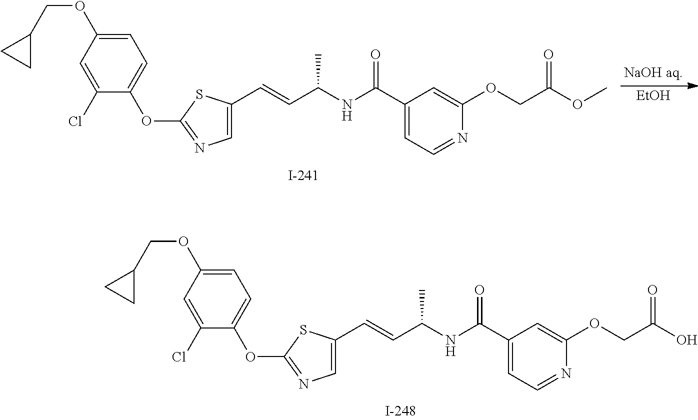 [SMILES: "EtOH" ; COC(=O)COc1cc(C(=O)N[C@@H](C)/C=C/c2cnc(Oc3ccc(OCC4CC4)cc3Cl)s2)ccn1 ; C[C@@H](/C=C/c1cnc(Oc2ccc(OCC3CC3)cc2Cl)s1)NC(=O)c1ccnc(OCC(=O)O)c1]

2 mol/L aqueous sodium hydroxide (0.20 mL, 0.400 mmol) was added to the methanol solution (1.5 mL) of Compound I-241 (72 mg, 0.132 mmol), and the mixture was stirred at room temperature for 2 hours. 2 mol/L hydrochloric acid was added to the mixture and the mixture was neutralized. The mixture was extracted with chloroform. The organic layer was dried over magnesium sulfate. The solvent was condensed under reduced pressure. The residue was suspended in chloroform and hexane, and the precipitated solids were filtered off, and dried under vacuum to obtain compound 1-248 (70 mg, yield 99.8%).

¹H-NMR (DMSO-d₆) δ: 12.8 (brs, 1H) 8.73 (d, J=7.9 Hz, 1H), 8.23 (d, J=5.4 Hz, 1H), 7.46 (d, J=8.9 Hz, 1H), 7.38 (d, J=5.2 Hz, 1H), 7.29 (s, 1H), 7.23-7.18 (m, 2H), 7.00 (dd, J=9.1, 2.7 Hz, 1H), 6.59 (d, J=15.4 Hz, 1H), 5.90 (dd, J=15.8, 5.7 Hz, 1H), 4.86 (s, 2H), 4.73-4.62 (m, 1H), 3.86 (d, J=6.9 Hz, 2H), 1.28 (d, J=6.9 Hz, 3H), 1.26-1.17 (m, 1H), 0.62-0.55 (m, 2H), 0.36-0.30 (m, 2H).

### Example 249

### Preparation of Compound I-249

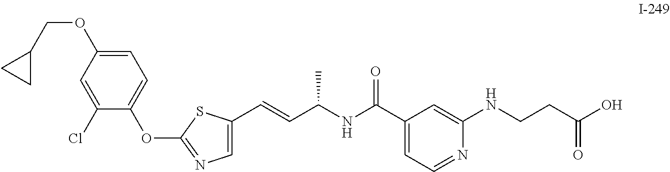 [SMILES: C[C@@H](/C=C/c1cnc(Oc2ccc(OCC3CC3)cc2Cl)s1)NC(=O)c1ccnc(NCCC(=O)O)c1]

Compound I-249 was obtained by using Compound I-247 instead of Compound I-241 in Example 248.

[M+H]=543, Method Condition 2: retention time 1.78 min

### Example 250

Compounds I-431-520 were obtained in the above similar Example. The structures and chemical data of Compounds of I-431-520 are showed.

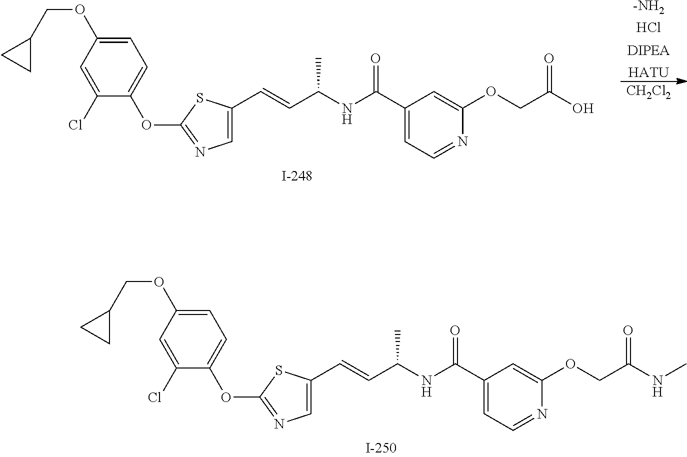

N-ethyldiisopropylamine (0.029 ml, 0.165 mmol) and HATU (32.6 mg, 0.086 mmol) were added to the dichloromethane solution of Compound I-248 (35 mg, 0.066 mmol) and methylammonium chloride (6.69 mg, 0.099 mmol), and the mixture was stirred at room temperature for 1 hour. Saturated sodium bicarbonate water was added to the mixture, and the mixture was extracted with chloroform. The organic layer was dried over magnesium sulfate. The solvent was condensed under reduced pressure. The residue was purified by silica gel chromatography (hexane-ethyl acetate) to afford Compound I-250 (18.6 mg, yield 52%).

¹H-NMR (DMSO-d₆) δ: 8.75 (d, J=7.8 Hz, 1H), 8.23 (d, J=5.3 Hz, 1H), 7.96 (d, J=4.6 Hz, 1H), 7.46 (d, J=9.0 Hz, 1H), 7.39 (d, J=5.5 Hz, 1H), 7.32 (s, 1H), 7.22-7.18 (m, 2H), 6.99 (dd, J=8.7, 2.6 Hz, 1H), 6.58 (d, J=15.7 Hz, 1H), 5.90 (dd, J=15.7, 5.9 Hz, 1H), 4.73 (s, 2H), 4.72-4.64 (m, 1H), 3.86 (d, J=6.9 Hz, 2H), 2.60 (d, J=4.6 Hz, 3H), 1.29 (d, J=7.0 Hz, 3H), 1.27-1.17 (m, 1H), 0.62-0.55 (m, 2H), 0.36-0.30 (m, 2H).

### Examples 251-430

Compounds I-251-430 were obtained in the above similar Example. The structures and chemical data of Compounds of 1-251-430 are showed.

TABLE 28

| | | LC/MS | | |
|---|---|---|---|---|
| | | | retention | |
| | | | time | |
| No. | Structure | [M + H] | (min) | condition |
| I-251 | 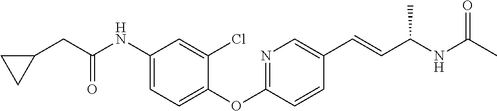 [SMILES: CC(=O)N[C@@H](C)/C=C/c1ccc(Oc2ccc(NC(=O)CC3CC3)cc2Cl)nc1] | 414 | 1.82 | 2 |
| I-252 | 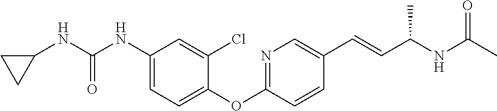 [SMILES: CC(=O)N[C@@H](C)/C=C/c1ccc(Oc2ccc(NC(=O)NC3CC3)cc2Cl)nc1] | 415 | 1.60 | 2 |
| I-253 | 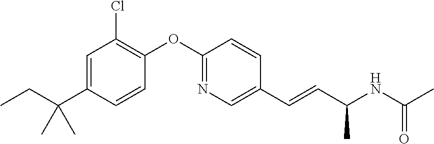 [SMILES: CCC(C)(C)c1ccc(Oc2ccc(/C=C/[C@H](C)NC(C)=O)cn2)c(Cl)c1] | 387 | 2.57 | 2 |
| I-254 | 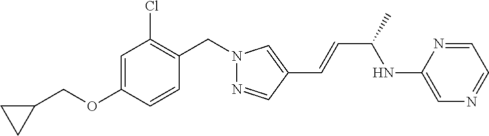 [SMILES: C[C@@H](/C=C/c1cnn(Cc2ccc(OCC3CC3)cc2Cl)c1)Nc1cnccn1] | 401 | 2.22 | 2 |
| I-255 | 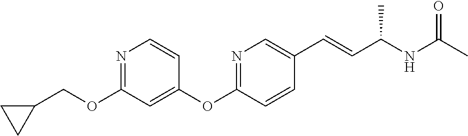 [SMILES: CC(=O)N[C@@H](C)/C=C/c1ccc(Oc2ccnc(OCC3CC3)c2)nc1] | 354 | 1.84 | 2 |
| I-256 | 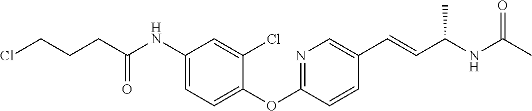 [SMILES: CC(=O)N[C@@H](C)/C=C/c1ccc(Oc2ccc(NC(=O)CCCCl)cc2Cl)nc1] | 436 | 1.87 | 2 |

TABLE 29

| | | | | |
|---|---|---|---|---|
| I-257 | 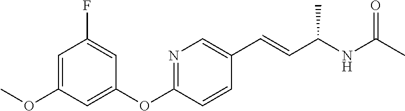 [SMILES: COc1cc(F)cc(Oc2ccc(/C=C/[C@H](C)NC(C)=O)cn2)c1] | 331 | 1.88 | 2 |
| I-258 | 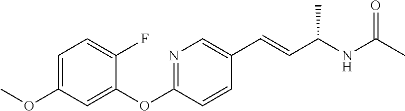 [SMILES: COc1ccc(F)c(Oc2ccc(/C=C/[C@H](C)NC(C)=O)cn2)c1] | 331 | 1.82 | 2 |
| I-259 | 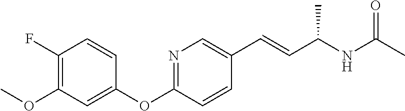 [SMILES: COc1cc(Oc2ccc(/C=C/[C@H](C)NC(C)=O)cn2)ccc1F] | 331 | 1.78 | 2 |
| I-260 | 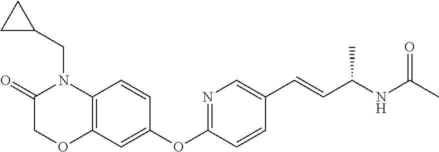 [SMILES: CC(=O)N[C@@H](C)/C=C/c1ccc(Oc2ccc3c(c2)OCC(=O)N3CC2CC2)nc1] | 408 | 1.85 | 2 |
| I-261 | 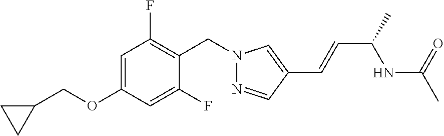 [SMILES: CC(=O)N[C@@H](C)/C=C/c1cnn(Cc2c(F)cc(OCC3CC3)cc2F)c1] | 376 | 1.04 | 2 |
| I-262 | 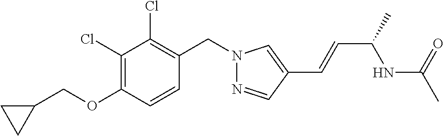 [SMILES: CC(=O)N[C@@H](C)/C=C/c1cnn(Cc2ccc(OCC3CC3)c(Cl)c2Cl)c1] | 408 | 2.12 | 2 |

TABLE 30

| | | | | |
|---|---|---|---|---|
| I-263 | 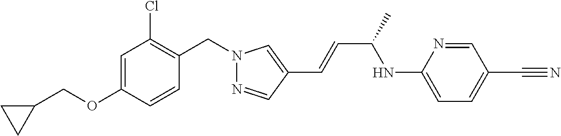 [SMILES: C[C@@H](/C=C/c1cnn(Cc2ccc(OCC3CC3)cc2Cl)c1)Nc1ccc(C#N)cn1] | 434 | 2.41 | 2 |
| I-264 | 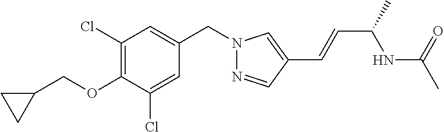 [SMILES: CC(=O)N[C@@H](C)/C=C/c1cnn(Cc2cc(Cl)c(OCC3CC3)c(Cl)c2)c1] | 408 | 2.11 | 2 |
| I-265 | 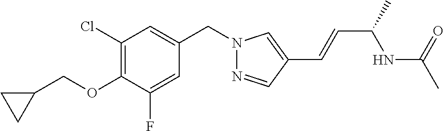 [SMILES: CC(=O)N[C@@H](C)/C=C/c1cnn(Cc2cc(F)c(OCC3CC3)c(Cl)c2)c1] | 392 | 2.04 | 2 |
| I-266 | 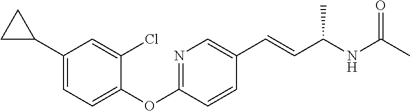 [SMILES: CC(=O)N[C@@H](C)/C=C/c1ccc(Oc2ccc(C3CC3)cc2Cl)nc1] | 357 | 2.24 | 2 |
| I-267 | 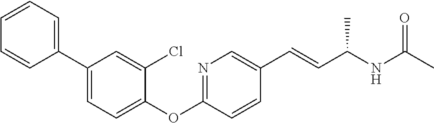 [SMILES: CC(=O)N[C@@H](C)/C=C/c1ccc(Oc2ccc(-c3ccccc3)cc2Cl)nc1] | 393 | 2.41 | 2 |
| I-268 | 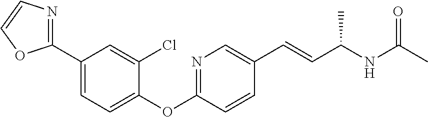 [SMILES: CC(=O)N[C@@H](C)/C=C/c1ccc(Oc2ccc(-c3ncco3)cc2Cl)nc1] | 384 | 1.91 | 2 |

TABLE 31

| | | | | |
|---|---|---|---|---|
| I-269 | 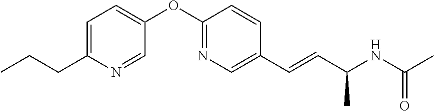 [SMILES: CCCc1ccc(Oc2ccc(/C=C/[C@H](C)NC(C)=O)cn2)cn1] | 326 | 1.35 | 2 |
| I-270 | 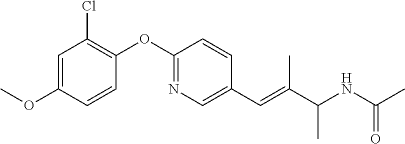 [SMILES: COc1ccc(Oc2ccc(/C=C(\C)C(C)NC(C)=O)cn2)c(Cl)c1] | 337 | 1.61 | 2 |
| I-271 | 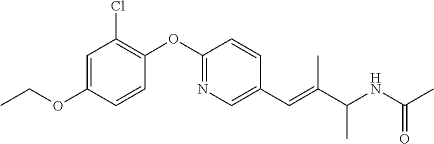 [SMILES: CCOc1ccc(Oc2ccc(/C=C(\C)C(C)NC(C)=O)cn2)c(Cl)c1] | 375 | 2.15 | 2 |
| I-272 | 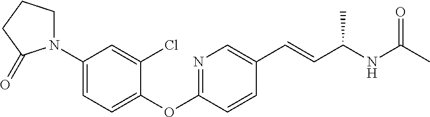 [SMILES: CC(=O)N[C@@H](C)/C=C/c1ccc(Oc2ccc(N3CCCC3=O)cc2Cl)nc1] | 400 | 1.70 | 2 |
| I-273 | 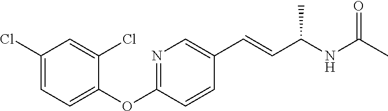 [SMILES: CC(=O)N[C@@H](C)/C=C/c1ccc(Oc2ccc(Cl)cc2Cl)nc1] | 351 | 2.17 | 2 |
| I-274 | 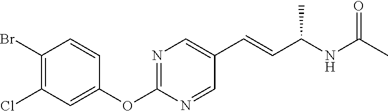 [SMILES: CC(=O)N[C@@H](C)/C=C/c1cnc(Oc2ccc(Br)c(Cl)c2)nc1] | 398 | 1.99 | 2 |

TABLE 32

| | | | | |
|---|---|---|---|---|
| I-275 | 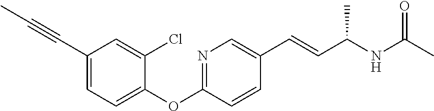 [SMILES: CC#Cc1ccc(Oc2ccc(/C=C/[C@H](C)NC(C)=O)cn2)c(Cl)c1] | 355 | 2.22 | 2 |
| I-276 | 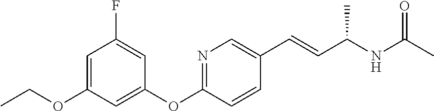 [SMILES: CCOc1cc(F)cc(Oc2ccc(/C=C/[C@H](C)NC(C)=O)cn2)c1] | 345 | 2.06 | 2 |
| I-277 | 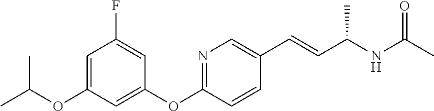 [SMILES: CC(=O)N[C@@H](C)/C=C/c1ccc(Oc2cc(F)cc(OC(C)C)c2)nc1] | 359 | 2.18 | 2 |
| I-278 | 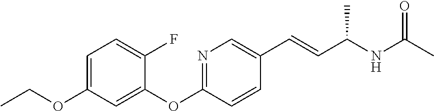 [SMILES: CCOc1ccc(F)c(Oc2ccc(/C=C/[C@H](C)NC(C)=O)cn2)c1] | 345 | 1.99 | 2 |
| I-279 | 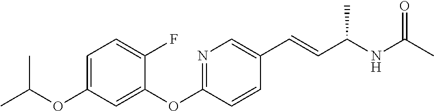 [SMILES: CC(=O)N[C@@H](C)/C=C/c1ccc(Oc2cc(OC(C)C)ccc2F)nc1] | 359 | 2.12 | 2 |
| I-280 | 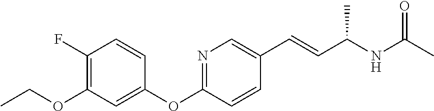 [SMILES: CCOc1cc(Oc2ccc(/C=C/[C@H](C)NC(C)=O)cn2)ccc1F] | 345 | 1.94 | 2 |

TABLE 33

| | | | | |
|---|---|---|---|---|
| I-281 | 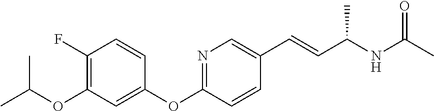 [SMILES: CC(=O)N[C@@H](C)/C=C/c1ccc(Oc2ccc(F)c(OC(C)C)c2)nc1] | 359 | 2.07 | 2 |
| I-282 | 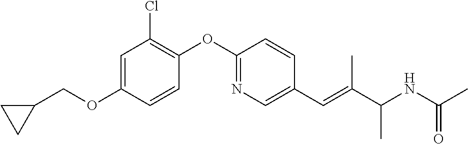 [SMILES: CC(=O)NC(C)/C(C)=C/c1ccc(Oc2ccc(OCC3CC3)cc2Cl)nc1] | 401 | 2.29 | 2 |
| I-283 | 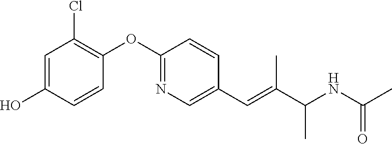 [SMILES: CC(=O)NC(C)/C(C)=C/c1ccc(Oc2ccc(O)cc2Cl)nc1] | 347 | 1.60 | 2 |
| I-284 | 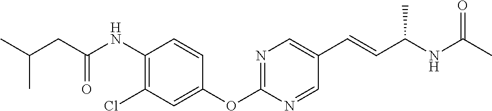 [SMILES: CC(=O)N[C@@H](C)/C=C/c1cnc(Oc2ccc(NC(=O)CC(C)C)c(Cl)c2)nc1] | 417 | 1.74 | 2 |
| I-285 | 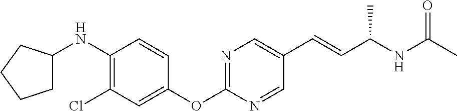 [SMILES: CC(=O)N[C@@H](C)/C=C/c1cnc(Oc2ccc(NC3CCCC3)c(Cl)c2)nc1] | 401 | 2.25 | 2 |
| I-286 | 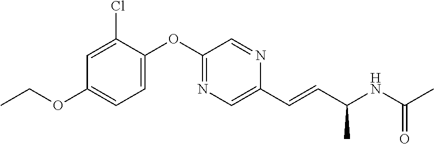 [SMILES: CCOc1ccc(Oc2cnc(/C=C/[C@H](C)NC(C)=O)cn2)c(Cl)c1] | 362 | 2.04 | 2 |

TABLE 34

| | | | | |
|---|---|---|---|---|
| I-287 | 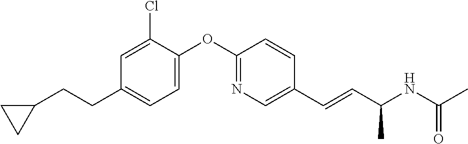 [SMILES: CC(=O)N[C@@H](C)/C=C/c1ccc(Oc2ccc(CCC3CC3)cc2Cl)nc1] | 385 | 2.49 | 2 |
| I-288 | 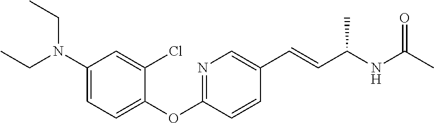 [SMILES: CCN(CC)c1ccc(Oc2ccc(/C=C/[C@H](C)NC(C)=O)cn2)c(Cl)c1] | 388 | 1.86 | 2 |
| I-289 | 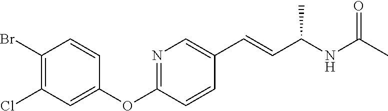 [SMILES: CC(=O)N[C@@H](C)/C=C/c1ccc(Oc2ccc(Br)c(Cl)c2)nc1] | 397 | 2.23 | 2 |
| I-290 | 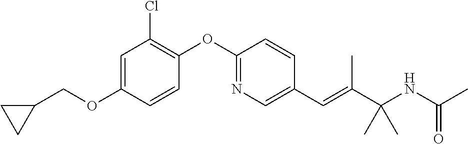 [SMILES: CC(=O)NC(C)(C)/C(C)=C/c1ccc(Oc2ccc(OCC3CC3)cc2Cl)nc1] | 415 | 2.39 | 2 |
| I-291 | 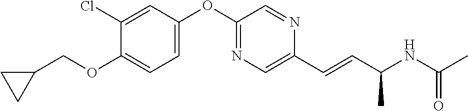 [SMILES: CC(=O)N[C@@H](C)/C=C/c1cnc(Oc2ccc(OCC3CC3)c(Cl)c2)cn1] | 388 | 2.17 | 2 |
| I-292 | 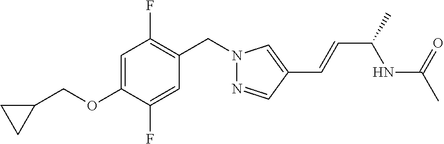 [SMILES: CC(=O)N[C@@H](C)/C=C/c1cnn(Cc2cc(F)c(OCC3CC3)cc2F)c1] | 376 | 1.86 | 2 |

TABLE 35

| | | | | |
|---|---|---|---|---|
| I-293 | 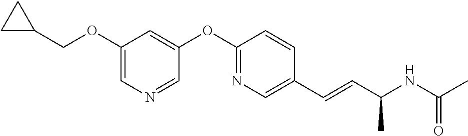 [SMILES: CC(=O)N[C@@H](C)/C=C/c1ccc(Oc2cncc(OCC3CC3)c2)nc1] | 354 | 1.60 | 2 |
| I-294 | 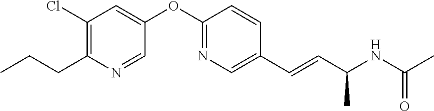 [SMILES: CCCc1ncc(Oc2ccc(/C=C/[C@H](C)NC(C)=O)cn2)cc1Cl] | 360 | 2.08 | 2 |
| I-295 | 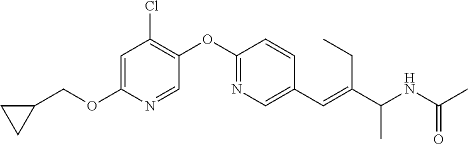 [SMILES: CC/C(=C\c1ccc(Oc2cnc(OCC3CC3)cc2Cl)nc1)C(C)NC(C)=O] | 415 | 2.40 | 2 |
| I-296 | 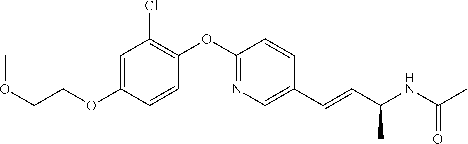 [SMILES: COCCOc1ccc(Oc2ccc(/C=C/[C@H](C)NC(C)=O)cn2)c(Cl)c1] | 391 | 1.84 | 2 |
| I-297 | 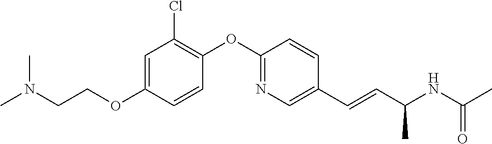 [SMILES: CC(=O)N[C@@H](C)/C=C/c1ccc(Oc2ccc(OCCN(C)C)cc2Cl)nc1] | 404 | 1.04 | 2 |
| I-298 | 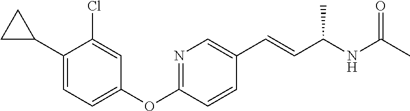 [SMILES: CC(=O)N[C@@H](C)/C=C/c1ccc(Oc2ccc(C3CC3)c(Cl)c2)nc1] | 357 | 2.27 | 2 |

TABLE 36

| | | | | |
|---|---|---|---|---|
| I-299 | 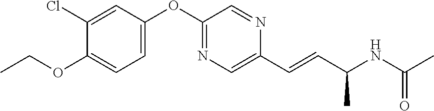 [SMILES: CCOc1ccc(Oc2cnc(/C=C/[C@H](C)NC(C)=O)cn2)cc1Cl] | 362 | 2.01 | 2 |
| I-300 | 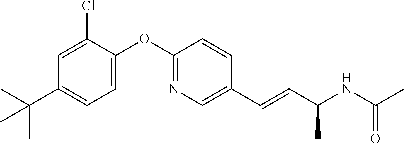 [SMILES: CC(=O)N[C@@H](C)/C=C/c1ccc(Oc2ccc(C(C)(C)C)cc2Cl)nc1] | 373 | 2.43 | 2 |
| I-301 | 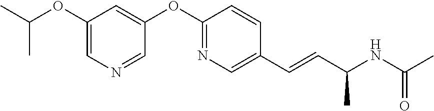 [SMILES: CC(=O)N[C@@H](C)/C=C/c1ccc(Oc2cncc(OC(C)C)c2)nc1] | 342 | 1.53 | 2 |
| I-302 | 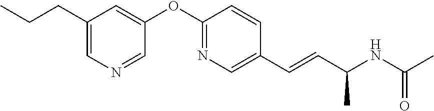 [SMILES: CCCc1cncc(Oc2ccc(/C=C/[C@H](C)NC(C)=O)cn2)c1] | 326 | 1.39 | 2 |
| I-303 | 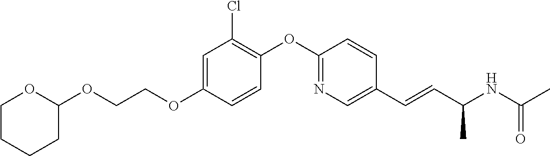 [SMILES: CC(=O)N[C@@H](C)/C=C/c1ccc(Oc2ccc(OCCOC3CCCCO3)cc2Cl)nc1] | 461 | 2.15 | 2 |
| I-304 | 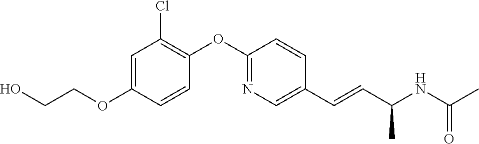 [SMILES: CC(=O)N[C@@H](C)/C=C/c1ccc(Oc2ccc(OCCO)cc2Cl)nc1] | 377 | 1.50 | 2 |

TABLE 37

| | | | | |
|---|---|---|---|---|
| I-305 | 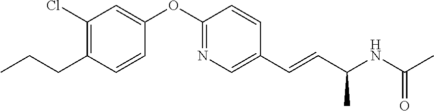 [SMILES: CCCc1ccc(Oc2ccc(/C=C/[C@H](C)NC(C)=O)cn2)cc1Cl] | 359 | 2.45 | 2 |
| I-306 | 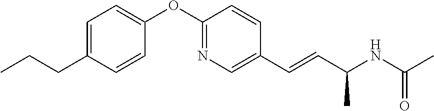 [SMILES: CCCc1ccc(Oc2ccc(/C=C/[C@H](C)NC(C)=O)cn2)cc1] | 325 | 2.22 | 2 |
| I-307 | 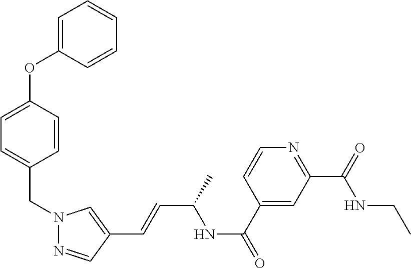 [SMILES: CCNC(=O)c1cc(C(=O)N[C@@H](C)/C=C/c2cnn(Cc3ccc(Oc4ccccc4)cc3)c2)ccn1] | 496 | 2.14 | 3 |
| I-308 | 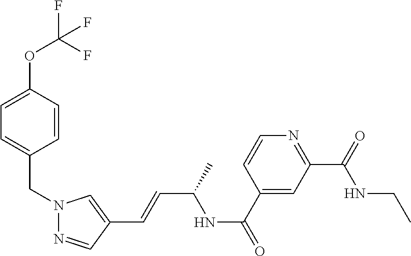 [SMILES: CCNC(=O)c1cc(C(=O)N[C@@H](C)/C=C/c2cnn(Cc3ccc(OC(F)(F)F)cc3)c2)ccn1] | 488 | 2.02 | 3 |
| I-309 | 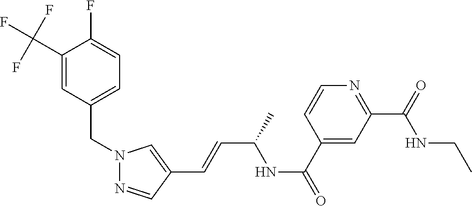 [SMILES: CCNC(=O)c1cc(C(=O)N[C@@H](C)/C=C/c2cnn(Cc3ccc(F)c(C(F)(F)F)c3)c2)ccn1] | 490 | 1.99 | 3 |
| I-310 | 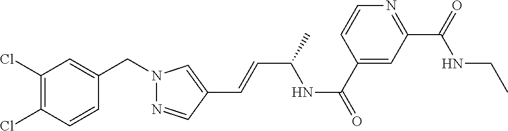 [SMILES: CCNC(=O)c1cc(C(=O)N[C@@H](C)/C=C/c2cnn(Cc3ccc(Cl)c(Cl)c3)c2)ccn1] | 472 | 2.03 | 3 |

TABLE 38

| | | | | |
|---|---|---|---|---|
| I-311 | 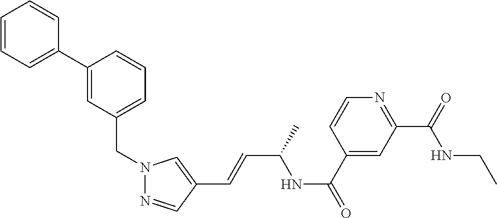 [SMILES: CCNC(=O)c1cc(C(=O)N[C@@H](C)/C=C/c2cnn(Cc3cccc(-c4ccccc4)c3)c2)ccn1] | 480 | 2.13 | 3 |
| I-312 | 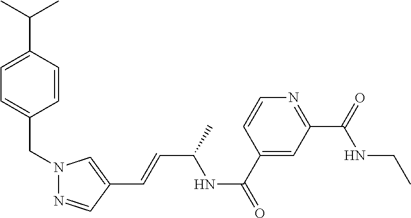 [SMILES: CCNC(=O)c1cc(C(=O)N[C@@H](C)/C=C/c2cnn(Cc3ccc(C(C)C)cc3)c2)ccn1] | 446 | 2.12 | 3 |
| I-313 | 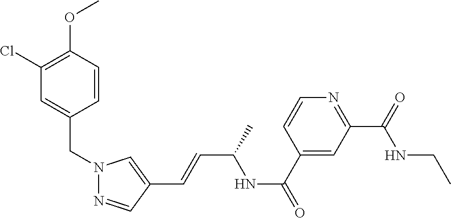 [SMILES: CCNC(=O)c1cc(C(=O)N[C@@H](C)/C=C/c2cnn(Cc3ccc(OC)c(Cl)c3)c2)ccn1] | 468 | 1.81 | 3 |
| I-314 | 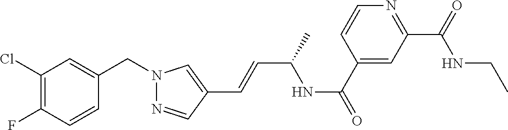 [SMILES: CCNC(=O)c1cc(C(=O)N[C@@H](C)/C=C/c2cnn(Cc3ccc(F)c(Cl)c3)c2)ccn1] | 456 | 1.90 | 3 |
| I-315 | 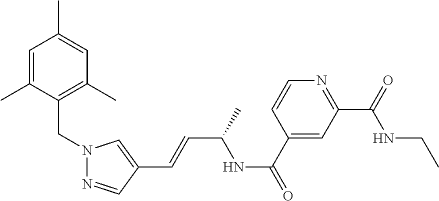 [SMILES: CCNC(=O)c1cc(C(=O)N[C@@H](C)/C=C/c2cnn(Cc3c(C)cc(C)cc3C)c2)ccn1] | 446 | 2.11 | 3 |
| I-316 | 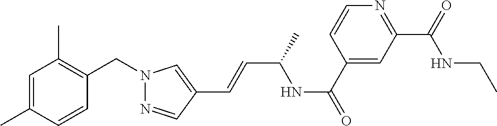 [SMILES: CCNC(=O)c1cc(C(=O)N[C@@H](C)/C=C/c2cnn(Cc3ccc(C)cc3C)c2)ccn1] | 432 | 1.97 | 3 |

TABLE 39

| | | | | |
|---|---|---|---|---|
| I-317 | 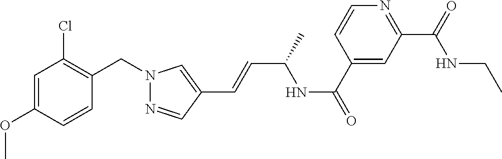 [SMILES: CCNC(=O)c1cc(C(=O)N[C@@H](C)/C=C/c2cnn(Cc3ccc(OC)cc3Cl)c2)ccn1] | 468 | 1.87 | 3 |
| I-318 | 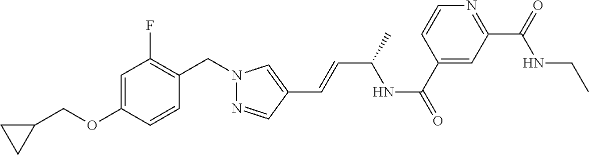 [SMILES: CCNC(=O)c1cc(C(=O)N[C@@H](C)/C=C/c2cnn(Cc3ccc(OCC4CC4)cc3F)c2)ccn1] | 492 | 2.06 | 3 |
| I-319 | 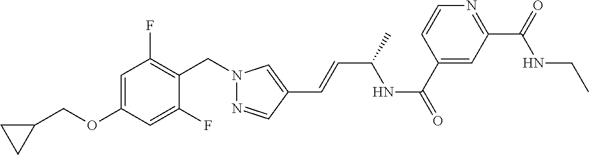 [SMILES: CCNC(=O)c1cc(C(=O)N[C@@H](C)/C=C/c2cnn(Cc3c(F)cc(OCC4CC4)cc3F)c2)ccn1] | 510 | 2.11 | 3 |
| I-320 | 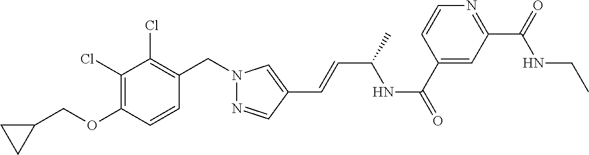 [SMILES: CCNC(=O)c1cc(C(=O)N[C@@H](C)/C=C/c2cnn(Cc3ccc(OCC4CC4)c(Cl)c3Cl)c2)ccn1] | 542 | 2.29 | 3 |
| I-321 | 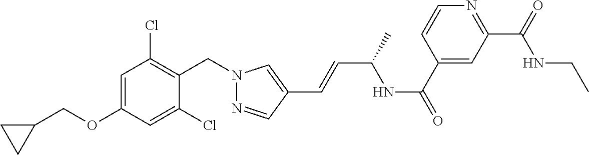 [SMILES: CCNC(=O)c1cc(C(=O)N[C@@H](C)/C=C/c2cnn(Cc3c(Cl)cc(OCC4CC4)cc3Cl)c2)ccn1] | 542 | 2.33 | 3 |
| I-322 | 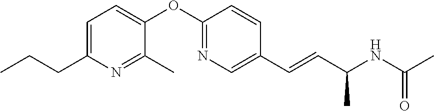 [SMILES: CCCc1ccc(Oc2ccc(/C=C/[C@H](C)NC(C)=O)cn2)c(C)n1] | 340 | 1.13 | 2 |

TABLE 40

| | | | | |
|---|---|---|---|---|
| I-323 | 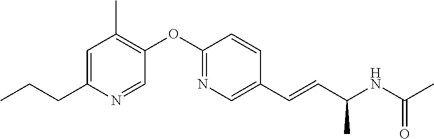 [SMILES: CCCc1cc(C)c(Oc2ccc(/C=C/[C@H](C)NC(C)=O)cn2)cn1] | 341 | 1.19 | 2 |
| I-324 | 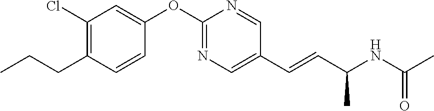 [SMILES: CCCc1ccc(Oc2ncc(/C=C/[C@H](C)NC(C)=O)cn2)cc1Cl] | 361 | 2.25 | 2 |
| I-325 | 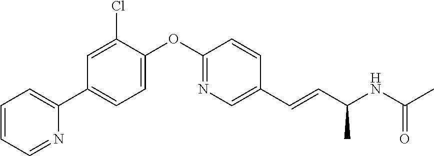 [SMILES: CC(=O)N[C@@H](C)/C=C/c1ccc(Oc2ccc(-c3ccccn3)cc2Cl)nc1] | 394 | 1.87 | 2 |
| I-326 | 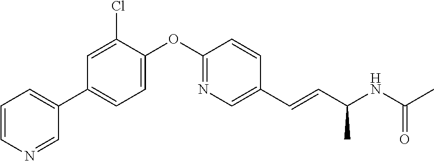 [SMILES: CC(=O)N[C@@H](C)/C=C/c1ccc(Oc2ccc(-c3cccnc3)cc2Cl)nc1] | 394 | 1.47 | 2 |
| I-327 | 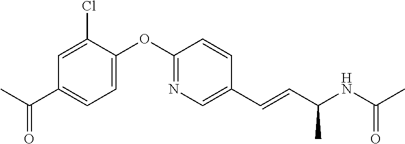 [SMILES: CC(=O)N[C@@H](C)/C=C/c1ccc(Oc2ccc(C(C)=O)cc2Cl)nc1] | 359 | 1.82 | 2 |
| I-328 | 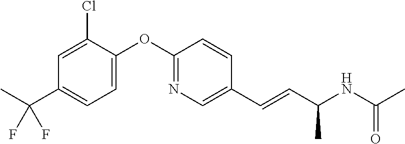 [SMILES: CC(=O)N[C@@H](C)/C=C/c1ccc(Oc2ccc(C(C)(F)F)cc2Cl)nc1] | 381 | 2.15 | 2 |

TABLE 41

| | | | | |
|---|---|---|---|---|
| I-329 | 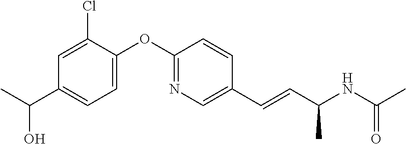 [SMILES: CC(=O)N[C@@H](C)/C=C/c1ccc(Oc2ccc(C(C)O)cc2Cl)nc1] | 361 | 1.63 | 2 |
| I-330 | 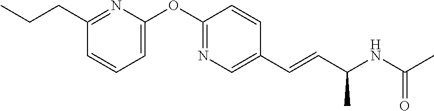 [SMILES: CCCc1cccc(Oc2ccc(/C=C/[C@H](C)NC(C)=O)cn2)n1] | 327 | 1.81 | 2 |
| I-331 | 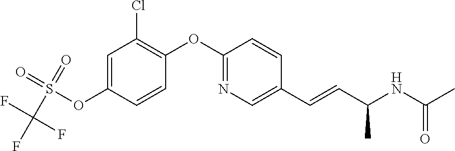 [SMILES: CC(=O)N[C@@H](C)/C=C/c1ccc(Oc2ccc(OS(=O)(=O)C(F)(F)F)cc2Cl)nc1] | 465 | 2.33 | 2 |
| I-332 | 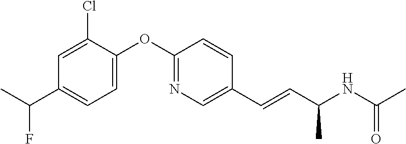 [SMILES: CC(=O)N[C@@H](C)/C=C/c1ccc(Oc2ccc(C(C)F)cc2Cl)nc1] | 363 | 2.07 | 2 |
| I-333 | 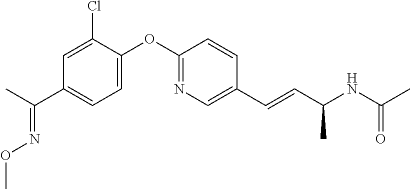 [SMILES: CO/N=C(\C)c1ccc(Oc2ccc(/C=C/[C@H](C)NC(C)=O)cn2)c(Cl)c1] | 388 | 2.22 | 2 |
| I-334 | 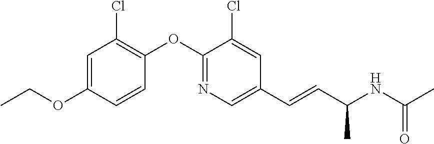 [SMILES: CCOc1ccc(Oc2ncc(/C=C/[C@H](C)NC(C)=O)cc2Cl)c(Cl)c1] | 397 | 2.32 | 2 |

TABLE 42

| | | | | |
|---|---|---|---|---|
| I-335 | 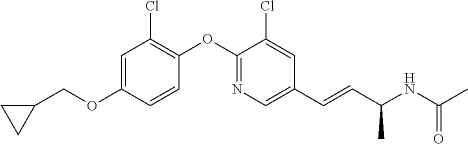 [SMILES: CC(=O)N[C@@H](C)/C=C/c1cnc(Oc2ccc(OCC3CC3)cc2Cl)c(Cl)c1] | 423 | 2.45 | 2 |
| I-336 | 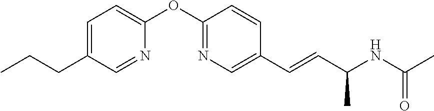 [SMILES: CCCc1ccc(Oc2ccc(/C=C/[C@H](C)NC(C)=O)cn2)nc1] | 326 | 1.82 | 2 |
| I-337 | 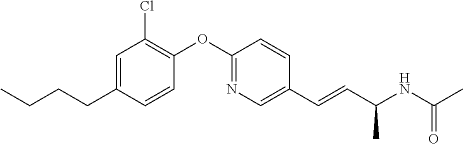 [SMILES: CCCCc1ccc(Oc2ccc(/C=C/[C@H](C)NC(C)=O)cn2)c(Cl)c1] | 373 | 2.53 | 2 |
| I-338 | 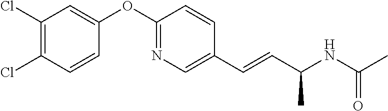 [SMILES: CC(=O)N[C@@H](C)/C=C/c1ccc(Oc2ccc(Cl)c(Cl)c2)nc1] | 352 | 2.19 | 2 |
| I-339 | 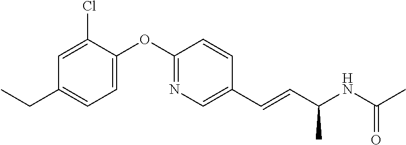 [SMILES: CCc1ccc(Oc2ccc(/C=C/[C@H](C)NC(C)=O)cn2)c(Cl)c1] | 345 | 2.23 | 2 |
| I-340 | 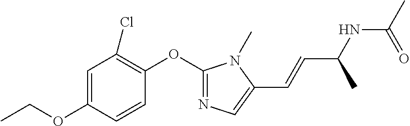 [SMILES: CCOc1ccc(Oc2ncc(/C=C/[C@H](C)NC(C)=O)n2C)c(Cl)c1] | 365 | 1.66 | 2 |

TABLE 43

| | | | | |
|---|---|---|---|---|
| I-341 | 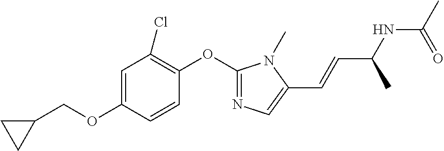 [SMILES: CC(=O)N[C@@H](C)/C=C/c1cnc(Oc2ccc(OCC3CC3)cc2Cl)n1C] | 391 | 1.83 | 2 |
| I-342 | 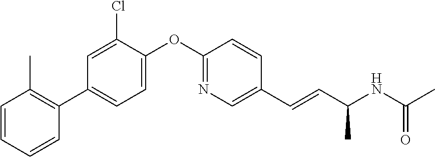 [SMILES: CC(=O)N[C@@H](C)/C=C/c1ccc(Oc2ccc(-c3ccccc3C)cc2Cl)nc1] | 407 | 2.53 | 2 |
| I-343 | 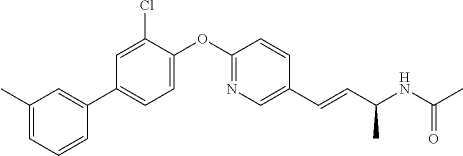 [SMILES: CC(=O)N[C@@H](C)/C=C/c1ccc(Oc2ccc(-c3cccc(C)c3)cc2Cl)nc1] | 407 | 2.57 | 2 |
| I-344 | 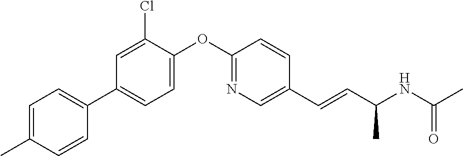 [SMILES: CC(=O)N[C@@H](C)/C=C/c1ccc(Oc2ccc(-c3ccc(C)cc3)cc2Cl)nc1] | 407 | 2.57 | 2 |
| I-345 | 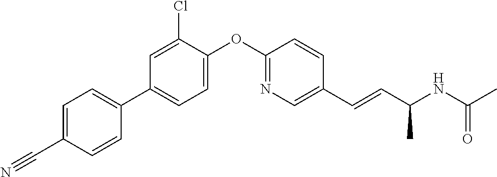 [SMILES: CC(=O)N[C@@H](C)/C=C/c1ccc(Oc2ccc(-c3ccc(C#N)cc3)cc2Cl)nc1] | 418 | 2.21 | 2 |
| I-346 | 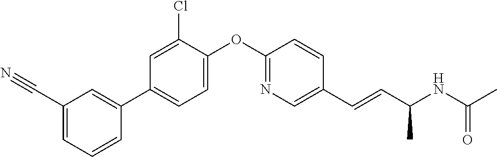 [SMILES: CC(=O)N[C@@H](C)/C=C/c1ccc(Oc2ccc(-c3cccc(C#N)c3)cc2Cl)nc1] | 418 | 2.22 | 2 |

TABLE 44

| | | | | |
|---|---|---|---|---|
| I-347 | 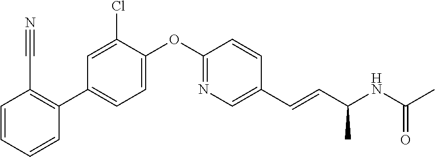 [SMILES: CC(=O)N[C@@H](C)/C=C/c1ccc(Oc2ccc(-c3ccccc3C#N)cc2Cl)nc1] | 418 | 2.17 | 2 |
| I-348 | 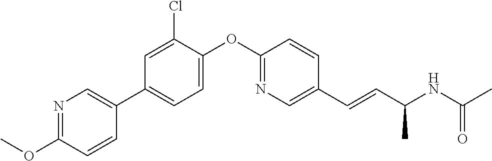 [SMILES: COc1ccc(-c2ccc(Oc3ccc(/C=C/[C@H](C)NC(C)=O)cn3)c(Cl)c2)cn1] | 424 | 2.16 | 2 |
| I-349 | 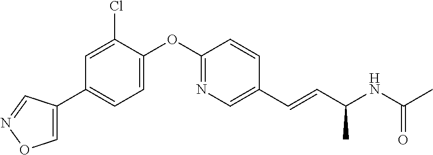 [SMILES: CC(=O)N[C@@H](C)/C=C/c1ccc(Oc2ccc(-c3cnoc3)cc2Cl)nc1] | 384 | 1.71 | 2 |
| I-350 | 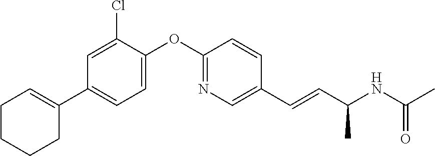 [SMILES: CC(=O)N[C@@H](C)/C=C/c1ccc(Oc2ccc(C3=CCCCC3)cc2Cl)nc1] | 397 | 2.69 | 2 |
| I-351 | 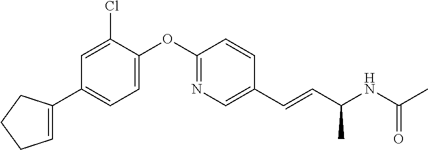 [SMILES: CC(=O)N[C@@H](C)/C=C/c1ccc(Oc2ccc(C3=CCCC3)cc2Cl)nc1] | 383 | 2.56 | 2 |
| I-352 | 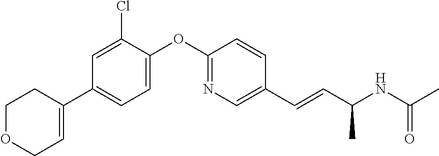 [SMILES: CC(=O)N[C@@H](C)/C=C/c1ccc(Oc2ccc(C3=CCOCC3)cc2Cl)nc1] | 399 | 1.99 | 2 |

TABLE 45

| | | | | |
|---|---|---|---|---|
| I-353 | 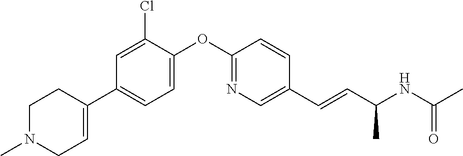 [SMILES: CC(=O)N[C@@H](C)/C=C/c1ccc(Oc2ccc(C3=CCN(C)CC3)cc2Cl)nc1] | 412 | 1.23 | 2 |
| I-354 | 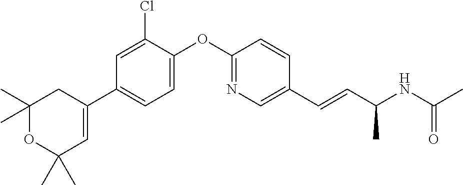 [SMILES: CC(=O)N[C@@H](C)/C=C/c1ccc(Oc2ccc(C3=CC(C)(C)OC(C)(C)C3)cc2Cl)nc1] | 455 | 2.55 | 2 |
| I-355 | 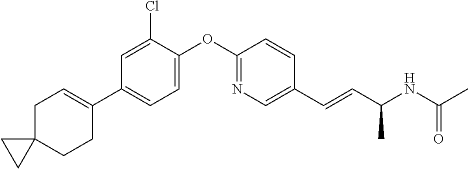 [SMILES: CC(=O)N[C@@H](C)/C=C/c1ccc(Oc2ccc(C3=CCC4(CC3)CC4)cc2Cl)nc1] | 423 | 2.81 | 2 |
| I-356 | 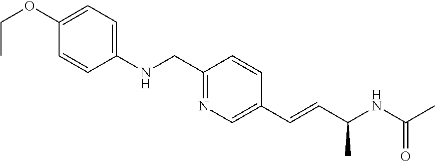 [SMILES: CCOc1ccc(NCc2ccc(/C=C/[C@H](C)NC(C)=O)cn2)cc1] | 340 | 1.15 | 2 |
| I-357 | 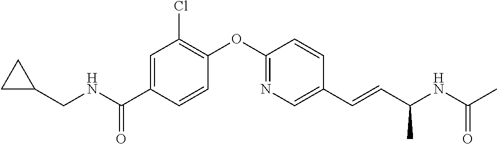 [SMILES: CC(=O)N[C@@H](C)/C=C/c1ccc(Oc2ccc(C(=O)NCC3CC3)cc2Cl)nc1] | 414 | 1.74 | 2 |
| I-358 | 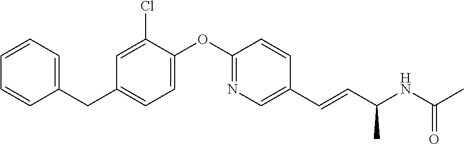 [SMILES: CC(=O)N[C@@H](C)/C=C/c1ccc(Oc2ccc(Cc3ccccc3)cc2Cl)nc1] | 407 | 2.42 | 2 |

TABLE 46

| | | | | |
|---|---|---|---|---|
| I-359 | 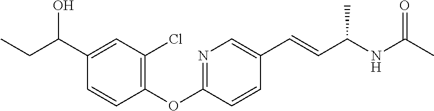 [SMILES: CCC(O)c1ccc(Oc2ccc(/C=C/[C@H](C)NC(C)=O)cn2)c(Cl)c1] | 375 | 1.78 | 2 |
| I-360 | 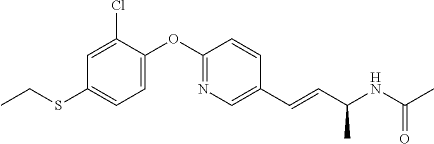 [SMILES: CCSc1ccc(Oc2ccc(/C=C/[C@H](C)NC(C)=O)cn2)c(Cl)c1] | 377 | 2.28 | 2 |
| I-361 | 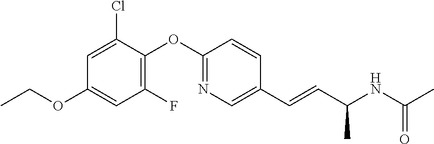 [SMILES: CCOc1cc(F)c(Oc2ccc(/C=C/[C@H](C)NC(C)=O)cn2)c(Cl)c1] | 379 | 2.13 | 2 |
| I-362 | 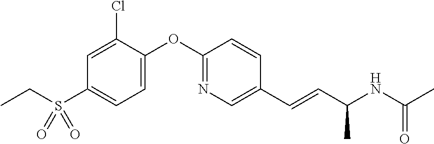 [SMILES: CCS(=O)(=O)c1ccc(Oc2ccc(/C=C/[C@H](C)NC(C)=O)cn2)c(Cl)c1] | 409 | 1.60 | 2 |
| I-363 | 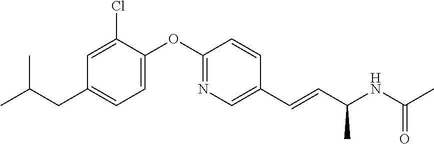 [SMILES: CC(=O)N[C@@H](C)/C=C/c1ccc(Oc2ccc(CC(C)C)cc2Cl)nc1] | 373 | 2.47 | 2 |
| I-364 | 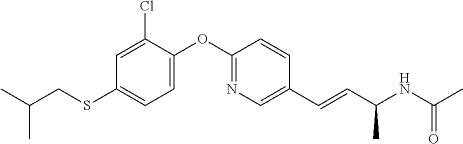 [SMILES: CC(=O)N[C@@H](C)/C=C/c1ccc(Oc2ccc(SCC(C)C)cc2Cl)nc1] | 405 | 2.56 | 2 |

TABLE 47

| | | | | |
|---|---|---|---|---|
| I-365 | 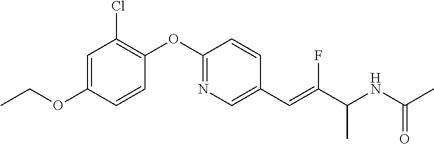 [SMILES: CCOc1ccc(Oc2ccc(/C=C(\F)C(C)NC(C)=O)cn2)c(Cl)c1] | 380 | 2.14 | 2 |
| I-366 | 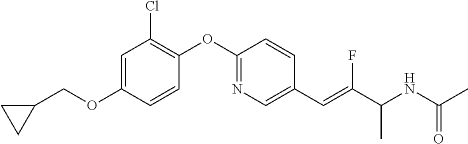 [SMILES: CC(=O)NC(C)/C(F)=C/c1ccc(Oc2ccc(OCC3CC3)cc2Cl)nc1] | 406 | 2.29 | 2 |
| I-367 | 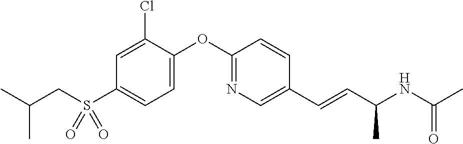 [SMILES: CC(=O)N[C@@H](C)/C=C/c1ccc(Oc2ccc(S(=O)(=O)CC(C)C)cc2Cl)nc1] | 437 | 2.01 | 2 |
| I-368 | 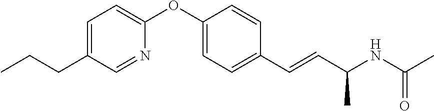 [SMILES: CCCc1ccc(Oc2ccc(/C=C/[C@H](C)NC(C)=O)cc2)nc1] | 325 | 2.12 | 3 |
| I-369 | 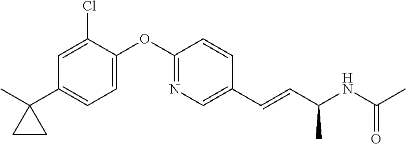 [SMILES: CC(=O)N[C@@H](C)/C=C/c1ccc(Oc2ccc(C3(C)CC3)cc2Cl)nc1] | 371 | 2.38 | 2 |
| I-370 | 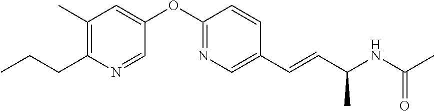 [SMILES: CCCc1ncc(Oc2ccc(/C=C/[C@H](C)NC(C)=O)cn2)cc1C] | 341 | 1.24 | 2 |

TABLE 48

| | | | | |
|---|---|---|---|---|
| I- 371 | 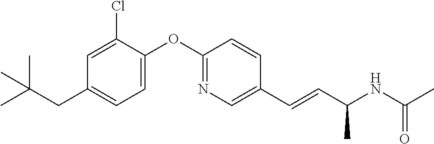 [SMILES: CC(=O)N[C@@H](C)/C=C/c1ccc(Oc2ccc(CC(C)(C)C)cc2Cl)nc1] | 387 | 2.60 | 2 |
| I-372 | 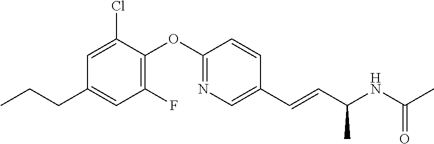 [SMILES: CCCc1cc(F)c(Oc2ccc(/C=C/[C@H](C)NC(C)=O)cn2)c(Cl)c1] | 377 | 2.44 | 2 |
| I-373 | 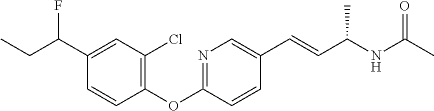 [SMILES: CCC(F)c1ccc(Oc2ccc(/C=C/[C@H](C)NC(C)=O)cn2)c(Cl)c1] | 377 | 2.21 | 2 |
| I-374 | 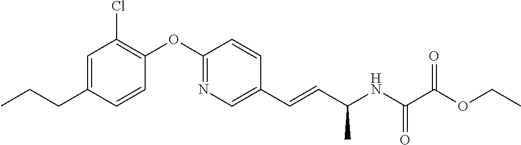 [SMILES: CCCc1ccc(Oc2ccc(/C=C/[C@H](C)NC(=O)C(=O)OCC)cn2)c(Cl)c1] | 418 | 2.62 | 2 |
| I-375 | 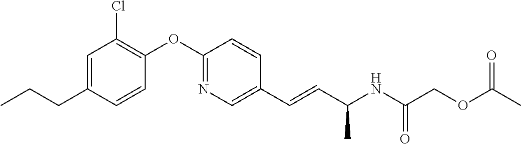 [SMILES: CCCc1ccc(Oc2ccc(/C=C/[C@H](C)NC(=O)COC(C)=O)cn2)c(Cl)c1] | 418 | 2.47 | 2 |
| I-376 | 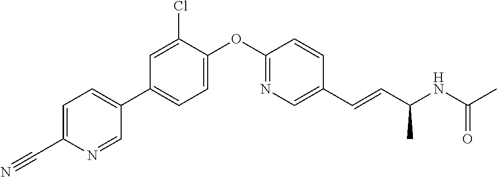 [SMILES: CC(=O)N[C@@H](C)/C=C/c1ccc(Oc2ccc(-c3ccc(C#N)nc3)cc2Cl)nc1] | 419 | 2.01 | 3 |

TABLE 49

| | | | | |
|---|---|---|---|---|
| I-377 | 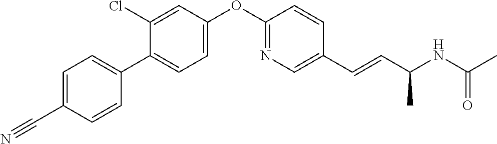 [SMILES: CC(=O)N[C@@H](C)/C=C/c1ccc(Oc2ccc(-c3ccc(C#N)cc3)c(Cl)c2)nc1] | 418 | 2.28 | 3 |
| I-378 | 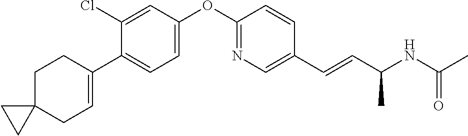 [SMILES: CC(=O)N[C@@H](C)/C=C/c1ccc(Oc2ccc(C3=CCC4(CC3)CC4)c(Cl)c2)nc1] | 423 | 2.96 | 3 |
| I-379 | 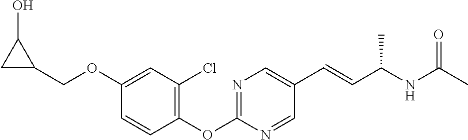 [SMILES: CC(=O)N[C@@H](C)/C=C/c1cnc(Oc2ccc(OCC3CC3O)cc2Cl)nc1] | 404 | 1.48 | 2 |
| I-380 | 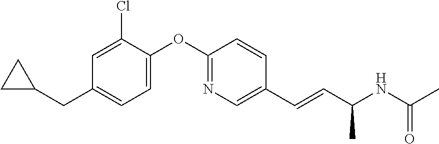 [SMILES: CC(=O)N[C@@H](C)/C=C/c1ccc(Oc2ccc(CC3CC3)cc2Cl)nc1] | 371 | 2.45 | 3 |
| I-381 | 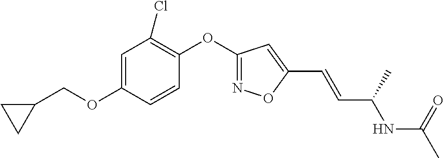 [SMILES: CC(=O)N[C@@H](C)/C=C/c1cc(Oc2ccc(OCC3CC3)cc2Cl)no1] | 378 | 2.18 | 2 |
| I-382 | 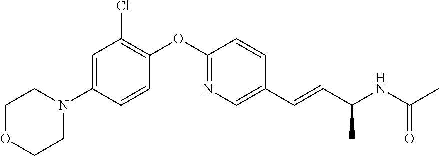 [SMILES: CC(=O)N[C@@H](C)/C=C/c1ccc(Oc2ccc(N3CCOCC3)cc2Cl)nc1] | 402 | 1.84 | 2 |

TABLE 50

| | | | | |
|---|---|---|---|---|
| I-383 | 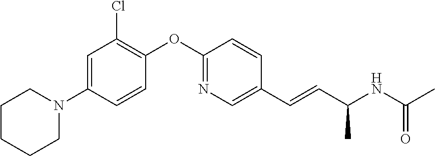 [SMILES: CC(=O)N[C@@H](C)/C=C/c1ccc(Oc2ccc(N3CCCCC3)cc2Cl)nc1] | 400 | 1.98 | 2 |
| I-384 | 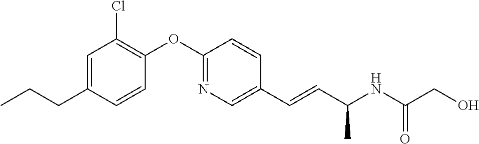 [SMILES: CCCc1ccc(Oc2ccc(/C=C/[C@H](C)NC(=O)CO)cn2)c(Cl)c1] | 376 | 2.27 | 2 |
| I-385 | 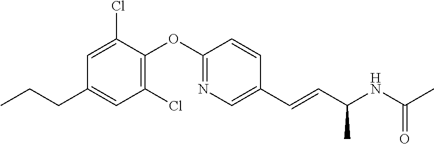 [SMILES: CCCc1cc(Cl)c(Oc2ccc(/C=C/[C@H](C)NC(C)=O)cn2)c(Cl)c1] | 393 | 2.64 | 3 |
| I-386 | 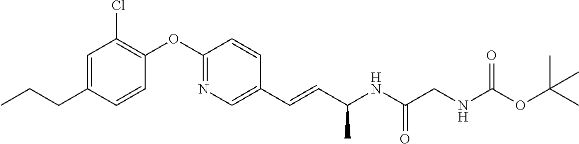 [SMILES: CCCc1ccc(Oc2ccc(/C=C/[C@H](C)NC(=O)CNC(=O)OC(C)(C)C)cn2)c(Cl)c1] | 475 | 2.63 | 2 |
| I-387 | 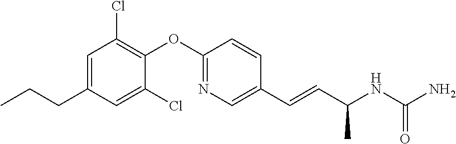 [SMILES: CCCc1cc(Cl)c(Oc2ccc(/C=C/[C@H](C)NC(N)=O)cn2)c(Cl)c1] | 394 | 2.41 | 3 |
| I-388 | 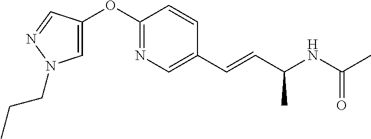 [SMILES: CCCn1cc(Oc2ccc(/C=C/[C@H](C)NC(C)=O)cn2)cn1] | 315 | 1.51 | 2 |

TABLE 51

| | | | | |
|---|---|---|---|---|
| I-389 | 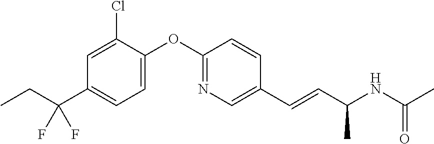 [SMILES: CCC(F)(F)c1ccc(Oc2ccc(/C=C/[C@H](C)NC(C)=O)cn2)c(Cl)c1] | 395 | 2.36 | 3 |
| I-390 | 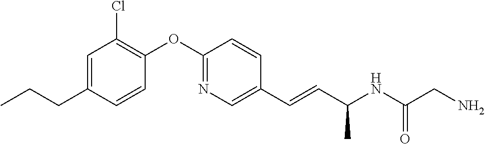 [SMILES: CCCc1ccc(Oc2ccc(/C=C/[C@H](C)NC(=O)CN)cn2)c(Cl)c1] | 375 | 1.72 | 2 |
| I-391 | 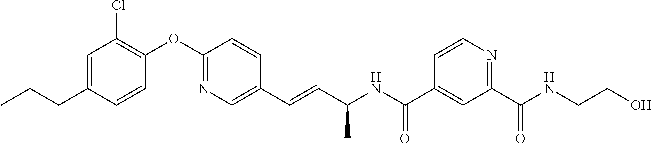 [SMILES: CCCc1ccc(Oc2ccc(/C=C/[C@H](C)NC(=O)c3ccnc(C(=O)NCCO)c3)cn2)c(Cl)c1] | 511 | 2.32 | 2 |
| I-392 | 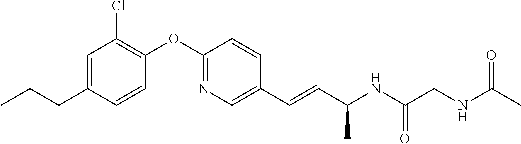 [SMILES: CCCc1ccc(Oc2ccc(/C=C/[C@H](C)NC(=O)CNC(C)=O)cn2)c(Cl)c1] | 416 | 2.20 | 2 |
| I-393 | 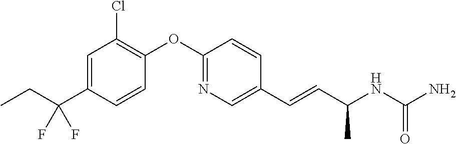 [SMILES: CCC(F)(F)c1ccc(Oc2ccc(/C=C/[C@H](C)NC(N)=O)cn2)c(Cl)c1] | 396 | 2.15 | 3 |
| I-394 | 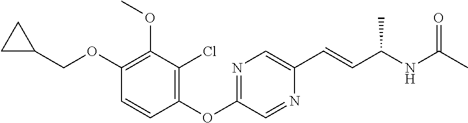 [SMILES: COc1c(OCC2CC2)ccc(Oc2cnc(/C=C/[C@H](C)NC(C)=O)cn2)c1Cl] | 418 | 2.14 | 2 |

TABLE 52

| | | | | |
|---|---|---|---|---|
| I-395 | 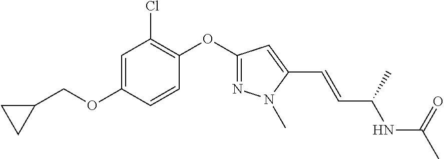 [SMILES: CC(=O)N[C@@H](C)/C=C/c1cc(Oc2ccc(OCC3CC3)cc2Cl)nn1C] | 390 | 2.06 | 2 |
| I-396 | 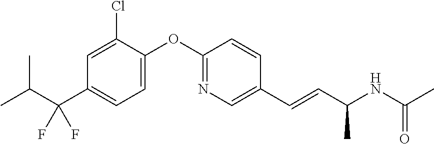 [SMILES: CC(=O)N[C@@H](C)/C=C/c1ccc(Oc2ccc(C(F)(F)C(C)C)cc2Cl)nc1] | 409 | 2.40 | 2 |
| I-397 | 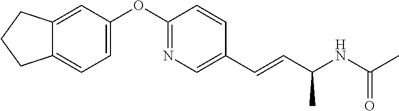 [SMILES: CC(=O)N[C@@H](C)/C=C/c1ccc(Oc2ccc3c(c2)CCC3)nc1] | 323 | 2.09 | 3 |
| I-398 | 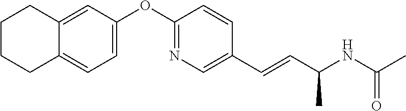 [SMILES: CC(=O)N[C@@H](C)/C=C/c1ccc(Oc2ccc3c(c2)CCCC3)nc1] | 338 | 2.22 | 3 |
| I-399 | 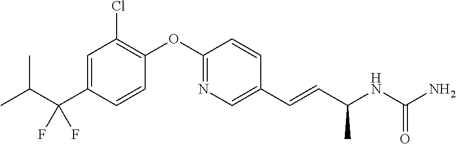 [SMILES: CC(C)C(F)(F)c1ccc(Oc2ccc(/C=C/[C@H](C)NC(N)=O)cn2)c(Cl)c1] | 410 | 2.29 | 3 |
| I-400 | 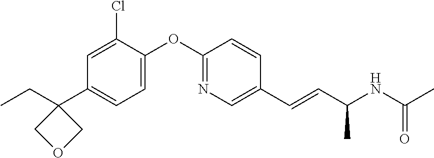 [SMILES: CCC1(c2ccc(Oc3ccc(/C=C/[C@H](C)NC(C)=O)cn3)c(Cl)c2)COC1] | 401 | 1.92 | 3 |

TABLE 53

| | | | | |
|---|---|---|---|---|
| I-401 | 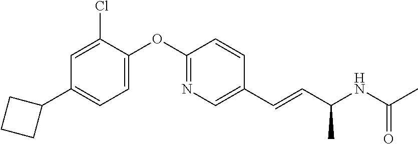 [SMILES: CC(=O)N[C@@H](C)/C=C/c1ccc(Oc2ccc(C3CCC3)cc2Cl)nc1] | 371 | 2.47 | 3 |
| I-402 | 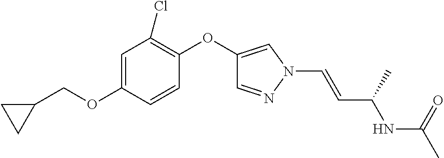 [SMILES: CC(=O)N[C@@H](C)/C=C/n1cc(Oc2ccc(OCC3CC3)cc2Cl)cn1] | 376 | 2.14 | 2 |
| I-403 | 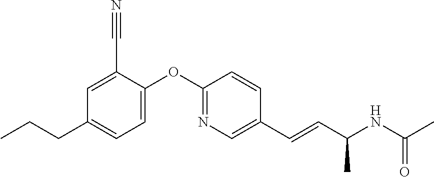 [SMILES: CCCc1ccc(Oc2ccc(/C=C/[C@H](C)NC(C)=O)cn2)c(C#N)c1] | 350 | 2.14 | 3 |
| I-404 | 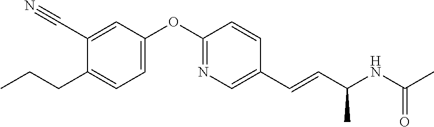 [SMILES: CCCc1ccc(Oc2ccc(/C=C/[C@H](C)NC(C)=O)cn2)cc1C#N] | 351 | 2.09 | 3 |
| I-405 | 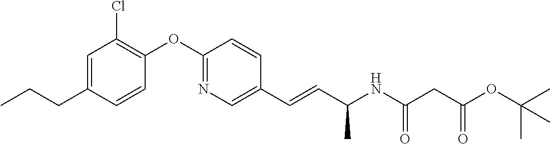 [SMILES: CCCc1ccc(Oc2ccc(/C=C/[C@H](C)NC(=O)CC(=O)OC(C)(C)C)cn2)c(Cl)c1] | 460 | 2.73 | 2 |
| I-406 | 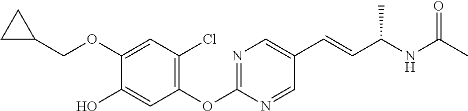 [SMILES: CC(=O)N[C@@H](C)/C=C/c1cnc(Oc2cc(O)c(OCC3CC3)cc2Cl)nc1] | 405 | 1.76 | 2 |

TABLE 54

| | | | | |
|---|---|---|---|---|
| I-407 | 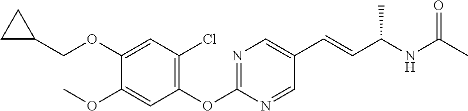 [SMILES: COc1cc(Oc2ncc(/C=C/[C@H](C)NC(C)=O)cn2)c(Cl)cc1OCC1CC1] | 418 | 1.93 | 2 |
| I-408 | 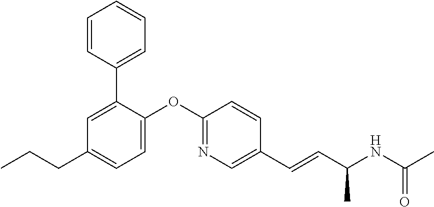 [SMILES: CCCc1ccc(Oc2ccc(/C=C/[C@H](C)NC(C)=O)cn2)c(-c2ccccc2)c1] | 401 | 2.59 | 3 |
| I-409 | 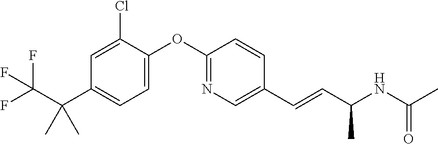 [SMILES: CC(=O)N[C@@H](C)/C=C/c1ccc(Oc2ccc(C(C)(C)C(F)(F)F)cc2Cl)nc1] | 427 | 2.38 | 3 |
| I-410 | 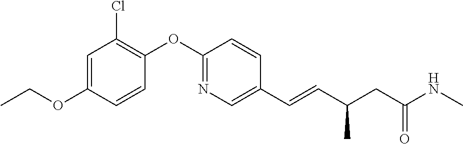 [SMILES: CCOc1ccc(Oc2ccc(/C=C/[C@H](C)CC(=O)NC)cn2)c(Cl)c1] | 367 | 2.16 | 2 |
| I-411 | 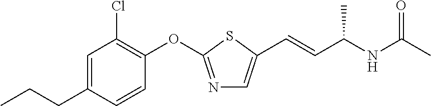 [SMILES: CCCc1ccc(Oc2ncc(/C=C/[C@H](C)NC(C)=O)s2)c(Cl)c1] | 366 | 2.37 | 2 |
| I-412 | 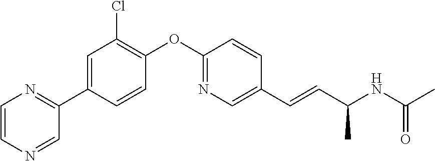 [SMILES: CC(=O)N[C@@H](C)/C=C/c1ccc(Oc2ccc(-c3cnccn3)cc2Cl)nc1] | 396 | 1.84 | 2 |

TABLE 55

| | | | | |
|---|---|---|---|---|
| I-413 | 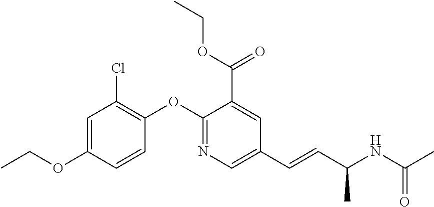 [SMILES: CCOC(=O)c1cc(/C=C/[C@H](C)NC(C)=O)cnc1Oc1ccc(OCC)cc1Cl] | 433 | 2.17 | 3 |
| I-414 | 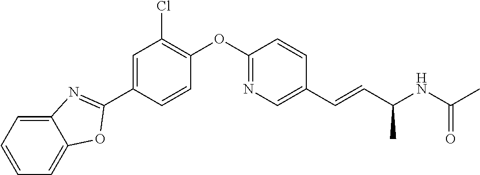 [SMILES: CC(=O)N[C@@H](C)/C=C/c1ccc(Oc2ccc(-c3nc4ccccc4o3)cc2Cl)nc1] | 434 | 2.40 | 3 |
| I-415 | 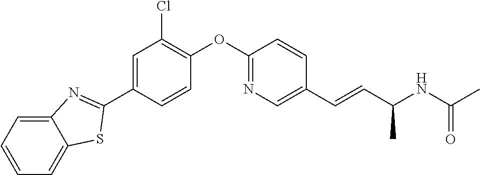 [SMILES: CC(=O)N[C@@H](C)/C=C/c1ccc(Oc2ccc(-c3nc4ccccc4s3)cc2Cl)nc1] | 450 | 2.55 | 3 |
| I-416 | 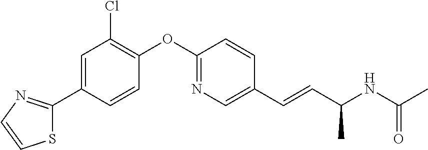 [SMILES: CC(=O)N[C@@H](C)/C=C/c1ccc(Oc2ccc(-c3nccs3)cc2Cl)nc1] | 400 | 2.02 | 3 |
| I-417 | 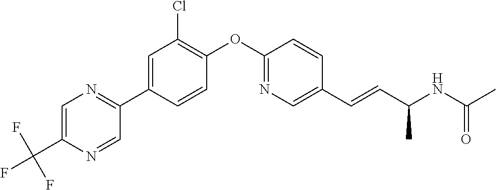 [SMILES: CC(=O)N[C@@H](C)/C=C/c1ccc(Oc2ccc(-c3cnc(C(F)(F)F)cn3)cc2Cl)nc1] | 463 | 2.38 | 3 |
| I-418 | 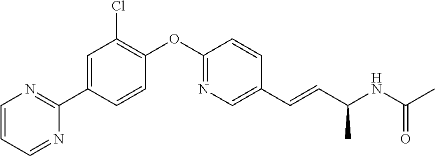 [SMILES: CC(=O)N[C@@H](C)/C=C/c1ccc(Oc2ccc(-c3ncccn3)cc2Cl)nc1] | 395 | 1.92 | 3 |

TABLE 56

| | | | | |
|---|---|---|---|---|
| I-419 | 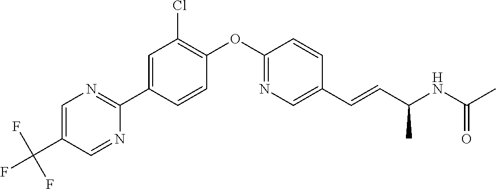 [SMILES: CC(=O)N[C@@H](C)/C=C/c1ccc(Oc2ccc(-c3ncc(C(F)(F)F)cn3)cc2Cl)nc1] | 463 | 2.47 | 3 |
| I-420 | 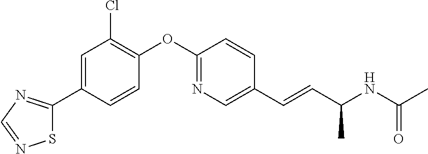 [SMILES: CC(=O)N[C@@H](C)/C=C/c1ccc(Oc2ccc(-c3ncns3)cc2Cl)nc1] | 401 | 1.98 | 3 |
| I-421 | 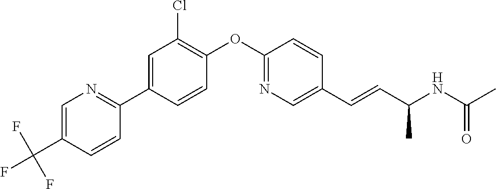 [SMILES: CC(=O)N[C@@H](C)/C=C/c1ccc(Oc2ccc(-c3ccc(C(F)(F)F)cn3)cc2Cl)nc1] | 462 | 2.47 | 3 |
| I-422 | 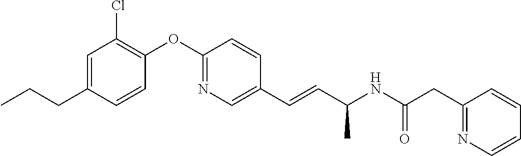 [SMILES: CCCc1ccc(Oc2ccc(/C=C/[C@H](C)NC(=O)Cc3ccccn3)cn2)c(Cl)c1] | 437 | 2.11 | 2 |
| I-423 | 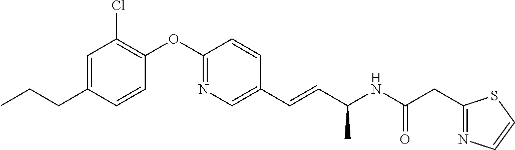 [SMILES: CCCc1ccc(Oc2ccc(/C=C/[C@H](C)NC(=O)Cc3nccs3)cn2)c(Cl)c1] | 443 | 2.48 | 2 |
| I-424 | 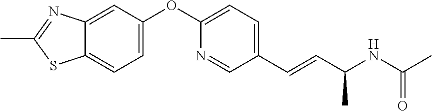 [SMILES: CC(=O)N[C@@H](C)/C=C/c1ccc(Oc2ccc3sc(C)nc3c2)nc1] | 354 | 1.64 | 3 |

TABLE 57

| | | | | |
|---|---|---|---|---|
| I-425 | 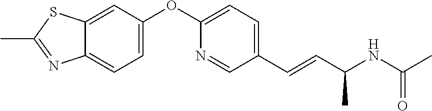 [SMILES: CC(=O)N[C@@H](C)/C=C/c1ccc(Oc2ccc3nc(C)sc3c2)nc1] | 354 | 1.61 | 3 |
| I-426 | 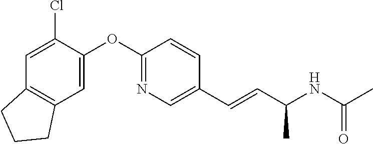 [SMILES: CC(=O)N[C@@H](C)/C=C/c1ccc(Oc2cc3c(cc2Cl)CCC3)nc1] | 357 | 2.24 | 3 |
| I-427 | 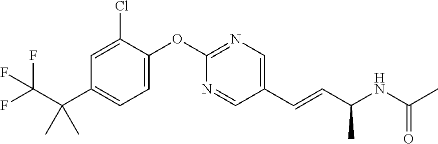 [SMILES: CC(=O)N[C@@H](C)/C=C/c1cnc(Oc2ccc(C(C)(C)C(F)(F)F)cc2Cl)nc1] | 428 | 2.17 | 2 |
| I-428 | 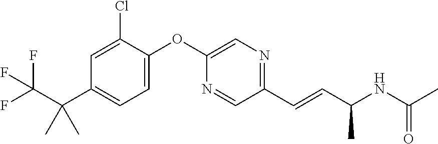 [SMILES: CC(=O)N[C@@H](C)/C=C/c1cnc(Oc2ccc(C(C)(C)C(F)(F)F)cc2Cl)cn1] | 428 | 2.33 | 2 |
| I-429 | 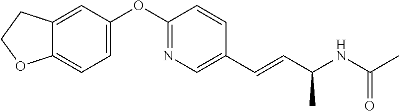 [SMILES: CC(=O)N[C@@H](C)/C=C/c1ccc(Oc2ccc3c(c2)CCO3)nc1] | 325 | 1.69 | 2 |
| I-430 | 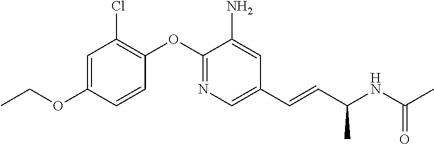 [SMILES: CCOc1ccc(Oc2ncc(/C=C/[C@H](C)NC(C)=O)cc2N)c(Cl)c1] | 376 | 1.86 | 2 |

### Example 253

### Preparation of Compound I-253

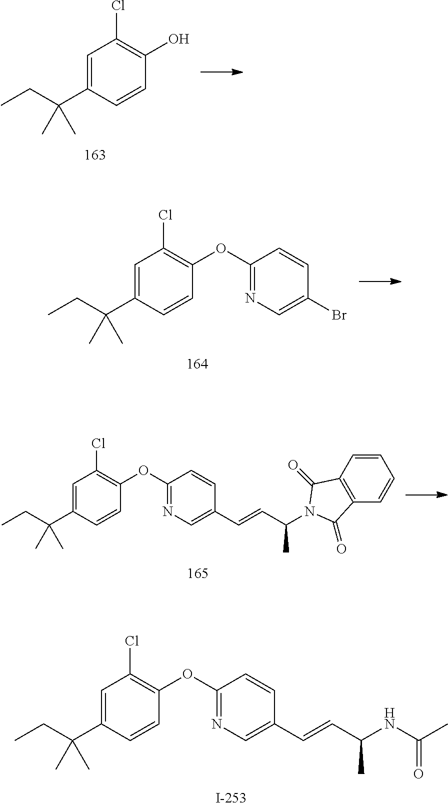 [SMILES: CCC(C)(C)c1ccc(O)c(Cl)c1 ; CCC(C)(C)c1ccc(Oc2ccc(Br)cn2)c(Cl)c1 ; CCC(C)(C)c1ccc(Oc2ccc(/C=C/[C@H](C)N3C(=O)c4ccccc4C3=O)cn2)c(Cl)c1 ; CCC(C)(C)c1ccc(Oc2ccc(/C=C/[C@H](C)NC(C)=O)cn2)c(Cl)c1]

### Step 1 Preparation of Compound 164

Compound 163 (400 mg, 2.01 mmol) and 2,5-dibromopyridine (477 mg, 2.01 mmol) were dissolved in NMP (4.00 mL). Cesium carbonate (1.31 g, 4.03 mmol) was added to the mixture, and the mixture was stirred at 140° C. for 8 hours. Water was added to the mixture, and the mixture was extracted with ethyl acetate. The organic layer was washed with water, and dried over magnesium sulfate. The solvent was condensed under reduced pressure. The residue was purified by silica gel chromatography (hexane-ethyl acetate) to afford Compound 164 (658 mg, yield 92%).

¹H NMR (CDCl₃) δ: 0.73 (t, J=8.0 Hz, 3H), 1.29 (s, 6H), 1.63 (m, 2H), 6.87 (d, J=8.0 Hz, 1H), 7.11 (d, J=8.0 Hz, 1H), 7.26 (dd, J=8.0, 4.0 Hz, 1H), 7.40 (s, 1H), 7.78 (dd, J=8.0, 4.0 Hz, 1H), 8.19 (d, J=4.0 Hz, 1H).

### Step 2 Preparation of Compound 165

2 mol/L sodium carbonate aqueous solution (1.80 mL) was added to the ethanol solution (6.50 mL) of Compound 164 (0.64 g, 1.80 mmol) and Compound 2 (0.59 g, 1.80 mmol) prepared in Reference Example 001. The atmosphere was replaced with nitrogen, and bis(triphenylphosphine) palladium(II) dichloride (0.13 g, 0.18 mmol) was added to the mixture. The mixture was subjected to microwave irradiation and stirred at 80° C. for 15 minutes. The mixture was diluted with chroloform (6.50 mL), WSCD (0.52 g, 2.71 mmol) was added to the mixture. The mixture was stirred at room temperature for 1 hour. Water was added to the mixture, and the mixture was extracted with ethyl acetate. The organic layer was dried over magnesium sulfate. The solvent was condensed under reduced pressure. The residue was purified by silica gel chromatography (hexane-ethyl acetate) to afford Compound 165 (0.48 g, yield 57%).

¹H-NMR (CDCl₃) δ: 0.72 (t, J=8.0 Hz, 3H), 1.28 (s, 6H), 1.64 (m, 2H), 1.66 (d, J 8.0 Hz, 3H), 5.08 (m, 1H), 6.55 (s, 2H), 6.88 (d, J=8.0 Hz, 1H), 7.11 (d, J=8.0 Hz, 1H), 7.24 (dd, J=8.0, 4.0 Hz, 1H), 7.39 (d, J=4.0 Hz, 1H), 7.71 (m, 4H), 7.78 (dd, J=12.0, 4.0 Hz, 1H), 7.84 (m, 4H), 8.08 (d, J=4.0 Hz, 1H).

### Step 3 Preparation of Compound I-253

Compound 165 (0.48 g, 1.00 mmol) was dissolved in ethanol (10 mL). Hydrazine monohydrate (0.49 mL, 10.0 mmol) was added to the mixture, and the mixture was stirred under heat refluxing for 2.5 hours. After the mixture was cooled to room temperature, the precipitated solid was filtrated, and the filtrate was condensed under reduced pressure. Saturated sodium hydrogen carbonate solution was added to the residue, and the mixture was extracted with ethyl acetate. The organic layer was washed with water, and dried over magnesium sulfate. The solvent was condensed under reduced pressure. The residue was purified by silica gel chromatography (chloroform-methanol). The residue was dissolved in pyridine (3.00 mL). Acetyl chloride (0.086 mL, 1.20 mmol) was added to the mixture while cooling in ice, and the mixture was stirred for 1 hour. Water was added to the mixture, and the mixture was extracted with ethyl acetate. The organic layer was washed with hydrochloric acid aqueous solution, saturated sodium hydrogen carbonate solution and water, and dried over magnesium sulfate. The solvent was condensed under reduced pressure. The residue was purified by silica gel chromatography (hexane-ethyl acetate) to afford Compound I-253 (0.29 g, yield 75%).

¹H-NMR (CDCl₃) δ: 0.73 (t, J=8.0 Hz, 3H), 1.29 (s, 6H), 1.33 (d, J=8.0 Hz, 3H), 1.64 (m, 2H), 2.01 (s, 3H), 4.74 (m, 1H), 5.44 (d, J=8.0 Hz, 1H), 6.10 (dd, J=12.0, 4.0 Hz, 1H), 6.44 (d, J=12.0 Hz, 1H), 6.89 (d, J=8.0 Hz, 1H), 7.12 (d, J=8.0 Hz, 1H), 7.24 (dd, J=8.0, 4.0 Hz, 1H), 7.40 (d, J=8.0 Hz, 1H), 7.73 (dd, J=8.0, 4.0 Hz, 1H), 8.09 (d, J=4.0 Hz, 1H).

[M+H]=387, Method Condition 2: retention time 2.57 min

### Example 267

### Preparation of Compound I-267

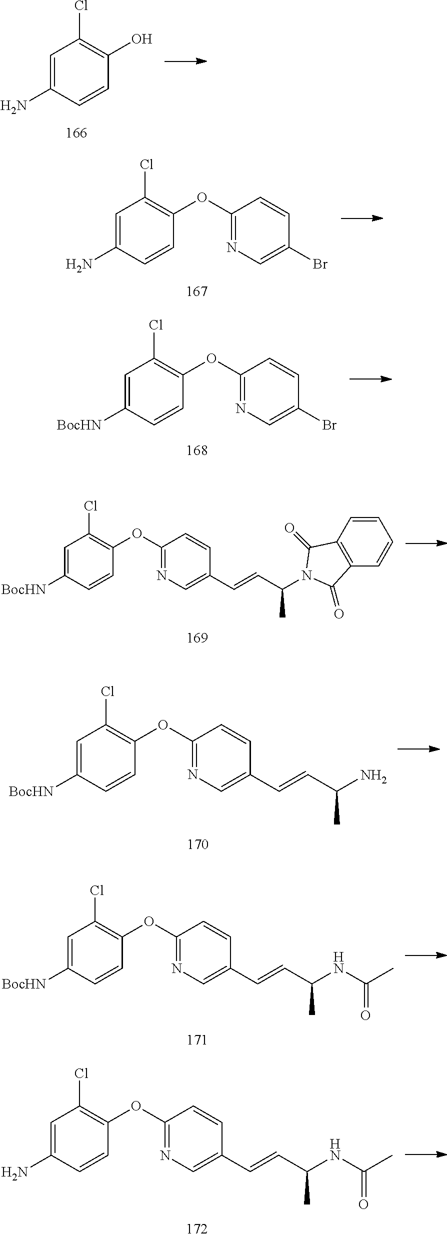

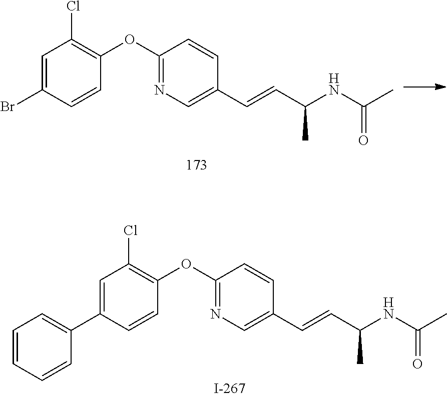 [SMILES: CC(=O)N[C@@H](C)/C=C/c1ccc(Oc2ccc(Br)cc2Cl)nc1 ; CC(=O)N[C@@H](C)/C=C/c1ccc(Oc2ccc(-c3ccccc3)cc2Cl)nc1]

### Step 1 Preparation of Compound 167

Compound 166 (4.36 g, 30.4 mmol) and 2,5-dibromopyridine (6.00 g, 25.3 mmol) was dissolved in DMSO (50 mL). Potassium carbonate (4.20 g, 30.4 mmol) was added to the mixture, the mixture was stirred at 150° C. for 5 hours. Water was added to the reaction mixture, and the reaction mixture was extracted with chloroform. The organic layer was washed with water, and dried over magnesium sulfate. The solvent was condensed under reduced pressure. The residue was purified by silica gel chromatography (hexane-ethyl acetate) to afford Compound 167 (3.92 g, yield 47%, purity 90%).

¹H-NMR (CDCl₃) δ: 3.69 (s, 2H), 6.57-6.61 (m, 1H), 6.77 (d, J=2.9 Hz, 1H), 6.83 (d, J=8.7 Hz, 1H), 6.97 (d, J=8.6 Hz, 1H), 7.73-7.76 (m, 1H), 8.17 (d, J=2.5 Hz, 1H).

### Step 5 Preparation of Compound 171

Compound 167 (3.90 g, 11.7 mmol) was dissolved in dioxane (20.0 mL). Di-tert-butyl-dicarbonate (3.84 g, 17.6 mmol) was added to the mixture, and the mixture was stirred at 60° C. for 7 hours. The solvent was condensed under reduced pressure. The residue was purified by silica gel chromatography (hexane-ethyl acetate) to afford Compound 168 (3.90 g, yield 83%).

¹H-NMR (DMSO-D₆) δ: 1.49 (s, 9H), 7.08 (d, J=8.9 Hz, 1H), 7.22 (d, J=8.9 Hz, 1H), 7.38 (dd, J=8.9, 2.2 Hz, 1H), 7.71 (d, J=2.2 Hz, 1H), 8.04-8.07 (m, 1H), 8.22 (d, J=2.7 Hz, 1H), 9.59 (s, 1H).

### Step 3 Preparation of Compound 169

2 mol/L sodium carbonate aqueous solution (5.00 mL) was added to the ethanol solution (20.0 mL) of Compound 168 (2.00 g, 5.00 mmol) and Compound 2 (2.24 g, 6.51 mmol) prepared in Reference Example 001. The atmosphere was replaced with nitrogen, and bis(triphenylphosphine) palladium(II) dichloride (0.351 g, 0.500 mmol) was added to the mixture. The mixture was subjected to microwave irradiation and stirred at 80° C. for 20 minutes. The mixture wad diluted with chloroform (40.0 mL), and WSCD (1.44 g, 7.51 mmol) was added to the mixture. The mixture was stirred at room temperature for 1 hour. Water was added to the mixture, and the mixture was extracted with ethyl acetate. The organic layer was dried over magnesium sulfate. The solvent was condensed under reduced pressure. The residue was purified by silica gel chromatography (hexane-ethyl acetate) to afford Compound 169 (2.10 g, yield 81%).

¹H-NMR (CDCl₃) δ: 1.51 (s, 9H), 1.66 (d, J=7.1 Hz, 3H), 5.07-5.09 (m, 1H), 6.48 (s, 1H), 6.53-6.55 (m, 2H), 6.88 (d, J=8.6 Hz, 1H), 7.10 (d, J=8.7 Hz, 1H), 7.20 (dd, J=8.8, 2.6 Hz, 1H), 7.62 (d, J=2.4 Hz, 1H), 7.69-7.84 (m, 5H), 8.04 (d, J=2.0 Hz, 1H).

### Step 4 Preparation of Compound 170

Compound 169 (2.09 g, 4.02 mmol) was dissolved in chloroform (15.0 mL). 40% methylamine aqueous solution (10 mL) was added to the mixture, and the mixture was stirred at room temperature for 2 hours. The insoluble matter was filtered. The filtrate condensed under reduced pressure to afford Compound 170 (1.66 g, yield 95%).

¹H-NMR (CDCl₃) δ: 1.25 (d, J=6.5 Hz, 3H), 1.52 (s, 9H), 3.66-3.68 (m, 1H), 6.13 (dd, J=15.9, 6.5 Hz, 1H), 6.40 (d, J=15.9 Hz, 1H), 6.52 (s, 1H), 6.89 (d, J=8.6 Hz, 1H), 7.11 (d, J=8.9 Hz, 1H), 7.21 (dd, J=8.6, 2.4 Hz, 1H), 7.63 (d, J=2.4 Hz, 1H), 7.73 (dd, J=8.6, 2.4 Hz, 1H), 8.06 (d, J=2.2 Hz, 1H).

### Step 5 Preparation of Compound 171

Compound 170 (1.66 g, 3.83 mmol) was dissolved in tetrahydrofuran (20.0 mL). Pyridine (0.465 g, 5.75 mmol) and acetyl chloride (0.41 mL, 5.75 mmol) were added to the mixture while cooling in ice, and the mixture was stirred for 10 minutes. Water was added to the mixture, and the mixture was extracted with ethyl acetate. The organic layer was washed with hydrochloric acid aqueous solution, saturated sodium hydrogen carbonate solution and water, and dried over magnesium sulfate. The solvent was condensed under reduced pressure. The residue was purified by silica gel chromatography (hexane-ethyl acetate) to afford Compound 171 (1.64 g, yield 99%).

¹H-NMR (CDCl₃) δ: 1.33 (d, J=6.9 Hz, 3H), 1.52 (s, 9H), 2.01 (s, 3H), 4.69-4.76 (m, 1H), 5.44 (d, J=7.9 Hz, 1H), 6.09 (dd, J=15.9, 5.7 Hz, 1H), 6.43 (d, J=15.9 Hz, 1H), 6.53 (s, 1H), 6.88 (d, J=8.5 Hz, 1H), 7.10 (d, J=8.7 Hz, 1H), 7.20 (dd, J=8.8, 2.6 Hz, 1H), 7.62 (d, J=2.3 Hz, 1H), 7.71 (dd, J=8.5, 2.4 Hz, 1H), 8.05 (d, J=2.3 Hz, 1H).

### Step 6 Preparation of Compound 172

Compound 171 (1.64 g, 3.80 mmol) was dissolved in chloroform (10.0 mL). Trifluoro acetic acid (5.00 mL) was added to the mixture, and the mixture was stirred at room temperature for 2 hours. The mixture was condensed under reduced pressure. Saturated sodium hydrogen carbonate solution was added to the residue, the mixture was extracted with ethyl acetate. The organic layer was washed with water and brine, and dried over magnesium sulfate. The solvent was condensed under reduced pressure. The residue was purified by silica gel chromatography (hexane-ethyl acetate) to afford Compound 172 (1.06 g, yield 81%).

¹H-NMR (CDCl₃) δ: 1.32 (d, J=6.9 Hz, 3H), 2.01 (s, 3H), 3.68 (s, 2H), 4.70-4.75 (m, 1H), 5.44 (d, J=8.1 Hz, 1H), 6.07 (dd, J=16.0, 5.6 Hz, 1H), 6.42 (d, J=15.9 Hz, 1H), 6.59 (dd, J=8.5, 2.7 Hz, 1H), 6.77 (d, J=2.7 Hz, 1H), 6.84 (d, J=8.7 Hz, 1H), 6.98 (d, J=8.5 Hz, 1H), 7.69 (dd, J=8.5, 2.4 Hz, 1H), 8.06 (d, J=2.4 Hz, 1H).

### Step 7 Preparation of Compound 173

Tert-butyl nitrite (1.51 mL, 12.6 mmol) and Compound 172 (3.35 g, 10.1 mmol) were added to the suspension of acetonitrile (50 mL) of copper bromide(II) (3.61 g, 16.2 mmol) while cooling in ice. The mixture was stirred for 10 minutes and stirre at room temperature for 2 hours. Hydrochloric acid aqueous solution was added to the mixture, and the mixture was extracted with ethyl acetate. The organic layer was washed with saturated sodium hydrogen carbonate solution and water, and dried over magnesium sulfate. The solvent was condensed under reduced pressure. The residue was purified by silica gel chromatography (hexane-ethyl acetate) to afford Compound 173 (1.87 g, yield 47%).

¹H-NMR (CDCl₃) δ: 1.33 (d, J=6.9 Hz, 3H), 2.01 (s, 3H), 4.71-4.75 (m, 1H), 5.41 (d, J=7.5 Hz, 1H), 6.10 (dd, J=15.9, 5.7 Hz, 1H), 6.43 (d, J=16.0 Hz, 1H), 6.94 (d, J=8.5 Hz, 1H), 7.08 (d, J=8.5 Hz, 1H), 7.42 (dd, J=8.4, 2.1 Hz, 1H), 7.61 (d, J=2.4 Hz, 1H), 7.74 (dd, J=8.5, 2.4 Hz, 1H), 8.04 (d, J=2.1 Hz, 1H).

### Step 8 Preparation of Compound I-267

2 mol/L sodium carbonate aqueous solution (0.061 mL) was added to the ethanol solution (1.0 mL) of Compound 173 (24 mg, 0.061 mmol) and phenyl boronic acid (8.9 mg, 0.073 mmol). The atmosphere was replaced with nitrogen, and bis(triphenylphosphine) palladium(II) dichloride (4.3 mg, 0.0061 mmol) was added to the mixture. The mixture was subjected to microwave irradiation, and stirred at 100° C. for 10 minutes. Water was added to the mixture, and the mixture was extracted with chloroform. The organic layer was washed with water and dried over magnesium sulfate. The solvent was condensed under reduced pressure. The residue was purified by silica gel chromatography (chloroform-methanol) to afford Compound I-267 (18 mg, yield 77%).

¹H-NMR (DMSO-D6) δ: 1.20 (d, J=6.9 Hz, 3H), 1.83 (s, 3H), 4.49 (dd, J=12.9, 6.6 Hz, 1H), 6.26 (dd, J=16.0, 5.5 Hz, 1H), 6.42 (d, J=16.2 Hz, 1H), 7.11 (d, J=8.5 Hz, 1H), 7.39 (t, J=8.0 Hz, 2H), 7.48 (t, J=7.3 Hz, 2H), 7.66-7.72 (m, 3H), 7.84 (d, J=2.0 Hz, 1H), 7.96-8.02 (m, 2H), 8.10 (d, J=2.0 Hz, 1H).

### Example 389

### Preparation of Compound I-389

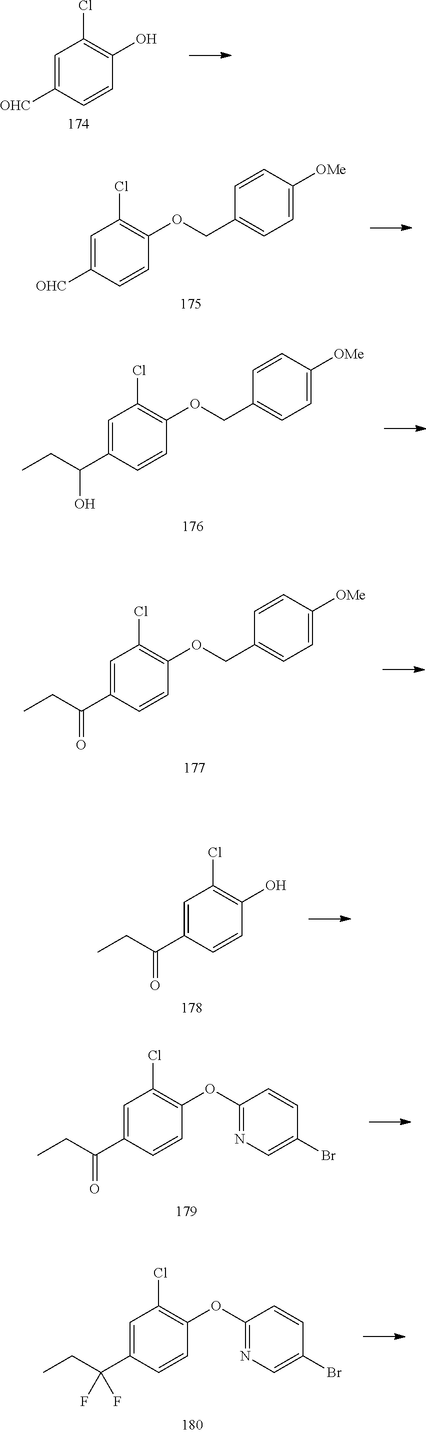

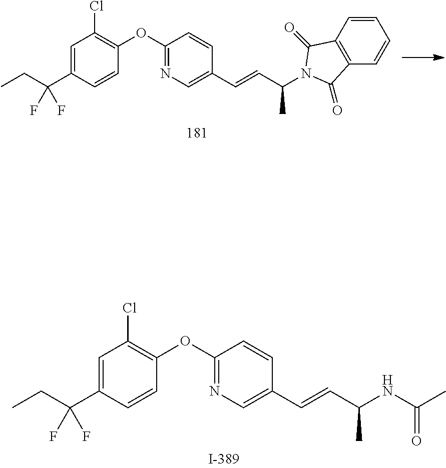 [SMILES: CCC(F)(F)c1ccc(Oc2ccc(/C=C/[C@H](C)N3C(=O)c4ccccc4C3=O)cn2)c(Cl)c1 ; CCC(F)(F)c1ccc(Oc2ccc(/C=C/[C@H](C)NC(C)=O)cn2)c(Cl)c1]

### Step 1 Preparation of Compound 175

Potassium carbonate (13.2 g, 96.0 mmol) was added to DMF solution (50 mL) of Compound 174 (10.0 g, 63.9 mmol) and 1-chloromethyl-4-methoxybenzene (13.0 g, 83.0 mmol), and the mixture was stirred overnight at room temperature. Water was added to the mixture, and the mixture was extracted with ethyl acetate. The organic layer was washed with water, and dried over magnesium sulfate. The solvent was condensed under reduced pressure. Hexane was added to the residue, the mixture was filtered off to afford Compound 175 (17.0 g, yield 96%).

¹H-NMR (CDCl₃) δ: 3.82 (s, 3H), 5.19 (s, 2H), 6.94 (m, 2H), 7.09 (d, J=8.0 Hz, 1H), 7.39 (m 2H), 7.74 (dd, J=8.0, 4.0 Hz, 1H), 7.93 (d, J=4.0 Hz, 1H), 9.85 (s, 1H).

### Step 2 Preparation of Compound 176

THF solution (36.2 mL, 36.2 mmol) of 1 mol/1 ethylmagnesium bromide was added dropwise to THF solution of Compound 175 (5.00 g, 18.1 mmol) in a nitrogen atmosphere. The mixture was stirred at room temperature for 4 hours. Saturated ammonium chloride was added to the mixture, and the mixture was extracted with ethyl acetate. The organic layer was washed with water and brine, and dried over magnesium sulfate. The solvent was condensed under reduced pressure to afford Compound 176 (5.60 g, yield 100%).

¹H-NMR (CDCl₃) δ: 0.90 (t, J=8.0 Hz, 3H), 1.60-1.80 (m, 3H), 3.81 (s, 3H), 4.52 (t, J=4.0 Hz, 1H), 5.08 (s, 2H), 6.92 (m, 2H), 6.93 (d, J=8.0 Hz, 1H), 7.15 (dd, J=8.0, 4.0 Hz, 1H), 7.37 (d, J=4.0 Hz, 1H), 7.38 (m, 2H).

### Step 3 Preparation of Compound 177

IBX (15.3 g, 54.3 mmol) was added to the ethyl acetate solution of Compound 176 (5.60 g, 18.1 mmol) and the mixture was stirred under heat refluxing for 6 hours. The insoluble matter was filtered. Saturated sodium hydrogen carbonate solution was added to the filtrate, and the mixture was separated into the organic layer and water layer. The organic layer was washed with water, and dried over magnesium sulfate. The solvent was condensed under reduced pressure. Diisopropylether was added to the residue. The precipitated solid was filtrated to afford Compound 177 (4.67 g, yield 84%)

¹H-NMR (CDCl₃) δ: 1.21 (t, J=8.0 Hz, 3H), 2.93 (m, 2H), 3.82 (s, 3H), 5.16 (s, 2H), 6.93 (d, J=8.0 Hz, 2H), 7.01 (d, J=8.0 Hz, 1H), 7.38 (d, J=8.0 Hz, 2H), 7.84 (dd, J=8.0, 4.0 Hz, 1H), 8.02 (d, J=4.0 Hz, 1H).

### Step 4 Preparation of Compound 178

Trifluoroacetic acid (46.0 mL) was added to the anisole solution (50.0 mL) of Compound 177 (4.65 g, 15.3 mmol) and the mixture was stirred overnight at room temperature. The mixture was condensed under reduced pressure. Hexane was added to the residue, and the precipitated solid was filtrated to afford Compound 178 (2.55 g, yield 91%).

¹H-NMR (CDCl₃) δ: 1.22 (t, J=8.0 Hz, 3H), 2.94 (q, J=8.0 Hz, 2H), 5.98 (s, 1H), 7.08 (d, J=8.0 Hz, 1H), 7.83 (dd, J=8.0, 4.0 Hz, 1H), 8.00 (d, J=4.0 Hz, 1H).

### Step 5 Preparation of Compound 179

Compound 178 (1.00 g, 5.42 mmol) and 2,5-dibromopyridine (7.70 g, 32.5 mmol) were dissolved in NMP (15.0 mL). Cesium carbonate (17.6 g, 54.2 mmol) was added to the mixture, and the mixture was stirred at 140° C. for 24 hours. Water was added to the mixture, and the mixture was extracted with ethyl acetate. The organic layer was washed with water, and dried over magnesium sulfate. The solvent was condensed under reduced pressure. The residue was purified by silica gel chromatography (hexane-ethyl acetate) to afford Compound 179 (0.465 g, yield 25%).

¹H-NMR (CDCl₃) δ: 1.24 (t, J=8.0 Hz, 3H), 3.00 (q, J=8.0 Hz, 2H), 6.98 (d, J=8.0 Hz, 1H), 7.28 (d, J=8.0 Hz, 1H), 7.84 (dd, J=8.0, 4.0 Hz, 1H), 7.92 (d, J=8.0, 4.0 Hz, 1H), 8.10 (d, J=4.0 Hz, 1H), 8.17 (d, J=4.0 Hz, 1H).

### Step 6 Preparation of Compound 180

Compound 179 (0.200 g, 0.587 mmol) and [bis (2-methoxyethyl)aminosulfa trifloride (0.650 g, 2.94 mmol) were stirred at 80° C. for 11 hours. Saturated sodium hydrogen carbonate solution was added to the mixture, the mixture was extracted with ethyl acetate. The organic layer was washed with brine and water, and dried over magnesium sulfate. The solvent was condensed under reduced pressure. The residue was purified by silica gel chromatography (hexane-ethyl acetate) to afford Compound 180 (0.157 g, yield 74%).

¹H-NMR (CDCl₃) δ: 1.03 (t, J=7.4 Hz, 3H), 2.16 (m, 2H), 6.95 (d, J=8.7 Hz, 1H), 7.23 (m, 1H), 7.41 (d, J=8.3 Hz, 1H), 7.59 (s, 1H), 7.82 (d, J=6.7 Hz, 1H) 8.16 (s, 1H).

### Step 7 Preparation of Compound 181

2 mol/L sodium carbonate aqueous solution (0.432 mL) was added to the ethanol solution (3.00 mL) of Compound 180 (0.157 g, 0.432 mmol) and Compound 2 (0.156 g, 0.476 mmol) prepared in Reference Example 001. The atmosphere was replaced with nitrogen, and bis(triphenylphosphine) palladium(II) dichloride (0.030 g, 0.043 mmol) was added to the mixture. The mixture was subjected to microwave irradiation and stirred at 80° C. for 15 minutes. The mixture was diluted with chloroform (6.00 mL), and WSCD (0.166 g, 0.865 mmol) was added to the mixture. The mixture was stirred at room temperature for 3 hours. Water was added to the mixture, and the mixture was extracted with chloroform. The organic layer was washed with brine and dried over magnesium sulfate. The solvent was condensed under reduced pressure. The residue was purified by silica gel chromatography (hexane-ethyl acetate) to afford Compound 181 (0.147 g, yield 70%).

¹H-NMR (CDCl₃) δ: 1.03 (t, J=7.4 Hz, 3H), 1.67 (d, J=7.0 Hz, 3H), 2.08-2.20 (m, 2H), 5.09 (m, 1H), 6.57 (m, 2H), 6.96 (d, J=8.5 Hz, 1H), 7.23 (d, J=8.4 Hz, 1H), 7.39 (d, J=8.5 Hz, 1H), 7.57 (s, 1H), 7.72 (m, 2H), 7.83 (m, 3H), 8.06 (s, 1H).

### Step 8 Preparation of Compound I-389

Compound 181 (0.147 g, 0.304 mmol) was dissolved in the mixture of dicloromethane (3.00 mL) and ethanol (0.50 mL). Hydrazine monohydrate (0.15 mL, 3.04 mmol) was added to the mixture, the mixture was stirred at 60° C. for 4 hours. Saturated sodium hydrogen carbonate solution was added to the mixture, and the mixture was extracted with chloroform. The organic layer was washed with brine, and dried over magnesium sulfate. The solvent was condensed under reduced pressure. The residue was dissolved in dichloromethane (3.00 mL). Pyridine (0.074 mL, 0.913 mmol) was added to the mixture while stirring it in an ice bath. Acetyl chloride (0.033 mL, 0.475 mmol) was added to the mixture, and the mixture was stirred for 0.5 hours. Saturated sodium hydrogen carbonate solution was added to the mixture, and the mixture was extracted with chloroform. The organic layer was washed with brine, and dried over magnesium sulfate. The solvent was condensed under reduced pressure. The residue was purified by silica gel chromatography (chloroform-methanol) to afford Compound I-389 (0.108 g, yield 90%).

¹H-NMR (CDCl₃) δ: 1.03 (t, J=7.4 Hz, 3H), 1.34 (d, J=6.8 Hz, 3H), 2.02 (s, 3H), 2.16 (m, 2H), 4.74 (m, 1H), 5.44 (d, J=7.8 Hz, 1H), 6.12 (dd, J=16.1, 5.6 Hz, 1H), 6.45 (d, J=16.1 Hz, 1H), 6.96 (d, J=8.5 Hz, 1H), 7.25 (m, 1H), 7.40 (d, J=8.4 Hz, 1H), 7.58 (s, 1H), 7.77 (d, J=8.3 Hz, 1H), 8.07 (s, 1H).

### Example 425

### Preparation of Compound I-425

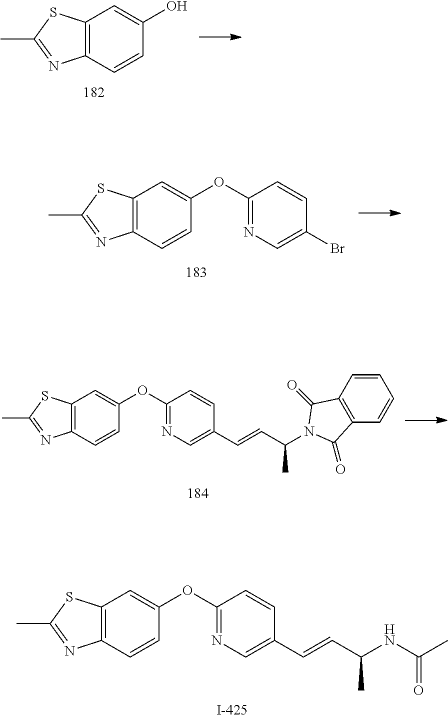 [SMILES: Cc1nc2ccc(O)cc2s1 ; Cc1nc2ccc(Oc3ccc(Br)cn3)cc2s1 ; Cc1nc2ccc(Oc3ccc(/C=C/[C@H](C)N4C(=O)c5ccccc5C4=O)cn3)cc2s1 ; CC(=O)N[C@@H](C)/C=C/c1ccc(Oc2ccc3nc(C)sc3c2)nc1]

### Step 1 Preparation of Compound 183

Compound 182 (0.300 g, 1.82 mmol) and 2,5-dibromopyridine (0.516 g, 2.18 mmol) were dissolved in NMP (2.00 mL). Cesium carbonate (1.78 G, 5.45 mmol) was added to the mixture, and the mixture was stirred at 140° C. for 5 hours. Water was added to the mixture, and the mixture was extracted with diethylether. The organic layer was washed with brine, and dried over magnesium sulfate. The solvent was condensed under reduced pressure. The residue was purified by silica gel chromatography (hexane-ethyl acetate) to afford Compound 183 (0.412 g, yield 71%).

¹H-NMR (CDCl₃) δ: 2.84 (s, 3H), 6.89 (d, J=8.7 Hz, 1H), 7.21 (dd, J=8.8, 2.4 Hz, 1H), 7.59 (d, J=2.3 Hz, 1H), 7.80 (dd, J=8.7, 2.5 Hz, 1H), 7.95 (d, J=8.8 Hz, 1H), 8.21 (d, J=2.0 Hz, 1H).

### Step 2 Preparation of Compound 184

2 mol/L sodium carbonate aqueous solution (0.311 mL) was added to the ethanol solution (4.00 mL) of Compound 183 (0.100 g, 0.311 mmol) and Compound 2 (0.122 g, 0.374 mmol) prepared in Reference Example 001. The atmosphere was replaced with nitrogen, and bis(triphenylphosphine)palladium(II) dichloride (0.0022 g, 0.031 mmol) was added to the mixture, and the mixture was subjected to microwave irradiation and stirred at 100° C. for 15 minutes. The mixture was diluted with chloroform, and WSCD (0.119 g, 0.623 mmol) was added to the mixture. The mixture was stirred at room temperature for 3 hours. Water was added to the mixture, and the mixture was extracted with chloroform. The organic layer was washed with brine and water, and dried over magnesium sulfate. The solvent was condensed under reduced pressure. The residue was purified by silica gel chromatography (ethyl acetate-hexane) to afford Compound 184 (0.079 g, yield 58%).

¹H-NMR (CDCl₃) δ: 1.67 (d, J=7.0 Hz, 3H), 2.83 (s, 3H), 5.06-5.13 (m, 1H), 6.57 (dd, J=22.0, 16.2 Hz, 2H), 6.90 (d, J=8.5 Hz, 1H), 7.21 (dd, J=8.8, 2.4 Hz, 1H), 7.58 (d, J=2.4 Hz, 1H), 7.71-7.74 (m, 2H), 7.82 (m, 3H), 7.93 (d, J=8.8 Hz, 1H), 8.1 (d, J=2.3 Hz, 1H).

### Step 3 Preparation of Compound I-425

Compound 184 (0.0793 g, 0.180 mmol) was dissolved in the mixture of dichloromethane (3.00 mL) and ethanol (0.50 mL). Hydrazine monohydrate (0.175 mL, 3.59 mmol) was added to the mixture, the mixture was stirred at 60° C. for 4.5 hours. Saturated sodium hydrogen carbonate solution was added to the mixture, and the mixture was extracted with chloroform. The organic layer was washed with brine, and dried over magnesium sulfate. The solvent was condensed under reduced pressure. The residue was dissolved in the methanol (2.00 mL). Acetic anhydride (0.025 mL, 0.269 mmol) was added to the mixture while stirring it in an ice bath, and the mixture was stirred for 2 hours. Saturated sodium hydrogen carbonate solution was added to the mixture, and the mixture was extracted with chloroform. The organic layer was washed with brine, dried over magnesium sulfate. The solvent was condensed under reduced pressure. The residue was purified by silica gel chromatography (chloroform-methanol) to afford Compound I-425 (0.0553 g, yield 87%).

¹H-NMR (CDCl₃) δ: 1.34 (d, J=6.8 Hz, 3H), 2.02 (t, J=15.7 Hz, 3H), 2.83 (s, 3H), 4.75 (dd, J=12.8, 6.8 Hz, 1H), 5.44 (d, J=8.0 Hz, 1H), 6.12 (dd, J=16.1, 5.5 Hz, 1H), 6.45 (d, J=16.1 Hz, 1H), 6.91 (d, J=8.5 Hz, 1H), 7.22 (dd, J=8.8, 2.5 Hz, 1H), 7.59 (d, J=2.3 Hz, 1H), 7.74 (dd, J=8.5, 2.5 Hz, 1H), 7.94 (d, J=8.8 Hz, 1H), 8.12 (d, J=2.3 Hz, 1H).

### Examples 431-520

Compounds I-431-520 were obtained in the above similar Example. The structures and chemical data of Compounds of I-431-520 are showed.

TABLE 58

| | | LCMS | | |
|---|---|---|---|---|
| | | | retention | |
| | | | time | |
| No. | Structure | [M + H] | (min) | condition |
| I-431 | 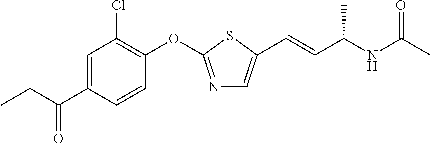 [SMILES: CCC(=O)c1ccc(Oc2ncc(/C=C/[C@H](C)NC(C)=O)s2)c(Cl)c1] | | | |
| I-432 | 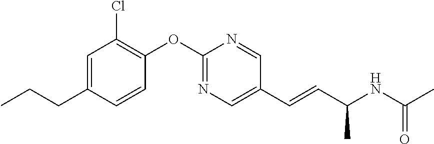 [SMILES: CCCc1ccc(Oc2ncc(/C=C/[C@H](C)NC(C)=O)cn2)c(Cl)c1] | 361.1 | 2.15 | 2 |
| I-433 | 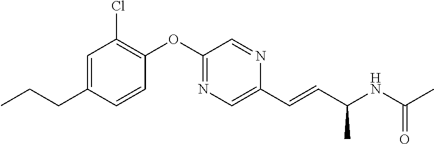 [SMILES: CCCc1ccc(Oc2cnc(/C=C/[C@H](C)NC(C)=O)cn2)c(Cl)c1] | 361.2 | 2.33 | 2 |
| I-434 | 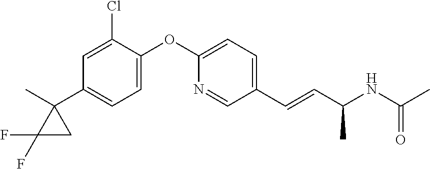 [SMILES: CC(=O)N[C@@H](C)/C=C/c1ccc(Oc2ccc(C3(C)CC3(F)F)cc2Cl)nc1] | 406.9 | 2.26 | 2 |
| I-435 | 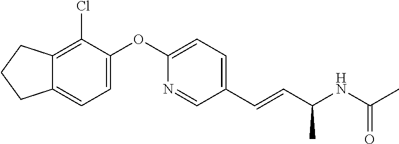 [SMILES: CC(=O)N[C@@H](C)/C=C/c1ccc(Oc2ccc3c(c2Cl)CCC3)nc1] | 356.9 | 2.24 | 2 |
| I-436 | 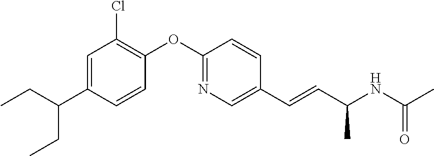 [SMILES: CCC(CC)c1ccc(Oc2ccc(/C=C/[C@H](C)NC(C)=O)cn2)c(Cl)c1] | 388.3 | 2.61 | 2 |
| I-437 | 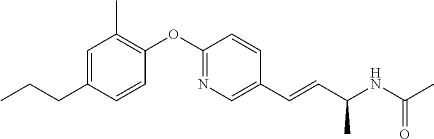 [SMILES: CCCc1ccc(Oc2ccc(/C=C/[C@H](C)NC(C)=O)cn2)c(C)c1] | 339.6 | 2.36 | 2 |

TABLE 59

| | | | | |
|---|---|---|---|---|
| I-438 | 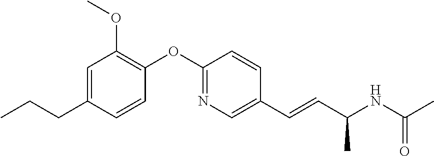 [SMILES: CCCc1ccc(Oc2ccc(/C=C/[C@H](C)NC(C)=O)cn2)c(OC)c1] | 355.5 | 2.16 | 2 |
| I-439 | 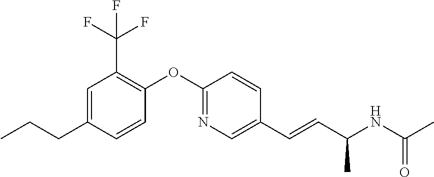 [SMILES: CCCc1ccc(Oc2ccc(/C=C/[C@H](C)NC(C)=O)cn2)c(C(F)(F)F)c1] | 393.5 | 2.44 | 2 |
| I-440 | 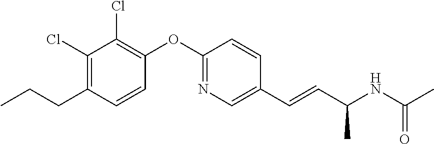 [SMILES: CCCc1ccc(Oc2ccc(/C=C/[C@H](C)NC(C)=O)cn2)c(Cl)c1Cl] | 394.7 | 2.55 | 2 |
| I-441 | 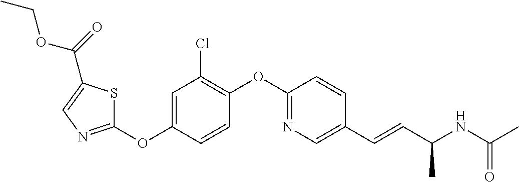 [SMILES: CCOC(=O)c1cnc(Oc2ccc(Oc3ccc(/C=C/[C@H](C)NC(C)=O)cn3)c(Cl)c2)s1] | 488.1 | 2.32 | 2 |
| I-442 | 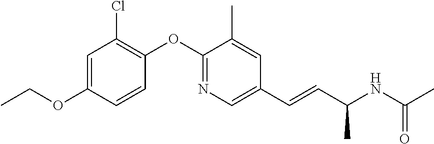 [SMILES: CCOc1ccc(Oc2ncc(/C=C/[C@H](C)NC(C)=O)cc2C)c(Cl)c1] | 375.4 | 2.27 | 2 |
| I-443 | 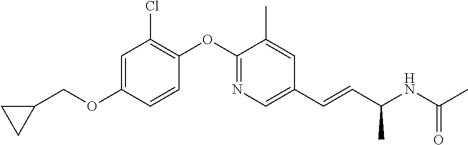 [SMILES: CC(=O)N[C@@H](C)/C=C/c1cnc(Oc2ccc(OCC3CC3)cc2Cl)c(C)c1] | 401.4 | 2.41 | 2 |
| I-444 | 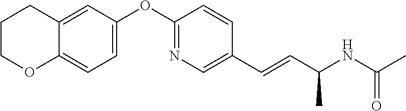 [SMILES: CC(=O)N[C@@H](C)/C=C/c1ccc(Oc2ccc3c(c2)CCCO3)nc1] | 339.5 | 1.84 | 2 |

TABLE 60

| | | | | |
|---|---|---|---|---|
| I-445 | 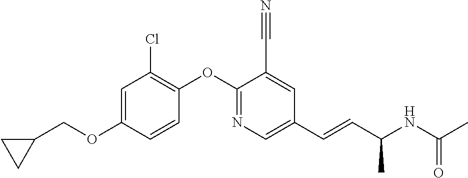 [SMILES: CC(=O)N[C@@H](C)/C=C/c1cnc(Oc2ccc(OCC3CC3)cc2Cl)c(C#N)c1] | 412.2 | 2.24 | 2 |
| I-445 | 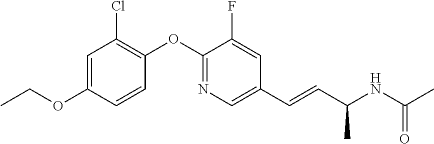 [SMILES: CCOc1ccc(Oc2ncc(/C=C/[C@H](C)NC(C)=O)cc2F)c(Cl)c1] | 379.1 | 2.19 | 2 |
| I-447 | 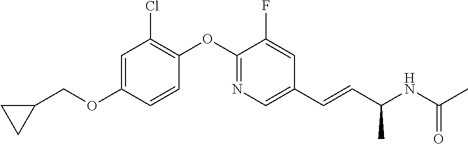 [SMILES: CC(=O)N[C@@H](C)/C=C/c1cnc(Oc2ccc(OCC3CC3)cc2Cl)c(F)c1] | 405.2 | 2.33 | 2 |
| I-448 | 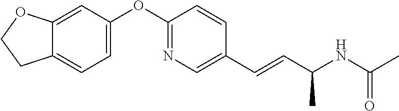 [SMILES: CC(=O)N[C@@H](C)/C=C/c1ccc(Oc2ccc3c(c2)OCC3)nc1] | 324.9 | 1.74 | 2 |
| I-449 | 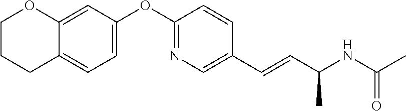 [SMILES: CC(=O)N[C@@H](C)/C=C/c1ccc(Oc2ccc3c(c2)OCCC3)nc1] | 338.9 | 1.89 | 2 |
| I-450 | 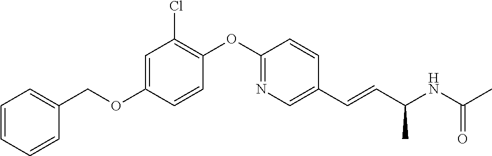 [SMILES: CC(=O)N[C@@H](C)/C=C/c1ccc(Oc2ccc(OCc3ccccc3)cc2Cl)nc1] | 423.2 | 2.29 | 2 |
| I-451 | 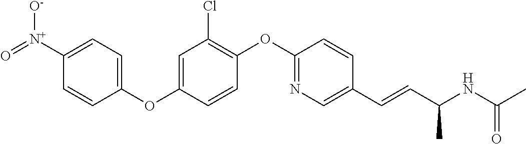 [SMILES: CC(=O)N[C@@H](C)/C=C/c1ccc(Oc2ccc(Oc3ccc([N+](=O)[O-])cc3)cc2Cl)nc1] | 454.2 | 2.23 | 2 |

TABLE 61

| | | | | |
|---|---|---|---|---|
| I-452 | 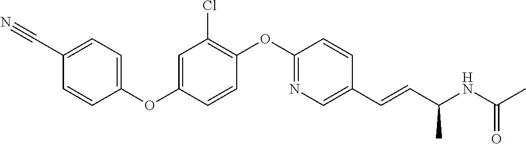 [SMILES: CC(=O)N[C@@H](C)/C=C/c1ccc(Oc2ccc(Oc3ccc(C#N)cc3)cc2Cl)nc1] | 434. | 2.2 | 2 |
| I-453 | 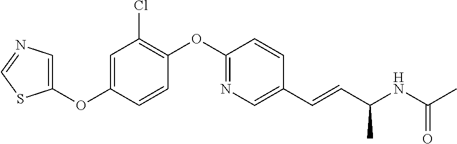 [SMILES: CC(=O)N[C@@H](C)/C=C/c1ccc(Oc2ccc(Oc3cncs3)cc2Cl)nc1] | 416.1 | 1.97 | 2 |
| I-454 | 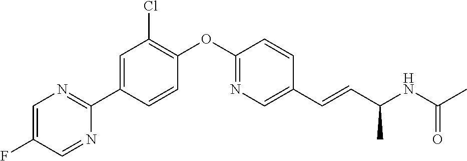 [SMILES: CC(=O)N[C@@H](C)/C=C/c1ccc(Oc2ccc(-c3ncc(F)cn3)cc2Cl)nc1] | 413.0 | 2.14 | 2 |
| I-455 | 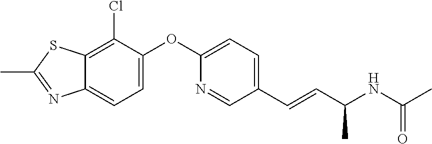 [SMILES: CC(=O)N[C@@H](C)/C=C/c1ccc(Oc2ccc3nc(C)sc3c2Cl)nc1] | 387.9 | 1.94 | 2 |
| I-456 | 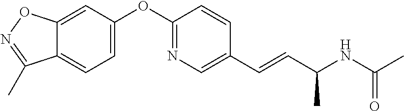 [SMILES: CC(=O)N[C@@H](C)/C=C/c1ccc(Oc2ccc3c(C)noc3c2)nc1] | 338.3 | 2.71 | 2 |
| I-457 | 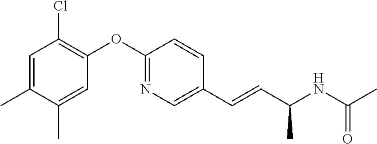 [SMILES: CC(=O)N[C@@H](C)/C=C/c1ccc(Oc2cc(C)c(C)cc2Cl)nc1] | 345.3 | 4.19 | 2 |
| I-458 | 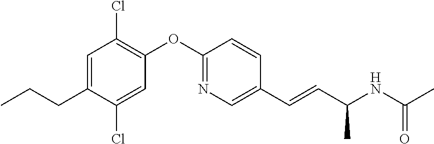 [SMILES: CCCc1cc(Cl)c(Oc2ccc(/C=C/[C@H](C)NC(C)=O)cn2)cc1Cl] | 393.3 | 5.23 | 2 |

TABLE 62

| | | | | |
|---|---|---|---|---|
| I-459 | 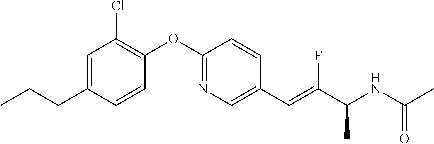 [SMILES: CCCc1ccc(Oc2ccc(/C=C(\F)[C@H](C)NC(C)=O)cn2)c(Cl)c1] | 377.2 | 2.46 | 2 |
| I-460 | 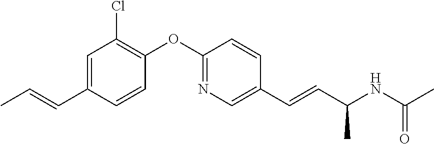 [SMILES: C/C=C/c1ccc(Oc2ccc(/C=C/[C@H](C)NC(C)=O)cn2)c(Cl)c1] | 356.9 | 2.3 | 2 |
| I-461 | 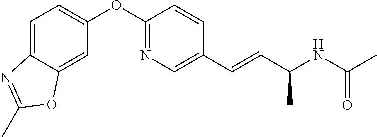 [SMILES: CC(=O)N[C@@H](C)/C=C/c1ccc(Oc2ccc3nc(C)oc3c2)nc1] | 338.0 | 1.58 | 2 |
| I-462 | 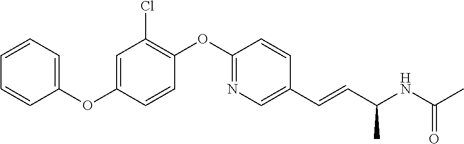 [SMILES: CC(=O)N[C@@H](C)/C=C/c1ccc(Oc2ccc(Oc3ccccc3)cc2Cl)nc1] | 409.2 | 2.38 | 2 |
| I-463 | 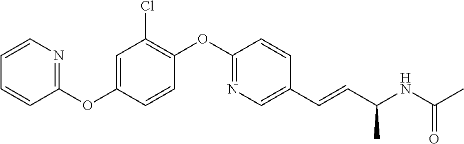 [SMILES: CC(=O)N[C@@H](C)/C=C/c1ccc(Oc2ccc(Oc3ccccn3)cc2Cl)nc1] | 410.3 | 2.04 | 2 |
| I-464 | 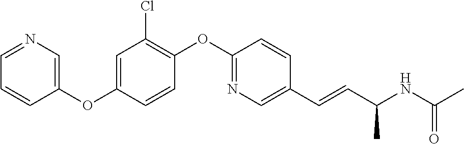 [SMILES: CC(=O)N[C@@H](C)/C=C/c1ccc(Oc2ccc(Oc3cccnc3)cc2Cl)nc1] | 410.4 | 1.74 | 2 |
| I-465 | 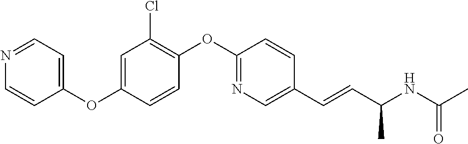 [SMILES: CC(=O)N[C@@H](C)/C=C/c1ccc(Oc2ccc(Oc3ccncc3)cc2Cl)nc1] | 410.4 | 1.27 | 2 |

TABLE 63

| | | | | |
|---|---|---|---|---|
| I-466 | 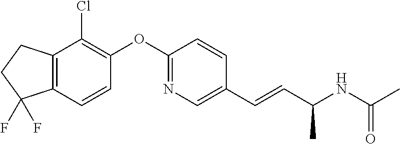 [SMILES: CC(=O)N[C@@H](C)/C=C/c1ccc(Oc2ccc3c(c2Cl)CCC3(F)F)nc1] | 393.0 | 2.18 | 2 |
| I-467 | 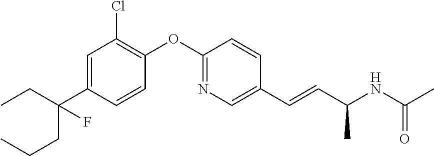 [SMILES: CCCC(F)(CC)c1ccc(Oc2ccc(/C=C/[C@H](C)NC(C)=O)cn2)c(Cl)c1] | | | |
| I-468 | 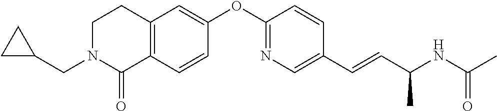 [SMILES: CC(=O)N[C@@H](C)/C=C/c1ccc(Oc2ccc3c(c2)CCN(CC2CC2)C3=O)nc1] | 406.1 | 3.23 | 2 |
| I-469 | 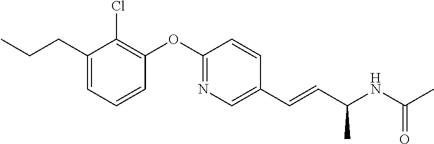 [SMILES: CCCc1cccc(Oc2ccc(/C=C/[C@H](C)NC(C)=O)cn2)c1Cl] | 359.3 | 4.58 | 2 |
| I-470 | 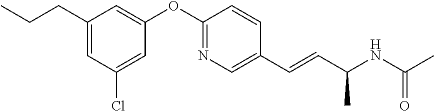 [SMILES: CCCc1cc(Cl)cc(Oc2ccc(/C=C/[C@H](C)NC(C)=O)cn2)c1] | 359.0 | 4.83 | 2 |
| I-471 | 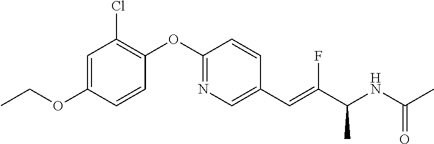 [SMILES: CCOc1ccc(Oc2ccc(/C=C(\F)[C@H](C)NC(C)=O)cn2)c(Cl)c1] | 379.0 | 2.16 | 2 |
| I-472 | 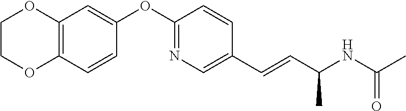 [SMILES: CC(=O)N[C@@H](C)/C=C/c1ccc(Oc2ccc3c(c2)OCCO3)nc1] | 341.0 | 1.68 | 2 |

TABLE 64

| | | | | |
|---|---|---|---|---|
| I-473 | 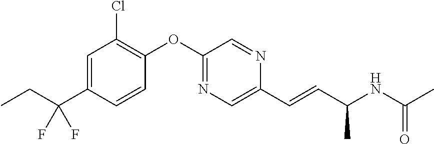 [SMILES: CCC(F)(F)c1ccc(Oc2cnc(/C=C/[C@H](C)NC(C)=O)cn2)c(Cl)c1] | 396.4 | 2.23 | 2 |
| I-474 | 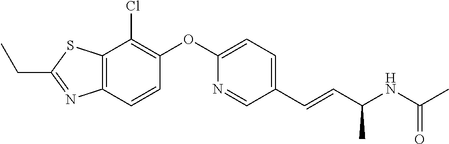 [SMILES: CCc1nc2ccc(Oc3ccc(/C=C/[C@H](C)NC(C)=O)cn3)c(Cl)c2s1] | 402.1 | 2.14 | 2 |
| I-475 | 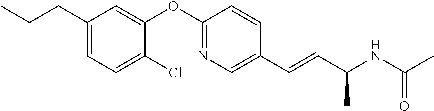 [SMILES: CCCc1ccc(Cl)c(Oc2ccc(/C=C/[C@H](C)NC(C)=O)cn2)c1] | 359.2 | 4.62 | 2 |
| I-476 | 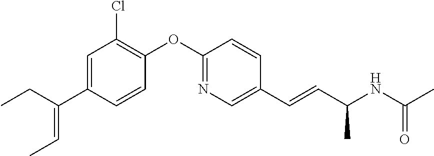 [SMILES: C/C=C(\CC)c1ccc(Oc2ccc(/C=C/[C@H](C)NC(C)=O)cn2)c(Cl)c1] | 385.1 | 2.43 | 2 |
| I-477 | 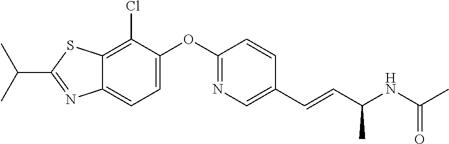 [SMILES: CC(=O)N[C@@H](C)/C=C/c1ccc(Oc2ccc3nc(C(C)C)sc3c2Cl)nc1] | 416.1 | 2.32 | 2 |
| I-478 | 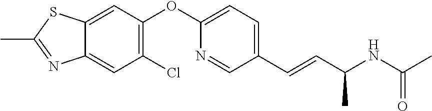 [SMILES: CC(=O)N[C@@H](C)/C=C/c1ccc(Oc2cc3sc(C)nc3cc2Cl)nc1] | 388.1 | 1.89 | 2 |
| I-479 | 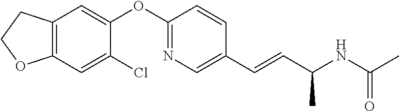 [SMILES: CC(=O)N[C@@H](C)/C=C/c1ccc(Oc2cc3c(cc2Cl)OCC3)nc1] | 359.2 | 1.83 | 2 |

TABLE 65

| | | | | |
|---|---|---|---|---|
| I-480 | 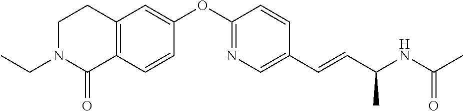 [SMILES: CCN1CCc2cc(Oc3ccc(/C=C/[C@H](C)NC(C)=O)cn3)ccc2C1=O] | 380.5 | 1.57 | 2 |
| I-481 | 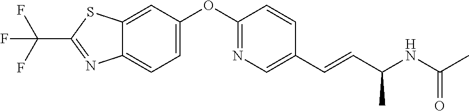 [SMILES: CC(=O)N[C@@H](C)/C=C/c1ccc(Oc2ccc3nc(C(F)(F)F)sc3c2)nc1] | 408.4 | 2.18 | 2 |
| I-482 | 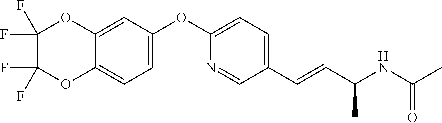 [SMILES: CC(=O)N[C@@H](C)/C=C/c1ccc(Oc2ccc3c(c2)OC(F)(F)C(F)(F)O3)nc1] | 413.0 | 2.31 | 2 |
| I-483 | 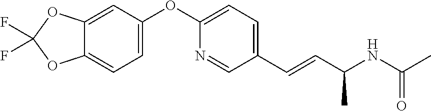 [SMILES: CC(=O)N[C@@H](C)/C=C/c1ccc(Oc2ccc3c(c2)OC(F)(F)O3)nc1] | 363.4 | 2.06 | 2 |
| I-484 | 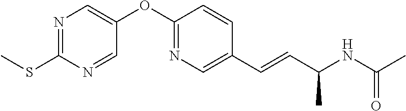 [SMILES: CSc1ncc(Oc2ccc(/C=C/[C@H](C)NC(C)=O)cn2)cn1] | 331.4 | 1.67 | 2 |
| I-485 | 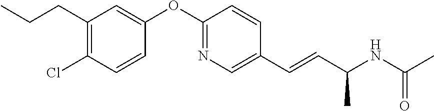 [SMILES: CCCc1cc(Oc2ccc(/C=C/[C@H](C)NC(C)=O)cn2)ccc1Cl] | 359.4 | 2.43 | 2 |
| I-486 | 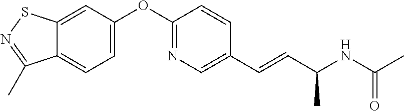 [SMILES: CC(=O)N[C@@H](C)/C=C/c1ccc(Oc2ccc3c(C)nsc3c2)nc1] | 354.4 | 1.82 | 2 |

TABLE 66

| | | | | |
|---|---|---|---|---|
| I-487 | 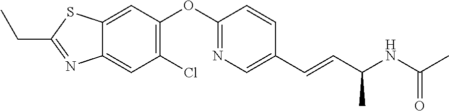 [SMILES: CCc1nc2cc(Cl)c(Oc3ccc(/C=C/[C@H](C)NC(C)=O)cn3)cc2s1] | 402.2 | 2.08 | 2 |
| I-488 | 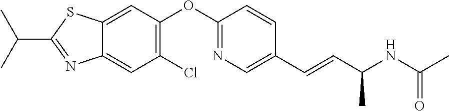 [SMILES: CC(=O)N[C@@H](C)/C=C/c1ccc(Oc2cc3sc(C(C)C)nc3cc2Cl)nc1] | 416.1 | 2.26 | 2 |
| I-489 | 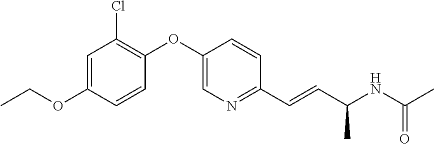 [SMILES: CCOc1ccc(Oc2ccc(/C=C/[C@H](C)NC(C)=O)nc2)c(Cl)c1] | 361.4 | 1.98 | 2 |
| I-490 | 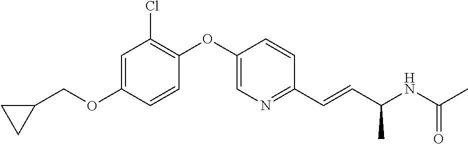 [SMILES: CC(=O)N[C@@H](C)/C=C/c1ccc(Oc2ccc(OCC3CC3)cc2Cl)cn1] | 387.4 | 2.14 | 2 |
| I-491 | 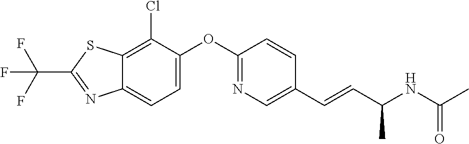 [SMILES: CC(=O)N[C@@H](C)/C=C/c1ccc(Oc2ccc3nc(C(F)(F)F)sc3c2Cl)nc1] | 442.3 | 2.44 | 2 |
| I-492 | 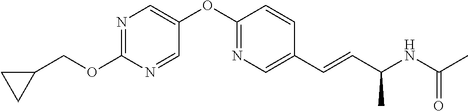 [SMILES: CC(=O)N[C@@H](C)/C=C/c1ccc(Oc2cnc(OCC3CC3)nc2)nc1] | 355.5 | 1.79 | 2 |
| I-493 | 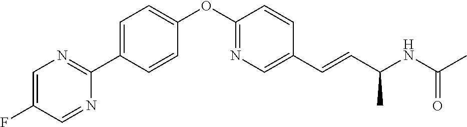 [SMILES: CC(=O)N[C@@H](C)/C=C/c1ccc(Oc2ccc(-c3ncc(F)cn3)cc2)nc1] | 379.5 | 1.92 | 2 |

TABLE 67

| | | | | |
|---|---|---|---|---|
| I-494 | 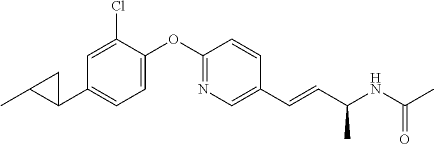 [SMILES: CC(=O)N[C@@H](C)/C=C/c1ccc(Oc2ccc(C3CC3C)cc2Cl)nc1] | 371.0 | 2.38 | 2 |
| I-495 | 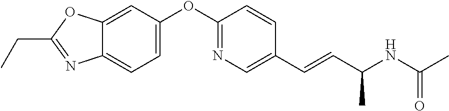 [SMILES: CCc1nc2ccc(Oc3ccc(/C=C/[C@H](C)NC(C)=O)cn3)cc2o1] | 352.4 | 1.75 | 2 |
| I-496 | 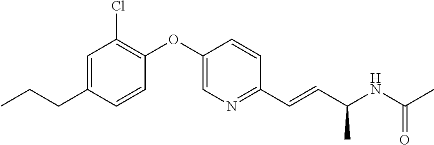 [SMILES: CCCc1ccc(Oc2ccc(/C=C/[C@H](C)NC(C)=O)nc2)c(Cl)c1] | 359.1 | 2.29 | 2 |
| I-497 | 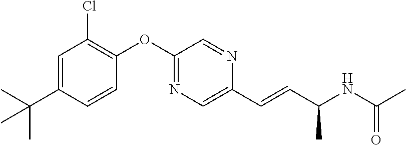 [SMILES: CC(=O)N[C@@H](C)/C=C/c1cnc(Oc2ccc(C(C)(C)C)cc2Cl)cn1] | 374.0 | 2.4 | 2 |
| I-498 | 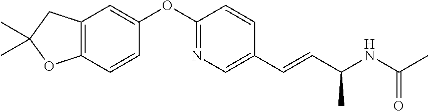 [SMILES: CC(=O)N[C@@H](C)/C=C/c1ccc(Oc2ccc3c(c2)CC(C)(C)O3)nc1] | 353.4 | 1.97 | 2 |
| I-499 | 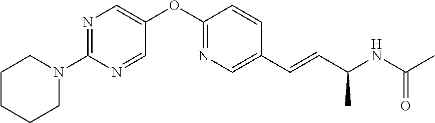 [SMILES: CC(=O)N[C@@H](C)/C=C/c1ccc(Oc2cnc(N3CCCCC3)nc2)nc1] | 368.4 | 2.05 | 2 |
| I-500 | 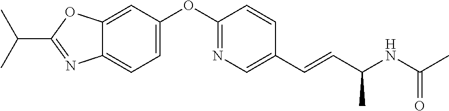 [SMILES: CC(=O)N[C@@H](C)/C=C/c1ccc(Oc2ccc3nc(C(C)C)oc3c2)nc1] | 366.6 | 1.91 | 2 |

TABLE 68

| | | | | |
|---|---|---|---|---|
| I-501 | 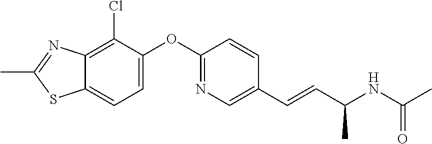 [SMILES: CC(=O)N[C@@H](C)/C=C/c1ccc(Oc2ccc3sc(C)nc3c2Cl)nc1] | 388.1 | 1.73 | 2 |
| I-502 | 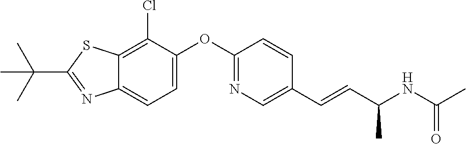 [SMILES: CC(=O)N[C@@H](C)/C=C/c1ccc(Oc2ccc3nc(C(C)(C)C)sc3c2Cl)nc1] | 430.9 | 2.5 | 2 |
| I-503 | 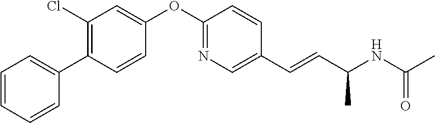 [SMILES: CC(=O)N[C@@H](C)/C=C/c1ccc(Oc2ccc(-c3ccccc3)c(Cl)c2)nc1] | 393.0 | 2.39 | 2 |
| I-504 | 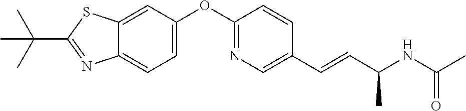 [SMILES: CC(=O)N[C@@H](C)/C=C/c1ccc(Oc2ccc3nc(C(C)(C)C)sc3c2)nc1] | 396.8 | 2.23 | 2 |
| I-505 | 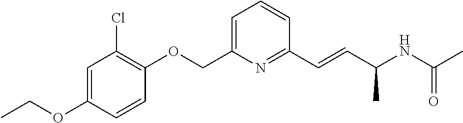 [SMILES: CCOc1ccc(OCc2cccc(/C=C/[C@H](C)NC(C)=O)n2)c(Cl)c1] | 375.3 | 2.06 | 2 |
| I-506 | 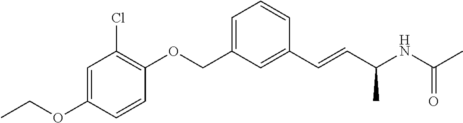 [SMILES: CCOc1ccc(OCc2cccc(/C=C/[C@H](C)NC(C)=O)c2)c(Cl)c1] | 374.0 | 2.33 | 2 |
| I-507 | 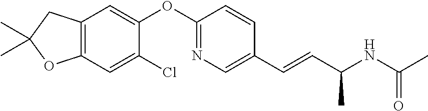 [SMILES: CC(=O)N[C@@H](C)/C=C/c1ccc(Oc2cc3c(cc2Cl)OC(C)(C)C3)nc1] | 387.0 | 2.17 | 2 |

TABLE 69

| | | | | |
|---|---|---|---|---|
| I-508 | 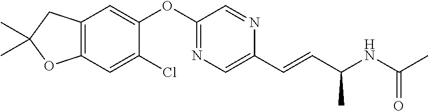 [SMILES: CC(=O)N[C@@H](C)/C=C/c1cnc(Oc2cc3c(cc2Cl)OC(C)(C)C3)cn1] | 388.1 | 2.14 | 2 |
| I-509 | 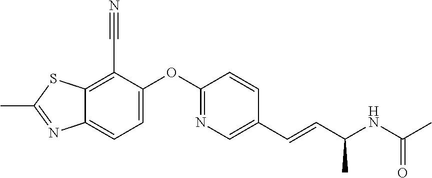 [SMILES: CC(=O)N[C@@H](C)/C=C/c1ccc(Oc2ccc3nc(C)sc3c2C#N)nc1] | 378.9 | 1.78 | 2 |
| I-510 | 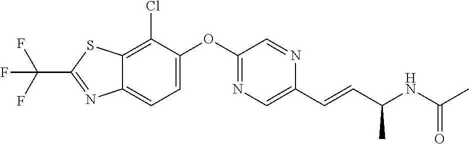 [SMILES: CC(=O)N[C@@H](C)/C=C/c1cnc(Oc2ccc3nc(C(F)(F)F)sc3c2Cl)cn1] | 443.1 | 2.37 | 2 |
| I-511 | 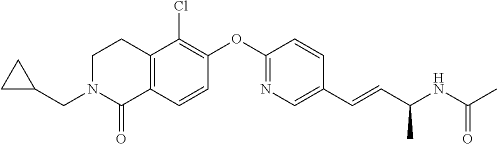 [SMILES: CC(=O)N[C@@H](C)/C=C/c1ccc(Oc2ccc3c(c2Cl)CCN(CC2CC2)C3=O)nc1] | 440.1 | 2 | 2 |
| I-512 | 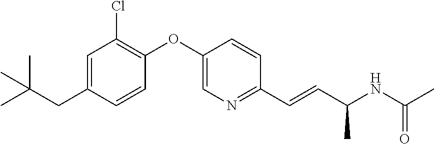 [SMILES: CC(=O)N[C@@H](C)/C=C/c1ccc(Oc2ccc(CC(C)(C)C)cc2Cl)cn1] | 387.0 | 2.54 | 2 |
| I-513 | 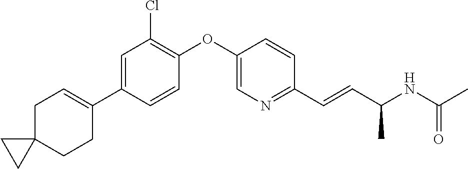 [SMILES: CC(=O)N[C@@H](C)/C=C/c1ccc(Oc2ccc(C3=CCC4(CC3)CC4)cc2Cl)cn1] | 423.1 | 2.7 | 2 |
| I-514 | 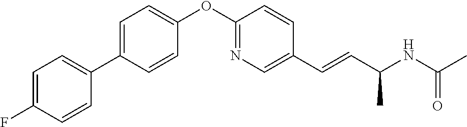 [SMILES: CC(=O)N[C@@H](C)/C=C/c1ccc(Oc2ccc(-c3ccc(F)cc3)cc2)nc1] | 377.4 | 2.25 | 2 |

TABLE 70

| | | | | |
|---|---|---|---|---|
| I-515 | 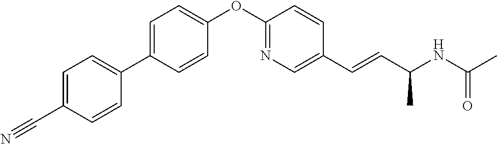 [SMILES: CC(=O)N[C@@H](C)/C=C/c1ccc(Oc2ccc(-c3ccc(C#N)cc3)cc2)nc1] | 384.0 | 2.07 | 2 |
| I-516 | 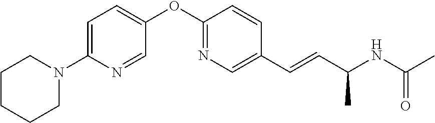 [SMILES: CC(=O)N[C@@H](C)/C=C/c1ccc(Oc2ccc(N3CCCCC3)nc2)nc1] | 367.1 | 1.28 | 2 |
| I-517 | 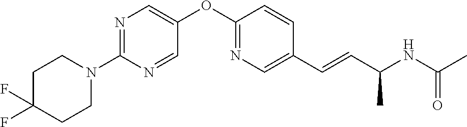 [SMILES: CC(=O)N[C@@H](C)/C=C/c1ccc(Oc2cnc(N3CCC(F)(F)CC3)nc2)nc1] | 404.2 | 2.01 | 2 |
| I-518 | 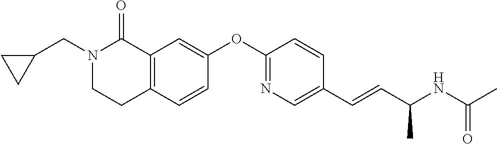 [SMILES: CC(=O)N[C@@H](C)/C=C/c1ccc(Oc2ccc3c(c2)C(=O)N(CC2CC2)CC3)nc1] | 406.5 | 1.84 | 2 |
| I-519 | 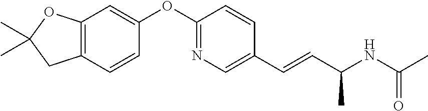 [SMILES: CC(=O)N[C@@H](C)/C=C/c1ccc(Oc2ccc3c(c2)OC(C)(C)C3)nc1] | 353.5 | 2.02 | 2 |
| I-520 | 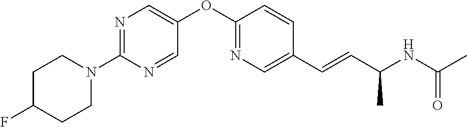 [SMILES: CC(=O)N[C@@H](C)/C=C/c1ccc(Oc2cnc(N3CCC(F)CC3)nc2)nc1] | 386.4 | 1.88 | 2 |

TABLE 71

| No. | 1H NMR |
|---|---|
| I-431 | 1H-NMR (CDCl3) δ: 1.23 (t, J = 8.0 Hz, 3H), 1.31 (d, J = 8.0 Hz, 3H), 2.02 (s, 3H), 2.99 (q, J = 8.0 |
| | Hz, 2H), 4.70 (m, 1H), 5.48 (d, J = 8.0 Hz, 1H), 5.82 (dd, J = 16.0, 8.0 Hz, 1H), 6.51 (d, J = 16.0 |
| | Hz, 1H), 7.03 (s, 1H), 7.47 (d, J = 8.0 Hz, 1H), 7.92 (dd, J = 8.0, 4.0 Hz, 1H), 8.09 (d, J = 4.0 Hz, 1H). |
| I-432 | 1H-NMR (CDCl3) δ: 0.97 (t, J =8.0 Hz, 3H), 1.34 (d, J = 8.0 Hz, 3H), 1.60-1.90 (m, 2H), 2.03 (s, |
| | 3H), 2.60 (t, J = 8.0 Hz, 2H), 4.74 (m, 1H), 5.51 (d, J = 8.0 Hz, 1H), 6.18 (dd, J = 16.0, 4.0 Hz, 1H), |
| | 6.40 (d, J = 16.0 Hz, 1H), 7.15 (m, 2H), 7.30 (d, J = 4.0 Hz, 1H), 8.52 (s, 2H). |
| I-433 | 1H-NMR (CDCl3) δ: 0.97 (t, J = 8.0 Hz, 3H), 1.35 (d, J = 8.0 Hz, 3H), 1.60-1.75 (m, 2H), 2.02 (s, |
| | 3H), 2.59 (t, J = 8.0 Hz, 2H), 4.80 (m, 1H), 5.51 (d, J = 8.0 Hz, 1H), 6.54 (d, J = 16.0 Hz, 1H), 6.67 |
| | (dd, J = 16.0, 4.0 Hz, 1H), 7.13 (m, 2H), 7.30 (d, J = 4.0 Hz, 1H), 7.97 (d, J = 4.0 Hz, 1H), 8.39 (d, |
| | J = 4.0 Hz, 1H). |
| I-435 | 1H-NMR (CDCl3) δ: 1.33 (d, J = 6.8 Hz, 3H), 2.02 (s, 3H), 2.11-2.18 (m, 2H), 2.99 (t, J = 7.5 Hz, |
| | 4H), 4.74 (m, 1H), 5.43 (d, J = 7.9 Hz, 1H), 6.09 (dd, J = 16.1, 5.8 Hz, 1H), 6.43 (d, J = 16.1 Hz, |
| | 1H), 6.91 (d, J = 8.5 Hz, 1H), 6.99 (d, J = 8.0 Hz, 1H), 7.13 (d, J = 8.0 Hz, 1H), 7.73 (dd, J = 8.6, |
| | 2.4 Hz, 1H), 8.07 (d, J = 2.3 Hz, 1H). |
| I-436 | 1H-NMR (CDCl3) δ: 0.81 (t, J = 8.0 Hz, 6H), 1.33 (d, J = 8.0 Hz, 3H), 1.40-2.10 (m, 4H), 2.02 (s, |
| | 3H), 2.34 (m, 1H), 4.74 (m, 1H), 5.45 (d, J = 8.0 Hz, 1H), 6.10 (dd, J = 16.0, 8.0 Hz, 1H), 6.44 (d, J = |
| | 16.0 Hz, 1H), 6.87 (d, J = 8.0 Hz, 1H), 7.05-7.15 (m, 2H), 7.23 (d, J = 4.0 Hz, 1H), 7.73 (dd, J = |
| | 8.0, 4.0 Hz, 1H), 8.09 (d, J = 4.0 Hz, 1H). |
| I-437 | 1H-NMR (CDCl3) δ: 0.96 (t, J = 7.3 Hz, 3H), 1.33 (d, J = 6.8 Hz, 3H), 1.62-1.70 (m, 3H), 2.01 (t, J = |
| | 17.0 Hz, 3H), 2.14 (d, J = 6.3 Hz, 3H), 2.56 (t, J = 7.7 Hz, 2H), 4.73 (d, J = 6.3 Hz, 1H), 5.49 (d, |
| | J = 7.9 Hz, 1H), 6.08 (dd, J = 16.1, 5.8 Hz, 1H), 6.43 (d, J = 15.9 Hz, 1H), 6.78 (d, J = 8.5 Hz, 1H), |
| | 6.94-7.07 (m, 3H), 7.68 (dd, J = 8.7, 2.4 Hz, 1H), 8.10 (d, J = 2.1 Hz, 1H). |
| I-438 | 1H-NMR (CDCl3) δ: 0.98 (t, J = 7.3 Hz, 3H), 1.32 (d, J = 6.9 Hz, 3H), 1.67 (dt, J = 23.3, 7.4 Hz, |
| | 3H), 2.01 (t, J = 18.2 Hz, 3H), 2.59 (t, J = 7.7 Hz, 2H), 3.75 (d, J = 5.4 Hz, 3H), 4.73 (dd, J = 12.9, |
| | 6.7 Hz, 1H), 5.49 (d, J = 8.0 Hz, 1H), 6.07 (dd, J = 16.0, 5.7 Hz, 1H), 6.43 (d, J = 16.1 Hz, 1H), |
| | 6.78-6.86 (m, 3H), 7.01 (t, J = 7.2 Hz, 1H), 7.68 (dd, J = 8.6, 2.4 Hz, 1H), 8.07 (d, J = 2.3 Hz, 1H). |
| I-439 | 1H-NMR (CDCl3) δ: 0.98 (dd, J = 9.9, 4.8 Hz, 3H), 1.33 (d, J = 6.8 Hz, 3H), 1.67 (td, J = 15.1, 7.5 |
| | Hz, 3H), 2.63 (t, J = 7.7 Hz, 2H), 4.74 (dd, J = 12.6, 6.8 Hz, 1H), 5.49 (d, J = 8.0 Hz, 1H), 6.10 |
| | (dd, J = 16.0, 5.7 Hz, 1H), 6.44 (d, J = 16.1 Hz, 1H), 6.93 (d, J = 8.5 Hz, 1H), 7.12 (d, J = 8.4 Hz, |
| | 1H), 7.74 (dd, J = 8.5, 2.5 Hz, 1H), 8.08 (d, J = 2.4 Hz, 1H). |
| I-440 | 1H-NMR (CDCl3) δ: 1.01 (t, J = 7.4 Hz, 3H), 1.15 (dd, J = 14.7, 6.4 Hz, 2H), 1.33 (d, J = 6.8 Hz, |
| | 3H), 1.68 (td, J = 13.9, 5.9 Hz, 3H), 1.99 (d, J = 19.6 Hz, 3H), 2.75 (t, J = 7.8 Hz, 2H), 4.74 (t, J = |
| | 6.5 Hz, 1H), 5.49 (d, J = 8.0 Hz, 1H), 6.10 (dd, J = 16.1, 5.8 Hz, 1H), 6.44 (d, J = 16.1 Hz, 1H), |
| | 6.92 (t, J = 6.1 Hz, 1H), 7.07 (t, J = 4.1 Hz, 1H), 7.17 (t, J = 6.9 Hz, 1H), 7.75 (dd, J = 8.7, 2.4 Hz, |
| | 1H), 8.05 (d, J = 2.3 Hz, 1H). |
| I-441 | 1H-NMR (DMSO-d6) δ: 1.20 (d, J = 6.8 Hz, 3H), 1.28 (t, J = 7.3 Hz, 3H), 1.84 (s, 3H), 4.30 (q, J = |
| | 7.3 Hz, 2H), 4.50 (m, 1H), 6.28 (dd, J = 16.0, 5.5 Hz, 1H), 6.43 (d, J = 16.0 Hz, 1H), 7.13 (d, J = |
| | 8.5 Hz, 1H), 7.44-7.51 (m, 2H), 7.81 (d, J = 2.8 Hz, 1H), 7.98-8.03 (m, 3H), 8.13 (d, J = 2.0 Hz, 1H). |

TABLE 72

| | |
|---|---|
| I-442 | 1H-NMR (CDCl3) δ: 1.32 (d, J = 7.0 Hz, 3H), 1.42 (t, J = 7.0 Hz, 3H), 2.01 (s, 3H), 2.38 (s, 3H), |
| | 4.02 (q, J = 7.0 Hz, 2H), 4.67-4.78 (m, 1H), 5.42 (d, J = 8.0 Hz, 1H), 6.08 (dd, J = 16.1, 5.8 Hz, |
| | 1H), 6.40 (d, J = 16.1 Hz, 1H), 6.84 (dd, J = 8.9, 2.9 Hz, 1H), 6.98 (d, J = 2.8 Hz, 1H), 7.12 (d, J = |
| | 8.8 Hz, 1H), 7.57 (d, J = 2.0 Hz, 1H), 7.85 (d, J = 2.0 Hz, 1H). |
| I-446 | 1H-NMR (CDCl3) δ: 1.33 (d, J = 6.8 Hz, 3H), 1.42 (t, J = 6.9 Hz, 3H), 2.02 (s, 3H), 4.02 (q, J = 7.0 |
| | Hz, 2H), 4.68-4.79 (m, 1H), 5.41 (d, J = 7.8 Hz, 1H), 6.08 (dd, J = 15.9, 5.6 Hz, 1H), 6.42 (d, J = |
| | 16.1 Hz, 1H), 6.84 (dd, J = 8.8, 2.8 Hz, 1H), 7.00 (d, J = 2.8 Hz, 1H), 7.14 (d, J = 9.0 Hz, 1H), 7.51 |
| | (dd, J = 10.5, 1.5 Hz, 1H), 7.78 (d, J = 1.5 Hz, 1H). |
| I-453 | 1H-NMR (CDCl3) δ: 1.33 (d, J = 7.0 Hz, 3H), 2.02 (s, 3H), 4.74 (m, 1H), 5.51 (d, J = 7.8 Hz, 1H), |
| | 6.11 (dd, J = 16.0, 5.8 Hz, 1H), 6.45 (d, J = 16.0 Hz, 1H), 6.87 (d, J = 3.8 Hz, 1H), 6.94 (d, J = 8.5 |
| | Hz, 1H), 7.22-7.29 (m, 2H), 7.46 (d, J = 2.3 Hz, 1H), 7.74 (dd, J = 8.5, 2.3 Hz, 1H), 8.07 (d, J = 2.0 |
| | Hz, 1H). |
| I-454 | 1H-NMR (CDCl3) δ: 1.34 (d, J = 6.5 Hz, 3H), 2.02 (s, 3H), 4.69-4.80 (m, 1H), 5.44 (d, J = 6.8 Hz, |
| | 1H), 6.12 (dd, J = 16.2, 5.4 Hz, 1H), 6.45 (d, J = 15.8 Hz, 1H), 6.98 (d, J = 8.5 Hz, 1H), 7.30 (d, J = |
| | 8.5 Hz, 1H), 7.77 (d, J = 8.0 Hz, 1H), 8.09 (s, 1H), 8.35 (d, J = 8.0 Hz, 1H), 8.55 (s, 1H), 8.65 (s, |
| | 2H). |
| I-455 | 1H-NMR (CDCl3) δ: 1.33 (d, J = 7.0 Hz, 3H), 2.01 (s, 3H), 2.85 (s, 3H), 4.74 (m, 1H), 5.41 (d, J = |
| | 7.8 Hz, 1H), 6.11 (dd, J = 16.1, 5.8 Hz, 1H), 6.44 (d, J = 15.8 Hz, 1H), 6.98 (d, J = 8.5 Hz, 1H), |
| | 7.31 (d, J = 8.8 Hz, 1H), 7.77 (dd, J = 8.5 Hz, J = 2.3 Hz, 1H), 7.87 (d, J = 8.5 Hz, 1H), 8.05 (d, J = |
| | 2.0 Hz, 1H). |
| I-456 | 1H-NMR (CDCl3) δ: 1.31 (t, J = 17.6 Hz, 3H), 1.62 (s, 3H), 2.04 (t, J = 6.5 Hz, 3H), 2.64 (s, 3H), |
| | 4.75 (dd, J = 13.7, 6.9 Hz, 1H), 5.47 (d, J = 8.0 Hz, 1H), 6.11 (dd, J = 16.0, 5.6 Hz, 1H), 6.45 (d, |
| | J = 16.1 Hz, 1H), 6.90 (d, J = 8.5 Hz, 1H), 7.09 (d, J = 8.7 Hz, 1H), 7.28 (d, J = 9.4 Hz, 1H), 7.64 |
| | (d, J = 8.5 Hz, 1H), 7.74 (t, J = 4.3 Hz, 1H), 8.11 (s, 1H). |
| I-457 | 1H-NMR (CDCl3) δ: 1.24-1.33 (m, 4H), 1.68 (s, 3H), 2.04 (t, J = 11.3 Hz, 3H), 2.23 (s, 6H), 4.73 |
| | (dd, J = 13.7, 6.8 Hz, 1H), 5.52 (d, J = 7.7 Hz, 1H), 6.09 (dd, J = 16.1, 5.6 Hz, 1H), 6.43 (d, J = |
| | 16.1 Hz, 1H), 6.93 (t, J = 18.6 Hz, 1H), 7.24 (d, J = 18.1 Hz, 1H), 7.69 (dd, J = 27.9, 10.2 Hz, 1H), |
| | 8.07 (s, 1H). |
| I-458 | 1H-NMR (CDCl3) δ: 0.95 (dd, J = 31.6, 24.3 Hz, 3H), 1.30 (dt, J = 33.2, 7.5 Hz, 3H), 1.66 (td, J = |
| | 14.8, 7.7 Hz, 4H), 2.03 (d, 3 = 12.4 Hz, 3H), 2.67 (t, J = 7.8 Hz, 2H), 4.74 (dd, J = 13.8, 6.9 Hz, |
| | 1H), 5.49 (d, J = 7.8 Hz, 1H), 6.11 (dd, J = 16.1, 5.6 Hz, 1H), 6.44 (d, J = 15.9 Hz, 1H), 6.93 (d, J = |
| | 8.5 Hz, 1H), 7.31 (s, 1H), 7.75 (d, J = 8.5 Hz, 1H), 8.07 (s, 1H). |
| I-459 | 1H-NMR (CDCl3) δ: 0.97 (t, J = 7.3 Hz, 3H), 1.40 (d, J = 7.0 Hz, 3H), 1.61-1.71 (m, 2H), 2.03 (s, |
| | 3H), 2.56-2.60 (m, 2H), 4.75-4.87 (m, 1H), 5.62-5.72 (m, 2H), 6.92 (brd, J = 8.8 Hz, 1H), 7.10 (s, |
| | 2H), 7.27 (d, J = 8.8 Hz, 1H), 7.90 (dd, J = 8.8, 2.1 Hz, 1H), 8.17 (brs, 1H). |
| I-460 | 1H-NMR (CDCl3) δ: 1.33 (d, J = 6.8 Hz, 3H), 1.89 (d, J = 6.3 Hz, 3H), 2.01 (s, 3H), 4.74 (d, J = |
| | 7.0 Hz, 1H), 5.42 (s, 1H), 6.10 (dd, J = 16.1, 5.8 Hz, 1H), 6.21 (dd, J = 15.6, 6.5 Hz, 1H), 6.34 (d, |
| | J = 15.8 Hz, 1H), 6.44 (d, J = 16.1 Hz, 1H), 6.91 (d, J = 8.5 Hz, 1H), 7.11 (d, J = 8.3 Hz, 1H), |
| | 7.23 (s, 1H), 7.41 (s, 1H), 7.73 (dd, J = 8.5, 2.5 Hz, 1H), 8.07 (d, J = 2.3 Hz, 1H). |

TABLE 73

| | |
|---|---|
| I-461 | 1H-NMR (CDCl3) δ: 1.34 (dd, J = 6.9, 3.1 Hz, 3H), 2.02 (d, J = 2.8 Hz, 3H), 2.32 (s, 3H), 4.75 (s, 1H), |
| | 5.44 (s, 1H), 6.13 (dt, J = 21.7, 7.5 Hz, 1H), 6.46 (dd, J = 16.1, 8.0 Hz, 1H), 6.91 (dd, J = 14.8, |
| | 8.5 Hz, 1H), 7.10 (t, J = 8.7 Hz, 1H), 7.20 (d, J = 9.0 Hz, 1H), 7.63 (d, J = 8.5 Hz, 1H), 7.75 (dd, J = 9.9, |
| | 7.7 Hz, 1H), 8.14 (d, J = 23.6 Hz, 1H). |
| I-462 | 1H-NMR (CDCl3) δ: 1.33 (d, J = 6.8 Hz, 3H), 2.02 (s, 3H), 4.74 (m, 1H), 5.44 (d, J = 8.0 Hz, 1H), |
| | 6.10 (dd, J = 15.8, 5.8 Hz, 1H), 6.44 (d, J = 15.8 Hz, 1H), 6.91-6.96 (m, 2H), 7.06-7.10 (m, 2H), |
| | 7.14-7.17 (m, 2H), 7.36-7.39 (m, 2H), 7.74 (dd, J = 8.5, 2.3 Hz, 1H), 8.08 (d, J = 2.0 Hz, 1H). |
| I-463 | 1H-NMR (CDCl3) δ: 1.33 (d, J = 7.0 Hz, 3H), 2.02 (s, 3H), 4.74 (m, 1H), 5.47 (d, J = 7.8 Hz, 1H), |
| | 6.11 (dd, J = 16.1, 5.8 Hz, 1H), 6.45 (d, J =16.1 Hz, 1H), 6.92-6.97 (m, 2H), 7.04 (m, 1H), 7.10 |
| | (dd, J = 8.8, 2.8 Hz, 1H), 7.23 (d, J = 8.8 Hz, 1H), 7.29 (d, J = 2.8 Hz, 1H), 7.70-7.76 (m, 2H), 8.09 |
| | (d, J = 1.8 Hz, 1H), 8.21 (d, J = 4.3 Hz, 1H). |
| I-464 | 1H-NMR (CDCl3) δ: 1.34 (d, J = 6.8 Hz, 3H), 2.02 (s, 3H), 4.74 (m, 1H), 5.45 (d, J = 7.9 Hz, 1H), |
| | 6.11 (dd, J = 16.1, 5.7 Hz, 1H), 6.45 (d, J = 16.1 Hz, 1H), 6.94-6.99 (m, 2H), 7.13 (d, J = 2.4 Hz, |
| | 1H), 7.20 (d, J = 8.8 Hz, 1H), 7.30-7.39 (m, 2H), 7.10 (dd, J = 8.8, 2.8 Hz, 1H), 7.23 (d, J = 8.8 Hz, |
| | 1H), 7.29 (d, J = 2.8 Hz, 1H), 7.70-7.76 (m, 2H), 7.76 (dd, J = 8.5, 1.8 Hz, 1H), 8.07 (s, 1H), 8.42- |
| | 8.47 (m, 2H). |
| I-465 | 1H-NMR (CDCl3) δ: 1.34 (d, J = 6.9 Hz, 3H), 2.03 (s, 3H), 4.75 (m, 1H), 5.49 (d, J = 7.9 Hz, 1H), |
| | 6.12 (dd, J = 16.1, 5.7 Hz, 1H), 6.46 (d, J = 16.1 Hz, 1H), 6.91 (d, J = 5.8 Hz, 2H), 6.98 (d, J = 8.5 |
| | Hz, 1H), 7.06 (dd, J = 8.5, 2.5 Hz, 1H), 7.23-7.27 (m, 2H), 7.78 (dd, J = 8.5, 2.0 Hz, 1H), 8.08 (m, |
| | 1H), 8.52 (d, J = 5.4 Hz, 1H). |
| I-466 | 1H-NMR (CDCl3) δ: 1.34 (d, J = 6.8 Hz, 3H), 2.02 (s, 3H), 2.62-2.69 (m, 2H), 3.08 (brs, 2H), 4.74 |
| | (m, 1H), 5.42 (d, J = 7.8 Hz, 1H), 6.11 (dd, J = 16.1, 5.8 Hz, 1H), 6.45 (d, J = 16.1 Hz, 1H), 6.97 (d, |
| | J = 8.5 Hz, 1H), 7.19 (d, J = 8.0 Hz, 1H), 7.48 (d, J = 8.0 Hz, 1H), 7.77 (brd, J = 8.5 Hz, 1H), 8.07 |
| | (s, 1H). |
| I-467 | 1H-NMR (CDCl3) δ: 0.84 (t, J = 7.4 Hz, 6H), 1.34 (d, J = 6.8 Hz, 3H), 1.79-2.08 (m, 4H), 2.02 (s, |
| | 3H), 4.69-4.80 (m, 1H), 5.46 (d, J = 7.9 Hz, 1H), 6.11 (dd, J = 5.7, 16.0 Hz, 1H), 6.45 (d, J = 15.8 |
| | Hz, 1H), 6.91 (d, J =8.5 Hz, 1H), 7.17-7.20 (m, 2H), 7.38-7.41 (m, 1H), 7.75 (dd, J = 2.4, 8.6 Hz, |
| | 1H), 8.09 (d, J = 2.3 Hz, 1H). |
| I-468 | 1H-NMR (CDCl3) δ: 0.30 (d, J = 4.8 Hz, 2H), 0.53 (d, J = 7.8 Hz, 2H), 1.34 (d, J = 6.8 Hz, 3H), |
| | 1.70 (s, 1H), 2.03 (s, 1H), 3.00 (t, J = 6.5 Hz, 1H), 3.47 (d, J = 6.9 Hz, 2H), 3.67 (t, J = 6.5 Hz, 2H), |
| | 4.76 (t, J = 6.8 Hz, 1H), 5.58 (d, J = 7.8 Hz, 1H), 6.13 (dd, J = 16.1, 5.6 Hz, 1H), 6.46 (d, J = 16.1 |
| | Hz, 1H), 6.98 (dd, J = 50.3, 9.0 Hz, 2H), 7.75 (d, J = 8.4 Hz, 1H), 8.12 (t, J = 7.8 Hz, 1H), 8.14 (s, 1H) |
| I-469 | 1H-NMR (CDCl3) δ: 0.99 (t, J = 7.3 Hz, 3H), 1.33 (d, J = 6.8 Hz, 3H), 1.67 (dd, J = 14.5, 8.1 Hz, |
| | 3H), 2.03 (d, 3 = 14.7 Hz, 3H), 2.75 (t, J = 7.7 Hz, 2H), 4.73 (dd, J = 13.7, 6.8 Hz, 1H), 5.51 (d, J = |
| | 7.8 Hz, 1H), 6.10 (dd, J = 15.9, 5.6 Hz, 1H), 6.44 (d, J = 16.1 Hz, 1H), 6.90 (d, J = 8.7 Hz, 1H), |
| | 7.05 (d, J = 5.6 Hz, 1H), 7.12 (d, J = 5.6 Hz, 1H), 7.15 (dd, J = 21.2, 7.4 Hz, 2H), 7.74 (d, J = 8.2 |
| | Hz, 1H), 8.07 (s, 1H). |
| I-470 | 1H-NMR (CDCl3) δ : 0.94 (t, J = 7.4 Hz, 3H), 1.24-1.35 (m, 4H), 1.63 (td, J = 14.9, 7.6 Hz, 4H), |
| | 2.04 ( t, J = 7.4 Hz, 3H), 2.56 (t, J = 7.7 Hz, 2H), 4.75 (dd, J = 13.6, 6.8 Hz, 1H), 5.47 (d, J = 8.0 |
| | Hz, 1H), 6.12 (dd, J = 16.1, 5.8 Hz, 1H), 6.46 (d, J = 16.1 Hz, 1H), 6.83(t, J =2.3 Hz, 1H), 7.97 (d, |
| | J = 15.1 Hz, 1H), 7.73 (dd, J = 8.5, 2.3 Hz, 1H), 8.14 (d, J = 2.3 Hz, 1H). |

TABLE 74

| | |
|---|---|
| I-471 | 1H-NMR (CDCl3) δ: 1.39-1.43 (m, 6H), 2.02 (s, 3H), 4.02 (q, J = 6.9 Hz, 2H), 4.74-4.87 (m, 1H), |
| | 5.62-5.72 (m, 2H), 6.84 (dd, J = 8.8, 2.5 Hz, 1H), 6.90 (d, J = 8.5 Hz, 1H), 6.99 (d, J = 2.6 Hz, 1H), |
| | 7.11 (d, J = 8.8 Hz, 1H), 7.89 (dd, J = 8.5, 2.0 Hz, 1H), 8.16 (brs, 1H). |
| I-473 | 1H-NMR (CDCl3) δ: 1.04 (t, J = 7.4 Hz, 3H), 1.36 (d, J = 7.0 Hz, 3H), 2.02 (s, 3H), 2.08-2.25 |
| | (m, 2H), 4.75-4.86 (m, 1H), 5.48 (d, J = 7.8 Hz, 1H), 6.55 (d, J = 15.6 Hz, 1H), 6.69 (dd, J = 15.7, |
| | 5.6 Hz, 1H), 7.28 (d, J = 8.8 Hz, 1H), 7.43 (d, J = 8.5 Hz, 1H), 7.60 (s, 1H), 7.96 (s, 1H), 8.43 (s, 1H). |
| I-475 | 1H-NMR (CDCl3) δ: 0.94 (t, J = 7.3 Hz, 3H), 1.30 (t, J = 15.7 Hz, 3H), 1.63 (td, J = 14.9, 7.2 Hz, |
| | 3H), 2.03 (d, J = 13.6 Hz, 2H), 2.57 (t, J = 7.7 Hz, 2H), 4.73 (dd, J = 13.6, 6.8 Hz, 1H), 5.48 (d, |
| | J = 7.8 Hz, 1H), 6.10 ( dd, J = 16.1, 5.8 Hz, 1H), 6.44 (d, J = 16.1 Hz, 1H), 6.94 (d, J =8 .3 Hz, 1H), |
| | 7.31 (d, J = 5.6 Hz, 1H), 7.73 (dd, J = 8.5, 2.3 Hz, 1H), 8.08 (d, J = 2.3 Hz, 1H). |
| I-476 | 1H-NMR (CDCl3) δ: 1.01 (t, J = 7.5 Hz, 3H), 1.33 (d, J = 6.8 Hz, 3H), 1.80 (d, J = 6.9 Hz, 3H), |
| | 2.02 (s, 3H), 2.49 (q, J = 7.4 Hz, 2H), 4.68-4.80 (m, 1H), 5.45 (d, J = 7.9 Hz, 1H), 5.76 (q, J = |
| | 6.8 Hz, 1H), 6.10 (dd, J = 5.6, 16.1 Hz, 1H), 6.44 (d, J = 15.9 Hz, 1H), 6.91 (d, J = 8.5 Hz, 1H), 7.12 |
| | (d, J = 8.3 Hz, 1H), 7.24-7.30 (m, 1H), 7.43 (d, J = 1.8 Hz, 1H), 7.74 (dd, J = 2.0, 8.5 Hz, 1H), 8.09 |
| | (d, J =1.8 Hz, 1H). |
| I-477 | 1H-NMR (CDCl3) δ: 1.33 (d, J = 6.8 Hz, 3H), 1.49 (d, J = 6.9 Hz, 6H), 2.02 (s, 3H), 3.39-3.46 |
| | (m, 1H), 4.74 (d, J = 7.3 Hz, 1H), 5.44 (d, J = 9.3 Hz, 1H), 6.11 (dd, J = 16.0, 5.6 Hz, 1H), 6.44 (d, |
| | J = 16.1 Hz, 1H), 6.98 (d, J = 8.7 Hz, 1H), 7.31 (d, J = 8.8 Hz, 1H), 7.77 (d, J = 8.8 Hz, 1H), 7.90 (d, |
| | J = 8.7 Hz, 1H), 8.05 (s, 1H). |
| I-478 | 1H-NMR (CDCl3) δ: 1.33 (d, J = 6.8 Hz, 3H), 2.01 (s, 3H), 2.83 (s, 3H), 4.74 (dd, J = 14.1, 6.8 Hz, 1H), |
| | 5.41 (d, J = 8.0 Hz, 1H), 6.11 (dd, J = 15.9, 5.4 Hz, 1H), 6.44 (d, J = 16.1 Hz, 1H), |
| | 6.98 (d, J = 8.5 Hz, 1H), 7.66 (s, 1H), 7.77 (d, J = 8.8 Hz, 1H), 8.05 (m, 2H). |
| I-479 | 1H-NMR (CDCl3) δ: 1.33 (d, J = 6.8 Hz, 3H), 2.01 (s, 3H), 3.21 (t, J = 8.7 Hz, 2H), 4.62 (t, J = |
| | 8.5 Hz, 2H), 4.74 (m, 1H), 5.43 (d, J = 7.5 Hz, 1H), 6.09 (dd, J = 16.1, 5.8 Hz, 1H), 6.44 (d, J = 16.1 |
| | Hz, 1H), 6.87-6.90 (m, 2H), 7.03 (s, 1H), 7.72 (d, J = 8.5 Hz, 1H), 8.08 (s, 1H). |
| I-480 | 1H-NMR (CDCl3) δ: 1.23 (dd, J = 20.6, 6.1 Hz, 3H), 2.03 (d, J = 8.5 Hz, 2H), 2.98 (t, J = 6.5 Hz, |
| | 1H), 3.55-3.65 (m, 2H), 4.75 (dd, J = 13.7, 6.9 Hz, 1H), 5.61 (d, J = 7.8 Hz, 1H), 6.13 (dd, J = 16.1, |
| | 5.8 Hz, 1H), 6.46 (d, J = 16.1 Hz, 1H), 6.91 (d, J = 9.0 Hz, 1H), 7.03 (d, 3 = 8.5 Hz, 1H), 7.74 (d, J = |
| | 8.5 Hz, 1H), 8.11 (t, J = 9.8 Hz, 1H). |
| I-481 | 1H-NMR (CDCl3) δ: 1.31 (dd, J = 31.4, 7.0 Hz, 2H), 1.63 (s, 1H), 2.04 (t, J = 7.0 Hz, 2H), 4.75 |
| | (dd, J = 13.6, 6.8 Hz, 1H), 5.48 (d, J = 7.5 Hz, 1H), 6.14 (dd, J = 16.1, 5.5 Hz, 1H), 6.46 (d, J = |
| | 16.1 Hz, 1H), 6.97 (d, J = 8.5 Hz, 1H), 7.39 (d, J = 8.8 Hz, 1H), 7.77 (t, J = 7.2 Hz, 1H), 8.16 (t, J = |
| | 18.8 Hz, 1H). |
| I-483 | 1H-NMR (CDCl3) δ: 1.34 (d, J = 6.9 Hz, 3H), 2.03 (s, 3H), 4.72-4.77 (m, 1H), 5.48 (d, J = 7.7 Hz, |
| | 1H), 6.12 (dd, J = 16.0, 5.6 Hz, 1H), 6.44 (d, J = 16.1 Hz, 1H), 6.83-6.91 (m, J = 22.6, 9.0 Hz, 3H), |
| | 7.06 (d, J = 8.7 Hz, 1H), 7.75 (d, J = 8.7 Hz, 1H), 8.09 (s, 1H). |

TABLE 75

| | |
|---|---|
| I-484 | 1H-NMR (CDCl3) δ: 1.34 (d, J = 6.8 Hz, 3H), 2.02 (s, 3H), 2.59 (s, 3H), 4.70-4.79 (m, 1H), 5.44 |
| | (brd, J = 7.5 Hz, 1H), 6.13 (dd, J = 16.1, 5.5 Hz, 1H), 6.45 (d, J = 16.1 Hz, 1H), 6.96 (d, J = 8.6 Hz, |
| | 1H), 7.77 (dd, J = 8.6, 1.9 Hz, 1H), 8.04 (d, J = 1.9 Hz, 1H), 8.48 (s, 2H). |
| I-485 | 1H-NMR (CDCl3) δ: 0.94 (dd, J = 25.7, 18.4 Hz, 3H), 1.24-1.35 (m, 4H), 1.65 (q, J = 7.5 Hz, 3H), |
| | 2.04 (d, J = 10.7 Hz, 3H), 2.68 (t, J = 7.7 Hz, 2H), 4.74 (dd, J = 13.7, 6.9 Hz, 1H), 5.49 (d, J = 7.8 Hz, |
| | 1H), 6.11 (dd, J = 16.0, 5.6 Hz, 1H), 6.45 (d, J = 16.1 Hz, 1H), 6.92 (d, J = 8.3 Hz, 1H), 7.31 |
| | (t, J = 17.1 Hz, 1H), 7.73 (d, J = 8.5 Hz, 1H), 8.12 ( s, 1H). |
| I-486 | 1H-NMR (CDCl3) δ: 1.24-1.35 (m, 4H), 2.04 (t, J = 6.8 Hz, 3H), 2.74 (s, 3H), 4.75 (dd, J = 13.7, |
| | 6.8 Hz, 1H), 5.56 (d, J = 8.0 Hz, 1H), 6.14 (dd, J = 16.1, 5.6 Hz, 1H), 6.46 (d, J = 16.1 Hz, 1H), |
| | 6.96 (d, J = 8.5 Hz, 1H), 7.64 (s, 1H), 7.77 (dd, J = 8.5, 1.9 Hz, 1H), 7.92 (d, J = 8.7 Hz, 1H), 8.12 |
| | (d, J = 1.6 Hz, 1H). |
| I-488 | 1H-NMR (CDCl3) δ: 1.33 (d, J = 6.8 Hz, 3H), 1.47 (d, J = 6.9 Hz, 6H), 2.02 (s, 3H), 3.38-3.43 (dd, |
| | J = 13.8, 6.9 Hz, 1H), 4.72-4.77 (m, 1H), 5.42 (d, J = 7.4 Hz, 1H), 6.11 (dd, J = 16.2, 5.6 Hz, 1H), |
| | 6.44 (d, J = 15.9 Hz, 1H), 6.98 (d, J = 8.5 Hz, 1H), 7.69 (s, 1H), 7.77 (d, J = 8 .0 Hz, 1H), 8.06 |
| | (d, J = 7.3 Hz, 2H). |
| I-489 | 1H-NMR (CDCl3) δ: 1.35 (d, J = 6.5 Hz, 3H), 1.43 (t, J = 6.9 Hz, 3H), 2.00 (s, 3H), 4.02 (q, J = 6.9 |
| | Hz, 2H), 4.74-4.85 (m, 1H), 5.46 (d, J = 7.5 Hz, 1H), 6.53-6.60 (m, 2H), 6.81 (d, J = 9.0 Hz, 1H), |
| | 6.97-7.04 (m, 2H), 7.06 (d, J = 8.3 Hz, 1H), 7.18 (d, J = 8.5 Hz, 1H), 8.26 (s, 1H). |
| I-490 | 1H-NMR (CDCl3) δ: 0.36 (q, J = 5.5 Hz, 2H), 0.67 (q, J = 5.5 Hz, 2H), 1.21-1.31 (m, 1H), 1.34 (d, |
| | J = 6.8 Hz, 3H), 2.00 (s, 3H), 3.79 (d, J = 6.8 Hz, 2H), 4.74-4.84 (m, 1H), 5.47 (d, J = 8.0 Hz, 1H), |
| | 6.54-6.58 (m, 2H), 6.82 (d, J = 9.0 Hz, 1H), 6.98-7.03 (m, 2H), 7.06 (d, J = 8.8 Hz, 1H), 7.18 (d, J = |
| | 8.5 Hz, 1H), 8.26 (s, 1H). |
| I-491 | 1H-NMR (CDCl3) δ: 1.34 (d, J = 6.8 Hz, 3H), 2.04 (d, J = 11.8 Hz, 3H), 4.74 (dd, J = 13.7, 6.8 Hz, |
| | 1H), 5.46 (d, J = 7.8 Hz, 1H), 6.13 (dd, J = 16.1, 5.6 Hz, 1H), 6.45 (d, J = 16.1 Hz, 1H), 7.04 (d, J = |
| | 8.5 Hz, 1H), 7.48 (d, J = 8.9 Hz, 1H), 7.81 (d, J = 8.5 Hz, 1H), 8.03 (s, 1H), 8.14 (d, J = 4.1 Hz, |
| | 1H). |
| 1-492 | 1H-NMR (CDCl3) δ: 0.36-0.40 (m, 2H), 0.60-0.65 (m, 2H), 1.30-1.41 (m, 4H), 2.02 (s, 3H), 4.20 |
| | (d, J = 7.3 Hz, 2H), 4.70-4.78 (m, 1H), 5.43 (brd, J = 8.0 Hz, 1H), 6.12 (dd, J = 16.0, 5.6 Hz, 1H), |
| | 6.45 (d, J = 16.0 Hz, 1H), 6.95 (d, J =8.4 Hz, 1H), 7.76 (dd, J = 8.4, 2.0 Hz, 1H), 8.03 (d, J = 2.0 |
| | Hz, 1H), 8.41 (s, 2H). |
| I-493 | 1H-NMR (CDCl3) δ: 1.34 (d, J = 6.5 Hz, 3H), 2.02 (s, 3H), 4.68-4.82 (m, 1H), 5.46 (d, J = 6.8 Hz, |
| | 1H), 6.13 (dd, J = 15.8, 5.3 Hz, 1H), 6.46 (d, J = 16.1 Hz, 1H), 6.92 (d, J = 8.3 Hz, 1H), 7.22 (d, J = |
| | 8.3 Hz, 2H), 7.75 (d, J = 7.8 Hz, 1H), 8.16 (s, 1H), 8.43 (d, J = 8.3 Hz, 2H), 8.64 (s, 2H). |
| I-494 | 1H-NMR (CDCl3) δ: 0.76 (dd, J = 13.7, 5.1 Hz, 1H), 0.87 (td, J = 9.5, 5.2 Hz, 1H), 1.05 (s, 1H), |
| | 1.18 (d, J = 5.8 Hz, 3H), 1.32 (d, J = 6.8 Hz, 3H), 1.53-1.58 (m, 1H), 2.00 (s, 3H), 4.73 (d, J = 6.8 |
| | Hz, 1H), 5.52 (s, 1H), 6.08 (dd, J = 15.9, 5.6 Hz, 1H), 6.43 (d, J = 16.1 Hz, 1H), 6.88 (d, J = 8.5 Hz, |
| | 1H), 6.96 (d, J = 8.3 Hz, 1H), 7.06 (d, J = 8.3 Hz, 1H), 7.11 (s, 1H), 7.71 (d, J = 8.5 Hz, 1H), 8.06 |
| | (s, 1H). |

TABLE 76

| | |
|---|---|
| I-495 | 1H-NMR (CDCl3) δ: 1.34 (d, J = 6.8 Hz, 3H), 1.44 (t, J = 7.5 Hz, 3H), 2.03 (d, J = 10.0 Hz, 3H), |
| | 2.96 (q, J = 7.5 Hz, 2H), 4.74 (d, J = 6.8 Hz, 1H), 5.45 (s, 1H), 6.11 (dd, J = 16.1, 5.8 Hz, 1H), 6.45 |
| | (d, J = 16.1 Hz, 1H), 6.89 (d, J = 8.5 Hz, 1H), 7.08 (d, J = 8.5 Hz, 1H), 7.29 (s, 1H), 7.65 (d, J = 8.5 Hz, |
| | 1H), 7.73 (d, J = 8.5 Hz, 1H), 8.11 (s, 1H). |
| I-496 | 1H-NMR (CDCl3) δ: .0.95 (t, J = 7.7 Hz, 3H), 1.35 (d, J = 6.8 Hz, 3H), 1.65 (sex, J = 7.7 Hz, 2H), |
| | 2.01 (s, 3H), 4.74-4.84 (m, 1H), 5.49 (d, J = 7.8 Hz, 1H), 6.55-6.61 (m, 2H), 6.95 (d, J = 8.0 Hz, 1H), |
| | 7.06 (d, J = 8.3 Hz, 1H), 7.12 (d, J = 8.5 Hz, 1H), 7.21 (d, J = 8.8 Hz, 1H), 7.29 (s, 1H), 8.29 |
| | (s, 1H). |
| I-497 | 1H-NMR (CDCl3) δ: 1.34-1.36 (m, 12H), 2.02 (s, 3H), 4.78-4.83 (m, 1H), 5.49 (d, J =7.9 Hz, 1H), |
| | 6.54 (d, J = 15.7 Hz, 1H), 6.67 (dd, J = 15.7, 5.6 Hz, 1H), 7.15 (d, J = 8.5 Hz, 1H), 7.34 (d, J = 8.4 Hz, |
| | 1H), 7.47 (s, 1H), 7.97 (s, 1H), 8.40 (s, 1H). |
| I-499 | 1H-NMR (CDCl3) δ: 1.34 (d, J = 7.0 Hz, 3H), 1.52-1.71 (m, 6H), 2.02 (s, 3H), 3.75-3.80 (m, 4H), |
| | 4.70-4.78 (m, 1H), 5.42 (brd, J = 8.3 Hz, 1H), 6.10 (dd, J = 16.1, 5.5 Hz, 1H), 6.44 (d, J = 16.1 Hz, |
| | 1H), 6.89 (d, J = 8.6 Hz, 1H), 7.72 (dd, J = 8.6, 1.6 Hz, 1H), 8.05 (d, J = 1.6 Hz, 1H), 8.21 (s, 2H). |
| I-500 | 1H-NMR (CDCl3) δ: 1.34 (d, J = 6.8 Hz, 3H), 1.45 (d, J = 6.8 Hz, 6H), 2.03 (d, J = 10.8 Hz, 3H), |
| | 4.74 (d, J = 7.0 Hz, 1H), 5.48 (s, 1H), 6.11 (dd, J = 16.1, 5.8 Hz, 1H), 6.45 (d, J = 16.1 Hz, 1H), |
| | 6.90 (d, J = 8.5 Hz, 1H), 7.08 (d, J = 8.5 Hz, 1H), 7.30 (s, 1H), 7.66 (d, J = 8.5 Hz, 1H), 7.74 (d, J = |
| | 8.5 Hz, 1H), 8.11 (s, 1H). |
| I-502 | 1H-NMR (CDCl3) δ: 1.33 (d, J = 6.8 Hz, 3H), 1.53 (s, 9H), 1.65 (s, 2H), 2.02 (s, 3H), 4.74 (dd, J = |
| | 13.7, 6.9 Hz, 1H), 5.48 (d, J = 8.0 Hz, 1H), 6.11 (dd, J = 16.0, 5.6 Hz, 1H), 6.44 (d, J = 16.1 Hz, |
| | 1H), 6.97 (d, J = 8.5 Hz, 1H), 7.29 (d, J = 8.5 Hz, 1H), 7.76 (d, J = 8.5 Hz, 1H), 7.91 (d, J = 8.7 Hz, |
| | 1H), 8.04 (s, 1H). |
| I-503 | 1H-NMR (CDCl3) δ: 1.35 (d, J = 6.8 Hz, 3H), 2.03 (s, 3H), 4.72-4.80 (m, 1H), 5.43 (brd, J = 7.5 |
| | Hz, 1H), 6.14 (dd, J =15.8, 5.5 Hz, 1H), 6.47 (d, J =15.8 Hz, 1H), 6.94 (d, J =8.3 Hz, 1H), 7.09- |
| | 7.22 (m, 1H), 7.35-7.43 (m, 6H), 7.75-7.78 (m, 1H), 8.16 (s, 1H). |
| I-504 | 1H-NMR (CDCl3) δ: 1.34 (d, J = 6.8 Hz, 3H), 1.54 (d, J = 22.6 Hz, 9H), 1.65 (s, 2H), 2.03 (d, J = 14.1 Hz, |
| | 3H), 4.74 (d, J = 6.5 Hz, 1H), 5.48 (d, J = 7.3 Hz, 1H), 6.11 (dd, J = 15.9, 5.4 Hz, 1H), |
| | 6.45 (d, J = 16.1 Hz, 1H), 6.89 (d, J = 8.3 Hz, 1H), 7.21(d, J = 8.3 Hz, 1H), 7.61 (s, 1H), 7.73 (d, J = 83 Hz, |
| | 1H), 7.98 (d, J = 8.8 Hz, 1H), 8.11 (s, 1H). |
| I-505 | 1H-NMR (CDCl3) δ: 1.37 (d, J = 6.8 Hz, 3H), 1.38 (t, J = 7.0 Hz, 3H), 2.02 (s, 3H), 3.97 (q, J = 6.9 Hz, |
| | 2H), 4.77-4.88 (m, 1H), 5.19 (s, 2H), 5.49 (d, J = 7.0 Hz, 1H), 6.59 (d, J = 15.8 Hz, 1H), 6.66-6.75 |
| | (m, 2H), 6.88 (d, J = 9.0 Hz, 1H), 6.98 (d, J = 1.8 Hz, 1H), 7.21 (d, J = 7.8 Hz, 1H), 7.48 (d, J = 7.5 Hz, |
| | 1H), 7.67 (t, J = 7.7 Hz, 1H). |
| I-506 | 1H-NMR (CDCl3) δ: 1.34 (d, J = 6.8 Hz, 3H), 1.38 (t, J = 6.8 Hz, 3H), 2.01 (s, 3H), 3.96 (q, J = 6.4 Hz, |
| | 2H), 4.71-4.80 (m, 1H), 5.06 (s, 2H), 5.47 (d, J = 7.0 Hz, 1H), 6.19 (dd, J = 15.9, 5.1 Hz, 1H), |
| | 6.52 (d, J = 16.1 Hz, 1H), 6.71 (d, J = 8.5 Hz, 1H), 6.87 (d, J = 8.5 Hz, 1H), 6.96 (s, 1H), 7.28-7.36 |
| | (m, 3H), 7.45 (s, 1H). |

TABLE 77

| | |
|---|---|
| I-507 | 1H-NMR (CDCl3) δ: 1.33 (d, J = 6.8 Hz, 3H), 1.49 (s, 3H), 2.02 (s, 3H), 3.01 (s, 2H), 4.71-4.77 |
| | (m, 1H), 5.43 (d, J = 7.4 Hz, 1H), 6.09 (dd, J = 16.1, 5.6 Hz, 1H), 6.44 (d, J = 16.1 Hz, 1H), 6.81 |
| | (s, 1H), 6.88 (d, J = 8.5 Hz, 1H), 6.98 (s, 1H), 7.73 (d, J = 8.8 Hz, 1H), 8.08 (s, 1H). |
| I-508 | 1H-NMR (CDCl3) δ: 1.35 (d, J = 6.9 Hz, 3H), 1.49 (s, 6H), 2.02 (s, 3H), 3.02 (s, 2H), 4.77-4.83 |
| | (m, 1H), 5.48 (d, J = 8.2 Hz, 1H), 6.54 (d, J = 15.7 Hz, 1H), 6.66 (dd, J = 15.7, 5.5 Hz, 1H), 6.82 |
| | (s, 1H), 6.99 (s, 1H), 7.97 (s, 1H), 8.37 (s, 1H). |
| I-509 | 1H-NMR (CDCl3) δ: 1.35 (d, J = 6.8 Hz, 3H), 2.03 (s, 3H), 2.89 (s, 3H), 4.73-4.78 (t, J = 9.9 Hz, |
| | 1H), 5.47 (d, J = 8.0 Hz, 1H), 6.14 (dd, J = 16.0, 5.6 Hz, 1H), 6.46 (d, J = 16.1 Hz, 1H), 7.09 |
| | (d, J = 8.5 Hz, 1H), 7.33 (d, J = 8.9 Hz, 1H), 7.82 (d, J = 8.7 Hz, 1H), 8.06 (s, 1H), 8.13 |
| | (d, J = 8.9 Hz, 1H). |
| I-510 | 1H-NMR (CDCl3) δ: 1.36 (d, J = 6.8 Hz, 3H), 1.63 (s, 1H), 2.03 (s, 3H), 4.81 (dd, J = 13.7, 6.9 Hz, |
| | 1H), 5.51 (d, J = 7.8 Hz, 1H), 6.56 (d, J = 15.6 Hz, 1H), 6.71 (dd, J = 15.7, 5.5 Hz, 1H), 7.50 |
| | (d, J = 8.9 Hz, 1H), 7.93 (s, 1H), 8.17 (d, J = 8.8 Hz, 1H), 8.51 (s, 1H). |
| I-511 | 1H-NMR (CDCl3) δ: 0.31 (d, J = 4.5 Hz, 2H), 0.55 (d, J = 7.7 Hz, 2H), 1.07 (d, J = 6.8 Hz, 1H), |
| | 1.10 (d, J = 6.3 Hz, 1H), 1.34 (d, J = 6.8 Hz, 3H), 1.67 (s, 3H), 2.02 (s, 3H), 3.16 (t, J = 5.6 Hz, |
| | 1H), 3.48 (d, J = 6.9 Hz, 1H), 3.70 (t, J = 6.6 Hz, 1H), 4.74 (dd, J = 13.4, 6.8 Hz, 1H), 5.53 (d, J = |
| | 7.9 Hz, 1H), 6.12 (dd, J = 16.1, 5.5 Hz, 1H), 6.45 (d, J =15.9 Hz, 1H), 6.98 (d, J =8.5 Hz, 1H), |
| | 7.15 (t, J = 9.3 Hz, 1H), 7.77 (d, J = 8.4 Hz, 1H), 8.07 (s, 1H), 8.11 (d, J = 8.4 Hz, 1H). |
| I-512 | 1H-NMR (CDCl3) δ: 0.92 (s, 9H), 1.35 (d, J = 6.8 Hz, 3H), 2.01 (s, 3H), 2.47 (s, 2H), 4.74-4.84 |
| | (m, 1H), 5.51 (d, J = 8.0 Hz, 1H), 6.54-6.62 (m, 2H), 6.94 (d, J = 8.3 Hz, 1H), 7.01 (d, J = 8.3 Hz, |
| | 1H), 7.14 (d, J = 8.4 Hz, 1H), 7.22 (d, J = 8.4 Hz, 2H), 7.23 (s, 1H), 8.27 (s, 1H). |
| I-513 | 1H-NMR (CDCl3) δ: 0.33-0.41 (m, 4H), 1.35 (d, J = 6.8 Hz, 3H), 1.56 (t, J = 6.0 Hz, 2H), 2.01 |
| | (s, 3H), 2.11 (br s, 2H), 2.47 (br s, 2H), 4.74-4.85 (m, 1H), 5.48 (d, J = 7.8 Hz, 1H), 6.14 (br s, 1H), |
| | 6.55-6.61 (m, 2H), 6.97 (d, J = 8.5 Hz, 1H), 7.14 (d, J = 8.5 Hz, 1H), 7.21 (d, J = 8.8 Hz, 1H), 7.29 |
| | (d, J = 8.5 Hz, 1H), 7.51 (s, 1H), 8.31 (s, 1H). |
| I--514 | 1H-NMR (CDCl3) δ: 1.34 (d, J = 6.0 Hz, 3H), 2.02 (s, 3H), 4.68-4.81 (m, 1H), 5.47 (d, J = 7.8 Hz, |
| | 1H), 6.12 (dd, J = 15.6, 4.8 Hz, 1H), 6.46 (d, J = 15.6 Hz, 1H), 6.91 (d, J = 8.0 Hz, 1H), 7.05-7.30 |
| | (m, 4H), 7.46-7.63 (m, 4H), 7.74 (d, J = 7.8 Hz, 1H), 8.15 (s, 1H). |
| I--515 | 1H-NMR (CDCl3) δ: 1.35 (d, J = 6.5 Hz, 3H), 2.02 (s, 3H), 4.68-4.81 (m, 1H), 5.47 (d, J = 7.8 Hz, |
| | 1H), 6.13 (dd, J = 15.8, 5.3 Hz, 1H), 6.46 (d, J = 16.1 Hz, 1H), 6.94 (d, J = 8.3 Hz, 1H), 7.24 |
| | (d, J = 8.3 Hz, 2H), 7.62 (d, J = 7.8 Hz, 2H), 7.65-7.81 (m, 5H), 8.14 (s, 1H). |
| I--516 | 1H-NMR (CDCl3) δ: 1.33 (d, J = 6.8 Hz, 3H), 1.52-1.69 (m, 6H), 2.02 (s, 3H), 3.47-3.54 (m, 4H), |
| | 4.69-4.78 (m, 1H), 5.42 (brd, J = 8.3 Hz, 1H), 6.09 (dd, J = 16.2, 5.6 Hz, 1H), 6.43 (d, J = 16.2 Hz, |
| | 1H), 6.68 (d, J = 9.0 Hz, 1H), 6.84 (d, J =8.8 Hz, 1H), 7.26-7.31 (m, 1H), 7.69 (d, J = 8.8 Hz, 1H), |
| | 8.05 (s, 1H), 8.08 (s, 1H). |

TABLE 78

| | |
|---|---|
| I-517 | 1H-NMR (CDCl3) δ: 1.34 (d, J = 6.8 Hz, 3H), 1.96-2.09 (m, 7H), 3.93-4.02 (m, 4H), 4.70-4.78 (m, |
| | 1H), 5.43 (brd, J = 6.5 Hz, 1H), 6.11 (dd, J = 16.3, 5.6 Hz, 1H), 6.44 (d, J = 16.3 Hz, 1H), 6.91 |
| | (d, J = 8.5 Hz, 1H), 7.74 (d, J = 8.5 Hz, 1H), 8.04 (s, 1H), 8.25 (s, 2H). |
| I-518 | 1H-NMR (CDCl3) δ: 0.30 (d, J = 4.8 Hz, 2H), 0.53 (d, J = 7.8 Hz, 2H), 1.09 (dd, J = 32.1, 6.3 Hz, |
| | 1H), 1.33 (d, J = 6.8 Hz, 3H), 1.70 (s, 3H), 2.02 (s, 3H), 3.01 (t, J = 6.5 Hz, 2H), 3.47 (d, J = 7.0 |
| | Hz, 2H), 3.67 (t, J = 6.6 Hz, 2H), 4.74 (dd, J = 13.6, 6.7 Hz, 1H), 5.60 (d, J = 7.8 Hz, 1H), 6.10 (dd, |
| | J = 16.1, 5.6 Hz, 1H), 6.43 (d, J = 16.1 Hz, 1H), 6.89 (d, J = 8.5 Hz, 1H), 7.23 (dd, J = 21.2, 12.8 |
| | Hz, 2H), 7.72 (d, J = 8.5 Hz, 1H), 7.81 (s, 1H), 8.09 (s, 1H). |
| I-519 | 1H-NMR (CDCl3) δ: 1.34 (d, J = 6.8 Hz, 3H), 1.49 (s, 6H), 2.02 (s, 3H), 3.00 (s, 2H), 4.72-4.77 |
| | (dd, J = 13.4, 6.5 Hz, 1H), 5.48 (d, J = 8.4 Hz, 1H), 6.10 (dd, J = 15.9, 5.5 Hz, 1H), 6.44 |
| | (d, J = 15.9 Hz, 1H), 6.50 (s, 1H), 6.57 (d, J = 7.9 Hz, 1H), 6.84 (d, J = 8.7 Hz, 1H), 7.10 |
| | (d, J = 7.9 Hz, 1H), 7.69 (d, J = 8.5 Hz, 1H), 8.13 (s, 1H). |
| I-520 | 1H-NMR (CDCl3) δ: 1.34 (d, J = 6.8 Hz, 3H), 1.88-2.02 (m, 7H), 3.86-3.94 (m, 4H), 4.72-4.77 (m, |
| | 1H), 4.80-4.98 (m, 1H), 5.43 (brd, J = 7.5 Hz, 1H), 6.10 (dd, J = 16.1, 5.5 Hz, 1H), 6.44 (d, J = 16.1 |
| | Hz, 1H), 6.90 (d, J = 8.2 Hz, 1H), 7.73 (d, J = 8.2 Hz, 1H), 8.04 (s, 1H), 8.23 (s, 2H). |

### Example 454

### Preparation of Compound I-454

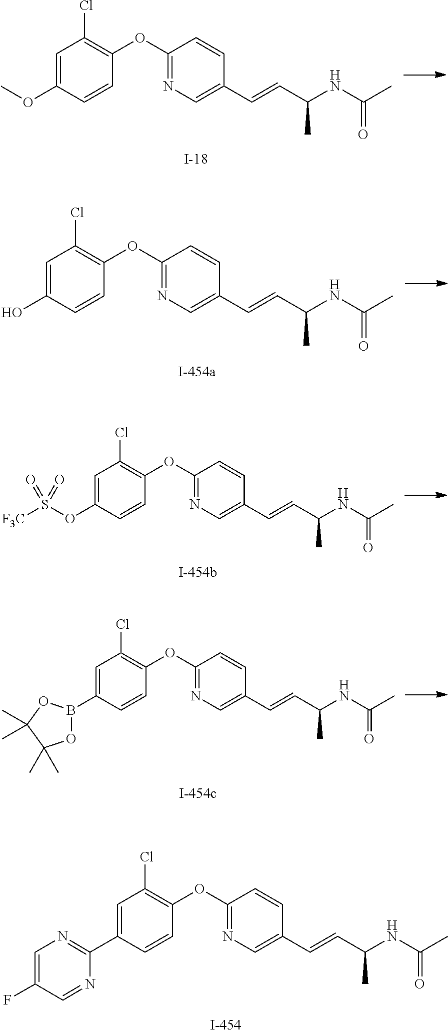

### Step 1 Preparation of Compound I-454

Compound I-18 (2.00 g, 5.77 mmol) was dissolved in dichloromethane (20.0 mL). The dichloromethane solution of 1.00 mol/L boron tribromide (17.3 mL, 17.3 mmol) was added to the mixture at −78° C., and the mixture was stirred at room temperature for 9 hours. Saturated sodium hydrogen carbonate solution was added to the mixture. The mixture was extracted with chloroform. The organic layer was washed with water and brine, dried over magnesium sulfate. The solvent was condensed under reduced pressure. The residue was purified by silica gel chromatography (chloroform-methanol) to afford Compound I-454a (1.54 g, yield 80%).

¹H-NMR (DMSO-D6) δ: 1.20 (d, J=6.8 Hz, 3H), 1.83 (s, 3H), 4.44-4.55 (m, 1H), 6.24 (dd, J=16.1, 5.5 Hz, 1H), 6.41 (d, J=16.1 Hz, 1H), 6.77 (dd, J=8.8, 2.8 Hz, 1H), 6.90 (d, J=2.8 Hz, 1H), 6.97 (d, J=8.5 Hz, 1H), 7.10 (d, J=8.8 Hz, 1H), 7.95 (dd, J=8.7, 2.4 Hz, 1H), 7.99 (d, J=8.0 Hz, 1H), 8.07 (d, J=2.3 Hz, 1H), 9.84 (s, 1H).

[M+H]=333, Method Condition 2: retention time 1.52 min

### Step 2 Preparation of Compound I-454b

Compound I-454a (0.780 g, 2.34 mmol) and 1,1,1-trifluoro-N-phenyl-N-(trifluoromethylsulfonyl)methanesulfone amide (1.26 g, 3.52 mmol) was dissolved in dichloromethane. Trietnylamine (0.650 mL, 4.69 mmol) was added to the mixture, and the mixture was stirred overnight at room temperature. Water was added to the mixture, and the mixture was extracted with ethyl acetate. The organic layer was washed with water and brine, dried over magnesium sulfate. The solvent was condensed under reduced pressure. The residue was purified by silica gel chromatography (hexane-ethyl acetate) to afford Compound I-454b (1.12 g, yield 100%).

¹H-NMR (CDCl₃) δ: 1.34 (d, J=6.8 Hz, 3H), 2.02 (s, 3H), 4.69-4.80 (m, 1H), 5.42 (d, J=8.3 Hz, 1H), 6.13 (dd, J=16.1, 5.8 Hz, 1H), 6.45 (d, J=16.1 Hz, 1H), 6.98 (d, J=8.5 Hz, 1H), 7.24 (dd, J=8.8, 2.8 Hz, 1H), 7.29 (d, J=9.0 Hz, 1H), 7.42 (d, J=2.8 Hz, 1H), 7.78 (dd, J=8.5, 2.3 Hz, 1H), 8.05 (d, J=2.5 Hz, 1H).

[M+H]=465, Method Condition 2: retention time 2.29 min

### Step 3 Preparation of Compound I-454c

The DMSO solution (6.00 mL) of Compound I-454b (0.600 g, 1.29 mmol), Bis(pinacolato)diboron (0.393 g, 1.55 mmol), (diphenylphosphino)ferrocene)palladium(II) dichloride dichloromethane complex (0.105 g, 0.129 mmol) and potassium acetate (0.380 g, 3.87 mmol) was reacted at 130° C. for 3 hours. Water was added to the mixture, and the mixture was extracted with ethyl acetate. The organic layer was washed with water and brine, and dried over magnesium sulfate. The solvent was condensed under reduced pressure. The residue was purified by silica gel chromatography (hexane-ethyl acetate) to afford Compound I-454c (0.470 g, yield 82%).

¹H-NMR (CDCl₃) δ: 1.33 (d, J=6.8 Hz, 3H), 1.34 (s, 12H), 2.02 (s, 3H), 4.69-4.79 (m, 1H), 5.46 (d, J=8.3 Hz, 1H), 6.10 (dd, J=16.1, 5.8 Hz, 1H), 6.44 (d, J=15.6 Hz, 1H), 6.92 (d, J=8.5 Hz, 1H), 7.19 (d, J=8.0 Hz, 1H), 7.71-7.77 (m, 2H), 7.92 (d, J=1.5 Hz, 1H), 8.07 (d, J=2.3 Hz, 1H).

[M+H]=443, Method Condition 2: retention time 2.46 min

### Step 4 Preparation of Compound I-454

The mixed solution of dioxane (1.2 mL) and water (0.40 mL) of Compound 8 (0.0040 g, 0.090 mmol), 2-chloro-5-fluoropyrimidine (0.014 g, 0.108 mmol), Tetrakis(triphenylphosphine)palladium (0.010 g, 0.009 mmol) and sodium carbonate (0.0192 g, 0.181 mmol) was reacted at 100° C. for 15 minutes. Water was added to the mixture, and the mixture was extracted with ethyl acetate. The organic layer was washed with water, and dried over magnesium sulfate. The solvent was condensed under reduced pressure to afford 1-454 (0.035 g, yield 95%).

¹H-NMR (CDCl₃) δ: 1.34 (d, J=6.5 Hz, 3H), 2.02 (s, 3H), 4.69-4.80 (m, 1H), 5.44 (d, J=6.8 Hz, 1H), 6.12 (dd, J=16.2, 5.4 Hz, 1H), 6.45 (d, J=15.8 Hz, 1H), 6.98 (d, J=8.5 Hz, 1H), 7.30 (d, J=8.5 Hz, 1H), 7.77 (d, J=8.0 Hz, 1H), 8.09 (s, 1H), 8.35 (d, J=8.0 Hz, 1H), 8.55 (s, 1H), 8.65 (s, 2H).

[M+H]=413, Method Condition 2: retention time 2.14 min

### Example 471

### Preparation of Compound I-471

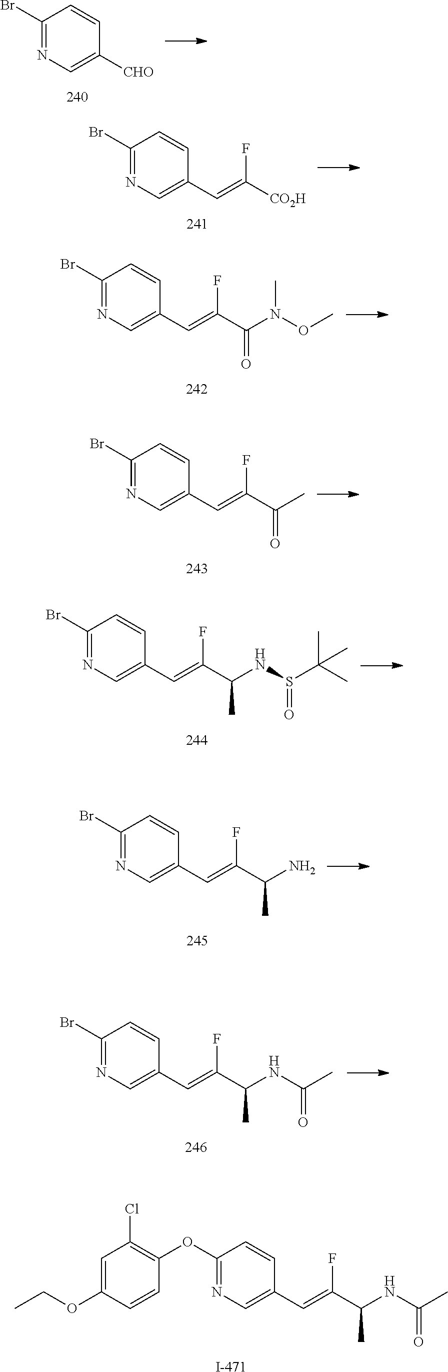

### Step 1 Preparation of Compound 241

THF solution of 0.75 mol/l isopropylmagnesiumbromide (45.9 mL, 34.4 mmol) was added dropwise to THF suspension of the 2-(diethoxyphosphoryl)-2-fluoroacetic acid (3.52 g, 16.4 mmol) while cooling in ice. The mixture was stirred for 1 hour while cooling in ice. THF solution (10.0 mL) of Compound 240 (3.00 g, 15.6 mmol) was added dropwise to the mixture, and the mixture was stirred at 40° C. for 3 hours. hydrochloric acid aqueous solution was added to the mixture, and the mixture was extracted with methyl ethyl ketone. The organic layer was washed with water and brine, and dried over magnesium sulfate. The solvent was condensed under reduced pressure to afford Compound 241 (3.91 g, yield 99%) as crude.

### Step 2 Preparation of Compound 242

Obtained Compound 241 was dissolved in DMF (30.0 mL), and N,O-dimethylhydroxylamine hydrochloride (1.68 g, 17.2 mmol), HATU (6.54 g, 17.2 mmol) and triethylamine (6.51 mL, 46.9 mmol) were added to the mixture, and the mixture was stirred overnight at room temperature. Water was added to the mixture, the mixture was extracted with ethyl acetate. The organic layer was washed with water, dried over magnesium sulfate. The solvent was condensed under reduced pressure. The residue was purified by silica gel chromatography (hexane-ethyl acetate) to afford Compound 242 (1.74 g, yield 39%).

¹H-NMR (CDCl₃) δ: 3.29 (s, 3H), 3.80 (s, 3H), 6.65 (d, J=36.4 Hz, 1H), 7.52 (d, J=8.5 Hz, 1H), 7.84 (dd, J=8.5, 2.1 Hz, 1H), 8.51 (d, J=2.1 Hz, 1H).

[M+H]=289.0, Method Condition 2: retention time 1.69 min

### Step 3 Preparation of Compound 243

Compound 242 (1.74 g, 6.02 mmol) was dissolved in THF (20.0 mL), and the diethylether solution of 3.0 mol/L methylmagnesiumbromide (3.00 mL, 9.00 mmol) was added dropwise while cooling in ice. The mixture was allowed to warm to room temperature, and stirred for 1 hour. The reaction was quenched by adding hydrochloric acid aqueous solution. The mixture wad diluted with water, and extracted with ethyl aceate. The organic layer was washed with water and brine, dried over magnesium sulfate. The solvent was condensed under reduced pressure to afford Compound 243 (1.51 g) as crude.

¹H-NMR (CDCl₃) δ: 2.43 (d, J=3.8 Hz, 3H), 6.76 (d, J=35.6 Hz, 1H), 7.55 (d, J=8.4 Hz, 1H), 7.89 (dd, J=8.4, 2.3 Hz, 1H), 8.56 (d, J=2.3 Hz, 1H).

[M+H]=245.8, Method Condition 2: retention time 1.69 min

### Step 4 Preparation of Compound 244

The obtained Compound 243 was dissolved in THF (20.0 mL). (R)-2-methylpropan-2-sulphinamide (10.0 g, 9.03 mmol) and tetraisopropyloxytitanium (2.73 mL, 9.03 mmol) was added to the mixture, the mixture was stirred overnight under heat refluxing. The mixture was cooled to −78° C., the THF solution of 1.02 mol/L diisobutylaluminum hydride (7.67 mL, 7.82 mmol), the mixture was stirred for 6 hours. Brine was added to the mixture, and the mixture was extracted with ethyl acetate. The organic layer was washed with brine, dried over magnesium sulfate. The solvent was condensed under reduced pressure to afford Compound 244 (2.56 g) as crude.

¹H-NMR (CDCl₃) δ: 1.24 (s, 9H), 1.49 (d, J=6.8 Hz, 3H), 3.48-3.55 (m, 1H), 4.05-4.16 (m, 1H), 5.79 (d, J=38.4 Hz, 1H), 7.44 (d, J=8.3 Hz, 1H), 7.76 (dd, J=8.3, 2.5 Hz, 1H), 8.38 (d, J=2.5 Hz, 1H).

[M+H]=350.7, Method Condition 2: retention time 1.88 min

### Step 5 Preparation of Compound 245

Compound 244 (2.10 g, 6.01 mmol) was dissolved in dichloromethane (8.00 mL). The dioxane solution of 4 mol/L hydrochloric acid was added to the mixture while cooling in ice, and the mixture was stirred for 1.5 hours. Ethyl acetate was added to the mixture, the precipitated solid was filtrated to afford Compound 245 (1.53 g, yield 90%).

### Step 6 Preparation of Compound 246

Compound 245 (2.09 g, 6.00 mmol) was dissolved in dichloromethane (10.0. mL). Pyridine (0.875 mL, 10.8 mmol) and acetic anhydride (0.853 mL, 9.02 mmol) was added to the mixture while cooling in ice, the mixture was stirred for 1.5 hours. Water was added to the mixture, the mixture was extracted with ethyl acetate. The organic layer was washed with water, dried over magnesium sulfate. The solvent was condensed under reduced pressure. Hexane and ethyl acetate were added to the mixture, and the precipitate was filtrated to afford Compound 246 (0.808 g, yield 47%).

¹H-NMR (CDCl₃) δ: 1.42 (d, J=7.0 Hz, 3H), 2.03 (s, 3H), 4.76-4.88 (m, 1H), 5.58-5.74 (m, 2H), 7.44 (d, J=8.3 Hz, 1H), 7.71 (dd, J=8.3, 2.3 Hz, 1H), 8.38 (d, J=2.3 Hz, 1H).

[M+H]=289.0, Method Condition 2: retention time 1.44 min

### Step 7 Preparation of Compound I-471

2-Chloro-4-ethoxyphenol (0.125 g, 0.724 mmol) was dissolved in dioxane (4.00. mL). N, N-dimethylaminoglycine (0.0172 g, 0.167 mmol), Compound 246 (0.160 g, 0.557 mmol), copper(I) iodide (0.0106 g, 0.056 mmol) and cesium carbonate (0.545 g, 1.67 mmol) was added to the mixture. The mixture was stirred under microwave irradiation at 150° C. for 1 hour. Water was added to the mixture, the mixture was extracted with ethyl acetate. The organic layer was washed with water and brine, dried over magnesium sulfate. The solvent was condensed under reduced pressure. The residue was purified by silica gel chromatography (hexane-ethyl acetate) to afford Compound I-471 (0.172 g, yield 82%).

¹H-NMR (CDCl₃) δ: 1.39-1.43 (m, 6H), 2.02 (s, 3H), 4.02 (q, J=6.9 Hz, 2H), 4.74-4.87 (m, 1H), 5.62-5.72 (m, 2H), 6.84 (dd, J=8.8, 2.5 Hz, 1H), 6.90 (d, J=8.5 Hz, 1H), 6.99 (d, J=2.6 Hz, 1H), 7.11 (d, J=8.8 Hz, 1H), 7.89 (dd, J=8.5, 2.0 Hz, 1H), 8.16 (brs, 1H).

[M+H]=379.0, Method Condition 2: retention time 2.16 min

### Example 521

### Preparation of Compound I′-1

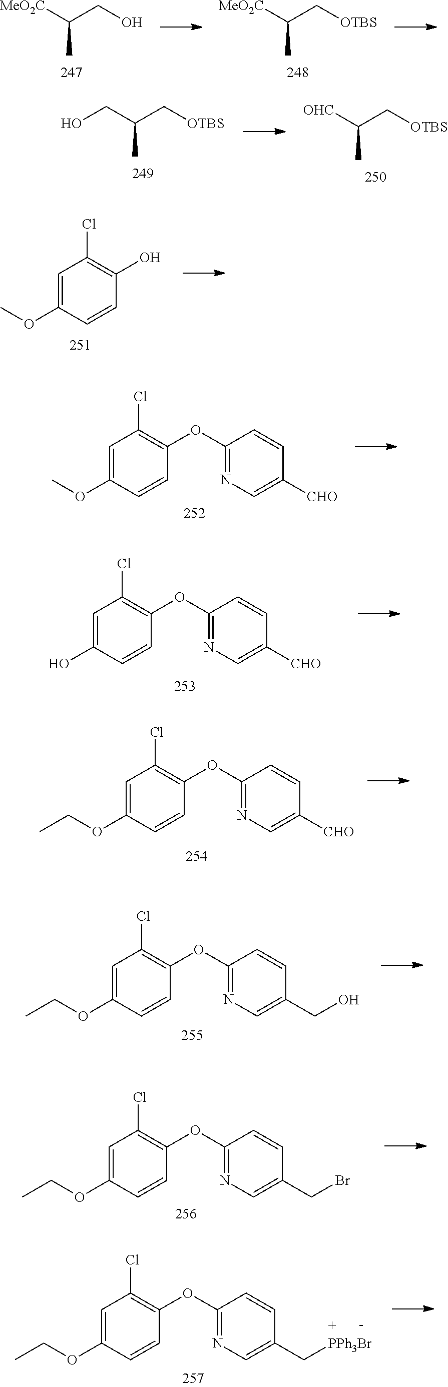

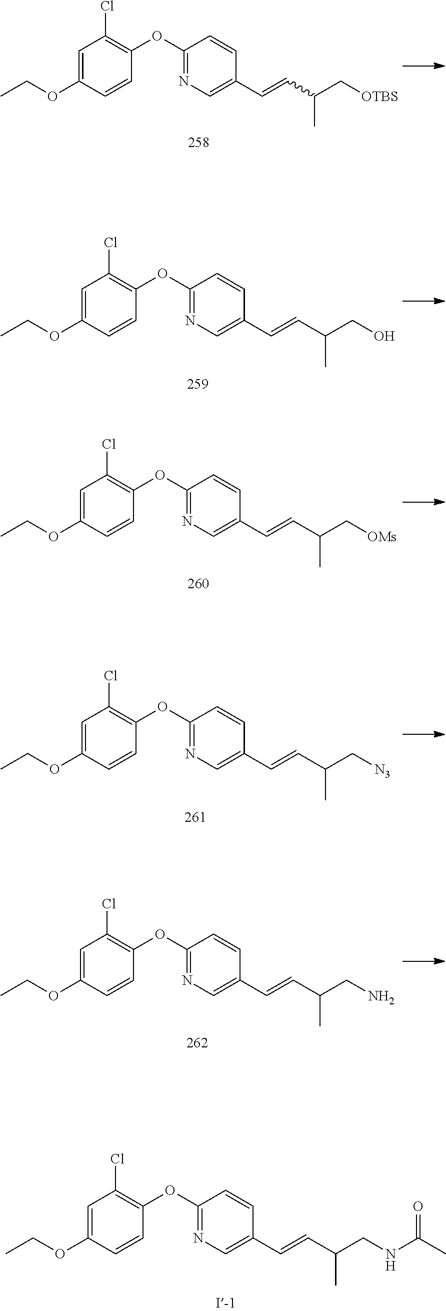 [SMILES: CCOc1ccc(Oc2ccc(C=CC(C)CO[Si](C)(C)C(C)(C)C)cn2)c(Cl)c1 ; CCOc1ccc(Oc2ccc(/C=C/C(C)CO)cn2)c(Cl)c1 ; CCOc1ccc(Oc2ccc(/C=C/C(C)COS(C)(=O)=O)cn2)c(Cl)c1 ; CCOc1ccc(Oc2ccc(/C=C/C(C)CN=[N+]=[N-])cn2)c(Cl)c1 ; CCOc1ccc(Oc2ccc(/C=C/C(C)CN)cn2)c(Cl)c1 ; CCOc1ccc(Oc2ccc(/C=C/C(C)CNC(C)=O)cn2)c(Cl)c1]

### Step 1 Preparation of Compound 248

To dichloromethane (40.0 mL) solution of Compound 247 (2.00 g, 16.9 mmol), tert-butyldimethylsilyl chloride (2.81 g, 18.6 mmol), imidazole (1.73 g, 25.4 mmol) and 4-N, N-dimethylaminopyridine (0.207 g, 1.69 mmol) were added to the mixture. The mixture was stirred overnight at room temperature. Water was added to the mixture, the mixture was extracted with dichloromethane. The organic layer was washed with water and brine, dried over magnesium sulfate. The solvent was condensed under reduced pressure. The residue was purified by silica gel chromatography (hexane-ethyl acetate) to afford Compound 248 (3.28 g, yield 83%).

¹H-NMR (CDCl₃) δ: 0.00 (s, 6H), 0.84 (s, 9H), 1.10 (d, J=7.0 Hz, 3H), 2.57-2.66 (m, 1H), 3.59-3.64 (m, 4.0H), 3.74 (dd, J=9.2, 7.3 Hz, 1H). [M+H]=233.0, Method Condition 2: retention time 2.82 min

### Step 2 Preparation of Compound 249

To dichloromethane (20.0 mL) solution of Compound 248 (1.35 g, 5.81 mmol), THF solution of 1.02 mol/L diisopropylaluminium hydride (1.42 mL, 14.5 mmol), the mixture was stirred at −78° C. for 30 minutes. Methanol was added to the mixture, and the insoluble matter was filtered. The filtrate was condensed under reduced pressure. The residue was purified by silica gel chromatography (chloroform-methanol) to afford Compound 249 (0.380 g, yield 32%).

¹H-NMR (CDCl₃) δ: 0.08 (s, 6H), 0.84 (d, J=7.0 Hz, 3H), 0.89-0.95 (m, 10H), 1.90-2.00 (m, 1H), 2.85 (dd, J=7.0, 4.0 Hz, 1H), 3.55 (dd, J=9.8, 8.0 Hz, 1H, 3.58-3.68 (m, 2H), 3.75 (dd, J=9.8, 4.5 Hz, 1H).

[M+H]=205.0, Method Condition: 2: retention time 2.43 min

### Step 3 Preparation of Compound 250

To dichloromethane (14.0 mL) solution of oxalyl chloride (0.244 mL, 2.79 mmol), DMSO (0.396 mL, 5.58 mmol), Compound 249 (0.340 g, 1.66 mmol) and triethylamine (1.68 mL, 12.1 mmol) were added, and the mixture was at −78° C. for 4 hours. Saturated ammonium chloride was added to the mixture, and the mixture was extracted with diethylether. The organic layer was washed with water and brine, dried over magnesium sulfate. The solvent was condensed under reduced pressure. The residue was purified by silica gel chromatography (hexane-ethyl acetate) to afford Compound 250 (0.214 g, yield 64%).

¹H-NMR (CDCl₃) δ: 0.05 (s, 6H), 0.88 (s, 9H), 1.09 (d, J=7.0 Hz, 3H), 2.49-2.58 (m, 1H), 3.79-3.88 (m, 2H), 9.74 (d, J=1.5 Hz, 1H).

### Step 4 Preparation of Compound 252

Compound 251 (2.60 g, 16.4 mmol) was dissolved in DMF (30.0 mL). The cesium carbonate (8.01 g, 24.6 mmol) and 6-bromonicotinaldehyde (3.05 g, 16.4 mmol) were added to the mixture, and the mixture was stirred at 100° C. for 30 minutes. Water was added to the mixture, and the mixture was extracted with diethylether. The organic layer was washed with water and brine, dried over magnesium sulfate. The solvent was condensed under reduced pressure. The residue was purified by silica gel chromatography (hexane-ethyl acetate) to afford Compound 252 (3.27 g, yield 76%).

[M+H]=264.1, Method Condition: 2: retention time 2.02 min

### Step 5 Preparation of Compound 253

Compound 252 (2.00 g, 7.59 mmol) was dissolved in dichloromethane (30.0 mL). The dichloromethane solution of 1.00 mol/L boron tribromide (30.3 mL, 30.3 mmol) was added to the mixture, and the mixture was stirred at room temperature for 2 hours. Saturated sodium hydrogen carbonate solution was added to the mixture. The organic layer was washed with water and brine, dried over magnesium sulfate. The solvent was condensed under reduced pressure to afford Compound 253 (2.03 g) as crude.

[M+1-1]=249.8, Method Condition: 2: retention time 1.58 min

### Step 7 Preparation of Compound 255

Compound 253 (2.03 g) was dissolved in DMF (30.0 mL). Potassium carbonate (2.62 g, 19.0 mmol) and iodoethane (0.797 mL, 9.86 mmol) were added to the mixture at 60° C. for 2 hours. Water was added to the mixture, and the mixture was extracted with diethylether. The organic layer was washed with water, dried over magnesium sulfate. The solvent was condensed under reduced pressure. The residue was purified by silica gel chromatography (hexane-ethyl acetate) to afford Compound 254 (1.62 g, yield 77%).

[M+H]=278.1, Method Condition: 2: retention time 2.22 min

### Step 7 Preparation of Compound 255

Compound 254 (0.555 g, 2.00 mmol) was dissolved in the mixture of THF (8.00 mL) and methanol (4.00 mL). Sodium borohydride (0.098 g, 2.60 mmol) was added to the mixture while stirring it in an ice bath. The mixture was stirred for 3 hours. Saturated ammonium chloride solution was added to the mixture, and the mixture was extracted with ethyl acetate. The organic layer was washed with water and brine, dried over magnesium sulfate. The solvent was condensed under reduced pressure to afford Compound 255 (0.596 g) as crude.

[M+H]=280.9, Method Condition: 2: retention time 1.86 min

Step 8 Preparation of Compound 256

Compound 255 (0.596 g) was dissolved in dichloromethane (10.0 mL). Tetrabromomethane (0.736 g, 2.20 mmol and polymer-supported triphenylphosphine (1.00 g, 3.00 mmol) were added to the solution, and the mixture was stirred at room overnight temperature. The insoluble matter was filtered, and the solvent was removed under reduced pressure. The residue was purified by silica gel chromatography (hexane-ethyl acetate) to afford Compound 256 (0.392 g, yield 57%).

[M+H]=344.0, Method Condition: 2: retention time 2.51 min

Step 9 Preparation of Compound 257

Compound 256 (0.596 g) was dissolved in toluene. Triphenylphosphine (0.325 g, 1.24 mmol) was added to the solution, and the mixture was stirred at 120° C. for 3 hours. The precipitated crystals were filtrated to afford Compound 257 (0.645 g) as crude.

[M+H]=525.4, Method Condition: 2: retention time 1.89 min

### Step 10 Preparation of Compound 258

Compound 257 (0.598 g, 0.990 mmol) was dissolved in THF. The THF solution of 1.09 mol/L sodium hexamethyldisilylamide (0.907 mL, 0.989 mmol) was added to the solution at −78° C., and the mixture was stirred at −78° C. for 0.5 hours. Compound 250 (0.212 g, 1.05 mmol) was added to the mixture overnight at room temperature. Water was added to the mixture, and the mixture was extracted with ethyl acetate. The organic layer was washed with water, dried over magnesium sulfate and condensed under reduced pressure. The residue was purified by silica gel chromatography (hexane-ethyl acetate) to afford Compound 258 (0.162 g, yield 32%).

[M+H]=448.4, Method Condition: 2: retention time 3.44 min

### Step 11 Preparation of Compound 259

Compound 258 (0.162 g) was dissolved in THF. The THF solution of 1.00 mol/L tetrabutylammonium fluoride (0.494 mL, 0.494 mmol) was added to the mixture, and the mixture was stirred overnight at room temperature. The mixture was removed under reduced pressure. The residue was purified by silica gel chromatography (hexane-ethyl acetate) to afford Compound 259 (0.024 g, yield 7.3%).

[M+H]=334.2, Method Condition: 2: retention time 2.29 min

### Step 12 Preparation of Compound 260

Compound 259 (0.024 g, 0.072 mmol) was dissolved in dichloromethane (0.500 mL). Pyridine (0.047 mL, 0.575 mmol) and methanesulfonyl chloride (0.022 mL, 0.288 mmol) were added to the solution, and the mixture was stirred overnight at room temperature. The mixture was removed under reduced pressure. The residue was purified by silica gel chromatography (hexane-ethyl acetate) to afford Compound 260 (0.019 g, yield 63%).

[M+H]=412.0, Method Condition: 2: retention time 2.47 min

### Step 13 Preparation of Compound 261

Compound 260 (0.019 g, 0.045 mmol) was dissolved in DMF (1.00 mL). Sodium azide (0.006 g, 0.093 mmol) was added to the solution, and the mixture was stirred overnight at 60° C. Water was added to the mixture, the mixture was extracted with ethyl acetate. The organic layer was washed with water, dried over magnesium sulfate and condensed under reduced pressure. The residue was purified by silica gel chromatography (hexane-ethyl acetate) to afford Compound 261 (0.017 g, yield 64%).

[M+H]=359.2, Method Condition: 2: retention time 2.82 min

### Step 14 Preparation of Compound 262

Compound 261 (0.017 g, 0.046 mmol) was dissolved in the mixture of THF (1.00 mL) and water (0.10 mL). Triphenylphosphine (0.0140 g, 0.053 mmol) was added to the solution, and the mixture was stirred at 60° C. for 2 hours. The mixture was removed under reduced pressure. The residue was purified by silica gel chromatography (hexane-ethyl acetate) to afford Compound 262 (0.029 g).

[M+H]=333.0, Method Condition: 2: retention time 1.58 min

### Step 15 Preparation of Compound I′-1

Compound 31 (0.0029 g) was dissolved in methanol (1.00 mL). Acetic anhydride (0.013 mL, 0.138 mL) was added to the mixture, the mixture was stirred overnight at room temperature. The mixture was removed under reduced pressure. The residue was purified by silica gel chromatography (hexane-ethyl acetate) to afford Compound I′-1 (0.008 g, yield 46%).

¹H-NMR (CDCl₃) δ: 1.11 (d, J=6.8 Hz, 3H), 1.42 (t, J=7.0 Hz, 3H), 2.46-2.56 (m, 1H), 3.08-3.15 (m, 1H), 3.36-3.42 (m, 1H), 4.02 (q, J=6.8 Hz, 2H), 5.48 (brs, 1H), 5.97 (dd, J=15.8, 8.0 Hz, 1H), 6.35 (d, J=15.8 Hz, 1H), 6.84 (dd, J=8.9, 2.9 Hz, 1H), 6.89 (d, J=8.5 Hz, 1H), 7.00 (d, J=2.9 Hz, 1H), 7.11 (d, J=8.9 Hz, 1H), 7.74 (dd, J=8.5, 2.4 Hz, 1H), 8.05 (d, J=2.4 Hz, 1H).

[M+H]=375.0, Method Condition: 2: Retention time 2.19 min

The Biological Test Examples of the present invention are described as follows.

### Preparation 1: Preparation of Recombinant Human ACC2

After a cDNA encoding human ACC2 (27-2458) was cloned from human kidney cDNA library (Clontech), human ACC2 gene containing His-tag sequence at 5′ terminus was inserted into pFastBacl (Invitrogen). Recombinant baculovirus was generated using Bac-to-Bac baculovirus expression system (Invitrogen) according to the manufacturer's protocol. To express human ACC2, Sf-9 cells were infected with recombinant baculovirus. After infected cells were disrupted, the filtrated lysate was subjected to Ni-affinity chromatography and anion-exchange chromatography. The fractions containing human ACC2 protein were pooled as recombinant human ACC2 solution.

### Preparation 2: Preparation of Recombinant Human ACC1

After a cDNA encoding human ACC1 (1-2346) was cloned from human liver cDNA library (BioChain), human ACC1 gene containing myc-tag and His-tag sequence at 3′ terminus was inserted into pIEXBAC3 (Novagen). Recombinant baculovirus was generated using FlashBACGOLD system (Oxford Expression Technologies) according to the manufacturer's protocol. To express human ACC1, Sf-9 cells were infected with recombinant baculovirus. After infected cells were disrupted, the filtrated lysate was subjected to Ni-affinity chromatography and anion-exchange chromatography. The fractions containing human ACC1 protein were pooled as recombinant human ACC1 solution.

### Test Example 1

### The Measurement of Inhibitory Activity on Human ACC1 and the ACC2

Recombinant human ACC1 and recombinant human ACC2, which were prepared by the method mentioned above, were preincubated with assay buffer solution (50 mM HEPES-KOH (pH 7.4), 10 mM magnesium chloride, 6-10 mM potassium citrate, 4 mM reduced form of glutathione, 1.5 mg/ml bovine serum albumin) for one hour. Then, 0.2 μL of each this invention compound solution (in DMSO) were dispensed to 384-well microplate, 5 μL of the preincubated enzyme solution and 5 μL of substrate solution (50 mM HEPES-KOH (pH 7.4), 1 mM ATP, 0.8 mM acetyl CoA and 25-50 mM potassium bicarbonate) were added to microplate. After centrifugation and shaking, the reaction mixtures were incubated in a humidified box at room temperature for 1 to 3 hours. After the incubation, the enzyme reactions were stopped by the addition of EDTA. Then, after the samples were cocrystallized with CHCA (α-cyano-4-hydroxy cinnamic acid) matrices on MALDI target plate, by using the matrix assist laser deionization time-of-flight mass spectrometer (MALDI-TOF MS), samples were measured in reflector negative mode. Deprotonated ions of acetyl CoA (AcCoA) of substrate and malonyl CoA (MalCoA) of the reaction product were detected, then, the conversion rates of acetyl CoA to malonyl CoA was calculated by the intensity of [MalCoA-H]-/(Intensity of [MalCoA-H]-+Intensity of [AcCoA-H]-) using each signal strength. The 50% inhibitory concentration (IC50) was calculated from the inhibition rate of the enzymatic reaction at each concentration of the compounds. In addition, potassium citrate concentrations in assay buffer solution, potassium hydrogen carbonate concentrations in substrate solution and incubation time were adjusted by each lot of enzyme.

The 50% inhibitory concentration (1050) on human ACCT of Compound I-1, I-30, I-60, I-100, I-130, I-160, I-180, I-210, I-250, I-300, I-320, I-390, I-420 and I-438 were measured, that of these compounds was more than 100 μM.

The inhibition activity on human ACC2 of the present compounds is described in the following tables 79-84.

TABLE 79

| No. | IC50 (μM) |
|---|---|
| I-2 | 0.016 |
| I-6 | 0.078 |
| I-7 | 0.031 |
| I-9 | 0.082 |
| I-13 | 0.052 |
| I-14 | 0.0069 |
| I-15 | 0.071 |
| I-16 | 0.031 |
| I-23 | 0.0052 |
| I-24 | 0.0044 |
| I-27 | 1.2 |
| I-28 | 0.097 |
| I-29 | 0.094 |
| I-32 | 0.014 |
| I-33 | 0.012 |
| I-34 | 7.1 |
| I-35 | 1.1 |
| I-36 | 0.0065 |
| I-37 | 0.037 |
| I-38 | 0.0091 |
| I-39 | 0.011 |
| I-43 | 0.080 |
| I-44 | 0.49 |
| I-45 | 0.083 |
| I-47 | 0.025 |
| I-48 | 0.037 |
| I-49 | 0.072 |
| I-52 | 0.054 |
| I-53 | 0.94 |
| I-54 | 0.43 |
| I-55 | 4.5 |
| I-56 | 0.31 |
| I-57 | 0.0022 |
| I-58 | 0.0070 |
| I-59 | 0.95 |
| I-62 | 0.31 |
| I-63 | 0.83 |
| I-64 | 5.9 |
| I-66 | 5.7 |
| I-67 | 1.2 |
| I-69 | 0.47 |
| I-72 | 0.23 |
| I-73 | 0.053 |
| I-74 | 0.057 |
| I-76 | 0.030 |
| I-77 | 0.048 |
| I-78 | 0.55 |
| I-82 | 0.16 |
| I-83 | 0.048 |
| I-84 | 0.46 |
| I-86 | 0.44 |
| I-87 | 0.35 |
| I-88 | 0.89 |
| I-94 | 0.14 |
| I-97 | 0.12 |
| I-103 | 0.97 |
| I-104 | 0.45 |
| I-105 | 0.20 |
| I-106 | 0.44 |
| I-113 | 0.48 |
| I-116 | 0.15 |
| I-117 | 0.038 |

TABLE 80

| No. | IC50 (μM) |
|---|---|
| I-119 | 0.061 |
| I-122 | 0.15 |
| I-123 | 0.18 |
| I-124 | 1.4 |
| I-125 | 0.32 |
| I-133 | 0.92 |
| I-134 | 1.8 |
| I-135 | 0.042 |
| I-136 | 0.012 |
| I-137 | 0.63 |
| I-138 | 2.3 |
| I-139 | 0.066 |
| I-143 | 0.80 |
| I-144 | 0.69 |
| I-148 | 0.88 |
| I-154 | 2.7 |
| I-155 | 0.58 |
| I-156 | 0.93 |
| I-157 | 0.26 |
| I-158 | 1.4 |
| I-159 | 0.22 |
| I-162 | 0.32 |
| I-163 | 0.28 |
| I-164 | 0.85 |
| I-165 | 0.49 |
| I-168 | 0.022 |
| I-169 | 0.37 |
| I-172 | 0.60 |
| I-173 | 0.059 |
| I-174 | 2.6 |
| I-177 | 0.018 |
| I-179 | 0.042 |
| I-183 | 0.66 |
| I-184 | 0.11 |
| I-188 | 1.8 |
| I-189 | 0.24 |
| I-192 | 0.081 |
| I-193 | 0.14 |
| I-194 | 0.30 |
| I-195 | 0.061 |
| I-196 | 1.1 |
| I-203 | 0.069 |
| I-204 | 0.038 |
| I-205 | 1.6 |
| I-206 | 0.0063 |
| I-208 | 0.0049 |
| I-209 | 0.0045 |
| I-214 | 0.018 |
| I-215 | 0.024 |
| I-216 | 0.069 |
| I-233 | 0.044 |
| I-235 | 0.30 |
| I-236 | 0.015 |
| I-237 | 0.087 |
| I-242 | 0.016 |
| I-243 | 0.41 |
| I-244 | 0.036 |
| I-246 | 0.073 |
| I-247 | 0.43 |
| I-248 | 0.58 |

| No. | IC50 (μM) |
|---|---|
| I-251 | 0.096 |
| I-253 | 0.017 |
| I-255 | 0.087 |
| I-256 | 0.050 |
| I-257 | 0.16 |
| I-258 | 0.45 |
| I-259 | 0.40 |
| I-260 | 0.15 |
| I-261 | 3.0 |
| I-262 | 0.66 |
| I-264 | 0.84 |
| I-265 | 1.1 |
| I-266 | 0.026 |
| I-267 | 0.044 |
| I-268 | 0.11 |
| I-269 | 0.12 |
| I-270 | 0.39 |
| I-271 | 0.079 |
| I-272 | 0.45 |
| I-273 | 0.099 |
| I-274 | 0.35 |
| I-275 | 0.017 |
| I-276 | 0.15 |
| I-277 | 0.23 |
| I-278 | 0.079 |
| I-279 | 0.059 |
| I-280 | 0.17 |
| I-281 | 0.13 |
| I-282 | 0.013 |
| I-283 | 3.2 |
| I-284 | 0.86 |
| I-285 | 0.096 |
| I-286 | 0.048 |
| I-287 | 0.018 |
| I-288 | 0.036 |
| I-289 | 0.028 |
| I-290 | 0.60 |
| I-291 | 0.018 |
| I-292 | 1.6 |
| I-293 | 1.3 |
| I-294 | 0.15 |
| I-295 | 2.8 |
| I-296 | 0.11 |
| I-298 | 0.058 |
| I-299 | 0.038 |
| I-300 | 0.018 |
| I-301 | 1.9 |
| I-302 | 0.51 |
| I-303 | 0.63 |
| I-304 | 1.4 |
| I-305 | 0.011 |
| I-306 | 0.024 |
| I-307 | 0.048 |
| I-308 | 0.48 |
| I-309 | 2.8 |
| I-310 | 1.3 |
| I-312 | 0.57 |
| I-314 | 0.96 |
| I-315 | 3.9 |
| I-317 | 0.35 |

TABLE 82

| No. | IC50 (μM) |
|---|---|
| I-318 | 0.067 |
| I-319 | 0.051 |
| I-320 | 0.23 |
| I-321 | 0.029 |
| I-322 | 0.49 |
| I-323 | 0.96 |
| I-324 | 0.17 |
| I-325 | 0.12 |
| I-326 | 0.44 |
| I-327 | 0.34 |
| I-328 | 0.035 |
| I-329 | 1.1 |
| I-330 | 3.9 |
| I-331 | 0.13 |
| I-332 | 0.092 |
| I-333 | 0.18 |
| I-334 | 0.047 |
| I-335 | 0.015 |
| I-336 | 2.0 |
| I-337 | 0.023 |
| I-338 | 0.033 |
| I-339 | 0.091 |
| I-341 | 0.45 |
| I-342 | 0.12 |
| I-343 | 0.046 |
| I-344 | 0.051 |
| I-345 | 0.052 |
| I-346 | 0.44 |
| I-347 | 0.30 |
| I-348 | 0.21 |
| I-349 | 6.6 |
| I-350 | 0.018 |
| I-351 | 0.042 |
| I-352 | 0.24 |
| I-353 | 2.6 |
| I-354 | 0.17 |
| I-355 | 0.019 |
| I-356 | 5.4 |
| I-357 | 0.44 |
| I-358 | 0.027 |
| I-359 | 0.23 |
| I-360 | 0.0091 |
| I-361 | 0.036 |
| I-362 | 0.35 |
| I-363 | 0.029 |
| I-364 | 0.013 |
| I-365 | 0.010 |
| I-366 | 0.015 |
| I-367 | 0.14 |
| I-368 | 0.11 |
| I-369 | 0.024 |
| I-370 | 0.28 |
| I-371 | 0.060 |
| I-372 | 0.033 |
| I-373 | 0.025 |
| I-374 | 0.78 |
| I-375 | 0.057 |
| I-376 | 1.1 |
| I-377 | 0.039 |
| I-378 | 0.016 |

TABLE 83

| No. | IC50 (μm) |
|---|---|
| I-379 | 3.7 |
| I-380 | 0.022 |
| I-381 | 0.36 |
| I-382 | 1.3 |
| I-383 | 0.029 |
| I-384 | 0.28 |
| I-385 | 0.16 |
| I-386 | 0.40 |
| I-387 | 0.56 |
| I-388 | 2.0 |
| I-389 | 0.024 |
| I-390 | 0.54 |
| I-391 | 0.047 |
| I-392 | 0.97 |
| I-393 | 0.048 |
| I-394 | 0.012 |
| I-395 | 4.2 |
| I-396 | 0.055 |
| I-397 | 0.015 |
| I-398 | 0.043 |
| I-399 | 0.068 |
| I-400 | 3.2 |
| I-401 | 0.056 |
| I-402 | 1.1 |
| I-403 | 0.45 |
| I-404 | 0.19 |
| I-405 | 1.2 |
| I-406 | 0.30 |
| I-407 | 2.1 |
| I-408 | 2.3 |
| I-409 | 0.025 |
| I-410 | 0.15 |
| I-411 | 0.077 |
| I-412 | 0.20 |
| I-414 | 0.036 |
| I-415 | 0.12 |
| I-416 | 0.17 |
| I-417 | 0.057 |
| I-418 | 0.097 |
| I-419 | 0.048 |
| I-420 | 0.12 |
| I-421 | 0.052 |
| I-422 | 1.3 |
| I-423 | 3.0 |
| I-424 | 0.14 |
| I-425 | 0.040 |
| I-426 | 0.026 |
| I-427 | 0.20 |
| I-428 | 0.17 |
| I-429 | 0.050 |
| I-430 | 2.7 |

TABLE 84

| No. | IC50 (μM) |
|---|---|
| I-431 | 0.085 |
| I-432 | 0.52 |
| I-433 | 0.049 |
| I-434 | 0.10 |
| I-435 | 0.024 |
| I-436 | 0.058 |
| I-437 | 0.033 |
| I-438 | 0.78 |
| I-439 | 0.082 |
| I-440 | 0.020 |
| I-441 | 0.69 |
| I-442 | 0.065 |
| I-443 | 0.022 |
| I-444 | 0.042 |
| I-445 | 0.26 |
| I-446 | 0.073 |
| I-447 | 0.025 |
| I-448 | 0.18 |
| I-449 | 0.13 |
| I-450 | 0.060 |
| I-451 | 0.025 |
| I-452 | 0.042 |
| I-453 | 0.73 |
| I-454 | 0.037 |
| I-455 | 0.030 |
| I-456 | 0.45 |
| I-457 | 0.31 |
| I-458 | 0.026 |
| I-459 | 0.012 |
| I-460 | 0.13 |
| I-461 | 1.5 |
| I-462 | 0.019 |
| I-463 | 0.068 |
| I-464 | 0.12 |
| I-465 | 0.12 |
| I-466 | 0.016 |
| I-467 | 0.043 |
| I-468 | 0.090 |
| I-469 | 0.036 |
| I-470 | 0.23 |
| I-471 | 0.016 |
| I-472 | 0.037 |
| I-473 | 0.039 |
| I-474 | 0.0097 |
| I-475 | 0.17 |
| I-476 | 0.24 |
| I-477 | 0.0069 |
| I-478 | 0.55 |
| I-479 | 0.44 |
| I-480 | 0.52 |
| I-481 | 0.013 |
| I-482 | 0.0081 |
| I-483 | 0.052 |
| I-484 | 0.16 |
| I-485 | 0.13 |
| I-486 | 0.11 |
| I-487 | 0.038 |
| I-488 | 0.011 |
| I-489 | 0.55 |
| I-490 | 0.042 |
| I-491 | 0.0058 |
| I-492 | 0.080 |
| I-493 | 0.15 |
| I-494 | 0.014 |
| I-495 | 0.021 |
| I-496 | 0.21 |
| I-497 | 0.051 |
| I-498 | 0.027 |
| I-499 | 0.096 |
| I-500 | 0.0089 |
| I-501 | 0.031 |
| I-502 | 0.0071 |
| I-503 | 0.0051 |
| I-504 | 0.0065 |
| I-505 | 0.045 |
| I-506 | 0.0082 |
| I-507 | 0.043 |
| I-508 | 0.23 |
| I-509 | 0.023 |
| I-510 | 0.0060 |
| I-511 | 0.088 |
| I-512 | 0.14 |
| I-513 | 0.027 |
| I-514 | 0.096 |
| I-515 | 0.86 |
| I-516 | 0.17 |
| I-517 | 0.047 |
| I-518 | 0.80 |
| I-519 | 0.059 |
| I-520 | 0.075 |
| I’-1 | 0.56 |

### Test Example 2

### CYP Inhibition Test

Using commercially available pooled human hepatic microsome, and employing, as markers, 7-ethoxyresorufin O-deethylation (CYP1A2), tolbutamide methyl-hydroxylation (CYP2C9), mephenytoin 4′-hydroxylation (CYP2C19), dextromethorphan 0-demethylation (CYP2D6), and terfenadine hydroxylation as typical substrate metabolism reactions of human main five CYP enzyme forms (CYP1A2, 2C9, 2C19, 2D6, 3A4), an inhibitory degree of each metabolite production amount by a test compound is assessed.

The reaction conditions are as follows: substrate, 0.5 μmol/L ethoxyresorufin (CYP1A2), 100 μmol/L tolbutamide (CYP2C9), 50 μmol/L S-mephenitoin (CYP2C19), 5 μmol/L dextromethorphan (CYP2D6), 1 μmol/L terfenadine (CYP3A4); reaction time, 15 minutes; reaction temperature, 37° C.; enzyme, pooled human hepatic microsome 0.2 mg protein/mL; test drug concentration, 1, 5, 10, 20 μmol/L (four points).

Each five kinds of substrates, human hepatic microsome, or a test drug in 50 mM Hepes buffer as a reaction solution is added to a 96-well plate at the composition as described above, NADPH, as a cofactor is added to initiate metabolism reactions as markers and, after the incubation at 37° C. for 15 minutes, a methanol/acetonitrile=1/1 (v/v) solution is added to stop the reaction. After the centrifugation at 3000 rpm for 15 minutes, resorufin (CYP1A2 metabolite) in the supernatant is quantified by a fluorescent multilabel counter and tributamide hydroxide (CYP2CP metabolite), mephenytoin 4′ hydroxide (CYP2C19 metabolite), dextromethorphan (CYP2D6 metabolite), and terfenadine alcohol (CYP3A4 metabolite) are quantified by LC/MS/MS.

Addition of only DMSO being a solvent dissolving a drug to a reaction system is adopted as a control (100%), remaining activity (%) is calculated at each concentration of a test drug added as the solution and IC50 is calculated by reverse presumption by a logistic model using a concentration and an inhibition rate.

### Test Example 3

### BA Test

An experimental material and a method for examining oral absorbability

(1) Animals used: rats or mice are used.
(2) Breeding condition: chow and sterilized tap water are allowed to be taken in freely.
(3) Setting of a dosage and grouping: a predetermined dosage is administered orally or intravenously. Groups are formed as shown below. (A dosage varied depending on each compound)
Oral administration 1-30 mg/kg (n=2 to 3) Intravenous administration 0.5-10 mg/kg (n=2 to 3)
(4) Preparation of administered liquid: In oral administration, a solution or suspension is administered. In intravenous administration, after solubilization, the administration is performed.
(5) Method of Administration: In oral administration, compulsory administration to the stomach is conducted using an oral probe.
In intravenous administration, administration from the caudal vein is conducted using a syringe with an injection needle.
(6) Evaluation item: Blood is chronologically collected, and then the concentration of a compound of the present inventionin blood plasma is measured using a LC/MS/MS.
(7) Statistical analysis: With regard to a shift in plasma concentration, the plasma concentration-time area under the curve (AUC) is calculated using a nonlinear least-squares program WinNonlin®. Bioavailability (BA) is calculated from the AUCs of the oral administration group and the intravenous administration group, respectively.

### Test Example 4

### Metabolism Stability Test

Using a commercially available pooled human hepatic microsomes, a test compound is reacted for a constant time, a remaining rate is calculated by comparing a reacted sample and an unreacted sample, thereby, a degree of metabolism in liver is assessed.

A reaction is performed (oxidative reaction) at 37° C. for 0 minute or 30 minutes in the presence of 1 mmol/L NADPH in 0.2 mL of a buffer (50 mmol/L Tris-HCl pH 7.4, 150 mmol/L potassium chloride, 10 mmol/L magnesium chloride) containing 0.5 mg protein/mL of human liver microsomes. After the reaction, 50 μL of the reaction solution is added to 100 μL of a methanol/acetonitrile=1/1 (v/v), mixed and centrifuged at 3000 rpm for 15 minutes. The test compound in the supernatant is quantified by LC/MS/MS, and a remaining amount of the test compound after the reaction is calculated, letting a compound amount at 0 minute reaction time to be 100%. Hydrolysis reaction is performed in the absence of NADPH and glucuronidation reaction is in the presence of 5 mM UDP-glucuronic acid in place of NADPH, followed by similar operations.

### Test Example 5

### CYP3A4 Fluorescent MBI Test

The CYP3A4 fluorescent MBI test is a test of investigating enhancement of CYP3A4 inhibition of a compound by a metabolism reaction, and the test is performed using, a reaction in which 7-benzyloxytrifluoromethylcoumarin (7-BFC) is debenzylated by the CYP3A4 enzyme to produce a metabolite, 7-hydroxytrifluoromethylcoumarin (HFC) emitting fluorescent light.

The reaction conditions are as follows: substrate, 5.6 μmol/L 7-BFC; pre-reaction time, 0 or 30 minutes; reaction time, 15 minutes; reaction temperature, 25° C. (room temperature); CYP3A4 content (expressed in Escherichia coli), at pre-reaction 62.5 pmol/mL, at reaction 6.25 pmol/mL (at 10-fold dilution); test drug concentration, 0.625, 1.25, 2.5, 5, 10, 20 μmol/L (six points).

An enzyme in a K-Pi buffer (pH 7.4) and a test drug solution as a pre-reaction solution are added to a 96-well plate at the composition of the pre-reaction, a part of it is transferred to another 96-well plate so that it is 1/10 diluted by a substrate in a K-Pi buffer, NADPH as a co-factor is added to initiate a reaction as an index (without preincubation) and, after a predetermined time of a reaction, acetonitrile/0.5 mol/L Tris (trishydroxyaminomethane)=4/1 is added to stop the reaction. In addition, NADPH is added to a remaining preincubation solution to initiate a preincubation (with preincubation) and, after a predetermined time of a preincubation, a part is transferred to another plate so that it is 1/10 diluted with a substrate and a K-Pi buffer to initiate a reaction as an index. After a predetermined time of a reaction, acetonitrile/0.5 mol/L Tris (trishydroxyaminomethane)=4/1 is added to stop the reaction. For the plate on which each index reaction has been performed, a fluorescent value of 7-HFC which is a metabolite is measured with a fluorescent plate reader. (Ex=420 nm, Em=535 nm).

Addition of only DMSO which is a solvent dissolving a drug to a reaction system is adopted as a control (100%), remaining activity (%) is calculated at each concentration of a test drug added as the solution, and IC₅₀ is calculated by reverse-presumption by a logistic model using a concentration and an inhibition rate. When a difference between IC₅₀ values is 5 μM or more, this is defined as (+) and, when the difference is 3 μM or less, this is defined as (−).

### Test Example 6

### Fluctuation Ames Test

20 μL of freezing-stored rat typhoid bacillus (Salmonella typhimurium TA98 strain, TA100 strain) is inoculated on 10 mL of a liquid nutrient medium (2.5% Oxoid nutrient broth No. 2), and this is cultured before shaking at 37° C. for 10 hours. 9 mL of a bacterial solution of the TA98 strain is centrifuged (2000×g, 10 minutes) to remove a culturing solution, the bacteria is suspended in 9 mL of a Micro F buffer (K₂HPO₄: 3.5 g/L, KH₂PO₄: 1 g/L, (NH₄)₂SO₄: 1 g/L, trisodium citrate dehydrate: 0.25 g/L, MgSO₄.7H₂O: 0.1 g/L), the suspension is added to 110 mL of an Exposure medium (Micro F buffer containing Biotin: 8 μg/mL, histidine 0.2 μg/mL, glucose: 8 mg/mL), and the TA100 strain is added to 120 mL of the Exposure medium relative to 3.16 mL of the bacterial solution to prepare a test bacterial solution. Each 12 μL of a test substance DMSO solution (8 stage dilution from maximum dose 50 mg/mL at 2-fold ratio), DMSO as a negative control, 50 μg/mL of 4-nitroquinoline-1-oxide DMSO solution for the TA98 strain, 0.25 μg/mL of 2-(2-furyl)-3-(5-nitro-2-furyl)acrylamide DMSO solution for the TA100 strain under the non-metabolism activating condition, 40 μg/mL of 2-aminoanthracene DMSO solution for the TA98 strain, 20 μg/mL of 2-aminoanthracene DMSO solution for the TA100 strain under the metabolism activating condition as a positive control, and 588 μL of the test bacterial solution (a mixed solution of 498 μl of the test bacterial solution and 90 μL of S9 mix under the metabolism activating condition) are mixed, and this is shaking-cultured at 37° C. for 90 minutes. 460 μL of the bacterial solution exposed to the test substance is mixed with 2300 μl, of an Indicator medium (Micro F buffer containing biotin: 8 μg/mL, histidine 0.2 μg/mL, glucose: 8 mg/mL, Bromo Cresol Purple: 37.5 μg/mL), each 50 μL is dispensed into microplate 48 wells/dose, and this is subjected to stationary culturing at 37° C. for 3 days. Since a well containing a bacterium which has obtained the proliferation ability by mutation of an amino acid (histidine) synthesizing enzyme gene turns from purple to yellow due to a pH change, the bacterium proliferation well which has turned to yellow in 48 wells per dose is counted, and is assessed by comparing with a negative control group. (−) means that mutagenicity is negative and (+) is positive.

### Test Example 7

### hERG Test

For the purpose of assessing risk of an electrocardiogram QT interval prolongation, effects on delayed rectifier K+ current (IKr), which plays an important role in the ventricular repolarization process of the compound of the present invention, is studied using HEK293 cells expressing human ether-a-go-go related gene (hERG) channel.

After a cell is retained at a membrane potential of −80 mV by whole cell patch clamp method using an automated patch clamp system (PatchXpress 7000A, Axon Instruments Inc.), IKr induced by depolarization pulse stimulation at +40 mV for 2 seconds and, further, repolarization pulse stimulation at −50 mV for 2 seconds is recorded. After the generated current was stabilized, extracellular solution (NaCl: 135 mmol/L, KCl: 5.4 mmol/L, NaH2PO4: 0.3 mmol/L, CaCl₂.2H₂O: 1.8 mmol/L, MgCl₂. 6H₂O: 1 mmol/L, glucose: 10 mmol/L, HEPES (4-(2-hydroxyethyl)-1-piperazineethanesulfonic acid): 10 mmol/L, pH=7.4) in which the test compound has been dissolved at an objective concentration is applied to the cell under the room temperature condition for 10 minutes. From the recording IKr, an absolute value of the tail peak current is measured based on the current value at the resting membrane potential using an analysis software (DataXpress ver. 1, Molecular Devices Corporation). Further, the % inhibition relative to the tail peak current before application of the test substance is calculated, and compared with the vehicle-applied group (0.1% dimethyl sulfoxide solution) to assess influence of the test substance on IKr.

### Test Example 8

### Powder Solubility Test

Appropriate amounts of the test substances are put into appropriate containers. To the respective containers are added 200 μL of JP-1 fluid (sodium chloride 2.0 g, hydrochloric acid 7.0 mL and water to reach 1000 mL), 200 μL of JP-2 fluid (phosphate buffer (pH 6.8) 500 mL and water 500 mL), and 200 μL of 20 mmol/L TCA (sodium taurocholate)/JP-2 fluid (TCA 1.08 g and water to reach 100 mL). In the case that the test compound is dissolved after the addition of the test fluid, the bulk powder is added as appropriate. The containers are sealed, and shaken for 1 hour at 37° C. The mixtures are filtered, and 100 μL of methanol is added to each of the filtrate (100 μL) so that the filtrates are two-fold diluted. The dilution ratio is changed if necessary. The dilutions are observed for bubbles and precipitates, and then the containers are sealed and shaken. Quantification is performed by HPLC with an absolute calibration method.

### Formulation Example

The following Formulation Examples are only exemplified and not intended to limit the scope of this invention.

### Formulation Example 1

### Tablets

| | | |
|---|---|---|
| Compound (I) | 15 | mg |
| Starch | 15 | mg |
| Lactose | 15 | mg |
| Crystalline cellulose | 19 | mg |
| Polyvinyl alcohol | 3 | mg |
| Distilled water | 30 | ml |
| Calcium stearate | 3 | mg |

All of the above ingredients except for calcium stearate are uniformly mixed. Then the mixture is crushed, granulated and dried to obtain a suitable size of granules. Next, calcium stearate is added to the granules. Finally, tableting is performed under a compression force.

### Formulation Example 2

### Capsules

| | | |
|---|---|---|
| Compound (I) | 10 | mg |
| Magnesium stearate | 10 | mg |
| Lactose | 80 | mg |

The above ingredients are mixed uniformly to obtain powders or fine granules, and then the obtained mixture is filled into capsules.

### Formulation Example 3

### Granule

| | | |
|---|---|---|
| Compound (I) | 30 | g |
| Lactose | 265 | g |
| Magnesium stearate | 5 | g |

After the above ingredients are mixed uniformly, the mixture is compressed, crushed, granulated and sieved to obtain a suitable size of granules.

### INDUSTRIAL APPLICABILITY

The compounds of this invention have an ACC2 antagonistic activity, and are very useful for treatment or prevention of a disease associated with ACC2.

1. A compound of formula (I′): 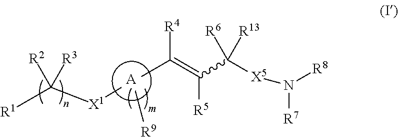
wherein R¹ is substituted or unsubstituted aryl or substituted or unsubstituted heteroaryl,
X¹ is —O—, —S—, —N(—R¹²)—, —C(—R²)(—R³)—, —O—C(—R²)(—R³)—, —S—C(—R²)(—R³)— or —N(—R¹²)—C(—R²)(—R³)—,
R² is each independently hydrogen, substituted or unsubstituted alkyl or halogen,
R³ is each independently hydrogen, substituted or unsubstituted alkyl or halogen,
R² and R³ on the same carbon atom may be taken together with the carbon atom to which they are attached to form substituted or unsubstituted ring,
R² and R³ may be taken together with the substituent on the aryl or heteroaryl ring of R¹ and the atom to which each R² and R³ are attached to form substituted or unsubstituted ring,
n is an integer from 0 to 3,
R¹² is hydrogen, substituted or unsubstituted alkyl, substituted or unsubstituted alkenyl or substituted or unsubstituted alkynyl,
R¹² may be taken together with the substituent on the aryl or heteroaryl ring of R¹ and the atom to which each is attached to form substituted or unsubstituted ring,
Ring A is aromatic carbocycle or aromatic heterocycle,
R⁹ is each independently substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted alkylsulfanyl, substituted or unsubstituted alkenylsulfanyl, substituted or unsubstituted alkynylsulfanyl, halogen, hydroxy, cyano, substituted or unsubstituted amino, substituted or unsubstituted carbamoyl, substituted or unsubstituted sulfamoyl, carboxy, substituted or unsubstituted alkylcarbonyl or substituted or unsubstituted alkyloxycarbonyl,
m is an integer from 0 to 4,
R⁴ and R⁵ is each independently hydrogen, substituted or unsubstituted alkyl, substituted or unsubstituted alkenyl, substituted or unsubstituted alkynyl, halogen, substituted or unsubstituted alkyloxy or substituted or unsubstituted alkyloxycarbonyl,
R⁶ is substituted or unsubstituted alkyl, substituted or unsubstituted alkenyl or substituted or unsubstituted alkynyl,
R¹³ is hydrogen, substituted or unsubstituted alkyl, substituted or unsubstituted alkenyl or substituted or unsubstituted alkynyl, or
R⁶ and R¹³ may be taken together with the adjacent carbon atom to form substituted or unsubstituted ring,
X⁵ is bond or —C(—R¹⁶)(—R¹⁷)—,
R¹⁶ and R¹⁷ is each independently hydrogen, substituted or unsubstituted alkyl or halogen,
R⁷ is hydrogen or substituted or unsubstituted alkyl,
R⁸ is substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted cycloalkenylcarbonyl, alkyloxycarbonyl, substituted or unsubstituted alkenyloxycarbonyl, substituted or unsubstituted alkynyloxycarbonyl, substituted or unsubstituted carbamoyl, substituted or unsubstituted sulfamoyl, substituted or unsubstituted amidino, substituted or unsubstituted arylcarbonyl, substituted or unsubstituted heteroarylcarbonyl, substituted or unsubstituted non-aromatic heterocyclyl carbonyl, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted amino, substituted or unsubstituted aryl, or substituted or unsubstituted heteroaryl, substituted or unsubstituted non-aromatic heterocyclyl, substituted or unsubstituted aryloxycarbonyl or substituted or unsubstituted sulfino,
the wavy line means that the group of formula: 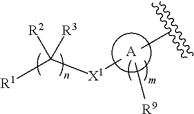
and the group of formula: 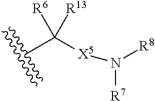
are located at E configuration, Z configuration or the mixture of these configurations in regard to the double bond between the carnon atom bonding R⁴ and the carbon atom bonding R⁵,
provided that the group of formula: 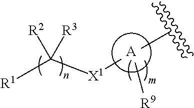
is not a group of formula: 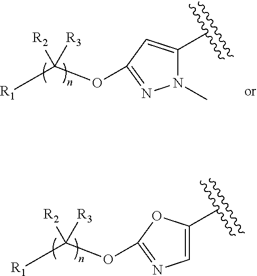
and the following compounds are excluded,

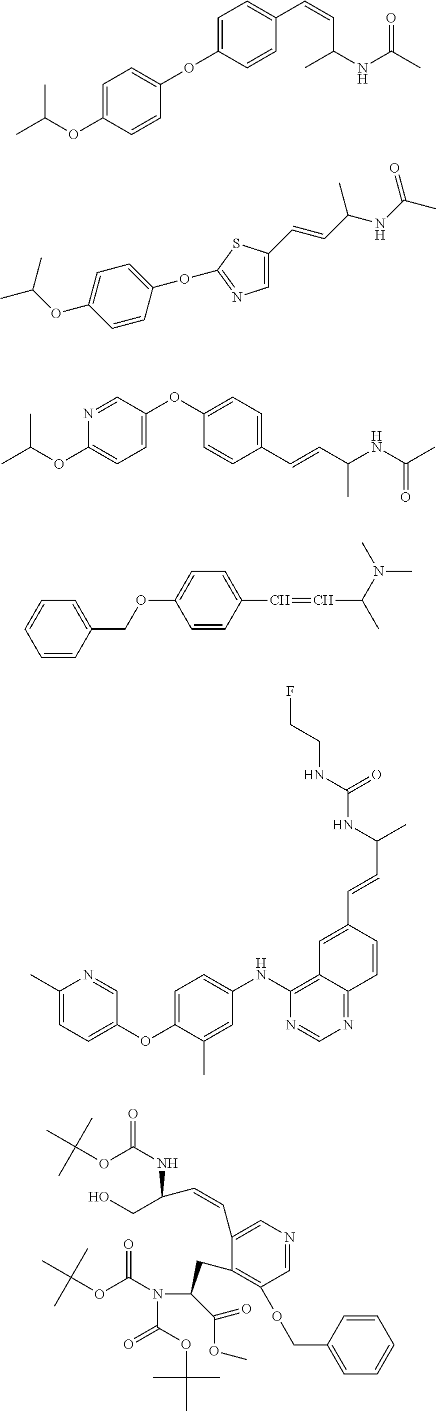

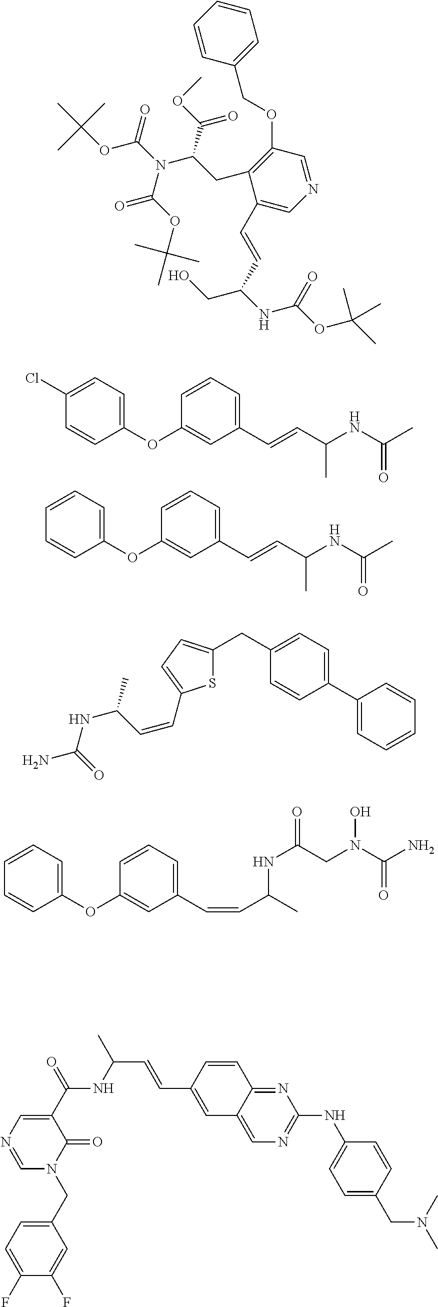

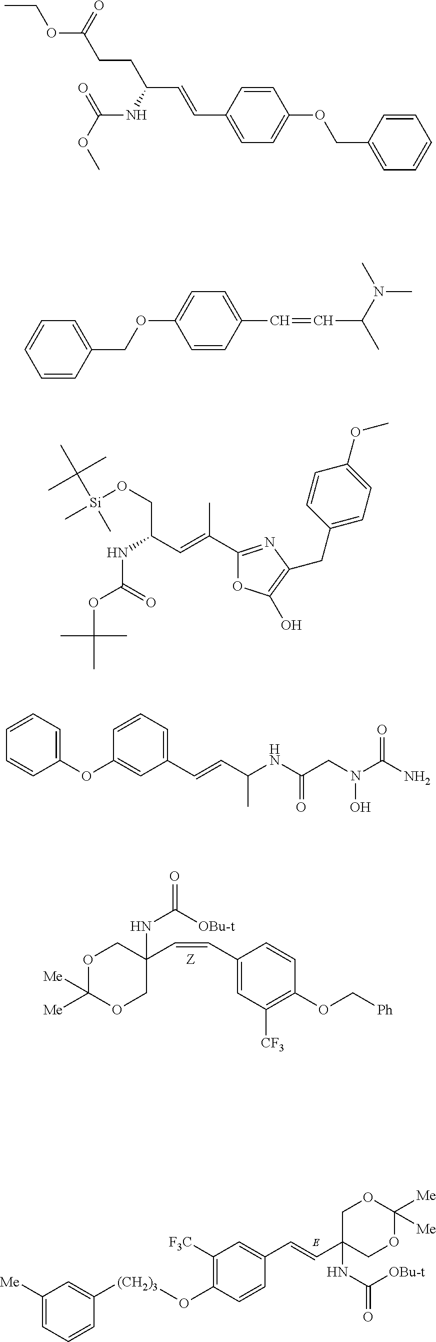

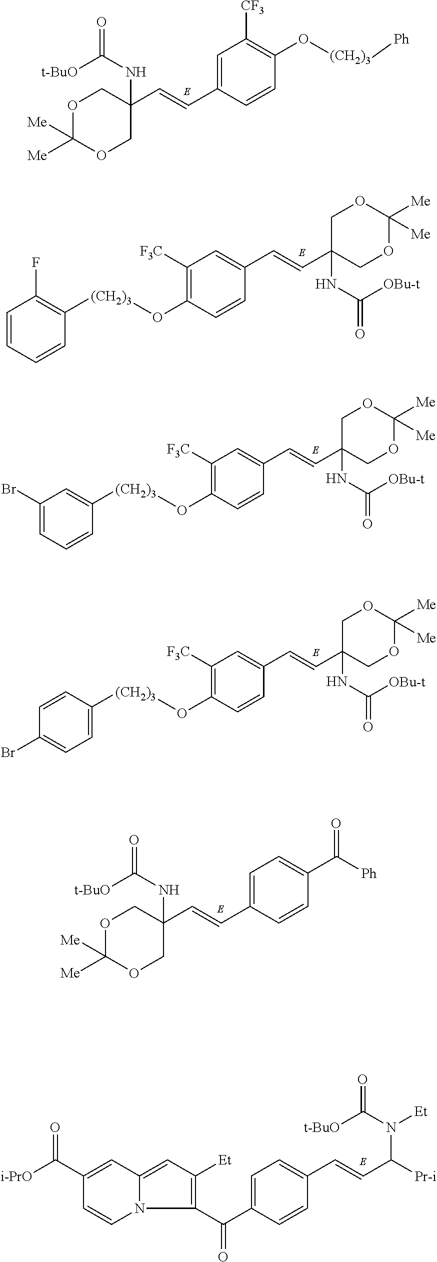

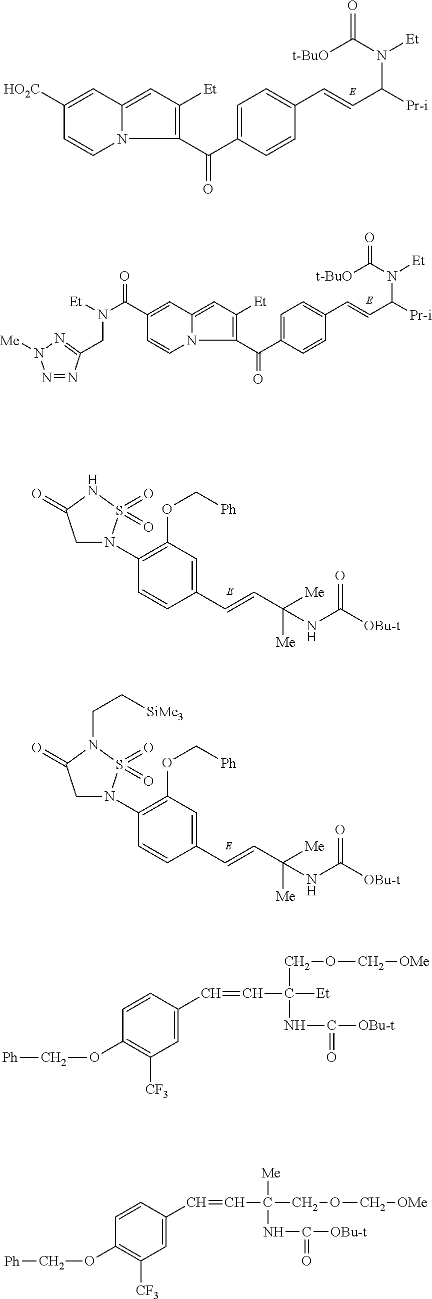

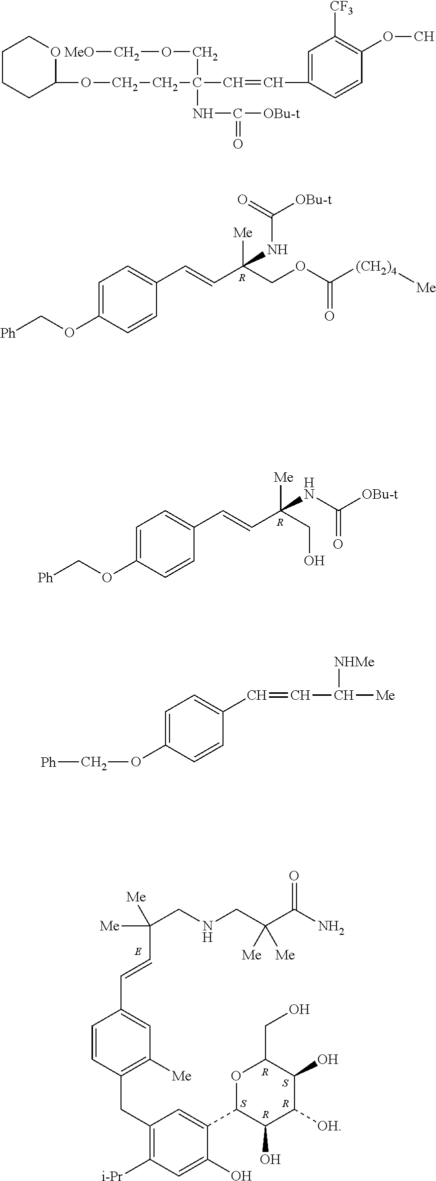

2. The compound or its pharmaceutically acceptable salt of claim 1, wherein R¹ is substituted or unsubstituted fused aryl or substituted or unsubstituted fused heteroaryl.

3. The compound or its pharmaceutically acceptable salt of claim 1, wherein R¹ is a group of formula: 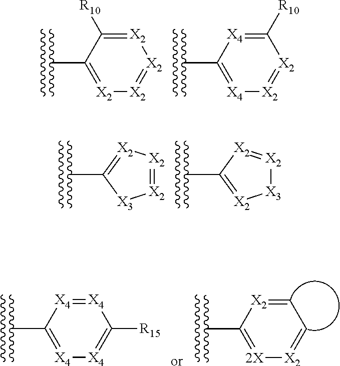
wherein X² is each independently —N═, —C(H)═ or —C(—R¹⁰)═,
X³ is —S—, —O—, —N(H)— or —N(—R¹¹)—,
X⁴ is each independently —N═ or —C(H)═,
R¹⁰ is each independently halogen, substituted or unsubstituted alkyl, substituted or unsubstituted alkenyl, substituted or unsubstituted alkynyl, substituted or unsubstituted amino, hydroxy, substituted or unsubstituted alkyloxy, substituted or unsubstituted alkylcarbonyloxy, mercapto, substituted or unsubstituted alkylsulfanyl, substituted or unsubstituted alkylamino, substituted or unsubstituted alkylcarbonylsulfanyl, cyano, substituted or unsubstituted non-aromatic heterocyclyl, trialkylsilyloxy, substituted or unsubstituted aryloxy, substituted or unsubstituted aryl, substituted or unsubstituted heteroaryl, substituted or unsubstituted cycloalkyl, substituted or unsubstituted cycloalkeyl, substituted or unsubstituted alkylsulfonyl or substituted or unsubstituted alkylsulfonyloxy,
R¹¹ is each independently substituted or unsubstituted alkyl, substituted or unsubstituted alkeyl or substituted or unsubstituted alkynyl,
R¹⁵ is substituted or unsubstituted C2 or more alkyl, substituted or unsubstituted aryl, substituted or unsubstituted aryloxy or substituted or unsubstituted non-aromatic heterocyclyl,
Ring P is substituted or unsubstituted 5-membered aromatic heterocycle, substituted or unsubstituted 5-membered non-aromatic carbocycle, substituted or unsubstituted 5-membered non-aromatic heterocycle, substituted or unsubstituted 6-membered non-aromatic carbocycle or substituted or unsubstituted 6-membered non-aromatic heterocycle.

4. The compound or its pharmaceutically acceptable salt of claim 3, wherein R¹ is a group of formula: 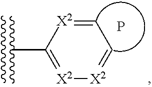
and the above formula: 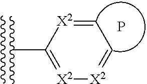
is a group of formula: 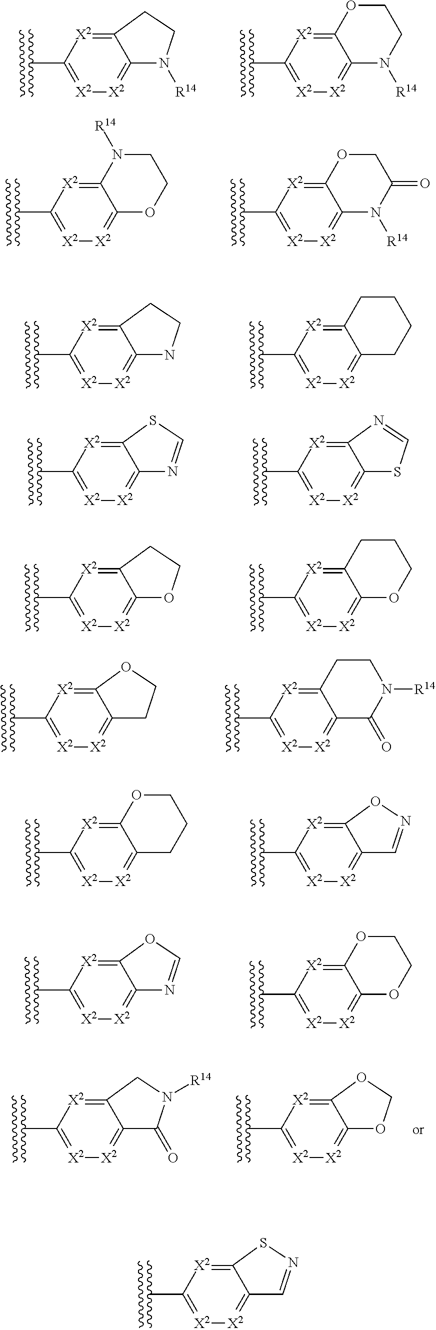
wherein X³ has the same meaning as claim 3,
R¹⁴ is hydrogen, substituted or unsubstituted alkyl, substituted or unsubstituted alkenyl or substituted or unsubstituted alkynyl,
the carbon atom on Ring P may be further substituted.

5. The compound or its pharmaceutically acceptable salt of claim 4, wherein X² is —C(H)═ or —C(—R¹⁰)═.

6. The compound or its pharmaceutically acceptable salt of claim 3, wherein R¹ is a group of formula: 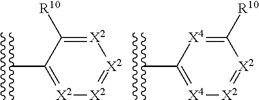
wherein R¹⁰, X² and X⁴ have the same meaning as claim 3.

7. The compound or its pharmaceutically acceptable salt of claim 6, wherein R¹ is a group of formula: 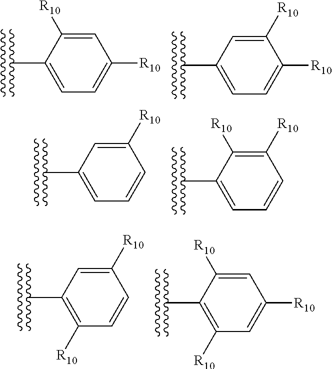
wherein R¹⁰ has the same meaning as claim 6.

8. The compound or its pharmaceutically acceptable salt of claim 3, wherein R¹⁰ is each independently halogen, substituted or unsubstituted alkyl, substituted or unsubstituted amino, substituted or unsubstituted alkyloxy, cyano, trialkylsilyloxy or substituted or unsubstituted aryloxy.

9. The compound or its pharmaceutically acceptable salt of claim 1, wherein R¹³ is hydrogen.

10. The compound or its pharmaceutically acceptable salt of claim 1, wherein R⁶ is substituted or unsubstituted alkyl.

11. The compound or its pharmaceutically acceptable salt of claim 10, wherein R⁶ is unsubstituted alkyl.

12. The compound or its pharmaceutically acceptable salt of claim 11, wherein R⁶ is methyl.

13. The compound or its pharmaceutically acceptable salt of claim 1, wherein R⁸ is substituted or unsubstituted alkylcarbonyl, substituted or unsubstituted alkyloxycarbonyl, substituted or unsubstituted carbamoyl, substituted or unsubstituted arylcarbonyl, substituted or unsubstituted heteroarylcarbonyl, substituted or unsubstituted non-aromatic heterocyclylcarbonyl, substituted or unsubstituted heteroaryl or substituted or unsubstituted aryloxycarbonyl.

14. The compound or its pharmaceutically acceptable salt of claim 13, wherein R⁸ is acetyl.

15. The compound or its pharmaceutically acceptable salt of claim 1, wherein X¹ is —O—.

16. The compound or its pharmaceutically acceptable salt of claim 1, wherein n is an integer from 1 to 3.

17. The compound or its pharmaceutically acceptable salt of claim 1, wherein n is 0.

18. The compound or its pharmaceutically acceptable salt of claim 1, wherein ring A is aromatic heterocycle.

19. The compound or its pharmaceutically acceptable salt of claim 18, wherein ring A is 6-membered aromatic heterocycle.

20. The compound or its pharmaceutically acceptable salt of claim 1, wherein ring A is pyrazole, thiazole, pyridine, pyrimidine, pyridazine, pyrazine or benzene.

21. The compound or its pharmaceutically acceptable salt of claim 1, wherein R⁴ and R⁵ is hydrogen.

22. The compound or its pharmaceutically acceptable salt of claim 1, wherein R⁷ is hydrogen.

23. The compound or its pharmaceutically acceptable salt of claim 1, wherein m is 0.

24. The compound or its pharmaceutically acceptable salt of claim 1, wherein X⁵ is bond.

25. The compound or its pharmaceutically acceptable salt of claim 1, wherein the configuration of the group of formula: 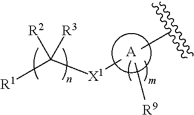
and the group of formula: 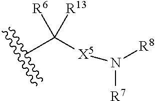
in the compound of formula (I′) is E configuration.

26. The compound or its pharmaceutically acceptable salt of claim 1, wherein the compound of formula (I′) is a group of formula (II′):

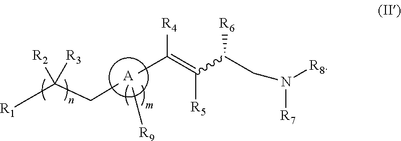

27. The compound or its pharmaceutically acceptable salt of claim 1, wherein the compound of formula (I′) is a compound of formula (III): 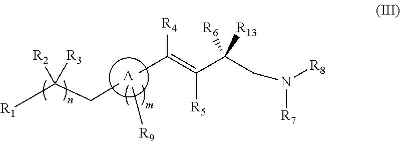
wherein R¹ is a group of formula: 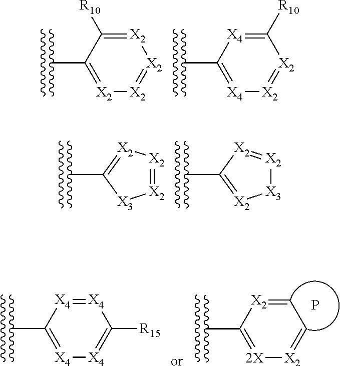
wherein X² is each independently —N═, —C(H)═ or —C(—R¹⁰)═,
X³ is —S—, —O—, —N(H)— or —N(—R¹¹)—,
X⁴ is each independently —N═ or —C(H)═,
R¹⁰ is each independently halogen, substituted or unsubstituted alkyl, substituted or unsubstituted alkenyl, substituted or unsubstituted alkynyl, substituted or unsubstituted amino, hydroxy, substituted or unsubstituted alkyloxy, substituted or unsubstituted alkylcarbonyloxy, mercapto, substituted or unsubstituted alkylsulfanyl, substituted or unsubstituted alkylamino, substituted or unsubstituted alkylcarbonylsulfanyl, cyano, substituted or unsubstituted non-aromatic heterocyclyl, trialkylsilyloxy, substituted or unsubstituted aryloxy, substituted or unsubstituted aryl, substituted or unsubstituted heteroaryl, substituted or unsubstituted cycloalkyl, substituted or unsubstituted cycloalkeyl, substituted or unsubstituted alkylsulfonyl or substituted or unsubstituted alkylsulfonyloxy,
wherein X¹ is —O—,
n is 0,
R⁴ and R⁵ are hydrogen,
R¹³ is hydrogen,
X⁵ is bond,
R⁷ is hydrogen.

28. The compound or its pharmaceutically acceptable salt of claim 27, wherein R⁶ is alkyl.

29. The compound or its pharmaceutically acceptable salt of claim 27, wherein R⁸ is substituted or unsubstituted alkylcarbonyl.

30. A pharmaceutical composition comprising the compound or its pharmaceutically acceptable salt of claim 1.

31. The pharmaceutical composition of claim 30 for treatment or prevention of a disease associated with ACC2.

32. A method for treatment or prevention of a disease associated with ACC2 characterized by administering the compound or its pharmaceutically acceptable salt of claim 1.

33. Use of the compound or its pharmaceutically acceptable salt of claim 1 for treatment or prevention of a disease associated with ACC2.

34. The compound or its pharmaceutically acceptable salt of claim 1 for treatment or prevention of a disease associated with ACC2.
